(19) United States
(12) Patent Application Publication
(10) Pub. No.: US 2026/0098012 A1
(43) Pub. Date: Apr. 9, 2026

(54) TETRAHYDROACRIDINONE ANALOGUES FOR TREATMENT AND PREVENTION OF MALARIA

(71) Applicants: University of Georgia Research Foundation, Inc., Athens, GA (US); Northeastern University, Boston, MA (US)

(72) Inventors: Dennis E. KYLE, Athens, GA (US); Steven MAHER, Bogart, GA (US); Ami H. Asakawa, Brighton, MA (US); Khaly D. Diagne, Malden, MA (US); Roman Manetsch, Boston, MA (US); Adriana A. Marin, Bogotá (CO); Anthony Marasciullo, Boston, MA (US); Caroline E. Consoli, Philadelphia, PA (US); Sagan Thomas de Castro, Carrboro, NC (US); Olivia Isabelle McWhorter, Roswell, GA (US); Meng CUI, Boston, MA (US)

(21) Appl. No.: 19/416,056

(22) Filed: Dec. 11, 2025

Related U.S. Application Data
(63) Continuation-in-part of application No. PCT/US2024/033508, filed on Jun. 12, 2024.
(60) Provisional application No. 63/507,750, filed on Jun. 13, 2023.

(51) Int. Cl. C07D 219/06; A61K 31/473; A61P 33/06; C07D 401/04; C07D 405/04; C07D 409/04; C07D 417/04

(52) U.S. Cl. CPC C07D 219/06; A61K 31/473; A61P 33/06; C07D 401/04; C07D 405/04; C07D 409/04; C07D 417/04

(57) ABSTRACT

In accordance with the purpose(s) of the present disclosure, as embodied and broadly described herein, the disclosure, in one aspect, relates to scaffold molecules that exhibit potent antimalarial activity against multiple strains of Plasmodium spp. in the parasite's various stages, such as, for example, the erythrocytic and exoerythrocytic stages, and pharmaceutical compositions comprising same. In one aspect, the compounds are tetrahydroacridinones having the structure I described herein. In another aspect, the compounds described herein can be used to treat or prevent malaria in a subject.

### CROSS-REFERENCE TO RELATED APPLICATIONS

This application is a continuation-in-part of PCT International Application No. PCT/US2024/033508, filed Jun. 12, 2024, which claims the benefit of and priority to U.S. Provisional Patent Application No. 63/507,750, filed on Jun. 13, 2023, the contents of which are incorporated by reference herein in their entireties.

### STATEMENT REGARDING FEDERALLY SPONSORED RESEARCH OR DEVELOPMENT

This invention was made with government support under one R01AI144464 awarded by the NIH. The government has certain rights in the invention.

### BACKGROUND

The World Health Organization reported that in 2021, there was an estimate of 247 million malaria cases, with approximately 619,000 deaths. Resistance to the current line of treatment, which includes an artemisinin derivative in combination with another blood schizonticide, is spreading, demonstrating the need for new classes of antimalarials. 1,2,3,4-tetrahydroacridin-9(10H)-ones (THAs) are a promising class of antimalarials and lack the induction of resistance that current antimalarials are facing, which is illustrated with their equipotency against two clinically relevant multidrug resistant strains of Plasmodium falciparum, W2 and TM90-C2B. In addition to this, THAs have an advantage of demonstrating activity against multiple stages of the parasite, such as the liver and transmitting stages. However, THAs lack aqueous solubility, which is an important property for the preparation of bioavailable compounds for oral delivery.

### SUMMARY

In accordance with the purpose(s) of the present disclosure, as embodied and broadly described herein, the disclosure, in one aspect, relates to scaffold molecules that exhibit potent antimalarial activity against multiple strains of Plasmodium spp. in the parasite's various stages such as, for example, the erythrocytic and exoerythrocytic stages, and pharmaceutical compositions comprising same.

In one aspect, compound having a formula represented by structure I or the pharmaceutically acceptable salt thereof:

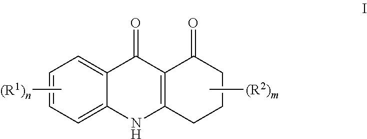

wherein
n is an integer from 1 to 4, where each R¹ is independently hydrogen, a halide, a substituted or unsubstituted linear or branched alkyl group, or an alkoxy group, and
m is an integer from 1 to 3, where each R² is hydrogen, a substituted or unsubstituted cycloalkyl group, a substituted or unsubstituted heterocycloalkyl group, a substituted or unsubstituted aryl group, or a substituted or unsubstituted heteroaryl group.

In other aspects, the compounds described herein can be used to treat or prevent malaria in a subject.

Other systems, methods, features, and advantages of the present disclosure will be or become apparent to one with skill in the art upon examination of the following drawings and detailed description. It is intended that all such additional systems, methods, features, and advantages be included within this description, be within the scope of the present disclosure, and be protected by the accompanying claims. In addition, all optional and preferred features and modifications of the described embodiments are usable in all aspects of the disclosure taught herein. Furthermore, the individual features of the dependent claims, as well as all optional and preferred features and modifications of the described embodiments are combinable and interchangeable with one another.

### BRIEF DESCRIPTION OF THE DRAWINGS

Many aspects of the present disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

FIGS. 1A-1F show the pharmacokinetic data for several compounds described herein.

Additional advantages of the invention will be set forth in part in the description which follows, and in part will be obvious from the description, or can be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### DETAILED DESCRIPTION

Many modifications and other embodiments disclosed herein will come to mind to one skilled in the art to which the disclosed compositions and methods pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the appended claims. The skilled artisan will recognize many variants and adaptations of the aspects described herein. These variants and adaptations are intended to be included in the teachings of this disclosure and to be encompassed by the claims herein.

Although specific terms are employed herein, they are used in a generic and descriptive sense only and not for purposes of limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure.

Any recited method can be carried out in the order of events recited or in any other order that is logically possible. That is, unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

While aspects of the present disclosure can be described and claimed in a particular statutory class, such as the system statutory class, this is for convenience only and one of skill in the art will understand that each aspect of the present disclosure can be described and claimed in any statutory class.

It is also to be understood that the terminology used herein is for the purpose of describing particular aspects only and is not intended to be limiting. Unless defined otherwise, all technical and scientific terms used herein have the same meaning as commonly understood by one of ordinary skill in the art to which the disclosed compositions and methods belong. It will be further understood that terms, such as those defined in commonly used dictionaries, should be interpreted as having a meaning that is consistent with their meaning in the context of the specification and relevant art and should not be interpreted in an idealized or overly formal sense unless expressly defined herein.

Prior to describing the various aspects of the present disclosure, the following definitions are provided and should be used unless otherwise indicated. Additional terms may be defined elsewhere in the present disclosure.

### Definitions

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by,” “comprising,” “comprises,” “comprised of,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of.

As used in the specification and the appended claims, the singular forms “a,” “an” and “the” include plural referents unless the context clearly dictates otherwise. Thus, for example, reference to “an excipient” includes, but is not limited to, mixtures or combinations of two or more such excipients, and the like.

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

When a range is expressed, a further aspect includes from the one particular value and/or to the other particular value. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less’ and should be interpreted to include the specific ranges of ‘about x’, ‘about y’, and ‘about z’ as well as the ranges of ‘less than x’, less than y’, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y’, and ‘greater than z’. In addition, the phrase “about ‘x’ to ‘y’”, where ‘x’ and ‘y’ are numerical values, includes “about ‘x’ to about ‘y’”.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range. Thus, for example, if a component is in an amount of about 1%, 2%, 3%, 4%, or 5%, where any value can be a lower and upper endpoint of a range, then any range is contemplated between 1% and 5% (e.g., 1% to 3%, 2% to 4%, etc.).

As used herein, the terms “about,” “approximate,” “at or about,” and “substantially” mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated ±10% variation unless otherwise indicated or inferred. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

As used herein, “IC₅₀,” is intended to refer to the concentration of a substance (e.g., a compound or a drug) that is required for 50% inhibition of a biological process, or component of a process. For example, IC₅₀ refers to the half maximal (50%) inhibitory concentration (IC) of a substance as determined in a suitable assay.

A residue of a chemical species, as used in the specification and concluding claims, refers to the moiety that is the resulting product of the chemical species in a particular reaction scheme or subsequent formulation or chemical product, regardless of whether the moiety is actually obtained from the chemical species. Thus, an ethylene glycol residue in a polyester refers to one or more —OCH₂CH₂O— units in the polyester, regardless of whether ethylene glycol was used to prepare the polyester. Similarly, a sebacic acid residue in a polyester refers to one or more —CO(CH₂)₈CO— moieties in the polyester, regardless of whether the residue is obtained by reacting sebacic acid or an ester thereof to obtain the polyester.

As used herein, the term “substituted” is contemplated to include all permissible substituents of organic compounds. In a broad aspect, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For purposes of this disclosure, the heteroatoms, such as nitrogen, can have hydrogen substituents and/or any permissible substituents of organic compounds described herein which satisfy the valences of the heteroatoms. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound, e.g., a compound that does not spontaneously undergo transformation such as by rearrangement, cyclization, elimination, etc. It is also contemplated that, in certain aspects, unless expressly indicated to the contrary, individual substituents can be further optionally substituted (i.e., further substituted, or unsubstituted).

The position of a substituent can be defined relative to the positions of other substituents in an aromatic ring. For example, as shown below in relationship to the “R” group, a second substituent can be “ortho,” “para,” or “meta” to the R group, meaning that the second substituent is bonded to a carbon labeled ortho, para, or meta as indicated below. Combinations of ortho, para, and meta substituents relative to a given group or substituent are also envisioned and should be considered to be disclosed.

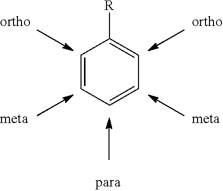

In defining various terms, “A¹,” “A²,” “A³,” and “A⁴” are used herein as generic symbols to represent various specific substituents. These symbols can be any substituent, not limited to those disclosed herein, and when they are defined to be certain substituents in one instance, they can, in another instance, be defined as some other substituents.

The term “aliphatic” or “aliphatic group,” as used herein, denotes a hydrocarbon moiety that may be straight-chain (i.e., unbranched), branched, or cyclic (including fused, bridging, and spirofused polycyclic) and may be completely saturated or may contain one or more units of unsaturation, but which is not aromatic. Unless otherwise specified, aliphatic groups contain 1-20 carbon atoms. Aliphatic groups include, but are not limited to, linear or branched, alkyl, alkenyl, and alkynyl groups, and hybrids thereof such as (cycloalkyl)alkyl, (cycloalkenyl)alkyl or (cycloalkyl)alkenyl.

The term “alkyl” as used herein is a branched or unbranched saturated hydrocarbon group of 1 to 24 carbon atoms, such as methyl, ethyl, n-propyl, isopropyl, n-butyl, isobutyl, s-butyl, t-butyl, n-pentyl, isopentyl, s-pentyl, neopentyl, hexyl, heptyl, octyl, nonyl, decyl, dodecyl, tetradecyl, hexadecyl, eicosyl, tetracosyl, and the like. The alkyl group can be cyclic or acyclic. The alkyl group can be branched or unbranched. The alkyl group can also be substituted or unsubstituted. For example, the alkyl group can be substituted with one or more groups including, but not limited to, alkyl, cycloalkyl, alkoxy, amino, ether, halide, hydroxy, nitro, silyl, sulfo-oxo, or thiol, as described herein. A “lower alkyl” group is an alkyl group containing from one to six (e.g., from one to four) carbon atoms. The term alkyl group can also be a C1 alkyl, C1-C2 alkyl, C1-C3 alkyl, C1-C4 alkyl, C1-C5 alkyl, C1-C6 alkyl, C1-C7 alkyl, C1-C8 alkyl, C1-C9 alkyl, C1-C10 alkyl, and the like up to and including a C1-C24 alkyl.

Throughout the specification “alkyl” is generally used to refer to both unsubstituted alkyl groups and substituted alkyl groups; however, substituted alkyl groups are also specifically referred to herein by identifying the specific substituent(s) on the alkyl group. For example, the term “halogenated alkyl” or “haloalkyl” specifically refers to an alkyl group that is substituted with one or more halide, e.g., fluorine, chlorine, bromine, or iodine. Alternatively, the term “monohaloalkyl” specifically refers to an alkyl group that is substituted with a single halide, e.g. fluorine, chlorine, bromine, or iodine. The term “polyhaloalkyl” specifically refers to an alkyl group that is independently substituted with two or more halides, i.e. each halide substituent need not be the same halide as another halide substituent, nor do the multiple instances of a halide substituent need to be on the same carbon. The term “alkoxyalkyl” specifically refers to an alkyl group that is substituted with one or more alkoxy groups, as described below. The term “aminoalkyl” specifically refers to an alkyl group that is substituted with one or more amino groups. The term “hydroxyalkyl” specifically refers to an alkyl group that is substituted with one or more hydroxy groups. When “alkyl” is used in one instance and a specific term such as “hydroxyalkyl” is used in another, it is not meant to imply that the term “alkyl” does not also refer to specific terms such as “hydroxyalkyl” and the like.

This practice is also used for other groups described herein. That is, while a term such as “cycloalkyl” refers to both unsubstituted and substituted cycloalkyl moieties, the substituted moieties can, in addition, be specifically identified herein; for example, a particular substituted cycloalkyl can be referred to as, e.g., an “alkylcycloalkyl.” Similarly, a substituted alkoxy can be specifically referred to as, e.g., a “halogenated alkoxy,” a particular substituted alkenyl can be, e.g., an “alkenylalcohol,” and the like. Again, the practice of using a general term, such as “cycloalkyl,” and a specific term, such as “alkylcycloalkyl,” is not meant to imply that the general term does not also include the specific term.

The term “cycloalkyl” as used herein is a non-aromatic carbon-based ring composed of at least three carbon atoms. Examples of cycloalkyl groups include, but are not limited to, cyclopropyl, cyclobutyl, cyclopentyl, cyclohexyl, norbornyl, and the like. The term “heterocycloalkyl” is a type of cycloalkyl group as defined above, and is included within the meaning of the term “cycloalkyl,” where at least one of the carbon atoms of the ring is replaced with a heteroatom such as, but not limited to, nitrogen, oxygen, sulfur, or phosphorus. The cycloalkyl group and heterocycloalkyl group can be substituted or unsubstituted. The cycloalkyl group and heterocycloalkyl group can be substituted with one or more groups including, but not limited to, alkyl, cycloalkyl, alkoxy, amino, ether, halide, hydroxy, nitro, silyl, sulfo-oxo, or thiol as described herein.

The term “alkanediyl” as used herein, refers to a divalent saturated aliphatic group, with one or two saturated carbon atom(s) as the point(s) of attachment, a linear or branched, cyclo, cyclic or acyclic structure, no carbon-carbon double or triple bonds, and no atoms other than carbon and hydrogen. The groups, —CH₂— (methylene), —CH₂CH₂—, —CH₂C(CH₃)₂CH₂—, and —CH₂CH₂CH₂— are non-limiting examples of alkanediyl groups.

The terms “alkoxy” and “alkoxyl” as used herein to refer to an alkyl or cycloalkyl group bonded through an ether linkage; that is, an “alkoxy” group can be defined as —OA¹ where A¹ is alkyl or cycloalkyl as defined above. “Alkoxy” also includes polymers of alkoxy groups as just described; that is, an alkoxy can be a polyether such as —OA¹-OA² or -OA¹-(OA²)ₐ-OA³, where “a” is an integer of from 1 to 200 and A¹, A², and A³ are alkyl and/or cycloalkyl groups.

The term “aryloxy” as used herein to refer to an aryl group bonded through an ether linkage; that is, an “aryloxy” group can be defined as —OA¹ where A¹ is aryl as defined herein.

The term “alkenyl” as used herein is a hydrocarbon group of from 2 to 24 carbon atoms with a structural formula containing at least one carbon-carbon double bond. Asymmetric structures such as (A¹A²)C═C(A³A⁴) are intended to include both the E and Z isomers. This can be presumed in structural formulae herein wherein an asymmetric alkene is present, or it can be explicitly indicated by the bond symbol C═C. The alkenyl group can be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol, as described herein.

The term “cycloalkenyl” as used herein is a non-aromatic carbon-based ring composed of at least three carbon atoms and containing at least one carbon-carbon double bound, i.e., C═C. Examples of cycloalkenyl groups include, but are not limited to, cyclopropenyl, cyclobutenyl, cyclopentenyl, cyclopentadienyl, cyclohexenyl, cyclohexadienyl, norbornenyl, and the like. The term “heterocycloalkenyl” is a type of cycloalkenyl group as defined above, and is included within the meaning of the term “cycloalkenyl,” where at least one of the carbon atoms of the ring is replaced with a heteroatom such as, but not limited to, nitrogen, oxygen, sulfur, or phosphorus. The cycloalkenyl group and heterocycloalkenyl group can be substituted or unsubstituted. The cycloalkenyl group and heterocycloalkenyl group can be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol as described herein.

The term “alkynyl” as used herein is a hydrocarbon group of 2 to 24 carbon atoms with a structural formula containing at least one carbon-carbon triple bond. The alkynyl group can be unsubstituted or substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol, as described herein.

The term “cycloalkynyl” as used herein is a non-aromatic carbon-based ring composed of at least seven carbon atoms and containing at least one carbon-carbon triple bound. Examples of cycloalkynyl groups include, but are not limited to, cyclooctynyl, cyclononynyl, and the like. The term “heterocycloalkynyl” is a type of cycloalkenyl group as defined above and is included within the meaning of the term “cycloalkynyl,” where at least one of the carbon atoms of the ring is replaced with a heteroatom such as, but not limited to, nitrogen, oxygen, sulfur, or phosphorus. The cycloalkynyl group and heterocycloalkynyl group can be substituted or unsubstituted. The cycloalkynyl group and heterocycloalkynyl group can be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol as described herein.

The term “aromatic group” as used herein refers to a ring structure having cyclic clouds of delocalized π electrons above and below the plane of the molecule, where the π clouds contain (4n+2) π electrons. A further discussion of aromaticity is found in Morrison and Boyd, Organic Chemistry, (5th Ed., 1987), Chapter 13, entitled “Aromaticity,” pages 477-497, incorporated herein by reference. The term “aromatic group” is inclusive of both aryl and heteroaryl groups.

The term “aryl” as used herein is a group that contains any carbon-based aromatic group including, but not limited to, benzene, naphthalene, phenyl, biphenyl, anthracene, and the like. The aryl group can be substituted or unsubstituted. The aryl group can be substituted with one or more groups including, but not limited to, alkyl, cycloalkyl, alkoxy, alkenyl, cycloalkenyl, alkynyl, cycloalkynyl, aryl, heteroaryl, aldehyde, —NH₂, carboxylic acid, ester, ether, halide, hydroxy, ketone, azide, nitro, silyl, sulfo-oxo, or thiol as described herein. The term “biaryl” is a specific type of aryl group and is included in the definition of “aryl.” In addition, the aryl group can be a single ring structure or comprise multiple ring structures that are either fused ring structures or attached via one or more bridging groups such as a carbon-carbon bond. For example, biaryl to two aryl groups that are bound together via a fused ring structure, as in naphthalene, or are attached via one or more carbon-carbon bonds, as in biphenyl. Fused aryl groups including, but not limited to, indene and naphthalene groups are also contemplated.

The term “aldehyde” as used herein is represented by the formula —C(O)H. Throughout this specification “C(O)” is a short hand notation for a carbonyl group, i.e., C═O.

The terms “amine” or “amino” as used herein are represented by the formula —NA¹A², where A¹ and A² can be, independently, hydrogen or alkyl, cycloalkyl, alkenyl, cycloalkenyl, alkynyl, cycloalkynyl, aryl, or heteroaryl group as described herein. A specific example of amino is —NH₂.

The term “alkylamino” as used herein is represented by the formula —NH(-alkyl) and —N(-alkyl)₂, where alkyl is a described herein. Representative examples include, but are not limited to, methylamino group, ethylamino group, propylamino group, isopropylamino group, butylamino group, isobutylamino group, (sec-butyl)amino group, (tert-butyl)amino group, pentylamino group, isopentylamino group, (tert-pentyl)amino group, hexylamino group, dimethylamino group, diethylamino group, dipropylamino group, diisopropylamino group, dibutylamino group, diisobutylamino group, di(sec-butyl)amino group, di(tert-butyl)amino group, dipentylamino group, diisopentylamino group, di(tert-pentyl)amino group, dihexylamino group, N-ethyl-N-methylamino group, N-methyl-N-propylamino group, N-ethyl-N-propylamino group and the like.

The term “carboxylic acid” as used herein is represented by the formula —C(O)OH.

The term “ester” as used herein is represented by the formula —OC(O)A¹ or —C(O)OA¹, where A¹ can be alkyl, cycloalkyl, alkenyl, cycloalkenyl, alkynyl, cycloalkynyl, aryl, or heteroaryl group as described herein.

The term “ether” as used herein is represented by the formula A¹OA², where A¹ and A² can be, independently, an alkyl, cycloalkyl, alkenyl, cycloalkenyl, alkynyl, cycloalkynyl, aryl, or heteroaryl group described herein.

The terms “halo,” “halogen” or “halide,” as used herein can be used interchangeably and refer to F, Cl, Br, or I.

The terms “pseudohalide,” “pseudohalogen” or “pseudohalo,” as used herein can be used interchangeably and refer to functional groups that behave substantially similar to halides. Such functional groups include, by way of example, cyano, thiocyanato, azido, trifluoromethyl, trifluoromethoxy, perfluoroalkyl, and perfluoroalkoxy groups.

The term “heteroalkyl” as used herein refers to an alkyl group containing at least one heteroatom. Suitable heteroatoms include, but are not limited to, O, N, Si, P and S, wherein the nitrogen, phosphorous and sulfur atoms are optionally oxidized, and the nitrogen heteroatom is optionally quaternized. Heteroalkyls can be substituted as defined above for alkyl groups.

The term “heteroaryl” as used herein refers to an aromatic group that has at least one heteroatom incorporated within the ring of the aromatic group. Examples of heteroatoms include, but are not limited to, nitrogen, oxygen, sulfur, and phosphorus, where N-oxides, sulfur oxides, and dioxides are permissible heteroatom substitutions. The heteroaryl group can be substituted or unsubstituted. The heteroaryl group can be substituted with one or more groups including, but not limited to, alkyl, cycloalkyl, alkoxy, amino, ether, halide, hydroxy, nitro, silyl, sulfo-oxo, or thiol as described herein. Heteroaryl groups can be monocyclic, or alternatively fused ring systems. Heteroaryl groups include, but are not limited to, furyl, imidazolyl, pyrimidinyl, tetrazolyl, thienyl, pyridinyl, pyrrolyl, N-methylpyrrolyl, quinolinyl, isoquinolinyl, pyrazolyl, triazolyl, thiazolyl, oxazolyl, isoxazolyl, oxadiazolyl, thiadiazolyl, isothiazolyl, pyridazinyl, pyrazinyl, benzofuranyl, benzodioxolyl, benzothiophenyl, indolyl, indazolyl, benzimidazolyl, imidazopyridinyl, pyrazolopyridinyl, and pyrazolopyrimidinyl. Further not limiting examples of heteroaryl groups include, but are not limited to, pyridinyl, pyridazinyl, pyrimidinyl, pyrazinyl, thiophenyl, pyrazolyl, imidazolyl, benzo[d]oxazolyl, benzo[d]thiazolyl, quinolinyl, quinazolinyl, indazolyl, imidazo[1,2-b]pyridazinyl, imidazo[1,2-a]pyrazinyl, benzo[c][1,2,5]thiadiazolyl, benzo[c][1,2,5]oxadiazolyl, and pyrido[2,3-b]pyrazinyl.

The terms “heterocycle” or “heterocyclyl,” as used herein can be used interchangeably and refer to single and multi-cyclic aromatic or non-aromatic ring systems in which at least one of the ring members is other than carbon. Thus, the term is inclusive of, but not limited to, “heterocycloalkyl,” “heteroaryl,” “bicyclic heterocycle,” and “polycyclic heterocycle.” Heterocycle includes pyridine, pyrimidine, furan, thiophene, pyrrole, isoxazole, isothiazole, pyrazole, oxazole, thiazole, imidazole, oxazole, including, 1,2,3-oxadiazole, 1,2,5-oxadiazole and 1,3,4-oxadiazole, thiadiazole, including, 1,2,3-thiadiazole, 1,2,5-thiadiazole, and 1,3,4-thiadiazole, triazole, including, 1,2,3-triazole, 1,3,4-triazole, tetrazole, including 1,2,3,4-tetrazole and 1,2,4,5-tetrazole, pyridazine, pyrazine, triazine, including 1,2,4-triazine and 1,3,5-triazine, tetrazine, including 1,2,4,5-tetrazine, pyrrolidine, piperidine, piperazine, morpholine, azetidine, tetrahydropyran, tetrahydrofuran, dioxane, and the like. The term heterocyclyl group can also be a C2 heterocyclyl, C2-C3 heterocyclyl, C2-C4 heterocyclyl, C2-C5 heterocyclyl, C2-C6 heterocyclyl, C2-C7 heterocyclyl, C2-C8 heterocyclyl, C2-C9 heterocyclyl, C2-C10 heterocyclyl, C2-C11 heterocyclyl, and the like up to and including a C2-C18 heterocyclyl. For example, a C2 heterocyclyl comprises a group which has two carbon atoms and at least one heteroatom, including, but not limited to, aziridinyl, diazetidinyl, dihydrodiazetyl, oxiranyl, thiiranyl, and the like. Alternatively, for example, a C5 heterocyclyl comprises a group which has five carbon atoms and at least one heteroatom, including, but not limited to, piperidinyl, tetrahydropyranyl, tetrahydrothiopyranyl, diazepanyl, pyridinyl, and the like. It is understood that a heterocyclyl group may be bound either through a heteroatom in the ring, where chemically possible, or one of carbons comprising the heterocyclyl ring.

The term “bicyclic heterocycle” or “bicyclic heterocyclyl” as used herein refers to a ring system in which at least one of the ring members is other than carbon. Bicyclic heterocyclyl encompasses ring systems wherein an aromatic ring is fused with another aromatic ring, or wherein an aromatic ring is fused with a non-aromatic ring. Bicyclic heterocyclyl encompasses ring systems wherein a benzene ring is fused to a 5- or a 6-membered ring containing 1, 2 or 3 ring heteroatoms or wherein a pyridine ring is fused to a 5- or a 6-membered ring containing 1, 2 or 3 ring heteroatoms. Bicyclic heterocyclic groups include, but are not limited to, indolyl, indazolyl, pyrazolo[1,5-a]pyridinyl, benzofuranyl, quinolinyl, quinoxalinyl, 1,3-benzodioxolyl, 2,3-dihydro-1,4-benzodioxinyl, 3,4-dihydro-2H-chromenyl, 1H-pyrazolo[4,3-c]pyridin-3-yl; 1H-pyrrolo[3,2-b]pyridin-3-yl; and 1H-pyrazolo[3,2-b]pyridin-3-yl.

The term “heterocycloalkyl” as used herein refers to an aliphatic, partially unsaturated or fully saturated, 3- to 14-membered ring system, including single rings of 3 to 8 atoms and bi- and tricyclic ring systems. The heterocycloalkyl ring-systems include one to four heteroatoms independently selected from oxygen, nitrogen, and sulfur, wherein a nitrogen and sulfur heteroatom optionally can be oxidized and a nitrogen heteroatom optionally can be substituted. Representative heterocycloalkyl groups include, but are not limited to, pyrrolidinyl, pyrazolinyl, pyrazolidinyl, imidazolinyl, imidazolidinyl, piperidinyl, piperazinyl, oxazolidinyl, isoxazolidinyl, morpholinyl, thiazolidinyl, isothiazolidinyl, and tetrahydrofuryl.

The term “hydroxyl” or “hydroxy” as used herein is represented by the formula —OH.

The term “ketone” as used herein is represented by the formula A¹C(O)A², where A¹ and A² can be, independently, an alkyl, cycloalkyl, alkenyl, cycloalkenyl, alkynyl, cycloalkynyl, aryl, or heteroaryl group as described herein.

The term “azide” or “azido” as used herein is represented by the formula —N₃.

The term “nitro” as used herein is represented by the formula —NO₂.

The term “nitrile” or “cyano” as used herein is represented by the formula —CN.

The term “silyl” as used herein is represented by the formula —SiA¹A²A³, where A¹, A², and A³ can be, independently, hydrogen or an alkyl, cycloalkyl, alkoxy, alkenyl, cycloalkenyl, alkynyl, cycloalkynyl, aryl, or heteroaryl group as described herein.

The term “sulfo-oxo” as used herein is represented by the formulas —S(O)A¹, —S(O)₂A¹, —OS(O)₂A¹, or —OS(O)₂OA¹, where A¹ can be hydrogen or an alkyl, cycloalkyl, alkenyl, cycloalkenyl, alkynyl, cycloalkynyl, aryl, or heteroaryl group as described herein. Throughout this specification “S(O)” is a shorthand notation for S═O. The term “sulfonyl” is used herein to refer to the sulfo-oxo group represented by the formula —S(O)₂A¹, where A¹ can be hydrogen or an alkyl, cycloalkyl, alkenyl, cycloalkenyl, alkynyl, cycloalkynyl, aryl, or heteroaryl group as described herein. The term “sulfone” as used herein is represented by the formula A¹S(O)₂A², where A¹ and A² can be, independently, an alkyl, cycloalkyl, alkenyl, cycloalkenyl, alkynyl, cycloalkynyl, aryl, or heteroaryl group as described herein. The term “sulfoxide” as used herein is represented by the formula A¹S(O)A², where A¹ and A² can be, independently, an alkyl, cycloalkyl, alkenyl, cycloalkenyl, alkynyl, cycloalkynyl, aryl, or heteroaryl group as described herein.

The term “thiol” as used herein is represented by the formula —SH.

“R¹,” “R²,” “R³,” . . . “Rⁿ,” where n is an integer, as used herein can, independently, possess one or more of the groups listed above. For example, if R¹ is a straight chain alkyl group, one of the hydrogen atoms of the alkyl group can optionally be substituted with a hydroxyl group, an alkoxy group, an alkyl group, a halide, and the like. Depending upon the groups that are selected, a first group can be incorporated within second group or, alternatively, the first group can be pendant (i.e., attached) to the second group. For example, with the phrase “an alkyl group comprising an amino group,” the amino group can be incorporated within the backbone of the alkyl group. Alternatively, the amino group can be attached to the backbone of the alkyl group. The nature of the group(s) that is (are) selected will determine if the first group is embedded or attached to the second group.

As described herein, compounds of the invention may contain “optionally substituted” moieties. In general, the term “substituted,” whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. In is also contemplated that, in certain aspects, unless expressly indicated to the contrary, individual substituents can be further optionally substituted (i.e., further substituted, or unsubstituted).

The term “stable,” as used herein, refers to compounds that are not substantially altered when subjected to conditions to allow for their production, detection, and, in certain aspects, their recovery, purification, and use for one or more of the purposes disclosed herein.

Suitable monovalent substituents on a substitutable carbon atom of an “optionally substituted” group are independently halogen; —(CH₂)₀₋₄R^{∘}; —(CH₂)₀₋₄OR^{∘}; —O(CH₂)₀₋₄R^{∘},
—O—(CH₂)₀₋₄C(O)OR^{∘}; —(CH₂)₀₋₄CH(OR^{∘})₂; —(CH₂)₀₋₄SR^{∘}; —(CH₂)₀₋₄Ph, which may be substituted with R^{∘}; —(CH₂)₀₋₄O(CH₂)₀₋₁Ph which may be substituted with R^{∘}; —CH═CHPh, which may be substituted with R^{∘}; —(CH₂)₀₋₄O(CH₂)₀₋₁-pyridyl which may be substituted with R^{∘}; —NO₂; —CN;
—N₃; —(CH₂)₀₋₄N(R^{∘})₂; —(CH₂)₀₋₄N(R^{∘})C(O)R^{∘}; —N(R^{∘})C(S)R^{∘};
—(CH₂)₀₋₄N(R^{∘})C(O)NR^{∘}₂; —N(R^{∘})C(S)NR^{∘}₂; —(CH₂)₀₋₄N(R^{∘})C(O)OR^{∘};
—N(R^{∘})N(R^{∘})C(O)R^{∘}; —N(R^{∘})N(R^{∘})C(O)NR^{∘}₂; —N(R^{∘})N(R^{∘})C(O)OR^{∘}; —(CH₂)₀₋₄C(O)R^{∘}; —C(S)R^{∘};
—(CH₂)₀₋₄C(O)OR^{∘}; —(CH₂)₀₋₄C(O)SR^{∘}; —(CH₂)₀₋₄C(O)OSiR^{∘}₃; —(CH₂)₀₋₄OC(O)R^{∘}; —OC(O)(CH₂)₀₋₄SR—, SC(S)SR^{∘}; —(CH₂)₀₋₄SC(O)R^{∘}; —(CH₂)₀₋₄C(O)NR^{∘}₂; —C(S)NR^{∘}₂; —C(S)SR^{∘}; —(CH₂)₀₋₄OC(O)NR^{∘}₂; —C(O)N(OR^{∘})R^{∘}; —C(O)C(O)R^{∘}; —C(O)CH₂C(O)R^{∘}; —C(NOR^{∘})R^{∘}; —(CH₂)₀₋₄SSR^{∘};
—(CH₂)₀₋₄S(O)₂R^{∘}; —(CH₂)₀₋₄S(O)₂OR^{∘}; —(CH₂)₀₋₄OS(O)₂R^{∘}; —S(O)₂NR^{∘}₂; —(CH₂)₀₋₄S(O)R^{∘}; —N(R^{∘})S(O)₂NR^{∘}₂; —N(R^{∘})S(O)₂R^{∘}; —N(OR^{∘})R^{∘}; —C(NH)NR^{∘}₂;
—P(O)₂R^{∘}; —P(O)R^{∘}₂; —OP(O)R^{∘}₂; —OP(O)(OR^{∘})₂; SiR^{∘}₃; —(C₁₋₄ straight or branched alkylene)O—N(R^{∘})₂; or —(C₁₋₄ straight or branched alkylene)C(O)O—N(R^{∘})₂, wherein each R^{∘} may be substituted as defined below and is independently hydrogen, C₁₋₆ aliphatic, —CH₂Ph, —O(CH₂)₀₋₁Ph, —CH₂-(5-6 membered heteroaryl ring), or a 5-6-membered saturated, partially unsaturated, or aryl ring having 0-4 heteroatoms independently selected from nitrogen, oxygen, or sulfur, or, notwithstanding the definition above, two independent occurrences of R^{∘}, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R^{∘} (or the ring formed by taking two independent occurrences of R^{∘} together with their intervening atoms), are independently halogen, —(CH₂)₀₋₂R^{I}, -(haloR^{I}), —(CH₂)₀₋₂OH, —(CH₂)₀₋₂OR^{I}, —(CH₂)₀₋₂CH(OR^{I})₂; —O(haloR^{I}), —CN, —N₃, —(CH₂)₀₋₂C(O)R^{I}, —(CH₂)₀₋₂C(O)OH, —(CH₂)₀₋₂C(O)OR^{I}, —(CH₂)₀₋₂SR^{I}, —(CH₂)₀₋₂SH, —(CH₂)₀₋₂NH₂,
—(CH₂)₀₋₂NHR^{I}, —(CH₂)₀₋₂NR^{I}₂, —NO₂, —SiR^{I}₃, —OSiR^{I}₃, —C(O)SR^{I}, —(C₁₋₄ straight or branched alkylene)C(O)OR^{I}, or —SSR^{I} wherein each R^{I} is unsubstituted or where preceded by “halo” is substituted only with one or more halogens, and is independently selected from C₁₋₄ aliphatic,
—CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{∘} include ═O and ═S.

Suitable divalent substituents on a saturated carbon atom of an “optionally substituted” group include the following: ═O, ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R* include halogen, —R^{I}, -(haloR^{I}), —OH,
—OR^{I}, —O(haloR^{I}), —CN, —C(O)OH, —C(O)OR^{I}, —NH₂, —NHR^{I}, —NR^{I}₂, or —NO₂, wherein each R^{I} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†},
—S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic which may be substituted as defined below, unsubstituted —OPh, or an unsubstituted 5-6-membered saturated, partially unsaturated, or aryl ring having 0-4 heteroatoms independently selected from nitrogen, oxygen, or sulfur, or, notwithstanding the definition above, two independent occurrences of Rt, taken together with their intervening atom(s) form an unsubstituted 3-12-membered saturated, partially unsaturated, or aryl mono- or bicyclic ring having 0-4 heteroatoms independently selected from nitrogen, oxygen, or sulfur.

Suitable substituents on the aliphatic group of R^{†} are independently halogen,
—R^{I}, -(haloR^{I}), —OH, —OR^{I}, —O(haloR^{I}), —CN, —C(O)OH, —C(O)OR^{I}, —NH₂, —NHR^{I}, —NR^{I}₂, or
—NO₂, wherein each R^{I} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

The term “leaving group” refers to an atom (or a group of atoms) with electron withdrawing ability that can be displaced as a stable species, taking with it the bonding electrons. Examples of suitable leaving groups include halides and sulfonate esters, including, but not limited to, triflate, mesylate, tosylate, and brosylate.

Compounds described herein can contain one or more double bonds and, thus, potentially give rise to cis/trans (E/Z) isomers, as well as other conformational isomers. Unless stated to the contrary, the invention includes all such possible isomers, as well as mixtures of such isomers.

Unless stated to the contrary, a formula with chemical bonds shown only as solid lines and not as wedges or dashed lines contemplates each possible isomer, e.g., each enantiomer and diastereomer, and a mixture of isomers, such as a racemic or scalemic mixture. Compounds described herein can contain one or more asymmetric centers and, thus, potentially give rise to diastereomers and optical isomers. Unless stated to the contrary, the present invention includes all such possible diastereomers as well as their racemic mixtures, their substantially pure resolved enantiomers, all possible geometric isomers, and pharmaceutically acceptable salts thereof. Mixtures of stereoisomers, as well as isolated specific stereoisomers, are also included. During the course of the synthetic procedures used to prepare such compounds, or in using racemization or epimerization procedures known to those skilled in the art, the products of such procedures can be a mixture of stereoisomers.

Many organic compounds exist in optically active forms having the ability to rotate the plane of plane-polarized light. In describing an optically active compound, the prefixes D and L or R and S are used to denote the absolute configuration of the molecule about its chiral center(s). The prefixes D and L or (+) and (−) are employed to designate the sign of rotation of plane-polarized light by the compound, with (−) or L meaning that the compound is levorotatory. A compound prefixed with (+) or D is dextrorotatory. For a given chemical structure, these compounds, called stereoisomers, are identical except that they are non-superimposable mirror images of one another. A specific stereoisomer can also be referred to as an enantiomer, and a mixture of such isomers is often called an enantiomeric mixture. A 50:50 mixture of enantiomers is referred to as a racemic mixture. Many of the compounds described herein can have one or more chiral centers and therefore can exist in different enantiomeric forms. If desired, a chiral carbon can be designated with an asterisk (*). When bonds to the chiral carbon are depicted as straight lines in the disclosed formulas, it is understood that both the (R) and (S) configurations of the chiral carbon, and hence both enantiomers and mixtures thereof, are embraced within the formula. As is used in the art, when it is desired to specify the absolute configuration about a chiral carbon, one of the bonds to the chiral carbon can be depicted as a wedge (bonds to atoms above the plane) and the other can be depicted as a series or wedge of short parallel lines is (bonds to atoms below the plane). The Cahn-Ingold-Prelog system can be used to assign the (R) or (S) configuration to a chiral carbon.

Compounds described herein comprise atoms in both their natural isotopic abundance and in non-natural abundance. The disclosed compounds can be isotopically-labeled or isotopically-substituted compounds identical to those described, but for the fact that one or more atoms are replaced by an atom having an atomic mass or mass number different from the atomic mass or mass number typically found in nature. Examples of isotopes that can be incorporated into compounds of the invention include isotopes of hydrogen, carbon, nitrogen, oxygen, sulfur, fluorine and chlorine, such as ²H, ³H, ¹³C, ¹⁴C ¹⁵N, ¹⁸O, ¹⁷O, ³⁵S, ¹⁸F, and ³⁶Cl, respectively. Compounds further comprise prodrugs thereof and pharmaceutically acceptable salts of said compounds or of said prodrugs which contain the aforementioned isotopes and/or other isotopes of other atoms are within the scope of this invention. Certain isotopically-labeled compounds of the present invention, for example those into which radioactive isotopes such as ³H and ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. Isotopically labeled compounds of the present invention and prodrugs thereof can generally be prepared by carrying out the procedures below, by substituting a readily available isotopically labeled reagent for a non-isotopically labeled reagent.

The compounds described in the invention can be present as a solvate. In some cases, the solvent used to prepare the solvate is an aqueous solution, and the solvate is then often referred to as a hydrate. The compounds can be present as a hydrate, which can be obtained, for example, by crystallization from a solvent or from aqueous solution. In this connection, one, two, three or any arbitrary number of solvent or water molecules can combine with the compounds according to the invention to form solvates and hydrates. Unless stated to the contrary, the invention includes all such possible solvates.

It is also appreciated that certain compounds described herein can be present as an equilibrium of tautomers. For example, ketones with an α-hydrogen can exist in an equilibrium of the keto form and the enol form.

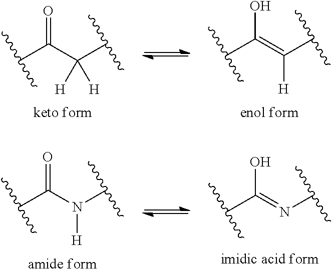 [SMILES: *CC(*)=O ; */C=C(/*)O ; *NC(*)=O ; */N=C(/*)O]

Likewise, amides with an N-hydrogen can exist in an equilibrium of the amide form and the imidic acid form. Unless stated to the contrary, the invention includes all such possible tautomers.

It is known that chemical substances form solids which are present in different states of order which are termed polymorphic forms or modifications. The different modifications of a polymorphic substance can differ greatly in their physical properties. The compounds according to the invention can be present in different polymorphic forms, with it being possible for particular modifications to be metastable. Unless stated to the contrary, the invention includes all such possible polymorphic forms.

In some aspects, a structure of a compound can be represented by a formula:

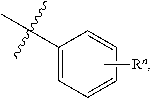

which is understood to be equivalent to a formula:

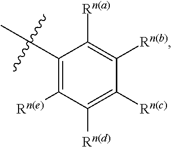

wherein n is typically an integer. That is, Rⁿ is understood to represent five independent substituents, R^{n(a)}, R^{n(b)}, R^{n(c)}, R^{n(d)} and R^{n(e)}. By “independent substituents,” it is meant that each R substituent can be independently defined. For example, if in one instance R^{n(a)} is halogen, then R^{n(b)} is not necessarily halogen in that instance.

As used herein, “administering” can refer to an administration that is oral, topical, intravenous, subcutaneous, transcutaneous, transdermal, intramuscular, intra-joint, parenteral, intra-arteriole, intradermal, intraventricular, intraosseous, intraocular, intracranial, intraperitoneal, intralesional, intranasal, intracardiac, intraarticular, intracavernous, intrathecal, intravireal, intracerebral, and intracerebroventricular, intratympanic, intracochlear, rectal, vaginal, by inhalation, by catheters, stents or via an implanted reservoir or other device that administers, either actively or passively (e.g. by diffusion) a composition the perivascular space and adventitia. For example, a medical device such as a stent can contain a composition or formulation disposed on its surface, which can then dissolve or be otherwise distributed to the surrounding tissue and cells. The term “parenteral” can include subcutaneous, intravenous, intramuscular, intra-articular, intra-synovial, intrasternal, intrathecal, intrahepatic, intralesional, and intracranial injections or infusion techniques. Administration can be continuous or intermittent. In various aspects, a preparation can be administered therapeutically; that is, administered to treat an existing disease or condition. In further various aspects, a preparation can be administered prophylactically; that is, administered for prevention of a disease or condition.

As used interchangeably herein, “subject,” “individual,” or “patient” can refer to a vertebrate organism, such as a mammal (e.g., human). “Subject” can also refer to a cell, a population of cells, a tissue, an organ, or an organism, preferably to human and constituents thereof.

As used herein, the terms “treating” and “treatment” can refer generally to obtaining a desired pharmacological and/or physiological effect. The effect can be, but does not necessarily have to be, prophylactic in terms of preventing or partially preventing a disease, symptom, or condition thereof, such as a hematological malignancy, breast cancer, and/or another solid malignancy. The effect can be therapeutic in terms of a partial or complete cure of a disease, condition, symptom or adverse effect attributed to the disease, disorder, or condition. The term “treatment” as used herein can include any treatment of a hematological malignancy, breast cancer, and/or another solid tumor in a subject, particularly a human and can include any one or more of the following: (a) preventing the disease from occurring in a subject which may be predisposed to the disease but has not yet been diagnosed as having it; (b) inhibiting the disease, i.e., arresting its development; and (c) relieving the disease, i.e., mitigating or ameliorating the disease and/or its symptoms or conditions. The term “treatment” as used herein can refer to both therapeutic treatment alone, prophylactic treatment alone, or both therapeutic and prophylactic treatment. Those in need of treatment (subjects in need thereof) can include those already with the disorder and/or those in which the disorder is to be prevented. As used herein, the term “treating”, can include inhibiting the disease, disorder or condition, e.g., impeding its progress; and relieving the disease, disorder, or condition, e.g., causing regression of the disease, disorder and/or condition. Treating the disease, disorder, or condition can include ameliorating at least one symptom of the particular disease, disorder, or condition, even if the underlying pathophysiology is not affected, e.g., such as treating the pain of a subject by administration of an analgesic agent even though such agent does not treat the cause of the pain.

As used herein, “therapeutic” can refer to treating, healing, and/or ameliorating a disease, disorder, condition, or side effect, or to decreasing in the rate of advancement of a disease, disorder, condition, or side effect.

As used herein, “effective amount” can refer to the amount of a disclosed compound or pharmaceutical composition provided herein that is sufficient to effect beneficial or desired biological, emotional, medical, or clinical response of a cell, tissue, system, animal, or human. An effective amount can be administered in one or more administrations, applications, or dosages. The term can also include within its scope amounts effective to enhance or restore to substantially normal physiological function.

For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. It is generally preferred that a maximum dose of the pharmacological agents of the invention (alone or in combination with other therapeutic agents) be used, that is, the highest safe dose according to sound medical judgment. It will be understood by those of ordinary skill in the art however, that a patient may insist upon a lower dose or tolerable dose for medical reasons, psychological reasons or for virtually any other reasons.

A response to a therapeutically effective dose of a disclosed compound and/or pharmaceutical composition, for example, can be measured by determining the physiological effects of the treatment or medication, such as the decrease or lack of disease symptoms following administration of the treatment or pharmacological agent. Other assays will be known to one of ordinary skill in the art and can be employed for measuring the level of the response. The amount of a treatment may be varied for example by increasing or decreasing the amount of a disclosed compound and/or pharmaceutical composition, by changing the disclosed compound and/or pharmaceutical composition administered, by changing the route of administration, by changing the dosage timing and so on. Dosage can vary, and can be administered in one or more dose administrations daily, for one or several days. Guidance can be found in the literature for appropriate dosages for given classes of pharmaceutical products.

As used herein, the term “prophylactically effective amount” refers to an amount effective for preventing onset or initiation of a disease or condition.

As used herein, the term “prevent” or “preventing” refers to precluding, averting, obviating, forestalling, stopping, or hindering something from happening, especially by advance action. It is understood that where reduce, inhibit, or prevent are used herein, unless specifically indicated otherwise, the use of the other two words is also expressly disclosed.

The term “pharmaceutically acceptable” describes a material that is not biologically or otherwise undesirable, i.e., without causing an unacceptable level of undesirable biological effects or interacting in a deleterious manner.

The term “pharmaceutically acceptable salts”, as used herein, means salts of the active principal agents which are prepared with acids or bases that are tolerated by a biological system or tolerated by a subject or tolerated by a biological system and tolerated by a subject when administered in a therapeutically effective amount. When compounds of the present disclosure contain relatively acidic functionalities, base addition salts can be obtained by contacting the neutral form of such compounds with a sufficient amount of the desired base, either neat or in a suitable inert solvent. Examples of pharmaceutically acceptable base addition salts include, but are not limited to, sodium, potassium, calcium, ammonium, organic amino, magnesium salt, lithium salt, strontium salt or a similar salt. When compounds of the present disclosure contain relatively basic functionalities, acid addition salts can be obtained by contacting the neutral form of such compounds with a sufficient amount of the desired acid, either neat or in a suitable inert solvent. Examples of pharmaceutically acceptable acid addition salts include, but are not limited to; those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like.

The term “pharmaceutically acceptable prodrug” or “prodrug” represents those prodrugs of the compounds of the present disclosure which are, within the scope of sound medical judgment, suitable for use in contact with the tissues of humans and lower animals without undue toxicity, irritation, allergic response, and the like, commensurate with a reasonable benefit/risk ratio, and effective for their intended use. Prodrugs of the present disclosure can be rapidly transformed in vivo to a parent compound having a structure of a disclosed compound, for example, by hydrolysis in blood. A thorough discussion is provided in T. Higuchi and V. Stella, Pro-drugs as Novel Delivery Systems, V. 14 of the A.C.S. Symposium Series, and in Edward B. Roche, ed., Bioreversible Carriers in Drug Design, American Pharmaceutical Association and Pergamon Press (1987).

As used herein, “dose,” “unit dose,” or “dosage” can refer to physically discrete units suitable for use in a subject, each unit containing a predetermined quantity of a disclosed compound and/or a pharmaceutical composition thereof calculated to produce the desired response or responses in association with its administration.

Certain materials, compounds, compositions, and components disclosed herein can be obtained commercially or readily synthesized using techniques generally known to those of skill in the art. For example, the starting materials and reagents used in preparing the disclosed compounds and compositions are either available from commercial suppliers such as Aldrich Chemical Co., (Milwaukee, Wis.), Acros Organics (Morris Plains, N.J.), Fisher Scientific (Pittsburgh, Pa.), or Sigma (St. Louis, Mo.) or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991); March's Advanced Organic Chemistry, (John Wiley and Sons, 4th Edition); and Larock's Comprehensive Organic Transformations (VCH Publishers Inc., 1989).

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; and the number or type of embodiments described in the specification.

Disclosed are the components to be used to prepare the compositions of the invention as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds cannot be explicitly disclosed, each is specifically contemplated and described herein. For example, if a particular compound is disclosed and discussed and a number of modifications that can be made to a number of molecules including the compounds are discussed, specifically contemplated is each and every combination and permutation of the compound and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the compositions of the invention. Thus, if there are a variety of additional steps that can be performed it is understood that each of these additional steps can be performed with any specific embodiment or combination of embodiments of the methods of the invention.

It is understood that the compositions disclosed herein have certain functions. Disclosed herein are certain structural requirements for performing the disclosed functions, and it is understood that there are a variety of structures that can perform the same function that are related to the disclosed structures, and that these structures will typically achieve the same result.

As used herein, the terms “optional” or “optionally” means that the subsequently described event or circumstance can or cannot occur, and that the description includes instances where said event or circumstance occurs and instances where it does not.

Unless otherwise specified, temperatures referred to herein are based on atmospheric pressure (i.e. one atmosphere).

### Compounds and Methods of Making and Using the Compounds

In one aspect, disclosed herein is a compound having a formula represented by structure I or the pharmaceutically acceptable salt thereof

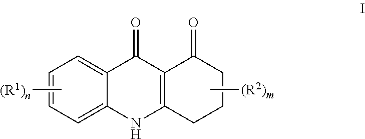

wherein
   n is an integer from 1 to 4, where each R¹ is independently hydrogen, a halide, a substituted or unsubstituted linear or branched alkyl group, or an alkoxy group, and
   m is an integer from 1 to 3, where each R² is hydrogen, a substituted or unsubstituted cycloalkyl group, a substituted or unsubstituted heterocycloalkyl group, a substituted or unsubstituted aryl group, a substituted or unsubstituted heteroaryl group, a substituted or unsubstituted biaryl group, or a substituted or unsubstituted biaryl ether group.

In one aspect, n in structure I is 1 or 2. In another aspect, R¹ in structure I is a C1-C6 alkyl group. In another aspect, R¹ in structure I is a methyl or ethyl group. In another aspect, R¹ in structure I is a halide. In another aspect, R¹ in structure I is bromide, chloride, iodide, or fluoride. In another aspect, R¹ in structure I is a C1-C6 alkoxy group. In another aspect, R¹ in structure I is a methoxy or ethoxy group. In another aspect, m in structure I is 1. In another aspect, R² in structure I is a substituted or unsubstituted phenyl group. In another aspect, R² in structure I is a phenyl group substituted with a halide, a substituted or unsubstituted alkyl group, an alkoxy group, or an aryloxy group. In another aspect, R² in structure I is a substituted or unsubstituted heterocycloalkyl group such as, for example, is a 5- or 6-membered ring. In another aspect, the unsubstituted heterocycloalkyl group is a tetrahydrofuran group or a tetrahydropyran group. In another aspect, R² in structure I is a substituted or unsubstituted heteroaryl group. In another aspect, the heteroaryl group is a pyridine group, a thiophene group, or a thiazole group. In another aspect, the heteroaryl group is substituted with a substituted or unsubstituted phenyl group. In another aspect, the phenyl group is substituted with a halide, a substituted or unsubstituted alkyl group, an alkoxy group, a fluoroalkoxy group, or a fluoroalkyl group. In another aspect, the phenyl group is substituted with —OCF₃. In another aspect, R² in structure I is a C3-C7 cycloalkyl group.

In another aspect, the compound has the structure II

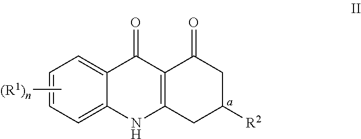

wherein the stereochemistry at carbon a is racemic or has an enantiomeric excess of at least 95% of the R or S enantiomer.

In one aspect, R² in structure II is a substituted or unsubstituted phenyl group. In another aspect, R² in structure II is a phenyl group substituted with a halide, a substituted or unsubstituted alkyl group, an alkoxy group, or an aryloxy group. In another aspect, R² in structure II is a substituted or unsubstituted heterocycloalkyl group such as, for example, a 5- or 6-membered ring. In another aspect, the unsubstituted heterocycloalkyl group is a tetrahydrofuran group or a tetrahydropyran group. In another aspect, R² in structure II is a substituted or unsubstituted heteroaryl group. In another aspect, the heteroaryl group is a pyridine group, a thiophene group, or a thiazole group. In another aspect, the heteroaryl group is substituted with a substituted or unsubstituted phenyl group. In another aspect, the phenyl group is substituted with a halide, a substituted or unsubstituted alkyl group, an alkoxy group, a fluoroalkoxy group, or a fluoroalkyl group. In another aspect, the phenyl group is substituted with —OCF₃. In another aspect, R² in structure II is a C3-C7 cycloalkyl group.

In one aspect, the stereochemistry at carbon a of formula II has an enantiomeric excess of at least 95% of the R or S enantiomer, at least 97% of the R or S enantiomer, at least 98% of the R or S enantiomer, at least 99% of the R or S enantiomer, or 100% of the R or S enantiomer. In one aspect, the compounds of formula II can be prepared as a racemic mixture initially then subsequently the enantiomers can be separated using chiral HPLC/SFC to isolate each enantiomer.

In another aspect, R² in structure II is

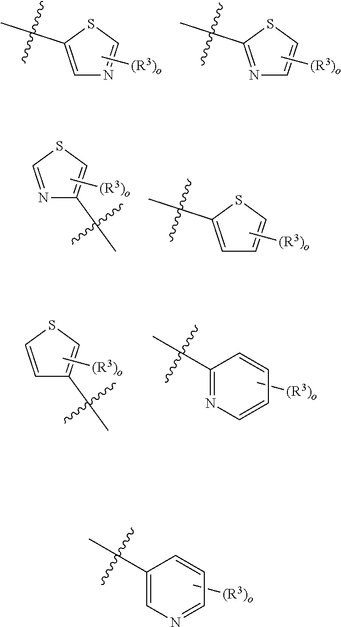

wherein o is 1 or 2 and R³ is hydrogen, a substituted or unsubstituted cycloalkyl group, a substituted or unsubstituted heterocycloalkyl group, a substituted or unsubstituted aryl group, a substituted or unsubstituted heteroaryl group, or a substituted or unsubstituted aryloxy group.

In one aspect, R³ in substituent R² is a phenyl group substituted with a fluoride, fluoroalkyl group and/or fluoroalkoxy group. In another aspect, R³ in substituent R² is a phenyl group substituted with —F, —CF₃ and/or —OCF₃.

### Pharmaceutical Compositions

In various aspects, the present disclosure relates to pharmaceutical compositions comprising a therapeutically effective amount of at least one disclosed compound, at least one product of a disclosed method, or a pharmaceutically acceptable salt thereof. As used herein, “pharmaceutically-acceptable carriers” means one or more of a pharmaceutically acceptable diluents, preservatives, antioxidants, solubilizers, emulsifiers, coloring agents, releasing agents, coating agents, sweetening, flavoring and perfuming agents, and adjuvants. The disclosed pharmaceutical compositions can be conveniently presented in unit dosage form and prepared by any of the methods well known in the art of pharmacy and pharmaceutical sciences.

In a further aspect, the disclosed pharmaceutical compositions comprise a therapeutically effective amount of at least one disclosed compound, at least one product of a disclosed method, or a pharmaceutically acceptable salt thereof as an active ingredient, a pharmaceutically acceptable carrier, optionally one or more other therapeutic agent, and optionally one or more adjuvant. The disclosed pharmaceutical compositions include those suitable for oral, rectal, topical, pulmonary, nasal, and parenteral administration, although the most suitable route in any given case will depend on the particular host, and nature and severity of the conditions for which the active ingredient is being administered. In a further aspect, the disclosed pharmaceutical composition can be formulated to allow administration orally, nasally, via inhalation, parenterally, paracancerally, transmucosally, transdermally, intramuscularly, intravenously, intradermally, subcutaneously, intraperitoneally, intraventricularly, intracranially and intratumorally.

As used herein, “parenteral administration” includes administration by bolus injection or infusion, as well as administration by intravenous, intramuscular, intraarterial, intrathecal, intracapsular, intraorbital, intracardiac, intradermal, intraperitoneal, transtracheal, subcutaneous, subcuticular, intraarticular, subcapsular subarachnoid, intraspinal, epidural and intrasternal injection and infusion.

In various aspects, the present disclosure also relates to a pharmaceutical composition comprising a pharmaceutically acceptable carrier or diluent and, as active ingredient, a therapeutically effective amount of a disclosed compound, a product of a disclosed method of making, a pharmaceutically acceptable salt, a hydrate thereof, a solvate thereof, a polymorph thereof, or a stereochemically isomeric form thereof. In a further aspect, a disclosed compound, a product of a disclosed method of making, a pharmaceutically acceptable salt, a hydrate thereof, a solvate thereof, a polymorph thereof, or a stereochemically isomeric form thereof, or any subgroup or combination thereof may be formulated into various pharmaceutical forms for administration purposes.

In practice, the compounds of the present disclosure, or pharmaceutically acceptable salts thereof, of the present disclosure can be combined as the active ingredient in intimate admixture with a pharmaceutical carrier according to conventional pharmaceutical compounding techniques. The carrier can take a wide variety of forms depending on the form of preparation desired for administration, e.g., oral or parenteral (including intravenous). Thus, the pharmaceutical compositions of the present disclosure can be presented as discrete units suitable for oral administration such as capsules, cachets or tablets each containing a predetermined amount of the active ingredient. Further, the compositions can be presented as a powder, as granules, as a solution, as a suspension in an aqueous liquid, as a non-aqueous liquid, as an oil-in-water emulsion or as a water-in-oil liquid emulsion. In addition to the common dosage forms set out above, the compounds of the present disclosure, and/or pharmaceutically acceptable salt(s) thereof, can also be administered by controlled release means and/or delivery devices. The compositions can be prepared by any of the methods of pharmacy. In general, such methods include a step of bringing into association the active ingredient with the carrier that constitutes one or more necessary ingredients. In general, the compositions are prepared by uniformly and intimately admixing the active ingredient with liquid carriers or finely divided solid carriers or both. The product can then be conveniently shaped into the desired presentation.

It is especially advantageous to formulate the aforementioned pharmaceutical compositions in unit dosage form for ease of administration and uniformity of dosage. The term “unit dosage form,” as used herein, refers to physically discrete units suitable as unitary dosages, each unit containing a predetermined quantity of active ingredient calculated to produce the desired therapeutic effect in association with the required pharmaceutical carrier. That is, a “unit dosage form” is taken to mean a single dose wherein all active and inactive ingredients are combined in a suitable system, such that the patient or person administering the drug to the patient can open a single container or package with the entire dose contained therein and does not have to mix any components together from two or more containers or packages. Typical examples of unit dosage forms are tablets (including scored or coated tablets), capsules or pills for oral administration; single dose vials for injectable solutions or suspension; suppositories for rectal administration; powder packets; wafers; and segregated multiples thereof. This list of unit dosage forms is not intended to be limiting in any way, but merely to represent typical examples of unit dosage forms.

The pharmaceutical compositions disclosed herein comprise a compound of the present disclosure (or pharmaceutically acceptable salts thereof) as an active ingredient, a pharmaceutically acceptable carrier, and optionally one or more additional therapeutic agents. In various aspects, the disclosed pharmaceutical compositions can include a pharmaceutically acceptable carrier and a disclosed compound, or a pharmaceutically acceptable salt thereof. In a further aspect, a disclosed compound, or pharmaceutically acceptable salt thereof, can also be included in a pharmaceutical composition in combination with one or more other therapeutically active compounds. The instant compositions include compositions suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions can be conveniently presented in unit dosage form and prepared by any of the methods well known in the art of pharmacy.

Techniques and compositions for making dosage forms useful for materials and methods described herein are described, for example, in the following references: Modern Pharmaceutics, Chapters 9 and 10 (Banker & Rhodes, Editors, 1979); Pharmaceutical Dosage Forms: Tablets (Lieberman et al., 1981); Ansel, Introduction to Pharmaceutical Dosage Forms 2nd Edition (1976); Remington's Pharmaceutical Sciences, 17th ed. (Mack Publishing Company, Easton, Pa., 1985); Advances in Pharmaceutical Sciences (David Ganderton, Trevor Jones, Eds., 1992); Advances in Pharmaceutical Sciences Vol 7. (David Ganderton, Trevor Jones, James McGinity, Eds., 1995); Aqueous Polymeric Coatings for Pharmaceutical Dosage Forms (Drugs and the Pharmaceutical Sciences, Series 36 (James McGinity, Ed., 1989); Pharmaceutical Particulate Carriers: Therapeutic Applications: Drugs and the Pharmaceutical Sciences, Vol 61 (Alain Rolland, Ed., 1993); Drug Delivery to the Gastrointestinal Tract (Ellis Horwood Books in the Biological Sciences. Series in Pharmaceutical Technology; J. G. Hardy, S. S. Davis, Clive G. Wilson, Eds.); Modern Pharmaceutics Drugs and the Pharmaceutical Sciences, Vol 40 (Gilbert S. Banker, Christopher T. Rhodes, Eds.).

The compounds described herein are typically to be administered in admixture with suitable pharmaceutical diluents, excipients, extenders, or carriers (termed herein as a pharmaceutically acceptable carrier, or a carrier) suitably selected with respect to the intended form of administration and as consistent with conventional pharmaceutical practices. The deliverable compound will be in a form suitable for oral, rectal, topical, intravenous injection or parenteral administration. Carriers include solids or liquids, and the type of carrier is chosen based on the type of administration being used. The compounds may be administered as a dosage that has a known quantity of the compound.

Because of the ease in administration, oral administration can be a preferred dosage form, and tablets and capsules represent the most advantageous oral dosage unit forms in which case solid pharmaceutical carriers are obviously employed. However, other dosage forms may be suitable depending upon clinical population (e.g., age and severity of clinical condition), solubility properties of the specific disclosed compound used, and the like. Accordingly, the disclosed compounds can be used in oral dosage forms such as pills, powders, granules, elixirs, tinctures, suspensions, syrups, and emulsions. In preparing the compositions for oral dosage form, any convenient pharmaceutical media can be employed. For example, water, glycols, oils, alcohols, flavoring agents, preservatives, coloring agents and the like can be used to form oral liquid preparations such as suspensions, elixirs and solutions; while carriers such as starches, sugars, microcrystalline cellulose, diluents, granulating agents, lubricants, binders, disintegrating agents, and the like can be used to form oral solid preparations such as powders, capsules and tablets. Because of their ease of administration, tablets and capsules are the preferred oral dosage units whereby solid pharmaceutical carriers are employed. Optionally, tablets can be coated by standard aqueous or nonaqueous techniques.

The disclosed pharmaceutical compositions in an oral dosage form can comprise one or more pharmaceutical excipient and/or additive. Non-limiting examples of suitable excipients and additives include gelatin, natural sugars such as raw sugar or lactose, lecithin, pectin, starches (for example corn starch or amylose), dextran, polyvinyl pyrrolidone, polyvinyl acetate, gum arabic, alginic acid, tylose, talcum, lycopodium, silica gel (for example colloidal), cellulose, cellulose derivatives (for example cellulose ethers in which the cellulose hydroxy groups are partially etherified with lower saturated aliphatic alcohols and/or lower saturated, aliphatic oxyalcohols, for example methyl oxypropyl cellulose, methyl cellulose, hydroxypropyl methyl cellulose, hydroxypropyl methyl cellulose phthalate), fatty acids as well as magnesium, calcium or aluminum salts of fatty acids with 12 to 22 carbon atoms, in particular saturated (for example stearates), emulsifiers, oils and fats, in particular vegetable (for example, peanut oil, castor oil, olive oil, sesame oil, cottonseed oil, corn oil, wheat germ oil, sunflower seed oil, cod liver oil, in each case also optionally hydrated); glycerol esters and polyglycerol esters of saturated fatty acids C₁₂H₂₄O₂ to C₁₈H₃₆O₂ and their mixtures, it being possible for the glycerol hydroxy groups to be totally or also only partly esterified (for example mono-, di- and triglycerides); pharmaceutically acceptable mono- or multivalent alcohols and polyglycols such as polyethylene glycol and derivatives thereof, esters of aliphatic saturated or unsaturated fatty acids (2 to 22 carbon atoms, in particular 10-18 carbon atoms) with monovalent aliphatic alcohols (1 to 20 carbon atoms) or multivalent alcohols such as glycols, glycerol, diethylene glycol, pentacrythritol, sorbitol, mannitol and the like, which may optionally also be etherified, esters of citric acid with primary alcohols, acetic acid, urea, benzyl benzoate, dioxolanes, glyceroformals, tetrahydrofurfuryl alcohol, polyglycol ethers with C1-C12-alcohols, dimethylacetamide, lactamides, lactates, ethyl carbonates, silicones (in particular medium-viscous polydimethyl siloxanes), calcium carbonate, sodium carbonate, calcium phosphate, sodium phosphate, magnesium carbonate and the like.

Other auxiliary substances useful in preparing an oral dosage form are those which cause disintegration (so-called disintegrants), such as: cross-linked polyvinyl pyrrolidone, sodium carboxymethyl starch, sodium carboxymethyl cellulose or microcrystalline cellulose. Conventional coating substances may also be used to produce the oral dosage form. Those that may for example be considered are: polymerizates as well as copolymerizates of acrylic acid and/or methacrylic acid and/or their esters; copolymerizates of acrylic and methacrylic acid esters with a lower ammonium group content (for example EudragitR RS), copolymerizates of acrylic and methacrylic acid esters and trimethyl ammonium methacrylate (for example EudragitR RL); polyvinyl acetate; fats, oils, waxes, fatty alcohols; hydroxypropyl methyl cellulose phthalate or acetate succinate; cellulose acetate phthalate, starch acetate phthalate as well as polyvinyl acetate phthalate, carboxy methyl cellulose; methyl cellulose phthalate, methyl cellulose succinate, -phthalate succinate as well as methyl cellulose phthalic acid half ester; zein; ethyl cellulose as well as ethyl cellulose succinate; shellac, gluten; ethylcarboxyethyl cellulose; ethacrylate-maleic acid anhydride copolymer; maleic acid anhydride-vinyl methyl ether copolymer; styrol-maleic acid copolymerizate; 2-ethyl-hexyl-acrylate maleic acid anhydride; crotonic acid-vinyl acetate copolymer; glutaminic acid/glutamic acid ester copolymer; carboxymethylethylcellulose glycerol monooctanoate; cellulose acetate succinate; polyarginine.

Plasticizing agents that may be considered as coating substances in the disclosed oral dosage forms are: citric and tartaric acid esters (acetyl-triethyl citrate, acetyl tributyl-, tributyl-, triethyl-citrate); glycerol and glycerol esters (glycerol diacetate, -triacetate, acetylated monoglycerides, castor oil); phthalic acid esters (dibutyl-, diamyl-, diethyl-, dimethyl-, dipropyl-phthalate), di-(2-methoxy- or 2-ethoxyethyl)-phthalate, ethylphthalyl glycolate, butylphthalylethyl glycolate and butylglycolate; alcohols (propylene glycol, polyethylene glycol of various chain lengths), adipates (diethyladipate, di-(2-methoxy- or 2-ethoxyethyl)-adipate; benzophenone; diethyl- and diburylsebacate, dibutylsuccinate, dibutyltartrate; diethylene glycol dipropionate; ethyleneglycol diacetate, -dibutyrate, -dipropionate; tributyl phosphate, tributyrin; polyethylene glycol sorbitan monooleate (polysorbates such as Polysorbar 50); sorbitan monooleate.

Moreover, suitable binders, lubricants, disintegrating agents, coloring agents, flavoring agents, flow-inducing agents, and melting agents may be included as carriers. The pharmaceutical carrier employed can be, for example, a solid, liquid, or gas. Examples of solid carriers include, but are not limited to, lactose, terra alba, sucrose, glucose, methylcellulose, dicalcium phosphate, calcium sulfate, mannitol, sorbitol talc, starch, gelatin, agar, pectin, acacia, magnesium stearate, and stearic acid. Examples of liquid carriers are sugar syrup, peanut oil, olive oil, and water. Examples of gaseous carriers include carbon dioxide and nitrogen.

In various aspects, a binder can include, for example, starch, gelatin, natural sugars such as glucose or beta-lactose, corn sweeteners, natural and synthetic gums such as acacia, tragacanth, or sodium alginate, carboxymethylcellulose, polyethylene glycol, waxes, and the like. Lubricants used in these dosage forms include sodium oleate, sodium stearate, magnesium stearate, sodium benzoate, sodium acetate, sodium chloride, and the like. In a further aspect, a disintegrator can include, for example, starch, methyl cellulose, agar, bentonite, xanthan gum, and the like.

In various aspects, an oral dosage form, such as a solid dosage form, can comprise a disclosed compound that is attached to polymers as targetable drug carriers or as a prodrug. Suitable biodegradable polymers useful in achieving controlled release of a drug include, for example, polylactic acid, polyglycolic acid, copolymers of polylactic and polyglycolic acid, caprolactones, polyhydroxy butyric acid, polyorthoesters, polyacetals, polydihydropyrans, polycyanoacylates, and hydrogels, preferably covalently crosslinked hydrogels.

Tablets may contain the active ingredient in admixture with non-toxic pharmaceutically acceptable excipients which are suitable for the manufacture of tablets. These excipients may be, for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets may be uncoated or they may be coated by known techniques to delay disintegration and absorption in the gastrointestinal tract and thereby provide a sustained action over a longer period.

A tablet containing a disclosed compound can be prepared by compression or molding, optionally with one or more accessory ingredients or adjuvants. Compressed tablets can be prepared by compressing, in a suitable machine, the active ingredient in a free-flowing form such as powder or granules, optionally mixed with a binder, lubricant, inert diluent, surface active or dispersing agent. Molded tablets can be made by molding in a suitable machine, a mixture of the powdered compound moistened with an inert liquid diluent.

In various aspects, a solid oral dosage form, such as a tablet, can be coated with an enteric coating to prevent ready decomposition in the stomach. In various aspects, enteric coating agents include, but are not limited to, hydroxypropylmethylcellulose phthalate, methacrylic acid-methacrylic acid ester copolymer, polyvinyl acetate-phthalate and cellulose acetate phthalate. Akihiko Hasegawa “Application of solid dispersions of Nifedipine with enteric coating agent to prepare a sustained-release dosage form” Chem. Pharm. Bull. 33:1615-1619 (1985). Various enteric coating materials may be selected on the basis of testing to achieve an enteric coated dosage form designed ab initio to have a preferable combination of dissolution time, coating thicknesses and diametral crushing strength (e.g., see S. C. Porter et al. “The Properties of Enteric Tablet Coatings Made From Polyvinyl Acetate-phthalate and Cellulose acetate Phthalate”, J. Pharm. Pharmacol. 22:42p (1970)). In a further aspect, the enteric coating may comprise hydroxypropyl-methylcellulose phthalate, methacrylic acid-methacrylic acid ester copolymer, polyvinyl acetate-phthalate and cellulose acetate phthalate.

In various aspects, an oral dosage form can be a solid dispersion with a water soluble or a water insoluble carrier. Examples of water soluble or water insoluble carrier include, but are not limited to, polyethylene glycol, polyvinylpyrrolidone, hydroxypropylmethyl-cellulose, phosphatidylcholine, polyoxyethylene hydrogenated castor oil, hydroxypropylmethylcellulose phthalate, carboxymethylethylcellulose, or hydroxypropylmethylcellulose, ethyl cellulose, or stearic acid.

In various aspects, an oral dosage form can be in a liquid dosage form, including those that are ingested, or alternatively, administered as a mouth wash or gargle. For example, a liquid dosage form can include aqueous suspensions, which contain the active materials in admixture with excipients suitable for the manufacture of aqueous suspensions. In addition, oily suspensions may be formulated by suspending the active ingredient in a vegetable oil, for example arachis oil, olive oil, sesame oil or coconut oil, or in a mineral oil such as liquid paraffin. Oily suspensions may also contain various excipients. The pharmaceutical compositions of the present disclosure may also be in the form of oil-in-water emulsions, which may also contain excipients such as sweetening and flavoring agents.

For the preparation of solutions or suspensions it is, for example, possible to use water, particularly sterile water, or physiologically acceptable organic solvents, such as alcohols (ethanol, propanol, isopropanol, 1,2-propylene glycol, polyglycols and their derivatives, fatty alcohols, partial esters of glycerol), oils (for example peanut oil, olive oil, sesame oil, almond oil, sunflower oil, soya bean oil, castor oil, bovine hoof oil), paraffins, dimethyl sulfoxide, triglycerides and the like.

In the case of a liquid dosage form such as a drinkable solutions, the following substances may be used as stabilizers or solubilizers: lower aliphatic mono- and multivalent alcohols with 2-4 carbon atoms, such as ethanol, n-propanol, glycerol, polyethylene glycols with molecular weights between 200-600 (for example 1 to 40% aqueous solution), diethylene glycol monoethyl ether, 1,2-propylene glycol, organic amides, for example amides of aliphatic C1-C6-carboxylic acids with ammonia or primary, secondary or tertiary C1-C4-amines or C1-C4-hydroxy amines such as urea, urethane, acetamide, N-methyl acetamide, N,N-diethyl acetamide, N,N-dimethyl acetamide, lower aliphatic amines and diamines with 2-6 carbon atoms, such as ethylene diamine, hydroxyethyl theophylline, tromethamine (for example as 0.1 to 20% aqueous solution), aliphatic amino acids.

In preparing the disclosed liquid dosage form can comprise solubilizers and emulsifiers such as the following non-limiting examples can be used: polyvinyl pyrrolidone, sorbitan fatty acid esters such as sorbitan trioleate, phosphatides such as lecithin, acacia, tragacanth, polyoxyethylated sorbitan monooleate and other ethoxylated fatty acid esters of sorbitan, polyoxyethylated fats, polyoxyethylated oleotriglycerides, linolizated oleotriglycerides, polyethylene oxide condensation products of fatty alcohols, alkylphenols or fatty acids or also 1-methyl-3-(2-hydroxyethyl)imidazolidone-(2). In this context, polyoxyethylated means that the substances in question contain polyoxyethylene chains, the degree of polymerization of which generally lies between 2 and 40 and in particular between 10 and 20. Polyoxyethylated substances of this kind may for example be obtained by reaction of hydroxyl group-containing compounds (for example mono- or diglycerides or unsaturated compounds such as those containing oleic acid radicals) with ethylene oxide (for example 40 Mol ethylene oxide per 1 Mol glyceride). Examples of oleotriglycerides are olive oil, peanut oil, castor oil, sesame oil, cottonseed oil, corn oil. See also Dr. H. P. Fiedler “Lexikon der Hillsstoffe für Pharmazie, Kostnetik und angrenzende Gebiete” 1971, pages 191-195.

In various aspects, a liquid dosage form can further comprise preservatives, stabilizers, buffer substances, flavor correcting agents, sweeteners, colorants, antioxidants and complex formers and the like. Complex formers which may be for example be considered are: chelate formers such as ethylene diamine retrascetic acid, nitrilotriacetic acid, diethylene triamine pentacetic acid and their salts.

It may optionally be necessary to stabilize a liquid dosage form with physiologically acceptable bases or buffers to a pH range of approximately 6 to 9. Preference may be given to as neutral or weakly basic a pH value as possible (up to pH 8).

In order to enhance the solubility and/or the stability of a disclosed compound in a disclosed liquid dosage form, a parenteral injection form, or an intravenous injectable form, it can be advantageous to employ α-, β- or γ-cyclodextrins or their derivatives, in particular hydroxyalkyl substituted cyclodextrins, e.g. 2-hydroxypropyl-β-cyclodextrin or sulfobutyl-β-cyclodextrin. Also co-solvents such as alcohols may improve the solubility and/or the stability of the compounds according to the present disclosure in pharmaceutical compositions.

In various aspects, a disclosed liquid dosage form, a parenteral injection form, or an intravenous injectable form can further comprise liposome delivery systems, such as small unilamellar vesicles, large unilamellar vesicles, and multilamellar vesicles. Liposomes can be formed from a variety of phospholipids, such as cholesterol, stearylamine, or phosphatidylcholines.

Pharmaceutical compositions of the present disclosure suitable injection, such as parenteral administration, such as intravenous, intramuscular, or subcutaneous administration. Pharmaceutical compositions for injection can be prepared as solutions or suspensions of the active compounds in water. A suitable surfactant can be included such as, for example, hydroxypropylcellulose. Dispersions can also be prepared in glycerol, liquid polyethylene glycols, and mixtures thereof in oils. Further, a preservative can be included to prevent the detrimental growth of microorganisms.

Pharmaceutical compositions of the present disclosure suitable for parenteral administration can include sterile aqueous or oleaginous solutions, suspensions, or dispersions. Furthermore, the compositions can be in the form of sterile powders for the extemporaneous preparation of such sterile injectable solutions or dispersions. In some aspects, the final injectable form is sterile and must be effectively fluid for use in a syringe. The pharmaceutical compositions should be stable under the conditions of manufacture and storage; thus, preferably should be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), vegetable oils, and suitable mixtures thereof.

Injectable solutions, for example, can be prepared in which the carrier comprises saline solution, glucose solution or a mixture of saline and glucose solution. Injectable suspensions may also be prepared in which case appropriate liquid carriers, suspending agents and the like may be employed. In some aspects, a disclosed parenteral formulation can comprise about 0.01-0.1 M, e.g. about 0.05 M, phosphate buffer. In a further aspect, a disclosed parenteral formulation can comprise about 0.9% saline.

In various aspects, a disclosed parenteral pharmaceutical composition can comprise pharmaceutically acceptable carriers such as aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include but not limited to water, alcoholic/aqueous solutions, emulsions or suspensions, including saline and buffered media. Parenteral vehicles can include mannitol, normal serum albumin, sodium chloride solution, Ringer's dextrose, dextrose and sodium chloride, lactated Ringer's and fixed oils. Intravenous vehicles include fluid and nutrient replenishers, electrolyte replenishers such as those based on Ringer's dextrose, and the like. Preservatives and other additives may also be present, such as, for example, antimicrobials, antioxidants, collating agents, inert gases and the like. In a further aspect, a disclosed parenteral pharmaceutical composition can comprise may contain minor amounts of additives such as substances that enhance isotonicity and chemical stability, e.g., buffers and preservatives. Also contemplated for injectable pharmaceutical compositions are solid form preparations that are intended to be converted, shortly before use, to liquid form preparations. Furthermore, other adjuvants can be included to render the formulation isotonic with the blood of the subject or patient.

In addition to the pharmaceutical compositions described herein above, the disclosed compounds can also be formulated as a depot preparation. Such long-acting formulations can be administered by implantation (e.g., subcutaneously or intramuscularly) or by intramuscular injection. Thus, for example, the compounds can be formulated with suitable polymeric or hydrophobic materials (e.g., as an emulsion in an acceptable oil) or ion exchange resins, or as sparingly soluble derivatives, e.g., as a sparingly soluble salt.

Pharmaceutical compositions of the present disclosure can be in a form suitable for topical administration. As used herein, the phrase “topical application” means administration onto a biological surface, whereby the biological surface includes, for example, a skin area (e.g., hands, forearms, elbows, legs, face, nails, anus and genital areas) or a mucosal membrane. By selecting the appropriate carrier and optionally other ingredients that can be included in the composition, as is detailed herein below, the compositions of the present invention may be formulated into any form typically employed for topical application. A topical pharmaceutical composition can be in a form of a cream, an ointment, a paste, a gel, a lotion, milk, a suspension, an aerosol, a spray, foam, a dusting powder, a pad, and a patch. Further, the compositions can be in a form suitable for use in transdermal devices. These formulations can be prepared, utilizing a compound of the present disclosure, or pharmaceutically acceptable salts thereof, via conventional processing methods. As an example, a cream or ointment is prepared by mixing hydrophilic material and water, together with about 5 wt % to about 10 wt % of the compound, to produce a cream or ointment having a desired consistency.

In the compositions suitable for percutaneous administration, the carrier optionally comprises a penetration enhancing agent and/or a suitable wetting agent, optionally combined with suitable additives of any nature in minor proportions, which additives do not introduce a significant deleterious effect on the skin. Said additives may facilitate the administration to the skin and/or may be helpful for preparing the desired compositions. These compositions may be administered in various ways, e.g., as a transdermal patch, as a spot-on, as an ointment.

Ointments are semisolid preparations, typically based on petrolatum or petroleum derivatives. The specific ointment base to be used is one that provides for optimum delivery for the active agent chosen for a given formulation, and, preferably, provides for other desired characteristics as well (e.g., emollience). As with other carriers or vehicles, an ointment base should be inert, stable, nonirritating and nonsensitizing. As explained in Remington: The Science and Practice of Pharmacy, 19th Ed., Easton, Pa.: Mack Publishing Co. (1995), pp. 1399-1404, ointment bases may be grouped in four classes: oleaginous bases; emulsifiable bases; emulsion bases; and water-soluble bases. Oleaginous ointment bases include, for example, vegetable oils, fats obtained from animals, and semisolid hydrocarbons obtained from petroleum. Emulsifiable ointment bases, also known as absorbent ointment bases, contain little or no water and include, for example, hydroxystearin sulfate, anhydrous lanolin and hydrophilic petrolatum. Emulsion ointment bases are either water-in-oil (W/O) emulsions or oil-in-water (O/W) emulsions, and include, for example, cetyl alcohol, glyceryl monostearate, lanolin and stearic acid. Preferred water-soluble ointment bases are prepared from polyethylene glycols of varying molecular weight.

Lotions are preparations that are to be applied to the skin surface without friction. Lotions are typically liquid or semiliquid preparations in which solid particles, including the active agent, are present in a water or alcohol base. Lotions are typically preferred for treating large body areas, due to the ease of applying a more fluid composition. Lotions are typically suspensions of solids, and oftentimes comprise a liquid oily emulsion of the oil-in-water type. It is generally necessary that the insoluble matter in a lotion be finely divided. Lotions typically contain suspending agents to produce better dispersions as well as compounds useful for localizing and holding the active agent in contact with the skin, such as methylcellulose, sodium carboxymethyl-cellulose, and the like.

Creams are viscous liquids or semisolid emulsions, either oil-in-water or water-in-oil. Cream bases are typically water-washable, and contain an oil phase, an emulsifier and an aqueous phase. The oil phase, also called the “internal” phase, is generally comprised of petrolatum and/or a fatty alcohol such as cetyl or stearyl alcohol. The aqueous phase typically, although not necessarily, exceeds the oil phase in volume, and generally contains a humectant. The emulsifier in a cream formulation is generally a nonionic, anionic, cationic or amphoteric surfactant. Reference may be made to Remington: The Science and Practice of Pharmacy, supra, for further information.

Pastes are semisolid dosage forms in which the bioactive agent is suspended in a suitable base. Depending on the nature of the base, pastes are divided between fatty pastes or those made from a single-phase aqueous gel. The base in a fatty paste is generally petrolatum, hydrophilic petrolatum and the like. The pastes made from single-phase aqueous gels generally incorporate carboxymethylcellulose or the like as a base. Additional reference may be made to Remington: The Science and Practice of Pharmacy, for further information.

Gel formulations are semisolid, suspension-type systems. Single-phase gels contain organic macromolecules distributed substantially uniformly throughout the carrier liquid, which is typically aqueous, but also, preferably, contain an alcohol and, optionally, an oil. Preferred organic macromolecules, i.e., gelling agents, are crosslinked acrylic acid polymers such as the family of carbomer polymers, e.g., carboxypolyalkylenes that may be obtained commercially under the trademark Carbopol™. Other types of preferred polymers in this context are hydrophilic polymers such as polyethylene oxides, polyoxyethylene-polyoxypropylene copolymers and polyvinylalcohol; modified cellulose, such as hydroxypropyl cellulose, hydroxyethyl cellulose, hydroxypropyl methylcellulose, hydroxypropyl methylcellulose phthalate, and methyl cellulose; gums such as tragacanth and xanthan gum; sodium alginate; and gelatin. In order to prepare a uniform gel, dispersing agents such as alcohol or glycerin can be added, or the gelling agent can be dispersed by trituration, mechanical mixing or stirring, or combinations thereof.

Sprays generally provide the active agent in an aqueous and/or alcoholic solution which can be misted onto the skin for delivery. Such sprays include those formulated to provide for concentration of the active agent solution at the site of administration following delivery, e.g., the spray solution can be primarily composed of alcohol or other like volatile liquid in which the active agent can be dissolved. Upon delivery to the skin, the carrier evaporates, leaving concentrated active agent at the site of administration.

Foam compositions are typically formulated in a single or multiple phase liquid form and housed in a suitable container, optionally together with a propellant which facilitates the expulsion of the composition from the container, thus transforming it into a foam upon application. Other foam forming techniques include, for example the “Bag-in-a-can” formulation technique. Compositions thus formulated typically contain a low-boiling hydrocarbon, e.g., isopropane. Application and agitation of such a composition at the body temperature cause the isopropane to vaporize and generate the foam, in a manner similar to a pressurized aerosol foaming system. Foams can be water-based or aqueous alkanolic, but are typically formulated with high alcohol content which, upon application to the skin of a user, quickly evaporates, driving the active ingredient through the upper skin layers to the site of treatment.

Skin patches typically comprise a backing, to which a reservoir containing the active agent is attached. The reservoir can be, for example, a pad in which the active agent or composition is dispersed or soaked, or a liquid reservoir. Patches typically further include a frontal water permeable adhesive, which adheres and secures the device to the treated region. Silicone rubbers with self-adhesiveness can alternatively be used. In both cases, a protective permeable layer can be used to protect the adhesive side of the patch prior to its use. Skin patches may further comprise a removable cover, which serves for protecting it upon storage.

Examples of patch configuration which can be utilized with the present invention include a single-layer or multi-layer drug-in-adhesive systems which are characterized by the inclusion of the drug directly within the skin-contacting adhesive. In such a transdermal patch design, the adhesive not only serves to affix the patch to the skin, but also serves as the formulation foundation, containing the drug and all the excipients under a single backing film. In the multi-layer drug-in-adhesive patch a membrane is disposed between two distinct drug-in-adhesive layers or multiple drug-in-adhesive layers are incorporated under a single backing film.

Examples of pharmaceutically acceptable carriers that are suitable for pharmaceutical compositions for topical applications include carrier materials that are well-known for use in the cosmetic and medical arts as bases for e.g., emulsions, creams, aqueous solutions, oils, ointments, pastes, gels, lotions, milks, foams, suspensions, aerosols and the like, depending on the final form of the composition. Representative examples of suitable carriers according to the present invention therefore include, without limitation, water, liquid alcohols, liquid glycols, liquid polyalkylene glycols, liquid esters, liquid amides, liquid protein hydrolysates, liquid alkylated protein hydrolysates, liquid lanolin and lanolin derivatives, and like materials commonly employed in cosmetic and medicinal compositions. Other suitable carriers according to the present invention include, without limitation, alcohols, such as, for example, monohydric and polyhydric alcohols, e.g., ethanol, isopropanol, glycerol, sorbitol, 2-methoxyethanol, diethyleneglycol, ethylene glycol, hexyleneglycol, mannitol, and propylene glycol; ethers such as diethyl or dipropyl ether; polyethylene glycols and methoxypolyoxyethylenes (carbowaxes having molecular weight ranging from 200 to 20,000); polyoxyethylene glycerols, polyoxyethylene sorbitols, stearoyl diacetin, and the like.

Topical compositions of the present disclosure can, if desired, be presented in a pack or dispenser device, such as an FDA-approved kit, which may contain one or more unit dosage forms containing the active ingredient. The dispenser device may, for example, comprise a tube. The pack or dispenser device may be accompanied by instructions for administration. The pack or dispenser device may also be accompanied by a notice in a form prescribed by a governmental agency regulating the manufacture, use, or sale of pharmaceuticals, which notice is reflective of approval by the agency of the form of the compositions for human or veterinary administration. Such notice, for example, may include labeling approved by the U.S. Food and Drug Administration for prescription drugs or of an approved product insert. Compositions comprising the topical composition of the invention formulated in a pharmaceutically acceptable carrier may also be prepared, placed in an appropriate container, and labeled for treatment of an indicated condition.

Another patch system configuration which can be used by the present invention is a reservoir transdermal system design which is characterized by the inclusion of a liquid compartment containing a drug solution or suspension separated from the release liner by a semi-permeable membrane and adhesive. The adhesive component of this patch system can either be incorporated as a continuous layer between the membrane and the release liner or in a concentric configuration around the membrane. Yet another patch system configuration which can be utilized by the present invention is a matrix system design which is characterized by the inclusion of a semisolid matrix containing a drug solution or suspension which is in direct contact with the release liner. The component responsible for skin adhesion is incorporated in an overlay and forms a concentric configuration around the semisolid matrix.

Pharmaceutical compositions of the present disclosure can be in a form suitable for rectal administration wherein the carrier is a solid. It is preferable that the mixture forms unit dose suppositories. Suitable carriers include cocoa butter and other materials commonly used in the art. The suppositories can be conveniently formed by first admixing the composition with the softened or melted carrier(s) followed by chilling and shaping in molds.

Pharmaceutical compositions containing a compound of the present disclosure, and/or pharmaceutically acceptable salts thereof, can also be prepared in powder or liquid concentrate form.

The pharmaceutical composition (or formulation) may be packaged in a variety of ways. Generally, an article for distribution includes a container that contains the pharmaceutical composition in an appropriate form. Suitable containers are well known to those skilled in the art and include materials such as bottles (plastic and glass), sachets, foil blister packs, and the like. The container may also include a tamper proof assemblage to prevent indiscreet access to the contents of the package. In addition, the container typically has deposited thereon a label that describes the contents of the container and any appropriate warnings or instructions.

The disclosed pharmaceutical compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration. The pack or dispenser may also be accompanied with a notice associated with the container in form prescribed by a governmental agency regulating the manufacture, use, or sale of pharmaceuticals, which notice is reflective of approval by the agency of the form of the drug for human or veterinary administration. Such notice, for example, may be the labeling approved by the U.S. Food and Drug Administration for prescription drugs, or the approved product insert. Pharmaceutical compositions comprising a disclosed compound formulated in a compatible pharmaceutical carrier may also be prepared, placed in an appropriate container, and labeled for treatment of an indicated condition.

The exact dosage and frequency of administration depends on the particular disclosed compound, a product of a disclosed method of making, a pharmaceutically acceptable salt, solvate, or polymorph thereof, a hydrate thereof, a solvate thereof, a polymorph thereof, or a stereochemically isomeric form thereof; the particular condition being treated and the severity of the condition being treated; various factors specific to the medical history of the subject to whom the dosage is administered such as the age; weight, sex, extent of disorder and general physical condition of the particular subject, as well as other medication the individual may be taking; as is well known to those skilled in the art. Furthermore, it is evident that said effective daily amount may be lowered or increased depending on the response of the treated subject and/or depending on the evaluation of the physician prescribing the compounds of the present disclosure.

Depending on the mode of administration, the pharmaceutical composition will comprise from 0.05 to 99% by weight, preferably from 0.1 to 70% by weight, more preferably from 0.1 to 50% by weight of the active ingredient, and, from 1 to 99.95% by weight, preferably from 30 to 99.9% by weight, more preferably from 50 to 99.9% by weight of a pharmaceutically acceptable carrier, all percentages being based on the total weight of the composition.

In one aspect, an appropriate dosage level will generally be about 0.01 to 1000 mg of a compound described herein per kg patient body weight per day and can be administered in single or multiple doses. In various aspects, the dosage level will be about 0.1 to about 500 mg/kg per day, about 0.1 to 250 mg/kg per day, or about 0.5 to 100 mg/kg per day. A suitable dosage level can be about 0.01 to 1000 mg/kg per day, about 0.01 to 500 mg/kg per day, about 0.01 to 250 mg/kg per day, about 0.05 to 100 mg/kg per day, or about 0.1 to 50 mg/kg per day. Within this range the dosage can be 0.05 to 0.5, 0.5 to 5.0 or 5.0 to 50 mg/kg per day. For oral administration, the compositions are preferably provided in the form of tablets containing 1.0 to 1000 mg of the active ingredient, particularly 1.0, 5.0, 10, 15, 20, 25, 50, 75, 100, 150, 200, 250, 300, 400, 500, 600, 750, 800, 900 and 1000 mg of the active ingredient for the symptomatic adjustment of the dosage of the patient to be treated. The compound can be administered on a regimen of 1 to 4 times per day, preferably once or twice per day. This dosing regimen can be adjusted to provide the optimal therapeutic response.

Such unit doses as described hereinabove and hereinafter can be administered more than once a day, for example, 2, 3, 4, 5 or 6 times a day. In various aspects, such unit doses can be administered 1 or 2 times per day, so that the total dosage for a 70 kg adult is in the range of 0.001 to about 15 mg per kg weight of subject per administration. In a further aspect, dosage is 0.01 to about 1.5 mg per kg weight of subject per administration, and such therapy can extend for a number of weeks or months, and in some cases, years. It will be understood, however, that the specific dose level for any particular patient will depend on a variety of factors including the activity of the specific compound employed; the age, body weight, general health, sex and diet of the individual being treated; the time and route of administration; the rate of excretion; other drugs that have previously been administered; and the severity of the particular disease undergoing therapy, as is well understood by those of skill in the area.

A typical dosage can be one 1 mg to about 100 mg tablet or 1 mg to about 300 mg taken once a day, or multiple times per day, or one time-release capsule or tablet taken once a day and containing a proportionally higher content of active ingredient. The time-release effect can be obtained by capsule materials that dissolve at different pH values, by capsules that release slowly by osmotic pressure, or by any other known means of controlled release.

It can be necessary to use dosages outside these ranges in some cases as will be apparent to those skilled in the art. Further, it is noted that the clinician or treating physician will know how and when to start, interrupt, adjust, or terminate therapy in conjunction with individual patient response.

The disclosed pharmaceutical compositions can further comprise other therapeutically active compounds, which are usually applied in the treatment of the above mentioned pathological or clinical conditions.

It is understood that the disclosed compositions can be prepared from the disclosed compounds. It is also understood that the disclosed compositions can be employed in the disclosed methods of using.

As already mentioned, the present disclosure relates to a pharmaceutical composition comprising a therapeutically effective amount of a disclosed compound, a product of a disclosed method of making, a pharmaceutically acceptable salt, a hydrate thereof, a solvate thereof, a polymorph thereof, and a pharmaceutically acceptable carrier. Additionally, the present disclosure relates to a process for preparing such a pharmaceutical composition, characterized in that a pharmaceutically acceptable carrier is intimately mixed with a therapeutically effective amount of a compound according to the present disclosure.

### Methods for Treatment and Prevention of Malaria in Subjects

Human malaria is caused by single-celled microorganisms of the Plasmodium group. It is spread exclusively through bites of infected female Anopheles mosquitoes. The mosquito bite introduces the parasites, and the parasites travel to the liver, where they mature and reproduce. Host infection initiates with sporozoite invasion of hepatocytes, followed by a dramatic parasite amplification event during liver stage (also referred to as exoerythrocytic form (EEF)). Following liver stage development, the parasites enter the blood stream (i.e., the blood stage), where they invade red blood cells (erythrocytes). Expansion of the blood stage results in the clinical manifestation of malaria, where symptoms such as fever, chills, headaches, muscle and joint pain, abdominal pain, nausea and vomiting, and diarrhea can occur.

In one aspect, disclosed herein is a method for the treatment or prevention of a malaria in a subject, the method including the step of administering to the subject a therapeutically effective amount of at least one disclosed compound, or a pharmaceutically acceptable salt thereof, or the disclosed pharmaceutical composition. In some aspects, the subject is a human. In another aspect, the subject has been diagnosed with a need for treatment of malaria prior to the administering step. In some aspects, the method further includes the step of identifying a subject in need of treatment of malaria.

In one aspect, the compounds described herein exhibit potent antimalarial activity against one or more strains of Plasmodium spp. in the parasite's various stages, such as, for example, the erythrocytic and exoerythrocytic stages. In one aspect, the malaria parasite is Plasmodium falciparum, Plasmodium vivax, Plasmodium knowlesi, Plasmodium ovale, or Plasmodium malariae. In another aspect, the compounds described herein inhibit or prevent the liver stage of malaria, the blood stage of malaria, or a combination thereof. The Examples demonstrate the ability of the compounds described herein can inhibit both liver and blood strategy. The ability of the compounds described herein to inhibit both liver and blood stages of malaria makes them an attractive alternative for the treatment and prevention of malaria.

In one aspect, the compound is administered orally to the subject. In another aspect, the compound is administered at a dosage of from about 50 mg per day to about 1,000 mg per day, or about 50 mg per day, 50 mg per day, 100 mg per day, 150 mg per day, 200 mg per day, 250 mg per day, 300 mg per day, 350 mg per day, 400 mg per day, 450 mg per day, 500 mg per day, 550 mg per day, 600 mg per day, 650 mg per day, 700 mg per day, 750 mg per day, 800 mg per day, 850 mg per day, 900 mg per day, 950 mg per day, or 1,000 mg per day, where any value can be a lower and upper endpoint of a range (e.g., 100 mg per day to 300 mg per day).

Now having described the aspects of the present disclosure, in general, the following Examples describe some additional aspects of the present disclosure. While aspects of the present disclosure are described in connection with the following examples and the corresponding text and figures, there is no intent to limit aspects of the present disclosure to this description. On the contrary, the intent is to cover all alternatives, modifications, and equivalents included within the spirit and scope of the present disclosure.

### Aspects

Aspect 1. A compound having a formula represented by a structure I or the pharmaceutically acceptable salt thereof:

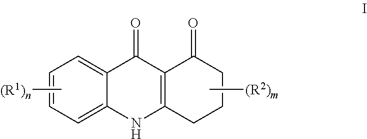

wherein
   n is an integer from 1 to 4, where each R¹ is independently hydrogen, a halide, a substituted or unsubstituted linear or branched alkyl group, or an alkoxy group, and
   m is an integer from 1 to 3, where each R² is hydrogen, a substituted or unsubstituted cycloalkyl group, a substituted or unsubstituted heterocycloalkyl group, a substituted or unsubstituted aryl group, or a substituted or unsubstituted heteroaryl group.

Aspect 2. The compound of Aspect 1, wherein n is 1 or 2.

Aspect 3. The compound of Aspect 1 or 2, wherein R¹ is a C1-C6 alkyl group.

Aspect 4. The compound of Aspect 1 or 2, wherein R¹ is a methyl or ethyl group.

Aspect 5. The compound of Aspect 1 or 2, wherein R¹ is a halide.

Aspect 6. The compound of Aspect 1 or 2, wherein R¹ is fluoride, bromide, iodide, or chloride.

Aspect 7. The compound of Aspect 1 or 2, wherein R¹ is a C1-C6 alkoxy group.

Aspect 8. The compound of Aspect 1 or 2, wherein R¹ is a methoxy or ethoxy group.

Aspect 9. The compound of any one of Aspects 1-8, wherein m is 1.

Aspect 10. The compound of any one of Aspects 1-9, wherein R² is a substituted or unsubstituted phenyl group.

Aspect 11. The compound of any one of Aspects 1-9, wherein R² is a phenyl group substituted with a halide, a substituted or unsubstituted alkyl group, an alkoxy group, or an aryloxy group.

Aspect 12. The compound of any one of Aspects 1-9, wherein R² is a substituted or unsubstituted heterocycloalkyl group.

Aspect 13. The compound of Aspect 12, wherein the substituted or unsubstituted heterocycloalkyl group is a 5- or 6-membered ring.

Aspect 14. The compound of Aspect 12, wherein the unsubstituted heterocycloalkyl group is a tetrahydrofuran group or a tetrahydropyran group.

Aspect 15. The compound of any one of Aspects 1-9, wherein R² is a substituted or unsubstituted heteroaryl group.

Aspect 16. The compound of Aspect 15, wherein the heteroaryl group is a pyridine group, a thiophene group, or a thiazole group.

Aspect 17. The compound of Aspect 15 or 16, wherein the heteroaryl group is substituted with a substituted or unsubstituted phenyl group.

Aspect 18. The compound of Aspect 17, where the phenyl group is substituted with a halide, a substituted or unsubstituted alkyl group, an alkoxy group, a fluoroalkoxy group, or a fluoroalkyl group.

Aspect 19. The compound of Aspect 17, where the phenyl group is substituted with —F, —CF₃, —OCF₃, or any combination thereof.

Aspect 20. The compound of any one of Aspects 1-9, wherein R² is a C3-C6 cycloalkyl group.

Aspect 21. The compound of Aspect 1, wherein the compound has the structure II

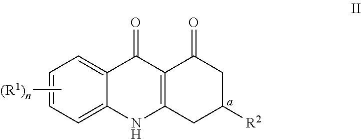

wherein the stereochemistry at carbon a is racemic or has an enantiomeric excess of at least 95% of the R or S enantiomer.

Aspect 22. The compound of Aspect 21, wherein R² is a substituted or unsubstituted phenyl group.

Aspect 23. The compound of Aspect 21, wherein R² is a phenyl group substituted with a halide, a substituted or unsubstituted alkyl group, an alkoxy group, or an aryloxy group.

Aspect 24. The compound of Aspect 21, wherein R² is a substituted or unsubstituted heterocycloalkyl group.

Aspect 25. The compound of Aspect 24, wherein the substituted or unsubstituted heterocycloalkyl group is a 5- or 6-membered ring.

Aspect 26. The compound of Aspect 24, wherein the unsubstituted heterocycloalkyl group is a tetrahydrofuran group or a tetrahydropyran group.

Aspect 27. The compound of Aspect 21, wherein R² is a substituted or unsubstituted heteroaryl group.

Aspect 28. The compound of Aspect 27, wherein the heteroaryl group is a pyridine group, a thiophene group, or a thiazole group.

Aspect 29. The compound of Aspect 27 or 28, wherein the heteroaryl group is substituted with a substituted or unsubstituted phenyl group.

Aspect 30. The compound of Aspect 29, where the phenyl group is substituted with a halide, a substituted or unsubstituted alkyl group, an alkoxy group, a fluoroalkoxy group, or a fluoroalkyl group.

Aspect 31. The compound of Aspect 29, where the phenyl group is substituted with —F, —CF₃, —OCF₃, or any combination thereof.

Aspect 32. The compound of Aspect 21, wherein R² is a C3-C6 cycloalkyl group.

Aspect 33. The compound of Aspect 21, wherein R² is

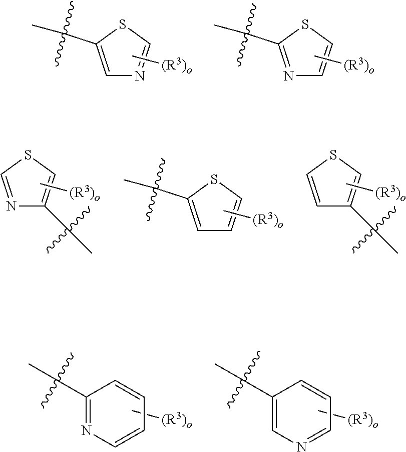

wherein o is 1 or 2 and R³ is hydrogen, a substituted or unsubstituted cycloalkyl group, a substituted or unsubstituted heterocycloalkyl group, a substituted or unsubstituted aryl group, a substituted or unsubstituted heteroaryl group, or a substituted or unsubstituted aryloxy group.

Aspect 34. The compound of Aspect 33, wherein R³ is a phenyl group substituted with a fluoroalkyl group or fluoroalkoxy group.

Aspect 35. The compound of Aspect 33, wherein R³ is a phenyl group substituted with —F, —CF₃, —OCF₃, or any combination thereof.

Aspect 36. The compound of Aspect 1, wherein the compound has one of the following structures

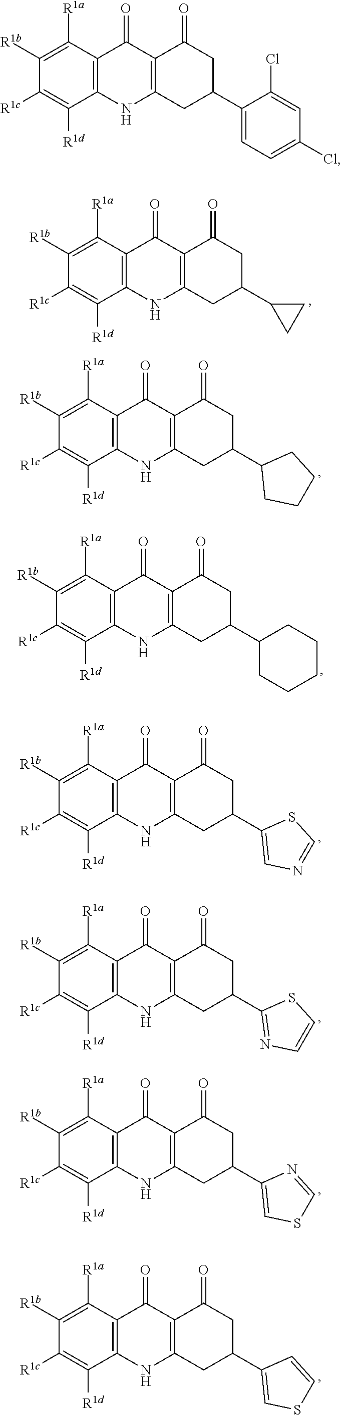

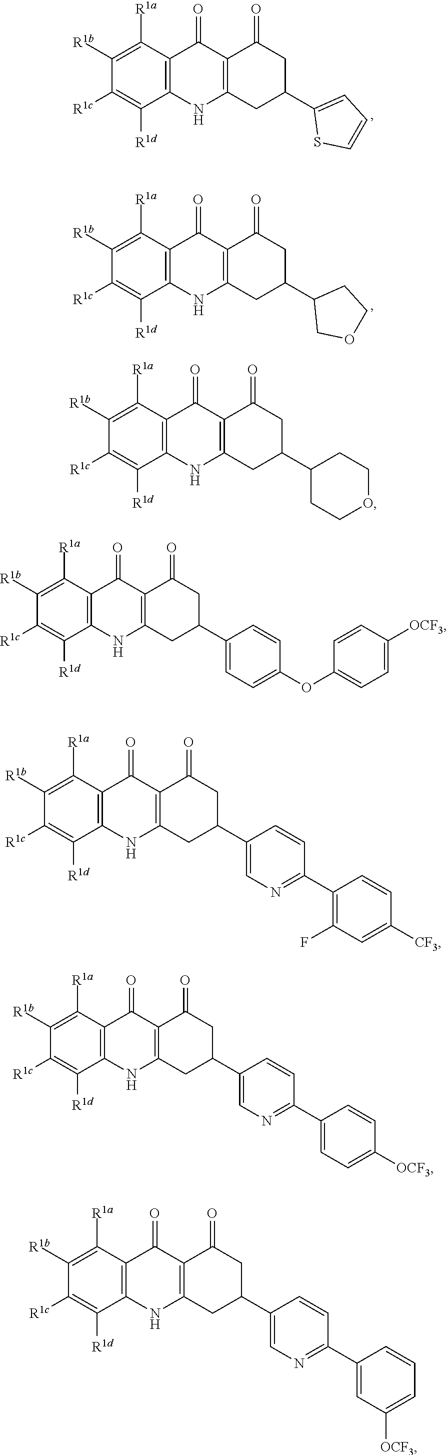

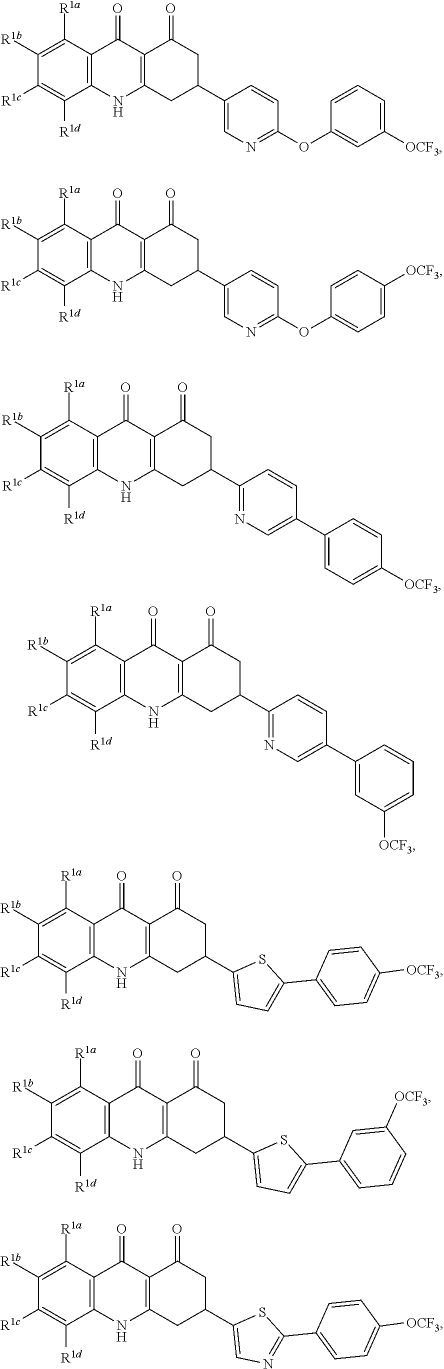

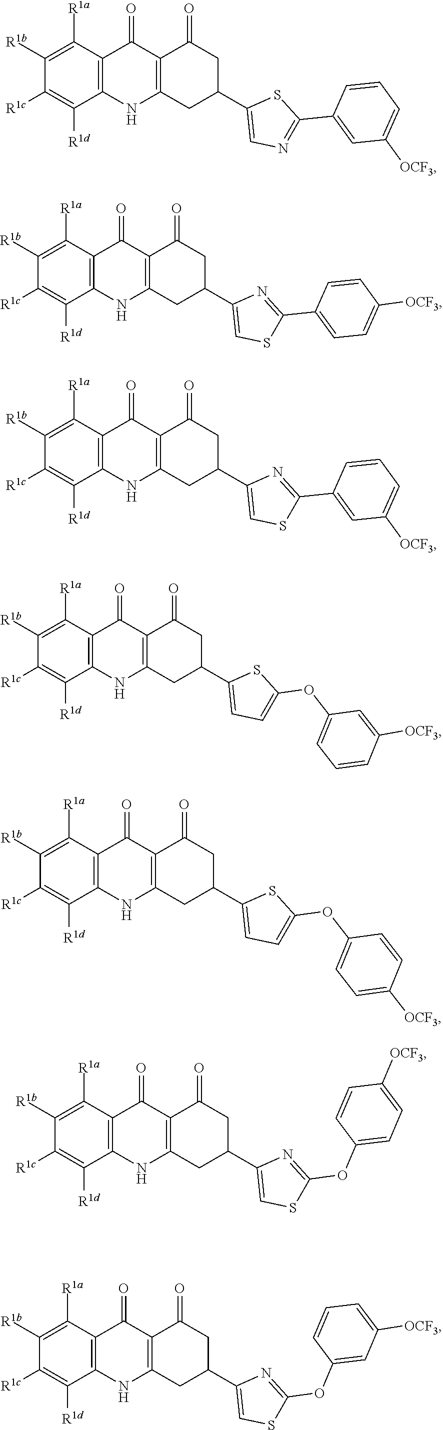

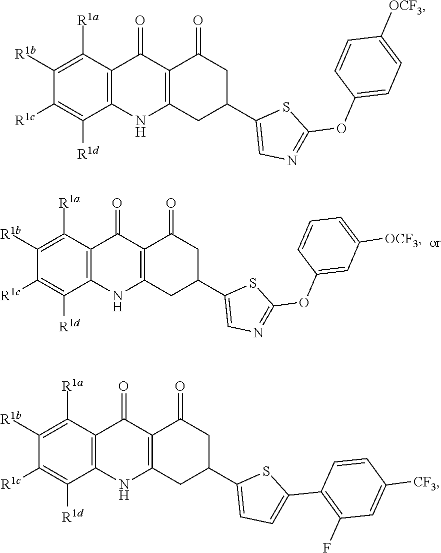

wherein R^{1a}, R^{1b}, R^{1c}, and R^{1d}, are each independently hydrogen, a halide, a substituted or unsubstituted linear or branched alkyl group, or an alkoxy group.

Aspect 37. The compound of Aspect 36, wherein the alkyl group is a methyl group, the alkoxy group is a methoxy group and the halide is chloride.

Aspect 38. The compound of Aspect 36, wherein R^{1c} is a methoxy group.

Aspect 39. The compound of Aspect 1, wherein the compound has the structure

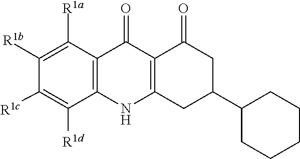

wherein R^{1c} is a methoxy group.

Aspect 40. The compound of Aspect 1, wherein the compound has the structure

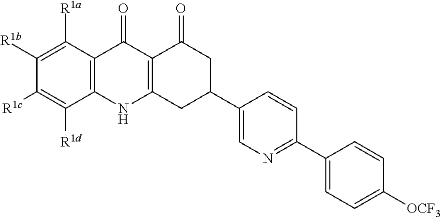

wherein R^{1b} is chloride and R^{1c} is a methoxy group.

Aspect 41. The compound of Aspect 1, wherein the compound has the structure

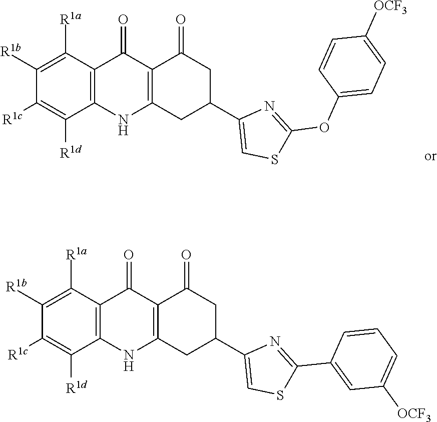

wherein R^{1b} is chloride and R^{1c} is a methoxy group.

Aspect 42. The compound of Aspect 1, wherein the compound has the structure

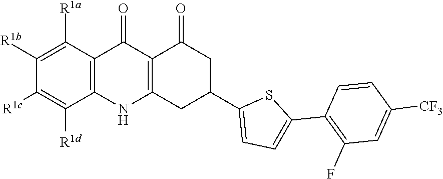

wherein R^{1c} is a methoxy group.

Aspect 43. The compound of Aspect 1, wherein the compound has the structure

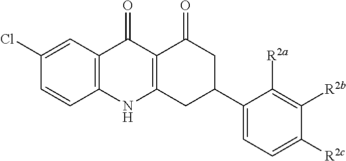

wherein R^{2a}, R^{2b}, and R^{2c}, are each independently hydrogen, a halide, a substituted or unsubstituted linear or branched alkyl group, or an alkoxy group.

Aspect 44. The compound of Aspect 43, wherein R^{2c} is a methoxy group.

Aspect 45. The compound of Aspect 1, wherein the compound is THA-1839, THA-1840, THA-1994, THA-2111, THA-2209, THA-2220, THA-2224, THA-2224, or THA-2225.

Aspect 46. A pharmaceutical composition comprising the compound of any one of claims 1 to 45 and a pharmaceutically-acceptable carrier.

Aspect 47. A method for treating or preventing malaria in a subject, the method comprising administering to the subject an effective amount of the compound of any one of Aspects 1 to 45.

Aspect 48. The method of Aspect 47, wherein the compound inhibits or prevents the liver stage of malaria, the blood stage of malaria, or a combination thereof.

Aspect 49. The method of Aspect 47, wherein the subject is infected with a strain of Plasmodium spp. that infects the subject with malaria.

Aspect 50. The method of Aspect 49, wherein the strain of Plasmodium spp. comprises Plasmodium falciparum, Plasmodium vivax, Plasmodium knowlesi, Plasmodium ovale, or Plasmodium malariaei.

### Examples

The following examples are put forth so as to provide those of ordinary skill in the art with a complete disclosure and description of how the compounds, compositions, articles, devices and/or methods claimed herein are made and evaluated and are intended to be purely exemplary of the disclosure and are not intended to limit the scope of what the inventors regard as their disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure.

### Synthetic Procedures

### Experimental

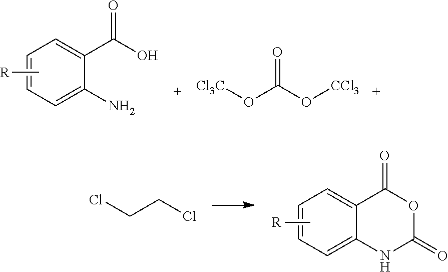

### General Procedure A: Formation of Isatoic Anhydride

2-necked round bottom flask was charged with an anthranilic acid (1 equivalence) in 1,2-dichloroethane (485 mM). To the above solution was added dropwise a solution of triphosgene (2 equivalences) in 1,2-dichloroethane (1.05 M) at room temperature. The resulting mixture was heated to 80° C. stirred for an additional 3 h then cooled in ice-water. The precipitate was collected by filtration, washed with ice cold dichloromethane, and dried to afford the product. (75-95%)

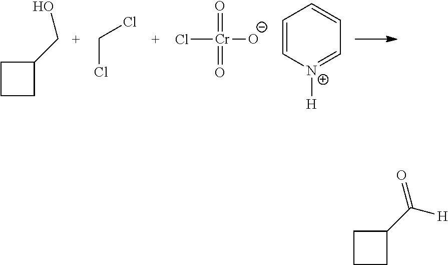

### General Procedure B: Pyridinium Chlorochromate Oxidation (Formation of Aldehyde)

To a solution of pyridinium chlorochromate (2 equivalences) in dichloromethane (300 mM) under an inert condition at 0° C., added alcohol (1 equivalence). The reaction mixture was stirred at room temperature for 4 hours. Once completed, the reaction mixture was filtered through a small plug of celite and the solvent was evaporated under reduced pressure. The crude product was used directly without further purification.

### General Procedure C: Swern Oxidation (Formation of Aldehyde)

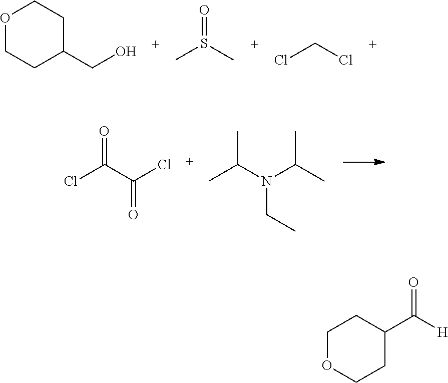

To an oven dried 2-neck round bottom flask, a solution of oxalyl chloride (1.5 equivalences) in dichloromethane (2 M) was added, sequentially, dimethyl sulfoxide (3 equivalences) and alcohol (1 equivalence) at −78° C. N,N-diisopropylethylamine (5.5 equivalences) was then added over 5 minutes and the reaction flask was removed from the bath and allowed to warm to 0° C. over 10 minutes. The reaction mixture was transferred to a separatory funnel charged with ice-cold 1M hydrochloric acid solution. The two phases were separated and the aqueous layer was further extracted with dichloromethane. The combined organic layers were washed with brine and dried over anhydrous sodium sulfate. The solvent was evaporated at reduced pressure. The crude product was used directly without further purification.

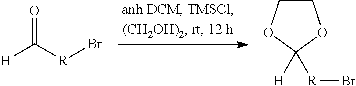

General Procedure D: Protection of Aldehyde with Cyclic Acetal

To an oven dried 2-neck round bottom flask, a solution of aldehyde (1 equivalence) in DCM (113 mM) was added under inert conditions. To this, ethylene glycol (3 equivalences) and TMSCI (1 equivalence) were added at room temperature The reaction mixture was stirred at room temperature for 12 hours. The mixture was extracted with extracted with DCM. The combined organic layers were washed with brine and dried over anhydrous sodium sulfate. The solvent was evaporated at reduced pressure. The crude product was used directly without further purification.

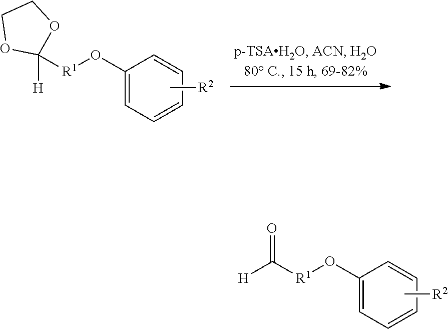

### General Procedure E: Deprotection of Aldehyde (Deprotection of Cyclic Acetal)

To a solution of protected biaryl ether (1 equivalence) in acetonitrile (54 mM) and water (273 mM), p-toluenesulfonic acid monohydrate (0.2 equivalence) was added at room temperature under inert condition. The reaction mixture was stirred at reflux for 4 hours and cooled to room temperature. Upon the completion of the reaction, the reaction was quenched with sodium bicarbonate. The mixture was extracted with ethyl acetate and brine. The combined organic layers were washed with brine and dried over anhydrous sodium sulfate. The solvent was evaporated at reduced pressure. The crude was saved under inert condition, as the crude is unstable under open air. The residue was purified through column chromatography to afford the product.

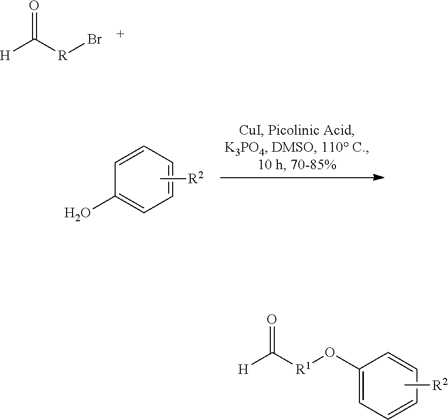

### General Procedure F: Ullman Coupling (Formation of Ether Linkage)

To an oven dried 2-neck round bottom flask, a solution of aldehyde (1 equivalence) and phenol (1.1 equivalences) in anhydrous dimethyl sulfoxide (250 mM) was added, sequentially, potassium phosphate (2 equivalences), picolinic acid (0.4 equivalence), and copper iodide (0.2 equivalence) under inert conditions. The reaction mixture was stirred at reflux overnight and cooled to room temperature. The mixture was extracted with ethyl acetate and brine. The combined organic layers were washed with brine and dried over anhydrous sodium sulfate. The solvent was evaporated at reduced pressure. The residue was purified through column chromatography to afford the product or 90% pure, depending on substrate.

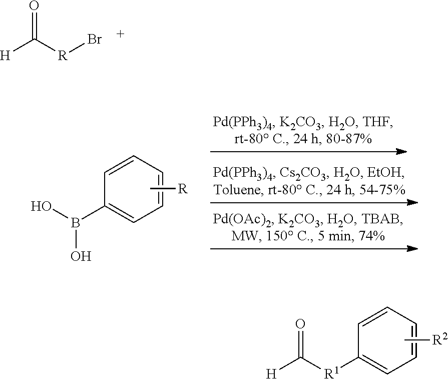

General Procedure G: Suzuki Coupling (Formation of the CH₂—CH₂ Bond)

To a flame dried reflux apparatus, a 2-neck round bottom was charged with aldehyde (1 equivalence), boronic acid (1.3 equivalences), and cesium carbonate (2 equivalences). To this, add anhydrous toluene (800 mM), anhydrous ethanol (800 mM), and deionized water (130 mM). Allow this solution to stir at room temperature for 5 minutes. To this reaction mixture, add tetrakis(triphenylphosphine)palladium (0) (0.11 equivalence). The reaction was stirred at reflux for 3 hours and cooled to room temperature upon completion. The mixture was filtered through a small plug of celite and washed with ethyl acetate. The ethanol was evaporated at reduced pressure. This mixture was extracted with ethyl acetate and the combined organic layers were washed with brine and dried over anhydrous sodium sulfate. The solvent was evaporated at reduced pressure. The residue was purified through column chromatography to afford the product or 90% pure, depending on substrate.

General Procedure H: Suzuki Coupling (Formation of the CH₂—CH₂ Bond)

To an oven dried 2-neck round bottom, a solution of potassium carbonate (3 equivalences) in anhydrous tetrahydrofuran (369 mM) and deionized water (986 mM) was added, sequentially, aldehyde (1 equivalence) and tetrakis(triphenylphosphine)palladium(0) (0.08 equivalence). Allow this solution to stir at room temperature for 1 hour under inert conditions. To this reaction mixture, add boronic acid (1.1 equivalences). The reaction was stirred at reflux overnight and cooled to room temperature upon completion. The mixture was filtered through a small plug of celite and washed with ethyl acetate. This mixture was extracted with ethyl acetate and the combined organic layers were washed with brine and dried over anhydrous sodium sulfate. The solvent was evaporated at reduced pressure. The residue was purified through column chromatography to afford the product or 90% pure, depending on substrate.

General Procedure I: Suzuki Coupling (Formation of the CH₂—CH₂ Bond)

A microwave vessel was charged with a solution of aldehyde (1 equivalence), boronic acid (1.2 equivalences), potassium carbonate (3 equivalences), palladium acetate (0.004 equivalence), and tetrabutylammonium bromide (1 equivalence) in deionized water (250 mM). The microwave reaction was set to 150° C. for 5 minutes. The reaction mixture was diluted with ethyl acetate and filtered through a small plug of celite. The solution was extracted with ethyl acetate. The combined organic layers were washed with brine and dried over anhydrous sodium sulfate. The solvent was evaporated at reduced pressure. The residue was purified through column chromatography to afford the product or 90% pure, depending on substrate.

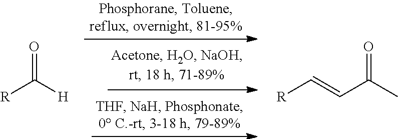

### General Procedure J for the Formation of the α,β-Unsaturated Ketone (Aldol Condensation)

To a solution of aldehyde (1 equivalence) in acetone (4 M) and water (1 M), added 5% aqueous sodium hydroxide solution (10 M). The reaction mixture was stirred at room temperature for 30 minutes. This mixture was extracted with ethyl acetate. The combined organic layers were washed with brine and dried over anhydrous sodium sulfate. The solvent was evaporated under reduced pressure.

### General Procedure K for the Formation of the α,β-Unsaturated Ketone (Wittig Reaction)

To a flame dried reflux apparatus, solution of aldehyde (1 equivalence) in toluene (500 mM) was added. To this solution, (acetylmethylene)triphenylphosphorane (1.2 equivalences) was added. The mixture was stirred at reflux overnight, cooled to room temperature, and evaporated under reduced pressure. The residue was purified through column chromatography to afford the enone. dx.doi.org/10.1021/jo5006729

### General Procedure L for the Formation of the α,β-Unsaturated Ketone (Wittig Reaction)

Under inert conditions, round bottom flask was charged with aldehyde (1 equivalences) in DCM (600 mM). To this solution, ylide was added (1.5 equivalences). The resulting mixture was left stirring at room temperature overnight. This was extracted with ethyl acetate and the combined organic layers were washed with brine. This was dried over anhydrous sodium sulfate. The solvent was evaporated at reduced pressure.

### General Procedure M for the Formation of the α,β-Unsaturated Ketone (Wittig Reaction)

Aldehyde (5 equivalences) was added to suspension of ylide (1 equivalence) in toluene (113 mM). This was left to stir at room temperature for 4 days This was extracted with ethyl acetate and the combined organic layers were washed with brine. This was dried over anhydrous sodium sulfate. The solvent was evaporated at reduced pressure.

### General Procedure N for the Formation of the α,β-Unsaturated Ketone (Horner-Wadsworth-Emmons Reaction)

A round bottom flask was charged with potassium carbonate (2.7 equivalences) and water (6 M). To the above solution was added phosphonate (0.9 equivalence) followed by carboxaldehyde (1 equivalence). The resulting mixture was stirred overnight at room temperature. This was extracted with diethyl ether. The combined organic layers were washed with brine and dried over anhydrous sodium sulfate. The solvent was evaporated at reduced pressure at room temperature.

### General Procedure O for the Formation of the α,β-Unsaturated Ketone (Horner-Wadsworth-Emmons Reaction)

A round bottom flask was charged with NaH (1.05 equivalences) in tetrahydrofuran (200 mM) under inert conditions at 0° C. To the above solution, add phosphonate (1.5 equivalences). Stir 30 min at room temperature. A solution of aldehyde (1 equivalence) in tetrahydrofuran (200 mM) was added dropwise. Stir overnight at room temperature. This was extracted with ethyl acetate and the combined organic layers were washed with brine. This was dried over anhydrous sodium sulfate. The solvent was evaporated at reduced pressure.

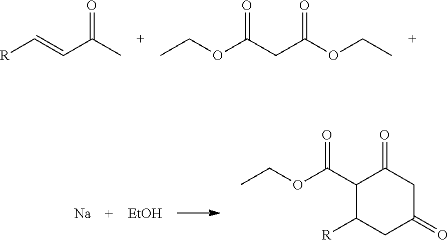

### General Procedure P for the Formation of Ethyl-2,4-Dioxocyclohexane-1-Carboxylate

Under inert conditions, 2-neck round bottom flask was charged with diethylmalonate. To the above was added dropwise an in situ generated sodium ethoxide. The mixture was stirred for 20 minutes at room temperature. This was followed by the addition of a solution of enone in ethanol. The solution was refluxed for 5 min to 18 hours, then allowed to cool to room temperature. After the completion of the reaction, acidify the reaction with 6M HCl to pH 6. This mixture was extracted with ethyl acetate. The combined organic layers were washed with brine and dried over anhydrous sodium sulfate. The solvent was evaporated under reduced pressure. Purify via column until 85-95% pure. Proceed without further purification.

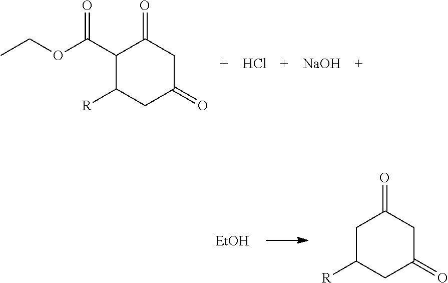

### General Procedure Q for the Formation of Cyclic Diketone

2-neck round bottom flask was charged with ethyl-2,4-dioxocyclohexane-1-carboxylate in ethanol. To the above was added dropwise a 2M solution of sodium hydroxide. The resulting mixture was refluxed for 2 hours. Upon confirmation the formation of intermediate carboxylic acid, a solution of 4M hydrochloric acid was added and further refluxed for an additional hour. After the completion of the reaction, the mixture was extracted with ethyl acetate. The combined organic layers were washed with brine and dried over anhydrous sodium sulfate. The solvent was evaporated under reduced pressure.

### General Procedure R: Synthesis of THAs

2-neck round bottom flask was charged with corresponding dione (0.9 equivalence) in anhydrous dimethylformamide (350 mM). To the above was added sodium hydride (1.1 equivalences). The resulting mixture was heated to 50° C. for 30 minutes. Upon cooling, this was followed by the addition of corresponding isatoic anhydride. This solution was refluxed for 2-4 hours, then allowed to cool to room temperature. After the completion of the reaction, acidify the reaction with 6M HCl to pH 3. This mixture was extracted with ethyl acetate. The combined organic layers were washed with brine and dried over anhydrous sodium sulfate. The solvent was evaporated under reduced pressure. Purify via recrystallization.

### Specific Synthetic Procedures and Characterization

### 6-chloro-2H-benzo[d][1,3]oxazine-2,4(1H)-dione (2)

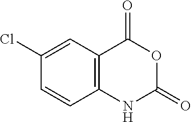 [SMILES: O=c1[nH]c2ccc(Cl)cc2c(=O)o1]

Following general procedure A, the title compound was prepared as a white powder.

LCMS (ESI-MS) m/z: [M+H]⁺ calcd for C₈H₅ClNO₃ 197.99; found 197.8. Retention time: 2.921 min.

TLC: (50% EtOAc in hexanes, R_{f}): 0.34 (UV, 254 nm, 280 nm).

¹H NMR (500 MHz, DMSO-d₆) δ 11.86 (s, 1H), 7.87 (d, J=2.4 Hz, 1H), 7.78 (dd, J=8.8, 2.4 Hz, 1H), 7.16 (d, J=8.8 Hz, 1H).

¹³C NMR (126 MHz, DMSO-d₆) δ 158.9, 146.7, 140.3, 136.6, 127.6, 127.1, 117.4, 112.0.

### (E)-4-(2,4-dichlorophenyl)but-3-en-2-one (4)

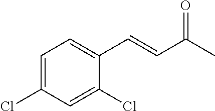

Following general procedure D, the title compound was prepared as a white powder.

LCMS (ESI-MS) m/z: [M+H]⁺ calcd for C₁₀H₉Cl₂O 215.00; found 215.0. Retention time: 3.514 min.

TLC: (10% EtOAc in hexanes, R_{f}): 0.24 (UV, 254 nm, 280 nm).

¹H NMR (500 MHz, DMSO-d₆) δ 7.93 (d, J=8.5 Hz, 1H), 7.77-7.69 (m, 2H), 7.48 (dd, J=8.5, 2.1 Hz, 1H), 6.93 (d, J=16.3 Hz, 1H), 2.36 (s, 3H).

¹³C NMR (126 MHz, DMSO-d₆) δ 198.0, 136.4, 135.9, 135.1, 131.6, 130.6, 129.9, 129.8, 128.4, 28.5.

### 2′,4′-dichloro-5-hydroxy-1,6-dihydro-[1,1′-biphenyl]-3(2H)-one (6)

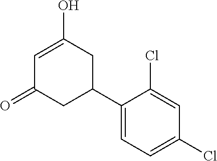 [SMILES: O=C1C=C(O)CC(c2ccc(Cl)cc2Cl)C1]

Following general procedures J and K, the title compound was prepared as a white powder.

LCMS (ESI-MS) m/z: [M+H]⁺ calcd for C₁₂H₁₁Cl₂O₂ 257.01; found 256.7. Retention time: 3.836 min.

TLC: (5% methanol in dichloromethane, R_{f}): 0.22 (UV, 254 nm, 280 nm).

¹H NMR (500 MHz, DMSO-d₆) δ 11.28 (s, 1H), 7.59 (d, J=2.2 Hz, 1H), 7.50 (d, J=8.5 Hz, 1H), 7.41 (dd, J=8.5, 2.2 Hz, 1H), 5.27 (s, 1H), 3.66-3.58 (m, 1H), 2.68-2.53 (m, 2H), 2.44-2.28 (m, 2H).

¹³C NMR (126 MHz, DMSO-d₆) δ 144.0, 128.9, 127.3, 127.0, 104.0, 39.2.

IR (thin film, cm⁻¹): v=2468, 2160, 1558, 1474, 1387, 1251, 1202, 1154, 1095, 1052, 907, 865, 833, 755, 360, 570, 462.

### 7-chloro-3-(2,4-dichlorophenyl)-3,4-dihydroacridine-1,9(2H,10H)-dione (7)

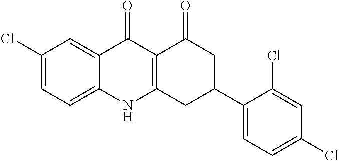 [SMILES: O=C1CC(c2ccc(Cl)cc2Cl)Cc2[nH]c3ccc(Cl)cc3c(=O)c21]

Following general procedure L, the title compound was prepared as a white solid.

TLC: (5% methanol in dichloromethane, R_{f}): 0.38 (UV, 254 nm, 280 nm).

¹H NMR (500 MHz, CDCl₃ and 3 drops of TFA-d) δ 8.50 (d, J=2.0 Hz, 1H), 8.12-8.01 (m, 2H), 7.50 (d, J=2.1 Hz, 1H), 7.34 (dd, J=8.4, 2.1 Hz, 1H), 7.25 (d, J=8.4 Hz, 1H), 4.21-4.08 (m, 1H), 3.76-3.60 (m, 2H), 3.23 (ddd, J=17.8, 4.0, 1.5 Hz, 1H), 3.14 (dd, J=17.8, 12.6 Hz, 1H).

¹³C NMR (126 MHz, CDCl₃ and 3 drops of TFA-d) δ 202.2, 172.4, 161.0, 139.0, 137.9, 136.5, 134.9, 134.8, 134.2, 130.4, 128.2, 127.6, 124.2, 122.1, 120.2, 108.1, 42.5, 34.6, 33.4.

### 7-chloro-3-(2-chlorophenyl)-3,4-dihydroacridine-1,9(2H,10H)-dione (8)

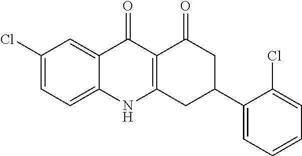 [SMILES: O=C1CC(c2ccccc2Cl)Cc2[nH]c3ccc(Cl)cc3c(=O)c21]

Following general procedure L, the title compound was prepared as a white powder.

LCMS (ESI-MS) m/z: [M+H]⁺ calcd for C₁₉H₁₄Cl₂NO₂ 358.03; found 358.0. Retention time: 2.877 min.

TLC: (5% methanol in dichloromethane, R_{f}): 0.32 (UV, 254 nm, 280 nm).

¹H NMR (500 MHz, CDCl₃, 1 drop of DMF-d, and 3 drops of TFA-d) δ 8.49 (d, J=2.1 Hz, 1H), 8.13-8.03 (m, 2H), 7.47 (dd, J=7.6, 1.7 Hz, 1H), 7.38-7.27 (m, 3H), 4.24-4.14 (m, 1H), 3.79-3.63 (m, 2H), 3.26 (ddd, J=17.8, 4.2, 1.6 Hz, 1H), 3.16 (dd, J=17.8, 12.5 Hz, 1H).

¹³C NMR (126 MHz, CDCl₃, 1 drop of DMF-d, and 3 drops of TFA-d) δ 202.7, 172.4, 164.7, 138.8, 138.1, 136.3, 136.2, 133.5, 130.6, 129.5, 127.9, 126.6, 124.1, 122.3, 120.3, 108.2, 42.7, 34.9, 33.5.

### 7-chloro-3-(3-chlorophenyl)-3,4-dihydroacridine-1,9(2H,10H)-dione (9)

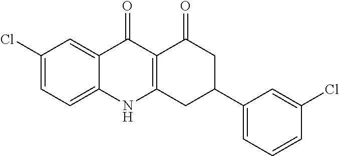 [SMILES: O=C1CC(c2cccc(Cl)c2)Cc2[nH]c3ccc(Cl)cc3c(=O)c21]

Following general procedure L, the title compound was prepared as a yellow solid.

TLC: (5% methanol in dichloromethane, R_{f}): 0.32 (UV, 254 nm, 280 nm).

¹H NMR (500 MHz, CDCl₃ and 3 drops of TFA-d) δ 8.49 (d, J=2.0 Hz, 1H), 8.12-8.03 (m, 2H), 7.40-7.32 (m, 2H), 7.27 (d, J=2.3 Hz, 1H), 7.22-7.14 (m, 1H), 3.82-3.51 (m, 3H), 3.30-3.07 (m, 2H).

¹³C NMR (126 MHz, CDCl₃ and 3 drops of TFA-d) δ 202.4, 172.4, 161.1, 141.0, 139.0, 138.0, 136.5, 135.3, 130.8, 128.7, 126.8, 124.7, 124.2, 122.2, 120.2, 108.2, 43.8, 37.9, 35.0.

### 7-chloro-3-(4-chlorophenyl)-3,4-dihydroacridine-1,9(2H,10H)-dione (10)

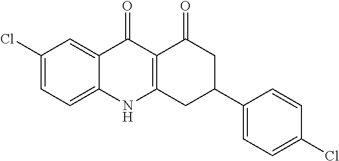 [SMILES: O=C1CC(c2ccc(Cl)cc2)Cc2[nH]c3ccc(Cl)cc3c(=O)c21]

Following general procedure L, the title compound was prepared as a white powder.

LCMS (ESI-MS) m/z: [M+H]⁺ calcd for C₁₉H₁₄Cl₂NO₂ 358.03; found 358.0. Retention time: 2.886 min.

TLC: (5% methanol in dichloromethane, R_{f}): 0.32 (UV, 254 nm, 280 nm).

¹H NMR (500 MHz, CDCl₃ and 3 drops of TFA-d) δ 8.49 (d, J=2.1 Hz, 1H), 8.12-8.02 (m, 2H), 7.39 (d, J=8.1 Hz, 2H), 7.20 (d, J=8.1 Hz, 2H), 3.79-3.66 (m, 2H), 3.61 (dd, J=18.4, 12.1 Hz, 1H), 3.26-3.11 (m, 2H).

¹³C NMR (126 MHz, CDCl₃ and 3 drops of TFA-d) δ 202.6, 172.4, 161.2, 139.0, 137.9, 137.5, 136.6, 134.4, 129.7, 127.8, 124.3, 122.2, 120.3, 108.2, 43.9, 37.7, 35.2.

### 7-chloro-3-(o-tolyl)-3,4-dihydroacridine-1,9(2H,10H)-dione (11)

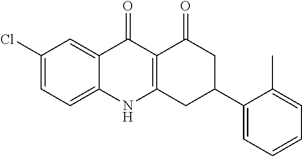 [SMILES: Cc1ccccc1C1CC(=O)c2c([nH]c3ccc(Cl)cc3c2=O)C1]

Following general procedure L, the title compound was prepared as a light brown solid.

LCMS (ESI-MS) m/z: [M+H]⁺ calcd for C₂₀H₁₇ClNO₂ 338.09; found 338.1. Retention time: 2.812 min.

TLC: (5% methanol in dichloromethane, R_{f}): 0.35 (UV, 254 nm, 280 nm).

¹H NMR (500 MHz, CDCl₃ and 3 drops of TFA-d) δ 8.50 (d, J=2.1 Hz, 1H), 8.09-8.01 (m, 2H), 7.31-7.26 (m, 3H), 7.25-7.20 (m, 1H), 4.01-3.91 (m, 1H), 3.65-3.54 (m, 2H), 3.20-3.12 (m, 2H), 2.35 (s, 3H).

¹³C NMR (126 MHz, CDCl₃ and 3 drops of TFA-d) δ 203.3, 172.5, 161.8, 139.0, 137.9, 137.2, 136.5, 135.5, 131.5, 128.2, 127.1, 124.6, 124.2, 122.1, 120.3, 108.2, 43.7, 34.7, 34.3, 19.0.

### 7-chloro-3-(m-tolyl)-3,4-dihydroacridine-1,9(2H,10H)-dione (12)

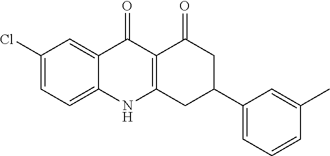 [SMILES: Cc1cccc(C2CC(=O)c3c([nH]c4ccc(Cl)cc4c3=O)C2)c1]

Following general procedure L, the title compound was prepared as a light brown solid.

LCMS (ESI-MS) m/z: [M+H]⁺ calcd for C₂₀H₁₇ClNO₂ 338.09; found 338.1. Retention time: 2.852 min.

TLC: (5% methanol in dichloromethane, R_{f}): 0.38 (UV, 254 nm, 280 nm).

¹H NMR (500 MHz, CDCl₃ and 3 drops of TFA-d) δ 8.50 (d, J=2.3 Hz, 1H), 8.12-7.95 (m, 2H), 7.29 (dd, J=8.8, 7.6 Hz, 1H), 7.17 (d, J=7.6 Hz, 1H), 7.10-6.99 (m, 2H), 3.77-3.56 (m, 3H), 3.29-3.14 (m, 2H), 2.37 (s, 3H).

¹³C NMR (126 MHz, CDCl₃ and 3 drops of TFA-d) δ 203.0, 172.5, 161.5, 139.4, 139.0, 138.8, 137.7, 136.5, 129.3, 129.1, 127.1, 124.2, 123.1, 121.8, 120.2, 108.2, 43.9, 38.1, 35.4, 21.1.

### 7-chloro-3-(p-tolyl)-3,4-dihydroacridine-1,9(2H,10H)-dione (13)

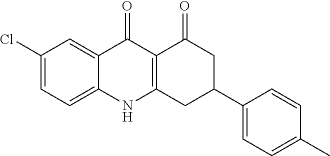 [SMILES: Cc1ccc(C2CC(=O)c3c([nH]c4ccc(Cl)cc4c3=O)C2)cc1]

Following general procedure L, the title compound was prepared as a yellowish-white powder.

LCMS (ESI-MS) m/z: [M+H]⁺ calcd for C₂₀H₁₇ClNO₂ 338.09; found 338.1. Retention time: 2.867 min.

TLC: (5% methanol in dichloromethane, R_{f}): 0.38 (UV, 254 nm, 280 nm).

¹H NMR (500 MHz, CDCl₃ and 3 drops of TFA-d) δ 8.48 (s, 1H), 8.09-8.02 (m, 2H), 7.21 (d, J=7.9 Hz, 2H), 7.15 (d, J=7.9 Hz, 2H), 3.77-3.55 (m, 3H), 3.28-3.10 (m, 2H), 2.36 (s, 3H).

¹³C NMR (126 MHz, CDCl₃ and 3 drops of TFA-d) δ 203.2, 172.4, 161.6, 138.9, 138.3, 137.9, 136.4, 136.1, 130.1, 126.3, 124.2, 122.2, 120.3, 108.3, 44.1, 37.8, 35.5, 21.0.

### 7-chloro-3-(2,4-dimethylphenyl)-3,4-dihydroacridine-1,9(2H,10H)-dione (14)

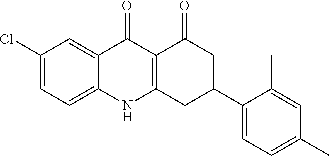 [SMILES: Cc1ccc(C2CC(=O)c3c([nH]c4ccc(Cl)cc4c3=O)C2)c(C)c1]

Following general procedure L, the title compound was prepared as a brown solid.

LCMS (ESI-MS) m/z: [M+H]⁺ calcd for C₂₁H₁₉ClNO₂ 352.10; found 352.1. Retention time: 3.033 min.

TLC: (5% methanol in dichloromethane, R_{f}): 0.32 (UV, 254 nm, 280 nm).

¹H NMR (500 MHz, CDCl₃ and 3 drops of TFA-d) δ 8.49 (d, J=1.9 Hz, 1H), 8.09-8.01 (m, 2H), 7.14-7.05 (m, 3H), 3.96-3.83 (m, 1H), 3.68-3.50 (m, 2H), 2.39-2.23 (m, 8H).

¹³C NMR (126 MHz, CDCl₃ and 3 drops of TFA-d) δ 203.3, 172.4, 161.8, 138.9, 138.0, 137.8, 136.4, 135.3, 134.2, 132.2, 127.6, 124.5, 124.2, 122.0, 120.2, 108.2, 43.8, 34.8, 34.0, 20.8, 18.8.

### 7-chloro-3-(2-methoxyphenyl)-3,4-dihydroacridine-1,9(2H,10H)-dione (15)

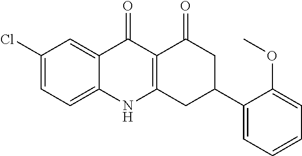 [SMILES: COc1ccccc1C1CC(=O)c2c([nH]c3ccc(Cl)cc3c2=O)C1]

Following general procedure L, the title compound was prepared as a tan-colored solid.

LCMS (ESI-MS) m/z: [M+H]⁺ calcd for C₂₀H₁₇ClNO₃ 354.08; found 354.1. Retention time: 2.791 min.

TLC: (5% methanol in dichloromethane, R_{f}): 0.35 (UV, 254 nm, 280 nm).

¹H NMR (500 MHz, CDCl₃ and 3 drops of TFA-d) δ 8.48 (s, 1H), 8.09-8.01 (m, 2H), 7.34 (dd, J=7.8, 7.2 Hz, 1H), 7.13 (d, J=7.2 Hz, 1H), 7.00-6.94 (m, 2H), 4.01-3.91 (m, 1H), 3.85 (s, 3H), 3.77 (dd, J=18.0, 12.2 Hz, 1H), 3.67 (dd, J=17.9, 4.0 Hz, 1H), 3.37 (dd, J=18.0, 12.2 Hz, 1H), 3.17 (dd, J=17.9, 4.0 Hz, 1H).

¹³C NMR (126 MHz, CDCl₃ and 3 drops of TFA-d) δ 203.9, 172.4, 162.4, 157.0, 138.7, 137.9, 136.2, 129.5, 127.1, 126.9, 124.1, 122.1, 121.1, 120.2, 111.1, 108.2, 55.2, 42.2, 34.1, 33.3.

### 7-chloro-3-(3-methoxyphenyl)-3,4-dihydroacridine-1,9(2H,10H)-dione (16)

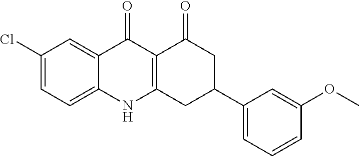 [SMILES: COc1cccc(C2CC(=O)c3c([nH]c4ccc(Cl)cc4c3=O)C2)c1]

Following general procedure L, the title compound was prepared as a white solid.

LCMS (ESI-MS) m/z: [M+H]⁺ calcd for C₂₀H₁₇ClNO₃ 354.08; found 354.1. Retention time: 2.666 min.

TLC: (5% methanol in dichloromethane, R_{f}): 0.35 (UV, 254 nm, 280 nm).

¹H NMR (500 MHz, CDCl₃ and 3 drops of TFA-d) δ 8.49 (d, J=1.9 Hz, 1H), 8.10-8.02 (m, 2H), 7.34 (dd, J=8.7, 8.2 Hz, 1H), 6.91 (dd, J=8.2, 2.1 Hz, 1H), 6.88 (d, J=8.7 Hz, 1H), 6.84 (d, J=2.1 Hz, 1H), 3.85 (s, 3H), 3.78-3.58 (m, 3H), 3.27-3.15 (m, 2H).

¹³C NMR (126 MHz, CDCl₃ and 3 drops of TFA-d) δ 202.9, 172.4, 161.4, 159.9, 140.8, 138.9, 137.9, 136.4, 130.7, 124.2, 122.2, 120.3, 119.0, 113.4, 112.9, 108.3, 55.5, 43.9, 38.1, 35.3.

### 7-chloro-3-(4-methoxyphenyl)-3,4-dihydroacridine-1,9(2H,10H)-dione (17)

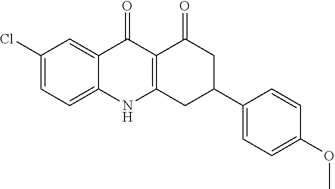 [SMILES: COc1ccc(C2CC(=O)c3c([nH]c4ccc(Cl)cc4c3=O)C2)cc1]

Following general procedure L, the title compound was prepared as a white powder.

TLC: (5% methanol in dichloromethane, R_{f}): 0.35 (UV, 254 nm, 280 nm).

¹H NMR (500 MHz, CDCl₃ and 3 drops of TFA-d) δ 8.48 (s, 1H), 8.11-8.01 (m, 2H), 7.20 (d, J=8.7 Hz, 2H), 6.95 (d, J=8.7 Hz, 2H), 3.85 (s, 3H), 3.76-3.66 (m, 2H), 3.63-3.56 (m, 1H), 3.26-3.11 (m, 2H).

¹³C NMR (126 MHz, CDCl₃ and 3 drops of TFA-d) δ 203.1, 172.4, 161.6, 159.1, 138.9, 137.9, 136.4, 131.6, 127.6, 124.2, 122.2, 120.2, 114.9, 108.3, 55.5, 44.2, 37.5, 35.6.

### 7-chloro-3-(2,4-dimethoxyphenyl)-3,4-dihydroacridine-1,9(2H,10H)-dione (18)

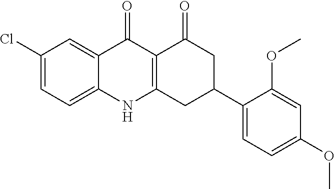 [SMILES: COc1ccc(C2CC(=O)c3c([nH]c4ccc(Cl)cc4c3=O)C2)c(OC)c1]

Following general procedure L, the title compound was prepared as a light brown solid.

LCMS (ESI-MS) m/z: [M+H]⁺ calcd for C₂₁H₁₉ClNO₄ 384.09; found 384.1. Retention time: 2.811 min.

TLC: (5% methanol in dichloromethane, R_{f}): 0.47 (UV, 254 nm, 280 nm).

¹H NMR (500 MHz, CDCl₃ and 3 drops of TFA-d) δ 8.47 (s, 1H), 8.05 (s, 2H), 7.08-6.99 (m, 1H), 6.55 (d, J=2.2 Hz, 1H), 6.50 (dd, J=8.4, 2.2 Hz, 1H), 3.92-3.79 (m, 7H), 3.72 (dd, J=17.8, 12.1 Hz, 1H), 3.63 (dd, J=17.8, 3.3 Hz, 1H), 3.34 (dd, J=17.8, 12.1 Hz, 1H), 3.14 (dd, J=17.8, 3.3 Hz, 1H).

¹³C NMR (126 MHz, CDCl₃ and 3 drops of TFA-d) δ 204.1, 172.4, 162.5, 158.1, 138.7, 137.8, 136.2, 127.7, 124.1, 122.0, 120.2, 119.8, 108.2, 104.8, 99.3, 55.6, 55.2, 42.4, 33.7, 33.6.

### 7-chloro-3-phenyl-3,4-dihydroacridine-1,9(2H,10H)-dione (19)

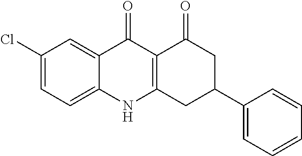 [SMILES: O=C1CC(c2ccccc2)Cc2[nH]c3ccc(Cl)cc3c(=O)c21]

Following general procedure L, the title compound was prepared as a tan-colored solid.

TLC: (5% methanol in dichloromethane, R_{f}): 0.37 (UV, 254 nm, 280 nm).

¹H NMR (500 MHz, DMSO-d₆) δ 12.22 (s, 1H), 8.04 (s, 1H), 7.74 (d, J=8.5 Hz, 1H), 7.58 (d, J=8.5 Hz, 1H), 7.46-7.33 (m, 4H), 7.27 (t, J=7.8 Hz, 1H), 3.52 (dd, J=16.4, 14.3 Hz, 1H), 3.10 (d, J=16.4 Hz, 1H), 2.79 (dd, J=16.4, 14.3 Hz, 1H), 2.59 (d, J=16.4 Hz, 1H).

### (E)-4-(2-chlorophenyl)but-3-en-2-one (32)

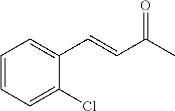

Following general procedure D, the title compound was prepared as a yellow oil.

LCMS (ESI-MS) m/z: [M+H]⁺ calcd for C₁₀H₁₀ClO 181.03; found 180.8. Retention time: 3.206 min.

TLC: (20% EtOAc in hexanes, R_{f}): 0.48 (UV, 254 nm, 280 nm).

¹H NMR (500 MHz, CDCl₃) δ 7.93 (d, J=16.3 Hz, 1H), 7.64 (dd, J=7.6, 2.0 Hz, 1H), 7.43 (dd, J=7.8, 1.5 Hz, 1H), 7.35-7.27 (m, 2H), 6.67 (d, J=16.3 Hz, 1H), 2.42 (s, 3H).

¹³C NMR (126 MHz, CDCl₃) δ 198.3, 139.2, 135.1, 132.6, 131.2, 130.2, 129.6, 127.5, 127.2, 27.2.

### (E)-4-(3-chlorophenyl)but-3-en-2-one (33)

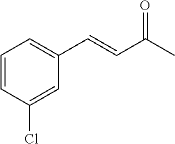

Following general procedure D, the title compound was prepared as a clear oil.

LCMS (ESI-MS) m/z: [M+H]⁺ calcd for C₁₀H₁₀ClO 181.03; found 181.1. Retention time: 3.088 min.

TLC: (5% EtOAc in hexanes, R_{f}): 0.28 (UV, 254 nm, 280 nm).

¹H NMR (399 MHz, CDCl₃) δ 7.54-7.24 (m, 5H), 6.65 (d, J=16.4 Hz, 1H), 2.34 (s, 3H).

¹³C NMR (126 MHz, CDCl₃) δ 197.8, 141.4, 136.2, 134.8, 130.2, 130.1, 128.1, 127.9, 126.3, 27.7.

### (E)-4-(4-chlorophenyl)but-3-en-2-one (34)

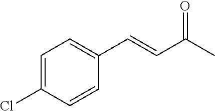

Following general procedure D, the title compound was prepared as a white powder.

LCMS (ESI-MS) m/z: [M+H]⁺ calcd for C₁₀H₁₀ClO 181.03; found 181.0. Retention time: 3.079 min.

TLC: (10% EtOAc in hexanes, R_{f}): 0.24 (UV, 254 nm, 280 nm).

¹H NMR (500 MHz, DMSO-d₆) δ 7.72 (d, J=8.2 Hz, 2H), 7.60 (d, J=16.7 Hz, 1H), 7.48 (d, J=8.2 Hz, 2H), 6.81 (d, J=16.7 Hz, 1H), 2.33 (s, 3H).

¹³C NMR (126 MHz, CDCl₃) δ 197.9, 141.8, 136.3, 132.9, 129.3, 129.2, 127.4, 27.6.

### (E)-4-(o-tolyl)but-3-en-2-one (35)

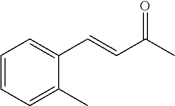

Following general procedure D, the title compound was prepared as a slightly yellow oil.

LCMS (ESI-MS) m/z: [M+H]⁺ calcd for C₁₁H₁₃O 161.09; found 160.9. Retention time: 3.935 min.

TLC: (17% EtOAc in hexanes, R_{f}): 0.60 (UV, 254 nm, 280 nm).

¹H NMR (399 MHz, DMSO-d₆) δ 7.78 (d, J=16.2 Hz, 1H), 7.67 (d, J=7.6 Hz, 1H), 7.42-7.14 (m, 3H), 6.70 (d, J=16.2 Hz, 1H), 2.40 (s, 3H), 2.34 (s, 3H).

¹³C NMR (126 MHz, CDCl₃) δ 198.2, 140.7, 137.8, 133.3, 130.8, 130.2, 128.0, 126.4, 126.3, 27.7, 19.7.

### (E)-4-(m-tolyl)but-3-en-2-one (36)

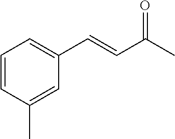

Following general procedure D, the title compound was prepared as a slightly yellow oil.

LCMS (ESI-MS) m/z: [M+H]⁺ calcd for C₁₁H₁₃O 161.09; found 161.0. Retention time: 3.981 min.

TLC: (17% EtOAc in hexanes, R_{f}): 0.55 (UV, 254 nm, 280 nm).

¹H NMR (399 MHz, DMSO-d₆) δ 7.57 (d, J=16.1 Hz, 1H), 7.54-7.44 (m, 2H), 7.30 (dd, J=8.2 Hz, 1H), 7.22 (d, J=7.9 Hz, 1H), 6.78 (d, J=16.1 Hz, 1H), 2.42-2.25 (m, 6H).

¹³C NMR (126 MHz, CDCl₃) δ 198.3, 143.6, 138.6, 134.3, 131.3, 128.9, 128.8, 126.9, 125.4, 27.4, 21.3.

### (E)-4-(p-tolyl)but-3-en-2-one (37)

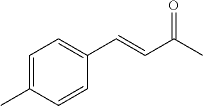

Following general procedure D, the title compound was prepared as a slightly yellow oil.

LCMS (ESI-MS) m/z: [M+H]⁺ calcd for C₁₁H₁₃O 161.09; found 161.1. Retention time: 3.075 min.

TLC: (17% EtOAc in hexanes, R_{f}): 0.35 (UV, 254 nm, 280 nm).

¹H NMR (500 MHz, DMSO-d₆) δ 7.61-7.54 (m, 3H), 7.24 (d, J=7.9 Hz, 2H), 6.73 (d, J=16.4 Hz, 1H), 2.31 (s, 6H).

¹³C NMR (126 MHz, CDCl₃) δ 198.4, 143.5, 141.0, 131.7, 129.7, 128.2, 126.2, 27.4, 21.5.

### (E)-4-(2,4-dimethylphenyl)but-3-en-2-one (38)

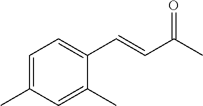

Following general procedure D, the title compound was prepared as a slightly yellow oil.

LCMS (ESI-MS) m/z: [M+H]⁺ calcd for C₁₂H₁₄O 175.10; found 175.1. Retention time: 3.307 min.

TLC: (20% EtOAc in hexanes, R_{f}): 0.53 (UV, 254 nm, 280 nm)

¹H NMR (500 MHz, CDCl₃) δ 7.79 (d, J=16.1 Hz, 1H), 7.48 (d, J=8.3 Hz, 1H), 7.06-6.99 (m, 2H), 6.63 (d, J=16.1 Hz, 1H), 2.42 (s, 3H), 2.37 (s, 3H), 2.33 (s, 3H).

¹³C NMR (126 MHz, CDCl₃) δ 198.2, 140.7, 140.5, 137.8, 131.6, 130.4, 127.2, 127.0, 126.3, 27.6, 21.2, 19.6.

### (E)-4-(2-methoxyphenyl)but-3-en-2-one (39)

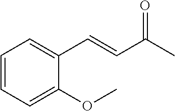

Following general procedure D, the title compound was prepared as a white powder.

LCMS (ESI-MS) m/z: [M+H]⁺ calcd for C₁₁H₁₃O₂ 177.08; found 177.1. Retention time: 2.866 min.

TLC: (17% EtOAc in hexanes, R_{f}): 0.51 (UV, 254 nm, 280 nm).

¹H NMR (399 MHz, DMSO-d₆) δ 7.78 (d, J=16.6 Hz, 1H), 7.70 (d, J=7.7 Hz, 1H), 7.42 (dd, J=9.6, 8.4 Hz, 1H), 7.10 (d, J=8.4 Hz, 1H), 6.99 (dd, J=9.6, 7.7 Hz, 1H), 6.83 (d, J=16.6 Hz, 1H), 3.87 (s, 3H), 2.31 (s, 3H).

¹³C NMR (126 MHz, CDCl₃) δ 198.9, 158.2, 138.6, 131.8, 128.2, 127.6, 123.2, 120.7, 111.1, 55.4, 27.1.

### (E)-4-(3-methoxyphenyl)but-3-en-2-one (40)

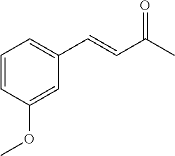

Following general procedure E, the title compound was prepared as a slightly yellow oil.

LCMS (ESI-MS) m/z: [M+H]⁺ calcd for C₁₁H₁₃O₂ 177.08; found 177.1. Retention time: 2.844 min.

TLC: (17% EtOAc in hexanes, R_{f}): 0.43 (UV, 254 nm, 280 nm).

¹H NMR (500 MHz, DMSO-d₆) δ 7.59 (d, J=16.3 Hz, 1H), 7.34 (dd, J=9.0, 8.2 Hz, 1H), 7.30-7.24 (m, 2H), 6.98 (d, J=9.0 Hz, 1H), 6.82 (d, J=16.3 Hz, 1H), 3.79 (s, 3H), 2.33 (s, 3H).

¹³C NMR (126 MHz, CDCl₃) δ 198.3, 159.9, 143.3, 135.8, 129.9, 127.3, 120.9, 116.3, 113.0, 55.2, 27.4.

### (E)-4-(4-methoxyphenyl)but-3-en-2-one (41)

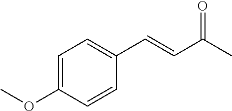

Following general procedure D, the title compound was prepared as a white powder.

LCMS (ESI-MS) m/z: [M+H]⁺ calcd for C₁₁H₁₃O₂ 177.08; found 177.1. Retention time: 2.716 min.

TLC: (17% EtOAc in hexanes, R_{f}): 0.52 (UV, 254 nm, 280 nm).

¹H NMR (399 MHz, DMSO-d₆) δ 7.67 (d, J=8.4 Hz, 2H), 7.58 (d, J=16.4 Hz, 1H), 6.99 (d, J=8.4 Hz, 2H), 6.68 (d, J=16.4 Hz, 1H), 3.80 (s, 3H), 2.30 (s, 3H).

¹³C NMR (126 MHz, CDCl₃) δ 198.4, 161.6, 143.2, 129.9, 127.0, 125.0, 114.4, 55.4, 27.4.

### (E)-4-(2,4-dimethoxyphenyl)but-3-en-2-one (42)

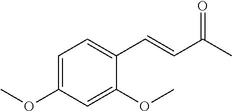

Following general procedure D, the title compound was prepared as a yellow oil.

LCMS (ESI-MS) m/z: [M+H]⁺ calcd for C₁₂H₁₅O₃ 207.09; found 207.1. Retention time: 2.899 min.

TLC: (20% EtOAc in hexanes, R_{f}): 0.22 (UV, 254 nm, 280 nm).

¹H NMR (500 MHz, CDCl₃) δ 7.81 (d, J=16.4 Hz, 1H), 7.49 (d, J=8.6 Hz, 1H), 6.68 (d, J=16.4 Hz, 1H), 6.52 (dd, J=8.6, 2.3 Hz, 1H), 6.46 (d, J=2.3 Hz, 1H), 3.88 (s, 3H), 3.85 (s, 3H), 2.36 (s, 3H).

¹³C NMR (126 MHz, CDCl₃) δ 199.1, 163.0, 159.8, 138.7, 129.8, 125.5, 116.4, 105.4, 98.4, 55.53, 55.50, 27.0.

### (E)-4-phenylbut-3-en-2-one (43)

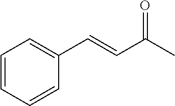

Following general procedure D, the title compound was prepared as an off-white powder.

LCMS (ESI-MS) m/z: [M+H]⁺ calcd for C₁₀H₁₁O 147.07; found 147.1. Retention time: 2.712 min.

TLC: (10% EtOAc in hexanes, R_{f}): 0.24 (UV, 254 nm, 280 nm).

¹H NMR (500 MHz, DMSO-d₆) δ 7.75-7.68 (m, 2H), 7.64 (d, J=16.7 Hz, 1H), 7.48-7.42 (m, 3H), 6.82 (d, J=16.7 Hz, 1H), 2.35 (s, 3H).

¹³C NMR (126 MHz, DMSO-d₆) δ 198.1, 143.1, 134.4, 130.4, 128.9, 128.3, 127.2, 27.3.

### 2′-chloro-5-hydroxy-1,6-dihydro-[1,1′-biphenyl]-3(2H)-one (56)

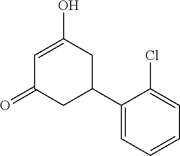 [SMILES: O=C1C=C(O)CC(c2ccccc2Cl)C1]

Following general procedures J and K, the title compound was prepared as a white solid.

LCMS (ESI-MS) m/z: [M+H]⁺ calcd for C₁₂H₁₂ClO₂ 223.04; found 223.2. Retention time: 2.687 min.

TLC: (3% methanol in dichloromethane, R_{f}): 0.23 (UV, 254 nm, 280 nm).

¹H NMR (399 MHz, CD₃COCD₃) δ 11.06 (s, 1H), 7.52 (d, J=7.4 Hz, 1H), 7.45 (d, J=8.5 Hz, 1H), 7.36 (dd, J=7.9, 7.4 Hz, 1H), 7.28 (dd, J=8.5, 7.9 Hz, 1H), 5.37 (s, 1H), 3.83-3.70 (m, 1H), 2.72-2.54 (m, 2H), 2.54-2.41 (m, 2H).

### 3′-chloro-5-hydroxy-1,6-dihydro-[1,1′-biphenyl]-3(2H)-one (57)

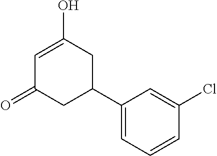 [SMILES: O=C1C=C(O)CC(c2cccc(Cl)c2)C1]

Following general procedures J and K, the title compound was prepared as a white solid.

LCMS (ESI-MS) m/z: [M+H]⁺ calcd for C₁₂H₁₂ClO₂ 223.04; found 222.8. Retention time: 3.623 min.

TLC: (5% methanol in dichloromethane, R_{f}): 0.25 (UV, 254 nm, 280 nm).

¹H NMR (500 MHz, DMSO-d₆) δ 11.22 (s, 1H), 7.44 (s, 1H), 7.39-7.27 (m, 3H), 5.29 (s, 1H), 3.44-3.29 (m, 1H), 2.75-2.55 (m, 2H), 2.40 (d, J=16.8 Hz, 2H).

¹³C NMR (126 MHz, DMSO-d₆) δ 146.6, 133.6, 130.8, 127.4, 127.0, 126.2, 104.0, 38.8.

IR (thin film, cm⁻¹): v=2509, 1595, 1498, 1363, 1310, 1223, 1139, 1081, 998, 886, 846, 781, 695, 612, 443.

### 4′-chloro-5-hydroxy-1,6-dihydro-[1,1′-biphenyl]-3(2H)-one (58)

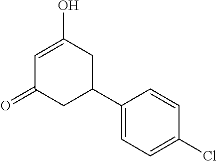 [SMILES: O=C1C=C(O)CC(c2ccc(Cl)cc2)C1]

Following general procedures J and K, the title compound was prepared as a white solid.

LCMS (ESI-MS) m/z: [M+H]⁺ calcd for C₁₂H₁₂ClO₂ 223.04; found 222.8. Retention time: 3.557 min.

TLC: (5% methanol in dichloromethane, R_{f}): 0.14 (UV, 254 nm, 280 nm).

¹H NMR (500 MHz, DMSO-d₆) δ 11.22 (s, 1H), 7.38 (s, 4H), 5.29 (s, 1H), 3.35-3.28 (m, 1H), 2.58 (s, 2H), 2.40 (s, 2H).

¹³C NMR (126 MHz, DMSO-d₆) δ 143.0, 131.6, 129.3, 128.8, 104.0, 38.5.

IR (thin film, cm⁻¹): v=2504, 1585, 1493, 1471, 1368, 1320, 1298, 1265, 1216, 1142, 1092, 1015, 831, 697, 581, 507, 437.

### 5-hydroxy-2′-methyl-1,6-dihydro-[1,1′-biphenyl]-3(2H)-one (59)

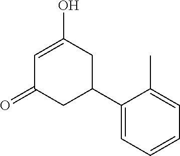

Following general procedures J and K, the title compound was prepared as a white solid.

LCMS (ESI-MS) m/z: [M+H]⁺ calcd for C₁₃H₁₅O₂ 203.10; found 202.9. Retention time: 3.409 min.

TLC: (5% methanol in dichloromethane, R_{f}): 0.24 (UV, 254 nm, 280 nm).

¹H NMR (500 MHz, DMSO-d₆) δ 11.20 (s, 1H), 7.35 (d, J=7.6 Hz, 1H), 7.22-7.08 (m, 3H), 5.31 (s, 1H), 3.53-3.45 (m, 1H), 2.66-2.52 (m, 2H), 2.39-2.24 (m, 5H).

¹³C NMR (126 MHz, DMSO-d₆) δ 141.9, 135.6, 130.8, 126.7, 126.7, 126.1, 103.8, 35.1, 19.3.

IR (thin film, cm⁻¹): v=2948, 1561, 1402, 1310, 1211, 1142, 758, 449.

### 5-hydroxy-3′-methyl-1,6-dihydro-[1,1′-biphenyl]-3(2H)-one (60)

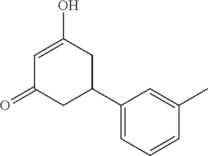

Following general procedures J and K, the title compound was prepared as a white powder.

LCMS (ESI-MS) m/z: [M+H]⁺ calcd for C₁₃H₁₅O₂ 203.10; found 202.9. Retention time: 3.484 min.

TLC: (5% methanol in dichloromethane, R_{f}): 0.17 (UV, 254 nm, 280 nm).

¹H NMR (399 MHz, DMSO-d₆) δ 11.20 (s, 1H), 7.26-7.09 (m, 3H), 7.04 (d, J=7.5 Hz, 1H), 5.27 (s, 1H), 3.30-3.16 (m, 1H), 2.76-2.52 (m, 2H), 2.44-2.32 (m, 2H), 2.28 (s, 3H).

¹³C NMR (126 MHz, DMSO-d₆) δ 143.95, 138.00, 128.84, 128.07, 127.69, 124.38, 103.98, 39.17, 21.56.

IR (thin film, cm⁻¹): v=2892, 1570, 1400, 1315, 1222, 1140, 870, 784, 702, 444.

### 5-hydroxy-4′-methyl-1,6-dihydro-[1,1′-biphenyl]-3(2H)-one (61)

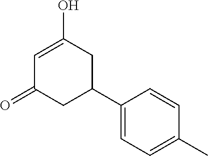

Following general procedures J and K, the title compound was prepared as a white solid.

LCMS (ESI-MS) m/z: [M+H]⁺ calcd for C₁₃H₁₅O₂ 203.10; found 202.9. Retention time: 3.490 min. [2M+H]⁺ calcd for C₂₆H₂₉O₄ 405.20; found 404.8. Retention time: 3.490.

TLC: (5% methanol in dichloromethane, R_{f}): 0.17 (UV, 254 nm, 280 nm).

¹H NMR (500 MHz, DMSO-d6) δ 11.17 (s, 1H), 7.21 (d, J=7.8 Hz, 2H), 7.12 (d, J=7.8 Hz, 2H), 5.29 (s, 1H), 3.30-3.21 (m, 1H), 2.65-2.51 (m, 2H), 2.37 (d, J=15.0 Hz, 2H), 2.27 (s, 3H).

¹³C NMR (126 MHz, DMSO-d₆) δ 141.0, 136.0, 129.4, 127.2, 104.0, 38.8, 21.0.

IR (thin film, cm⁻¹): v=2859, 1578, 1514, 1425, 1366, 1323, 1301, 1219, 1143, 812, 738, 583, 503, 441.

### 5-(2,4-dimethylphenyl)cyclohexane-1,3-dione (62)

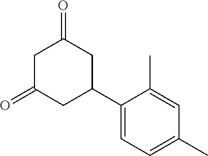 [SMILES: Cc1ccc(C2CC(=O)CC(=O)C2)c(C)c1]

Following general procedures J and K, the title compound was prepared as a white solid.

LCMS (ESI-MS) m/z: [M+H]⁺ calcd for C₁₄H₁₇O₂ 217.12; found 216.9. Retention time: 432.8 min. [2M+H]⁺ calcd for C₂₈H₃₃O₄ 433.24; found 432.8. Retention time: 3.689.

TLC: (5% methanol in dichloromethane, R_{f}): 0.17 (UV, 254 nm, 280 nm).

¹H NMR (500 MHz, CD₃OD) δ 7.19 (d, J=8.5 Hz, 1H), 7.06-6.97 (m, 2H), 4.92 (s, 2H), 3.62-3.52 (m, 1H), 2.65 (dd, J=17.1, 11.9 Hz, 2H), 2.46 (dd, J=17.1, 4.5 Hz, 2H), 2.32 (s, 3H), 2.28 (s, 3H).

¹³C NMR (126 MHz, CD₃OD) δ 139.1, 137.2, 136.3, 132.3, 128.0, 126.3, 36.3, 20.9, 19.2.

IR (thin film, cm⁻¹): v=2946, 1557, 1402, 1313, 1213, 1141, 1030, 816, 737, 590, 540, 448.

### 5-(2-methoxyphenyl)cyclohexane-1,3-dione (63)

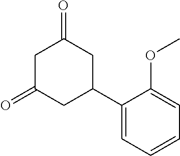 [SMILES: COc1ccccc1C1CC(=O)CC(=O)C1]

Following general procedures J and K, the title compound was prepared as a white solid.

LCMS (ESI-MS) m/z: [M+H]⁺ calcd for C₁₃H₁₅O₃ 219.09; found 218.9. Retention time: 3.334 min. [2M+H]⁺ calcd for C₂₆H₂₉O₆ 437.18; found 436.8. Retention time: 3.334.

TLC: (5% methanol in dichloromethane, R_{f}): 0.17 (UV, 254 nm, 280 nm).

¹H NMR (500 MHz, CD₃OD) δ 7.29-7.20 (m, 2H), 6.99 (d, J=8.1 Hz, 1H), 6.94 (dd, J=8.3, 7.8 Hz, 1H), 4.91 (s, 2H), 3.87 (s, 3H), 3.75-3.66 (m, 1H), 2.72 (dd, J=16.9, 11.7 Hz, 2H), 2.52 (dd, J=16.9, 4.5 Hz, 2H).

¹³C NMR (126 MHz, CD₃OD) δ 158.5, 131.8, 129.1, 128.0, 121.7, 111.8, 55.7, 35.1.

IR (thin film, cm⁻¹): v=2942, 1559, 1492, 1462, 1401, 1320, 1294, 1220, 1143, 1122, 1053, 1028, 835, 752, 646, 591, 442.

### 5-hydroxy-3′-methoxy-1,6-dihydro-[1,1′-biphenyl]-3(2H)-one (64)

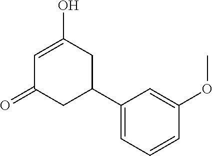 [SMILES: COc1cccc(C2CC(=O)C=C(O)C2)c1]

Following general procedures J and K, the title compound was prepared as a white solid.

LCMS (ESI-MS) m/z: [M+H]⁺ calcd for C₁₃H₁₅O₃ 219.09; found 218.9. Retention time: 3.212 min. [2M+H]⁺ calcd for C₂₆H₂₉O₆ 437.18; found 436.8. Retention time: 3.212.

TLC: (5% methanol in dichloromethane, R_{f}): 0.16 (UV, 254 nm, 280 nm).

¹H NMR (500 MHz, DMSO-d₆) δ 11.19 (s, 1H), 7.23 (dd, J=9.4, 8.0 Hz, 1H), 6.95-6.88 (m, 2H), 6.80 (dd, J=8.3, 2.4 Hz, 1H), 5.30 (s, 1H), 3.74 (s, 3H), 3.31-3.23 (m, 1H), 2.71-2.53 (m, 2H), 2.48-2.28 (m, 2H).

¹³C NMR (126 MHz, DMSO-d₆) δ 159.8, 145.6, 129.9, 119.5, 113.3, 112.3, 103.9, 55.4, 39.2.

IR (thin film, cm⁻¹): v=2943, 1583, 1488, 1400, 1365, 1309, 1268, 1223, 1154, 1049, 867, 780, 698, 564, 447, 416.

### 5-hydroxy-4′-methoxy-1,6-dihydro-[1,1′-biphenyl]-3(2H)-one (65)

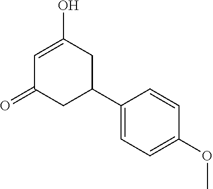 [SMILES: COc1ccc(C2CC(=O)C=C(O)C2)cc1]

Following general procedures J and K, the title compound was prepared as a white solid.

LCMS (ESI-MS) m/z: [M+H]⁺ calcd for C₁₃H₁₅O₃ 219.09; found 218.9. Retention time: 3.155 min.

TLC: (5% methanol in dichloromethane, R_{f}): 0.22 (UV, 254 nm, 280 nm).

¹H NMR (500 MHz, DMSO-d₆) δ 11.16 (s, 1H), 7.25 (d, J=8.2 Hz, 2H), 6.88 (d, J=8.2 Hz, 2H), 5.29 (s, 1H), 3.73 (s, 3H), 3.30-3.18 (m, 1H), 2.59-2.51 (m, 2H), 2.37 (d, J=16.6 Hz, 2H).

¹³C NMR (126 MHz, DMSO-d₆) δ 158.3, 136.0, 128.3, 114.3, 104.0, 55.4, 38.4.

IR (thin film, cm⁻¹): v=2835, 1596, 1512, 1367, 1321, 1293, 1220, 1181, 1139, 1029, 829, 764, 597, 523, 443.

### 5-(2,4-dimethoxyphenyl)cyclohexane-1,3-dione (66)

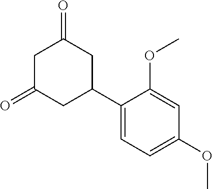 [SMILES: COc1ccc(C2CC(=O)CC(=O)C2)c(OC)c1]

Following general procedures J and K, the title compound was prepared as an off-white and slightly yellow powder.

LCMS (ESI-MS) m/z: [M+H]⁺ calcd for C₁₄H₁₇O₄ 249.10; found 248.9. Retention time: 3.350 min.

TLC: (5% methanol in dichloromethane, R_{f}): 0.22 (UV, 254 nm, 280 nm).

¹H NMR (500 MHz, CD₃OD) δ 7.00 (d, J=8.4 Hz, 1H), 6.43 (d, J=2.5 Hz, 1H), 6.38 (dd, J=8.4, 2.5 Hz, 1H), 4.76 (s, 2H), 3.72 (s, 3H), 3.67 (s, 3H), 3.52-3.41 (m, 1H), 2.55 (dd, J=17.0, 11.7 Hz, 2H), 2.37 (dd, J=17.0, 4.5 Hz, 2H).

¹³C NMR (126 MHz, CD₃OD) δ 161.3, 159.4, 128.5, 124.3, 105.5, 99.6, 55.8, 55.7, 39.4, 34.7.

### 5-hydroxy-1,6-dihydro-[1,1′-biphenyl]-3(2H)-one (67)

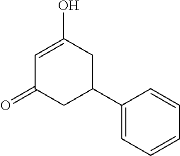 [SMILES: O=C1C=C(O)CC(c2ccccc2)C1]

Following general procedures J and K, the title compound was prepared as a white solid.

LCMS (ESI-MS) m/z: [M+H]⁺ calcd for C₁₂H₁₃O₂ 189.08; found 188.9. Retention time: 3.152 min.

TLC: (5% methanol in dichloromethane, R_{f}): 0.24 (UV, 254 nm, 280 nm).

¹H NMR (500 MHz, DMSO-d₆) δ 11.19 (s, 1H), 7.37-7.29 (m, 4H), 7.26-7.20 (m, 1H), 5.29 (s, 1H), 3.33-3.25 (m, 1H), 2.68-2.53 (m, 2H), 2.47-2.31 (m, 2H).

¹³C NMR (126 MHz, DMSO-d₆) δ 144.0, 128.9, 127.3, 127.0, 104.0, 39.2.

IR (thin film, cm⁻¹): v=3028, 2018, 1571, 1400, 1334, 1217, 1142, 833, 760, 698, 498, 447.

### 3-(2,4-dichlorophenyl)-3,4-dihydroacridine-1,9(2H,10H)-dione (68)

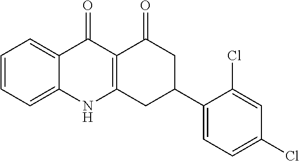 [SMILES: O=C1CC(c2ccc(Cl)cc2Cl)Cc2[nH]c3ccccc3c(=O)c21]

Following general procedure L, the title compound was prepared as a tan-colored solid.

LCMS (ESI-MS) m/z: [M+H]⁺ calcd for C₁₉H₁₄Cl₂NO₂ 358.03; found 358.0. Retention time: 2.887 min.

TLC: (5% methanol in dichloromethane, R_{f}): 0.34 (UV, 254 nm, 280 nm).

¹H NMR (500 MHz, CDCl₃ and 3 drops of TFA-d) δ 8.58 (d, J=8.2 Hz, 1H), 8.17 (dd, J=8.5, 7.8 Hz, 1H), 8.04 (d, J=8.5 Hz, 1H), 7.91 (dd, J=8.2, 7.8 Hz, 1H), 7.50 (d, J=2.1 Hz, 1H), 7.35 (dd, J=8.3, 2.1 Hz, 1H), 7.24 (d, J=8.3 Hz, 1H), 4.22-4.11 (m, 1H), 3.75-3.61 (m, 2H), 3.27-3.10 (m, 2H).

¹³C NMR (126 MHz, CDCl₃ and 3 drops of TFA-d) δ 202.5, 161.1, 139.5, 138.8, 135.1, 135.0, 134.4, 130.6, 129.9, 128.3, 127.7, 125.5, 120.4, 42.6, 34.8, 33.6.

### 8-chloro-3-(2,4-dichlorophenyl)-3,4-dihydroacridine-1,9(2H,10H)-dione (69)

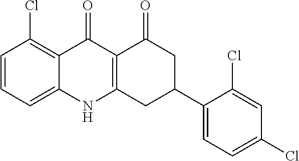 [SMILES: O=C1CC(c2ccc(Cl)cc2Cl)Cc2[nH]c3cccc(Cl)c3c(=O)c21]

Following general procedure L, the title compound was prepared as a yellow solid.

TLC: (5% methanol in dichloromethane, R_{f}): 0.40 (UV, 254 nm, 280 nm).

¹H NMR (500 MHz, CDCl₃ and 3 drops of TFA-d) δ 8.04-7.94 (m, 2H), 7.87 (dd, J=7.4, 1.5 Hz, 1H), 7.50 (d, J=2.1 Hz, 1H), 7.35 (dd, J=8.4, 2.1 Hz, 1H), 7.24 (d, J=8.4 Hz, 1H), 4.19-4.09 (m, 1H), 3.72-3.60 (m, 2H), 3.24 (ddd, J=17.7, 4.2, 1.4 Hz, 1H), 3.15 (dd, J=17.7, 12.4 Hz, 1H).

¹³C NMR (126 MHz, CDCl₃ and 3 drops of TFA-d) δ 202.8, 175.1, 161.3, 141.5, 137.6, 135.2, 135.0, 134.8, 134.3, 132.6, 130.5, 128.2, 127.6, 119.6, 117.2, 108.0, 42.6, 34.5, 33.2.

### 6-chloro-3-(2,4-dichlorophenyl)-3,4-dihydroacridine-1,9(2H,10H)-dione (70)

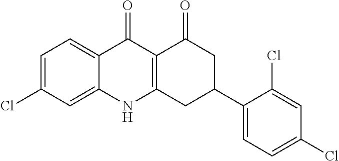 [SMILES: O=C1CC(c2ccc(Cl)cc2Cl)Cc2[nH]c3cc(Cl)ccc3c(=O)c21]

Following general procedure L, the title compound was prepared as a white solid.

LCMS (ESI-MS) m/z: [M+H]⁺ calcd for C₁₉H₁₃Cl₃NO₂ 391.99; found 392.0. Retention time: 3.230 min.

TLC: (5% methanol in dichloromethane, R_{f}): 0.44 (UV, 254 nm, 280 nm).

¹H NMR (500 MHz, CDCl₃ and 3 drops of TFA-d) δ 8.48 (d, J=8.9 Hz, 1H), 8.07 (d, J=1.8 Hz, 1H), 7.82 (dd, J=8.9, 1.8 Hz, 1H), 7.50 (d, J=2.2 Hz, 1H), 7.34 (dd, J=8.4, 2.2 Hz, 1H), 7.25 (d, J=8.4 Hz, 1H), 4.20-4.08 (m, 1H), 3.76-3.62 (m, 2H), 3.22 (dd, J=17.7, 3.6 Hz, 1H), 3.12 (dd, J=17.7, 12.6 Hz, 1H).

¹³C NMR (126 MHz, CDCl₃ and 3 drops of TFA-d) δ 202.3, 173.0, 162.2, 145.8, 140.1, 134.98, 134.95, 134.2, 130.8, 130.4, 128.2, 127.7, 126.5, 120.2, 117.6, 107.9, 42.6, 34.6, 33.4.

### 5-chloro-3-(2,4-dichlorophenyl)-3,4-dihydroacridine-1,9(2H,10H)-dione (71)

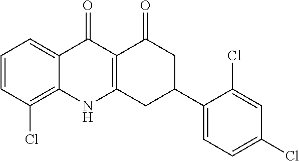 [SMILES: O=C1CC(c2ccc(Cl)cc2Cl)Cc2[nH]c3c(Cl)cccc3c(=O)c21]

Following general procedure L, the title compound was prepared as a white solid.

LCMS (ESI-MS) m/z: [M+H]⁺ calcd for C₁₉H₁₃Cl₃NO₂ 391.99; found 392.0. Retention time: 3.402 min.

TLC: (5% methanol in dichloromethane, R_{f}): 0.49 (UV, 254 nm, 280 nm).

¹H NMR (500 MHz, CDCl₃ and 3 drops of TFA-d) δ 8.53 (dd, J=8.4, 1.3 Hz, 1H), 8.21 (dd, J=7.8, 1.3 Hz, 1H), 7.85 (dd, J=8.4, 7.8 Hz, 1H), 7.49 (d, J=2.1 Hz, 1H), 7.34 (dd, J=8.4, 2.1 Hz, 1H), 7.25 (d, J=8.4 Hz, 1H), 4.20-4.10 (m, 1H), 3.96 (ddd, J=18.2, 4.1, 1.3 Hz, 1H), 3.76 (dd, J=18.1, 11.7 Hz, 1H), 3.28-3.13 (m, 2H).

¹³C NMR (126 MHz, CDCl₃ and 3 drops of TFA-d) δ 202.6, 173.9, 163.4, 138.2, 136.1, 134.99, 134.97, 134.2, 130.4, 129.7, 128.3, 127.7, 125.4, 124.3, 121.0, 108.4, 34.4, 34.3, 33.5.

### 3-(2,4-dichlorophenyl)-8-methyl-3,4-dihydroacridine-1,9(2H,10H)-dione (72)

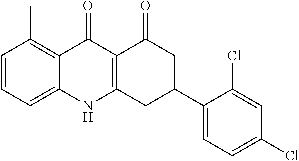 [SMILES: Cc1cccc2[nH]c3c(c(=O)c12)C(=O)CC(c1ccc(Cl)cc1Cl)C3]

Following general procedure L, the title compound was prepared as a tan-colored solid.

TLC: (5% methanol in dichloromethane, R_{f}): 0.43 (UV, 254 nm, 280 nm).

¹H NMR (500 MHz, DMSO-d₆) δ 11.84 (s, 1H), 7.67 (d, J=2.1 Hz, 1H), 7.55-7.47 (m, 3H), 7.34 (d, J=8.2 Hz, 1H), 7.08 (d, J=7.3 Hz, 1H), 3.83-3.75 (m, 1H), 3.30 (d, J=11.4 Hz, 1H), 3.08 (d, J=16.0 Hz, 1H), 2.79-2.68 (m, 4H), 2.55 (d, J=16.0 Hz, 1H).

### 3-(2,4-dichlorophenyl)-7-methyl-3,4-dihydroacridine-1,9(2H,10H)-dione (73)

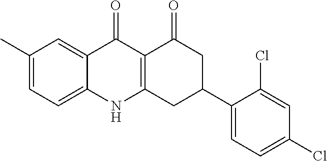 [SMILES: Cc1ccc2[nH]c3c(c(=O)c2c1)C(=O)CC(c1ccc(Cl)cc1Cl)C3]

Following general procedure L, the title compound was prepared as a white solid.

LCMS (ESI-MS) m/z: [M+H]⁺ calcd for C₂₀H₁₆Cl₂NO₂ 372.05; found 372.1. Retention time: 3.099 min.

TLC: (5% methanol in dichloromethane, R_{f}): 0.45 (UV, 254 nm, 280 nm).

¹H NMR (500 MHz, CDCl₃ and 3 drops of TFA-d) δ 8.34 (s, 1H), 8.00 (dd, J=8.6, 1.9 Hz, 1H), 7.94 (d, J=8.6 Hz, 1H), 7.52 (d, J=2.2 Hz, 1H), 7.36 (dd, J=8.4, 2.2 Hz, 1H), 7.27 (d, J=8.4 Hz, 1H), 4.21-4.12 (m, 1H), 3.75-3.61 (m, 2H), 3.29-3.10 (m, 2H), 2.68 (s, 3H).

¹³C NMR (126 MHz, CDCl₃ and 3 drops of TFA-d) δ 202.4, 173.1, 159.8, 141.2, 140.6, 137.8, 135.1, 135.0, 134.3, 130.5, 128.3, 127.6, 124.3, 120.1, 119.5, 107.8, 42.6, 34.8, 33.5, 21.6.

### 3-(2,4-dichlorophenyl)-6-methyl-3,4-dihydroacridine-1,9(2H,10H)-dione (74)

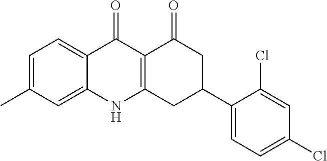 [SMILES: Cc1ccc2c(=O)c3c([nH]c2c1)CC(c1ccc(Cl)cc1Cl)CC3=O]

Following general procedure L, the title compound was prepared as a yellow solid.

LCMS (ESI-MS) m/z: [M+H]⁺ calcd for C₂₀H₁₆Cl₂NO₂ 372.05; found 371.7. Retention time: 4.040 min.

TLC: (5% methanol in dichloromethane, R_{f}): 0.44 (UV, 254 nm, 280 nm).

¹H NMR (500 MHz, CDCl₃ and 3 drops of TFA-d) δ 8.42 (d, J=8.5 Hz, 1H), 7.80 (s, 1H), 7.69 (dd, J=8.5, 1.5 Hz, 1H), 7.50 (d, J=2.2 Hz, 1H), 7.34 (dd, J=8.4, 2.2 Hz, 1H), 7.25 (d, J=8.4 Hz, 1H), 4.20-4.08 (m, 1H), 3.73-3.59 (m, 2H), 3.20 (dd, J=17.6, 4.6 Hz, 1H), 3.10 (dd, J=17.6, 12.5 Hz, 1H), 2.68 (s, 3H).

¹³C NMR (126 MHz, CDCl₃ and 3 drops of TFA-d) δ 202.2, 172.9, 160.7, 151.7, 139.9, 135.1, 134.8, 134.2, 131.7, 130.4, 128.2, 127.6, 124.9, 119.7, 117.2, 107.4, 42.7, 34.7, 33.3, 22.6.

### 3-(2,4-dichlorophenyl)-5-methyl-3,4-dihydroacridine-1,9(2H,10H)-dione (75)

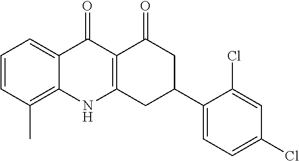 [SMILES: Cc1cccc2c(=O)c3c([nH]c12)CC(c1ccc(Cl)cc1Cl)CC3=O]

Following general procedure L, the title compound was prepared as a yellow solid.

LCMS (ESI-MS) m/z: [M+H]⁺ calcd for C₂₀H₁₆Cl₂NO₂ 372.05; found 372.1. Retention time: 3.291 min.

TLC: (5% methanol in dichloromethane, R_{f}): 0.41 (UV, 254 nm, 280 nm).

¹H NMR (500 MHz, CDCl₃ and 3 drops of TFA-d) δ 8.44 (d, J=8.3 Hz, 1H), 7.98 (d, J=7.3 Hz, 1H), 7.79 (dd, J=8.3, 7.3 Hz, 1H), 7.49 (d, J=2.2 Hz, 1H), 7.33 (dd, J=8.4, 2.2 Hz, 1H), 7.24 (d, J=8.4 Hz, 1H), 4.17-4.07 (m, 1H), 3.87 (ddd, J=18.0, 4.1, 1.6 Hz, 1H), 3.69 (dd, J=17.9, 11.6 Hz, 1H), 3.27-3.09 (m, 2H), 2.76 (s, 3H).

### 3-(2,4-dichlorophenyl)-8-methoxy-3,4-dihydroacridine-1,9(2H,10H)-dione (76)

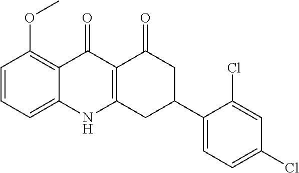 [SMILES: COc1cccc2[nH]c3c(c(=O)c12)C(=O)CC(c1ccc(Cl)cc1Cl)C3]

Following general procedure L, the title compound was prepared as a tan-colored solid.

TLC: (5% methanol in dichloromethane, R_{f}): 0.42 (UV, 254 nm, 280 nm).

¹H NMR (500 MHz, CDCl₃ and 3 drops of TFA-d) δ 8.02 (dd, J=8.4 Hz, 1H), 7.53 (d, J=8.4 Hz, 1H), 7.49 (d, J=2.1 Hz, 1H), 7.33 (dd, J=8.4, 2.1 Hz, 1H), 7.24 (d, J=8.4 Hz, 1H), 7.18 (d, J=8.4 Hz, 1H), 4.18-4.06 (m, 4H), 3.67-3.54 (m, 2H), 3.19 (ddd, J=17.6, 4.2, 1.3 Hz, 1H), 3.10 (dd, J=17.6, 12.4 Hz, 1H).

¹³C NMR (126 MHz, CDCl₃ and 3 drops of TFA-d) δ 202.4, 175.6, 160.88, 160.82, 141.2, 139.5, 135.1, 134.8, 134.3, 130.4, 128.2, 127.6, 111.9, 110.6, 109.5, 107.3, 57.0, 42.6, 34.5, 33.1.

### 3-(2,4-dichlorophenyl)-7-methoxy-3,4-dihydroacridine-1,9(2H,10H)-dione (77)

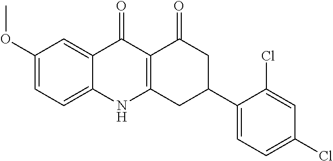 [SMILES: COc1ccc2[nH]c3c(c(=O)c2c1)C(=O)CC(c1ccc(Cl)cc1Cl)C3]

Following general procedure L, the title compound was prepared as a tan-colored solid.

LCMS (ESI-MS) m/z: [M+H]⁺ calcd for C₂₀H₁₆Cl₂NO₃ 388.04; found 388.1. Retention time: 3.022 min.

TLC: (5% methanol in dichloromethane, R_{f}): 0.44 (UV, 254 nm, 280 nm).

¹H NMR (500 MHz, CDCl₃ and 3 drops of TFA-d) δ 7.98 (d, J=9.1 Hz, 1H), 7.79-7.70 (m, 2H), 7.50 (d, J=2.2 Hz, 1H), 7.34 (dd, J=8.4, 2.2 Hz, 1H), 7.25 (d, J=8.4 Hz, 1H), 4.21-4.09 (m, 1H), 4.04 (s, 3H), 3.72-3.56 (m, 2H), 3.21 (dd, J=17.6, 3.5 Hz, 1H), 3.11 (dd, J=17.7, 12.6 Hz, 1H).

¹³C NMR (126 MHz, CDCl₃ and 3 drops of TFA-d) δ 202.4, 171.7, 160.2, 157.7, 135.1, 134.87, 134.84, 134.2, 130.4, 130.3, 128.2, 127.6, 122.1, 120.9, 107.6, 102.9, 56.3, 42.8, 34.7, 33.1.

### 3-(2,4-dichlorophenyl)-6-methoxy-3,4-dihydroacridine-1,9(2H,10H)-dione (78)

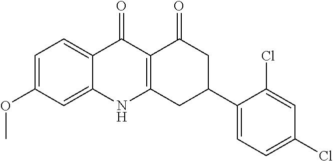 [SMILES: COc1ccc2c(=O)c3c([nH]c2c1)CC(c1ccc(Cl)cc1Cl)CC3=O]

Following general procedure L, the title compound was prepared as a tan-colored solid.

LCMS (ESI-MS) m/z: [M+H]⁺ calcd for C₂₀H₁₆Cl₂NO₃ 388.04; found 387.7. Retention time: 4.011 min.

TLC: (5% methanol in dichloromethane, R_{f}): 0.44 (UV, 254 nm, 280 nm).

¹H NMR (500 MHz, CDCl₃ and 3 drops of TFA-d) δ 8.39 (d, J=9.3 Hz, 1H), 7.49 (d, J=2.3 Hz, 1H), 7.40 (dd, J=9.3, 2.3 Hz, 1H), 7.34 (dd, J=7.9, 2.2 Hz, 2H), 7.25 (d, J=8.5 Hz, 1H), 4.19-4.08 (m, 1H), 4.08-4.00 (m, 3H), 3.67-3.59 (m, 2H), 3.18 (dd, J=17.6, 3.8 Hz, 1H), 3.07 (dd, J=17.6, 12.6 Hz, 1H).

¹³C NMR (126 MHz, CDCl₃ and 3 drops of TFA-d) δ 202.0, 171.8, 168.0, 160.7, 142.7, 135.2, 134.8, 134.2, 130.4, 128.2, 127.7, 126.7, 121.9, 113.1, 107.0, 100.0, 56.7, 42.6, 34.8, 33.3.

### 3-(2,4-dichlorophenyl)-5-methoxy-3,4-dihydroacridine-1,9(2H,10H)-dione (79)

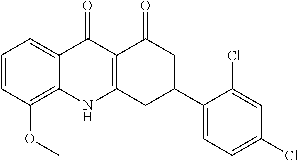 [SMILES: COc1cccc2c(=O)c3c([nH]c12)CC(c1ccc(Cl)cc1Cl)CC3=O]

Following general procedure L, the title compound was prepared as a tan-colored solid.

LCMS (ESI-MS) m/z: [M+H]⁺ calcd for C₂₀H₁₆Cl₂NO₃ 388.04; found 388.0. Retention time: 3.019 min.

TLC: (5% methanol in dichloromethane, R_{f}): 0.45 (UV, 254 nm, 280 nm).

¹H NMR (500 MHz, CDCl₃ and 3 drops of TFA-d) δ 8.08 (dd, J=8.4, 1.0 Hz, 1H), 7.80 (dd, J=8.8, 8.4 Hz, 1H), 7.55-7.47 (m, 2H), 7.34 (dd, J=8.4, 2.2 Hz, 1H), 7.26 (d, J=8.4 Hz, 1H), 4.19-4.03 (m, 4H), 3.88 (ddd, J=17.9, 4.0, 1.6 Hz, 1H), 3.68 (dd, J=17.9, 11.8 Hz, 1H), 3.27-3.06 (m, 2H).

### 3-(2,4-dichlorophenyl)-7-iodo-3,4-dihydroacridine-1,9(2H,10H)-dione (80)

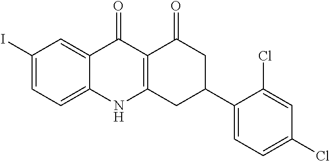 [SMILES: O=C1CC(c2ccc(Cl)cc2Cl)Cc2[nH]c3ccc(I)cc3c(=O)c21]

Following general procedure L, the title compound was prepared as a white powder.

TLC: (5% methanol in dichloromethane, R_{f}): 0.39 (UV, 254 nm, 280 nm).

¹H NMR (500 MHz, CDCl₃, 1 drop of DMF-d, and 3 drops of TFA-d) δ 8.89 (d, J=1.9 Hz, 1H), 8.38 (dd, J=8.9, 1.9 Hz, 1H), 7.84 (d, J=8.9 Hz, 1H), 7.50 (d, J=2.1 Hz, 1H), 7.34 (dd, J=8.4, 2.1 Hz, 1H), 7.25 (d, J=8.4 Hz, 1H), 4.19-4.09 (m, 1H), 3.71 (ddd, J=17.7, 4.1, 1.6 Hz, 1H), 3.64 (dd, J=17.8, 11.5 Hz, 1H), 3.23 (ddd, J=17.7, 4.1, 1.6 Hz, 1H), 3.17-3.07 (m, 1H).

¹³C NMR (126 MHz, CDCl₃, 1 drop of DMF-d, and 3 drops of TFA-d) δ 202.2, 172.0, 164.7, 161.2, 146.9, 138.7, 134.9, 134.2, 133.8, 130.4, 128.2, 127.6, 121.9, 120.6, 108.2, 95.1, 42.6, 34.6, 33.4.

### 7-bromo-3-(2,4-dichlorophenyl)-3,4-dihydroacridine-1,9(2H,10H)-dione (81)

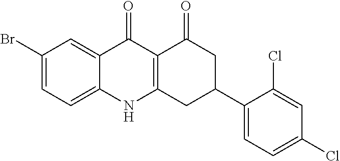 [SMILES: O=C1CC(c2ccc(Cl)cc2Cl)Cc2[nH]c3ccc(Br)cc3c(=O)c21]

Following general procedure L, the title compound was prepared as a tan-colored solid.

TLC: (5% methanol in dichloromethane, R_{f}): 0.39 (UV, 254 nm, 280 nm).

¹H NMR (500 MHz, CDCl₃, 1 drop of DMF-d, and 3 drops of TFA-d) δ 8.67 (d, J=2.1 Hz, 1H), 8.21 (dd, J=8.9, 2.1 Hz, 1H), 8.01 (d, J=8.9 Hz, 1H), 7.50 (d, J=2.1 Hz, 1H), 7.34 (dd, J=8.4, 2.1 Hz, 1H), 7.25 (d, J=8.4 Hz, 1H), 4.21-4.08 (m, 1H), 3.76-3.60 (m, 2H), 3.23 (ddd, J=17.7, 4.1, 1.6 Hz, 1H), 3.13 (dd, J=17.8, 12.6 Hz, 1H).

¹³C NMR (126 MHz, CDCl₃, 1 drop of DMF-d, and 3 drops of TFA-d) δ 202.3, 172.2, 164.6, 161.1, 141.5, 138.4, 134.97, 134.91, 134.2, 130.4, 128.2, 127.6, 127.4, 124.1, 122.3, 120.5, 37.8, 34.4, 32.5.

### 3-(2,4-dichlorophenyl)-7-fluoro-3,4-dihydroacridine-1,9(2H,10H)-dione (82)

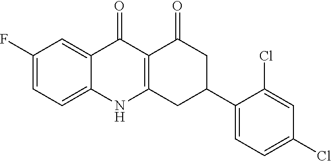 [SMILES: O=C1CC(c2ccc(Cl)cc2Cl)Cc2[nH]c3ccc(F)cc3c(=O)c21]

Following general procedure L, the title compound was prepared as a white powder.

TLC: (5% methanol in dichloromethane, R_{f}): 0.39 (UV, 254 nm, 280 nm).

¹H NMR (500 MHz, CDCl₃ and 3 drops of TFA-d) δ 8.17 (dd, J=7.5, 2.8 Hz, 1H), 8.13 (dd, J=9.3, 4.2 Hz, 1H), 7.91 (ddd, J=9.3, 7.5, 2.8 Hz, 1H), 7.50 (d, J=2.1 Hz, 1H), 7.35 (dd, J=8.4, 2.1 Hz, 1H), 7.25 (d, J=8.4 Hz, 1H), 4.22-4.10 (m, 1H), 3.76-3.60 (m, 2H), 3.24 (ddd, J=17.7, 4.1, 1.5 Hz, 1H), 3.15 (dd, J=17.8, 12.5 Hz, 1H).

¹³C NMR (126 MHz, CDCl₃ and 3 drops of TFA-d) δ 202.4, 173.0 (d, J=4.3 Hz), 163.0, 160.5 (d, J=1.9 Hz), 136.3, 135.1, 134.9, 134.3, 130.6, 128.38 (d, J=25.9 Hz), 128.38, 127.6, 123.4 (d, J=9.0 Hz), 121.0 (d, J=9.6 Hz), 110.0 (d, J=24.6 Hz), 108.0, 42.7, 34.7, 33.5.

¹⁹F NMR (471 MHz, CDCl₃ and 3 drops of TFA-d) δ −105.01.

### 8-chloro-2H-benzo[d][1,3]oxazine-2,4(1H)-dione (98)

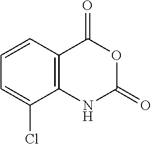 [SMILES: O=c1[nH]c2c(Cl)cccc2c(=O)o1]

Following general procedure A, the title compound was prepared as a white powder.

LCMS (ESI-MS) m/z: [M+H]⁺ calcd for C₈H₅ClNO₃ 197.99; found 197.8. Retention time: 1.418 min.

TLC: (20% EtOAc in hexanes, R_{f}): 0.16 (UV, 254 nm, 280 nm).

¹H NMR (500 MHz, DMSO-d₆) δ 11.40 (s, 1H), 7.91 (dd, J=7.9, 1.4 Hz, 1H), 7.87 (dd, J=8.0, 1.4 Hz, 1H), 7.25 (dd, J=7.9, 8.0 Hz, 1H).

¹³C NMR (126 MHz, DMSO-d₆) δ 159.6, 147.1, 138.8, 137.1, 128.4, 124.4, 119.3, 113.3.

### 7-chloro-2H-benzo[d][1,3]oxazine-2,4(1H)-dione (99)

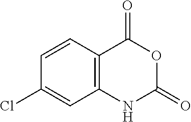 [SMILES: O=c1[nH]c2cc(Cl)ccc2c(=O)o1]

Following general procedure A, the title compound was prepared as a white powder.

LCMS (ESI-MS) m/z: [M+H]⁺ calcd for C₈H₅ClNO₃ 197.99; found 197.8. Retention time: 2.741 min.

TLC: (20% EtOAc in hexanes, R_{f}): 0.16 (UV, 254 nm, 280 nm).

¹H NMR (500 MHz, DMSO-d₆) δ 11.85 (s, 1H), 7.91 (d, J=8.6 Hz, 1H), 7.29 (dd, J=8.6, 2.0 Hz, 1H), 7.14 (d, J=2.0 Hz, 1H).

¹³C NMR (126 MHz, DMSO-d₆) δ 159.5, 147.3, 142.9, 141.6, 131.3, 124.1, 115.2, 109.9.

### 5-chloro-2H-benzo[d][1,3]oxazine-2,4(1H)-dione (100)

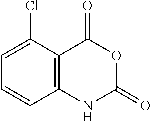 [SMILES: O=c1[nH]c2cccc(Cl)c2c(=O)o1]

Following general procedure A, the title compound was prepared as a white powder.

LCMS (ESI-MS) m/z: [M+H]⁺ calcd for C₈H₅ClNO₃ 197.99; found 197.8. Retention time: 2.219 min.

TLC: (50% EtOAc in hexanes, R_{f}): 0.28 (UV, 254 nm, 280 nm).

¹H NMR (500 MHz, DMSO-d₆) δ 11.86 (s, 1H), 7.65 (dd, J=8.8, 8.1 Hz, 1H), 7.30 (dd, J=8.1, 0.8 Hz, 1H), 7.11 (d, J=8.8 Hz, 1H).

¹³C NMR (126 MHz, DMSO-d₆) δ 156.9, 147.1, 144.4, 137.0, 135.4, 126.1, 115.0, 108.5.

### 8-methyl-2H-benzo[d][1,3]oxazine-2,4(1H)-dione (101)

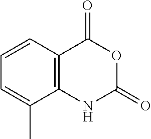

Following general procedure A, the title compound was prepared as a white powder.

LCMS (ESI-MS) m/z: [M+H]⁺ calcd for C₉H₅NO₃ 178.04; found 177.9. Retention time: 1.674 min.

TLC: (50% EtOAc in hexanes, R_{f}): 0.29 (UV, 254 nm, 280 nm).

¹H NMR (500 MHz, DMSO-d₆) δ 11.01 (s, 1H), 7.76 (d, J=8.2 Hz, 1H), 7.57 (d, J=7.5 Hz, 1H), 7.14 (dd, J=8.2, 7.5 Hz, 1H), 2.32 (s, 3H).

¹³C NMR (126 MHz, DMSO-d₆) δ 160.4, 147.7, 140.1, 138.3, 127.1, 124.8, 123.6, 110.8, 17.4.

### 7-methyl-2H-benzo[d][1,3]oxazine-2,4(1H)-dione (102)

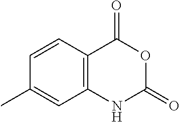

Following general procedure A, the title compound was prepared as a white powder.

LCMS (ESI-MS) m/z: [M+H]⁺ calcd for C₉H₈NO₃ 178.04; found 177.9. Retention time: 2.263 min.

TLC: (50% EtOAc in hexanes, R_{f}): 0.21 (UV, 254 nm, 280 nm).

¹H NMR (500 MHz, DMSO-d₆) δ 11.68 (s, 1H), 7.78 (d, J=8.3 Hz, 1H), 7.07 (d, J=8.3 Hz, 1H), 6.93 (s, 1H), 2.37 (s, 3H).

¹³C NMR (126 MHz, DMSO-d₆) δ 160.1, 148.6, 147.7, 141.9, 129.3, 125.2, 115.5, 108.1, 22.0.

### 6-methyl-2H-benzo[d][1,3]oxazine-2,4(1H)-dione (103)

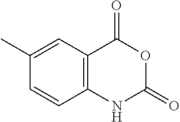

Following general procedure A, the title compound was prepared as an off-white and slightly yellow powder.

LCMS (ESI-MS) m/z: [M+H]⁺ calcd for C₉H₈NO₃ 178.04; found 177.9. Retention time: 2.508 min.

TLC: (50% EtOAc in hexanes, R_{f}): 0.21 (UV, 254 nm, 280 nm).

¹H NMR (500 MHz, DMSO-d₆) δ 11.63 (s, 1H), 7.70 (d, J=2.2 Hz, 1H), 7.55 (dd, J=8.4, 2.2 Hz, 1H), 7.05 (d, J=8.4 Hz, 1H), 2.32 (s, 3H).

¹³C NMR (126 MHz, DMSO-d₆) δ 159.9, 147.1, 139.2, 137.9, 132.9, 128.3, 115.3, 110.0, 20.0.

### 5-methyl-2H-benzo[d][1,3]oxazine-2,4(1H)-dione (104)

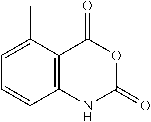

Following general procedure A, the title compound was prepared as a white powder.

LCMS (ESI-MS) m/z: [M+H]⁺ calcd for C₉H₈NO₃ 178.04; found 177.9. Retention time: 2.555 min.

TLC: (50% EtOAc in hexanes, R_{f}): 0.24 (UV, 254 nm, 280 nm).

¹H NMR (500 MHz, DMSO-d₆) δ 11.59 (s, 1H), 7.56 (dd, J=8.6, 7.9 Hz, 1H), 7.05 (d, J=7.9 Hz, 1H), 6.98 (d, J=8.6 Hz, 1H), 2.59 (s, 3H).

¹³C NMR (126 MHz, DMSO-d₆) δ 158.9, 147.1, 142.5 (2×C), 135.8, 125.9, 113.3, 108.6, 21.7.

### 8-methoxy-2H-benzo[d][1,3]oxazine-2,4(1H)-dione (105)

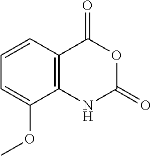

Following general procedure A, the title compound was prepared as a white powder.

LCMS (ESI-MS) m/z: [M+H]⁺ calcd for C₉H₈NO₄ 194.04; found 193.9. Retention time: 1.284 min.

TLC: (50% EtOAc in hexanes, R_{f}): 0.24 (UV, 254 nm, 280 nm).

¹H NMR (500 MHz, DMSO-d₆) δ 11.26 (s, 1H), 7.47 (d, J=8.1 Hz, 1H), 7.38 (d, J=7.8 Hz, 1H), 7.20 (dd, J=8.1, 7.8 Hz, 1H), 3.89 (s, 3H).

¹³C NMR (126 MHz, DMSO-d₆) δ 159.8, 146.8, 146.2, 131.5, 123.4, 119.6, 117.4, 110.8, 56.3.

### 7-methoxy-2H-benzo[d][1,3]oxazine-2,4(1H)-dione (106)

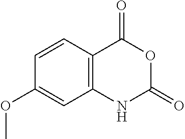

Following general procedure A, the title compound was prepared as an off-white, slightly tan powder.

LCMS (ESI-MS) m/z: [M+H]⁺ calcd for C₉H₈NO₄ 194.04; found 193.9. Retention time: 1.581 min.

TLC: (50% EtOAc in hexanes, R_{f}): 0.24 (UV, 254 nm, 280 nm).

¹H NMR (500 MHz, DMSO-d₆) δ 11.64 (s, 1H), 7.82 (d, J=8.9 Hz, 1H), 6.82 (dd, J=8.9, 2.4 Hz, 1H), 6.58 (d, J=2.4 Hz, 1H), 3.85 (s, 3H).

¹³C NMR (126 MHz, DMSO-d₆) δ 165.8, 159.2, 147.4, 143.6, 131.0, 111.6, 102.8, 98.5, 55.9.

### 6-methoxy-2H-benzo[d][1,3]oxazine-2,4(1H)-dione (107)

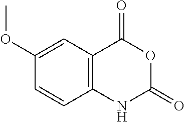

Following general procedure A, the title compound was prepared as an off-white, slightly tan powder.

LCMS (ESI-MS) m/z: [M+H]⁺ calcd for C₉H₈NO₄ 194.04; found 193.9. Retention time: 1.295 min

TLC: (50% EtOAc in hexanes, R_{f}): 0.31 (UV, 254 nm, 280 nm).

¹H NMR (500 MHz, DMSO-d₆) δ 11.61 (s, 1H), 7.38 (d, J=9.4 Hz, 1H), 7.34 (s, 1H), 7.11 (d, J=9.4 Hz, 1H), 3.80 (s, 3H).

¹³C NMR (126 MHz, DMSO-d₆) δ 160.3, 155.6, 147.4, 135.9, 126.2, 117.4, 111.1, 110.3, 56.2.

### 5-methoxy-2H-benzo[d][1,3]oxazine-2,4(1H)-dione (108)

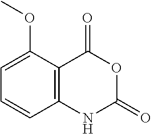

Following general procedure A, the title compound was prepared as an off-white, slightly tan powder.

LCMS (ESI-MS) m/z: [M+H]⁺ calcd for C₉H₈NO₄ 194.04; found 193.9. Retention time: 0.541 min.

TLC: (50% EtOAc in hexanes, R_{f}): 0.14 (UV, 254 nm, 280 nm).

¹H NMR (500 MHz, DMSO-d₆) δ 11.57 (s, 1H), 7.63 (dd, J=8.6, 8.2 Hz, 1H), 6.82 (d, J=8.6 Hz, 1H), 6.68 (d, J=8.2 Hz, 1H), 3.88 (s, 3H).

¹³C NMR (126 MHz, DMSO-d₆) δ 161.1, 155.6, 147.3, 143.2, 137.6, 106.9, 105.9, 99.3, 56.2.

### 6-iodo-2H-benzo[d][1,3]oxazine-2,4(1H)-dione (109)

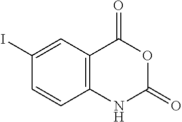 [SMILES: O=c1[nH]c2ccc(I)cc2c(=O)o1]

Following general procedure A, the title compound was prepared as a white powder.

LCMS (ESI-MS) m/z: [M+H]⁺ calcd for C₈H₅INO₃ 289.92; found 289.6. Retention time: 3.294 min.

TLC: (50% EtOAc in hexanes, R_{f}): 0.24 (UV, 254 nm, 280 nm).

¹H NMR (500 MHz, DMSO-d₆) δ 11.82 (s, 1H), 8.13 (d, J=2.0 Hz, 1H), 8.02 (dd, J=8.6, 2.0 Hz, 1H), 6.96 (d, J=8.6 Hz, 1H).

¹³C NMR (126 MHz, DMSO-d₆) δ 159.1, 147.2, 145.2, 141.4, 136.9, 118.0, 113.1, 86.5.

### 6-bromo-2H-benzo[d][1,3]oxazine-2,4(1H)-dione (110)

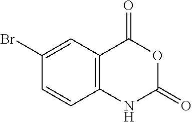 [SMILES: O=c1[nH]c2ccc(Br)cc2c(=O)o1]

Following general procedure A, the title compound was prepared as a white powder.

LCMS (ESI-MS) m/z: [M+H]⁺ calcd for C₈H₅BrNO₃ 241.94; found 241.7. Retention time: 3.093 min.

TLC: (50% EtOAc in hexanes, R_{f}): 0.24 (UV, 254 nm, 280 nm).

¹H NMR (500 MHz, DMSO-d₆) δ 11.86 (s, 1H), 7.99 (d, J=2.3 Hz, 1H), 7.90 (dd, J=8.7, 2.3 Hz, 1H), 7.10 (d, J=8.7 Hz, 1H).

¹³C NMR (126 MHz, DMSO-d₆) δ 159.3, 147.2, 141.1, 139.8, 131.0, 118.1, 115.0, 112.9.

### 6-fluoro-2H-benzo[d][1,3]oxazine-2,4(1H)-dione (111)

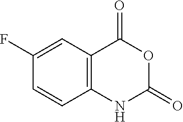 [SMILES: O=c1[nH]c2ccc(F)cc2c(=O)o1]

Following general procedure A, the title compound was prepared as a white powder.

LCMS (ESI-MS) m/z: [M+H]⁺ calcd for C₈H₅FNO₃ 182.02; found 181.8. Retention time: 1.194 min.

TLC: (50% EtOAc in hexanes, R_{f}): 0.35 (UV, 254 nm, 280 nm).

¹H NMR (500 MHz, DMSO-d₆) δ 11.80 (s, 1H), 7.74-7.60 (m, 2H), 7.19 (dd, J=9.2, 4.6 Hz, 1H).

¹³C NMR (126 MHz, DMSO-d₆) δ 159.6 (d, J_{C—F}=3.3 Hz), 157.9 (d, J_{C—F}=241.3 Hz), 147.2, 138.6 (d, J_{C—F}=1.5 Hz), 125.2 (d, J_{C—F}=24.5 Hz), 118.0 (d, J_{C—F}=8.4 Hz), 114.4 (d, J_{C—F}=24.3 Hz), 111.9 (d, J_{C—F}=24.4 Hz).

¹⁹F NMR (471 MHz, DMSO-d₆) δ −118.76-−118.85 (m).

### 2H-benzo[d][1,3]oxazine-2,4(1H)-dione (112)

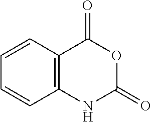 [SMILES: O=c1[nH]c2ccccc2c(=O)o1]

Following general procedure A, the title compound was prepared as a white powder.

LCMS (ESI-MS) m/z: [M+H]⁺ calcd for C₈H₆NO₃ 164.03; found 163.9. Retention time: 0.711 min.

TLC: (50% EtOAc in hexanes, R_{f}): 0.38 (UV, 254 nm, 280 nm).

¹H NMR (500 MHz, DMSO-d₆) δ 11.72 (s, 1H), 7.91 (d, J=7.9 Hz, 1H), 7.78-7.69 (m, 1H), 7.25 (dd, J=8.4, 7.9 Hz, 1H), 7.15 (d, J=8.1 Hz, 1H).

¹³C NMR (126 MHz, DMSO-d₆) δ 160.3, 147.5, 141.8, 137.4, 129.4, 123.9, 115.8, 110.7.

### 3-cyclopropyl-3,4-dihydroacridine-1,9(2H,10H)-dione (113)

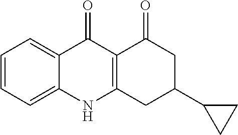 [SMILES: O=C1CC(C2CC2)Cc2[nH]c3ccccc3c(=O)c21]

Following general procedure L, the title compound was prepared as a white powder.

LCMS (ESI-MS) m/z: [M+H]⁺ calcd for C₁₆H₁₆NO₂ 254.11; found 253.9. Retention time: 2.888 min.

TLC: (5% methanol in dichloromethane, R_{f}): 0.34 (UV, 254 nm, 280 nm).

¹H NMR (500 MHz, DMSO-d₆) δ 12.77 (s, 1H), 8.10 (d, J=8.4 Hz, 1H), 7.75-7.65 (m, 2H), 7.41-7.32 (m, 1H), 3.16 (dd, J=16.9, 3.9 Hz, 1H), 2.97 (dd, J=16.7, 10.4 Hz, 1H), 2.49 (dd, J=16.9, 3.9 Hz, 1H), 2.41 (dd, J=16.7, 10.4 Hz, 1H), 1.50-1.37 (m, 1H), 0.82-0.71 (m, 1H), 0.51-0.39 (m, 2H), 0.27-0.13 (m, 2H).

### 8-chloro-3-cyclopropyl-3,4-dihydroacridine-1,9(2H,10H)-dione (114)

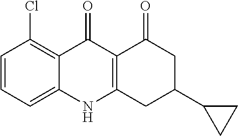 [SMILES: O=C1CC(C2CC2)Cc2[nH]c3cccc(Cl)c3c(=O)c21]

Following general procedure L, the title compound was prepared as a tan-colored solid.

TLC: (5% methanol in dichloromethane, R_{f}): 0.40 (UV, 254 nm, 280 nm).

¹H NMR (500 MHz, CDCl₃ and 3 drops of TFA-d) δ 8.04-7.93 (m, 2H), 7.84 (dd, J=5.7, 3.1 Hz, 1H), 3.59 (dd, J=18.0, 4.1 Hz, 1H), 3.29 (dd, J=18.0, 10.9 Hz, 1H), 3.13 (dd, J=18.0, 4.1 Hz, 1H), 2.87 (dd, J=18.0, 10.9 Hz, 1H), 1.77-1.59 (m, 1H), 0.86-0.74 (m, 1H), 0.74-0.56 (m, 2H), 0.38-0.19 (m, 2H).

### 7-chloro-3-cyclopropyl-3,4-dihydroacridine-1,9(2H,10H)-dione (115)

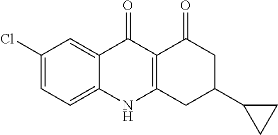 [SMILES: O=C1CC(C2CC2)Cc2[nH]c3ccc(Cl)cc3c(=O)c21]

Following general procedure L, the title compound was prepared as a white powder.

TLC: (5% methanol in dichloromethane, R_{f}): 0.40 (UV, 254 nm, 280 nm).

¹H NMR (500 MHz, CDCl₃ and 3 drops of TFA-d) δ 8.46 (s, 1H), 8.10-8.00 (m, 2H), 3.70-3.50 (m, 1H), 3.30 (dd, J=18.0, 11.0 Hz, 1H), 3.17-3.09 (m, 1H), 2.85 (dd, J=18.0, 11.0 Hz, 1H), 1.72-1.61 (m, 1H), 0.86-0.77 (m, 1H), 0.70-0.61 (m, 2H), 0.33-0.22 (m, 2H).

¹³C NMR (126 MHz, CDCl₃ and 3 drops of TFA-d) δ 203.5, 172.4, 162.0, 138.8, 137.9, 136.3, 124.2, 122.2, 120.2, 108.4, 43.4, 38.7, 34.3, 15.6, 3.9, 3.8.

### 6-chloro-3-cyclopropyl-3,4-dihydroacridine-1,9(2H,10H)-dione (116)

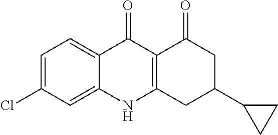 [SMILES: O=C1CC(C2CC2)Cc2[nH]c3cc(Cl)ccc3c(=O)c21]

Following general procedure L, the title compound was prepared as a white powder.

LCMS (ESI-MS) m/z: [M+H]⁺ calcd for C₁₆H₁₅ClNO₂ 288.07; found 287.9. Retention time: 3.412 min.

TLC: (5% methanol in dichloromethane, R_{f}): 0.19 (UV, 254 nm, 280 nm).

¹H NMR (500 MHz, CDCl₃ and 3 drops of TFA-d) δ 8.45 (d, J=8.9 Hz, 1H), 8.05 (d, J=1.8 Hz, 1H), 7.79 (dd, J=8.9, 1.8 Hz, 1H), 3.61 (ddd, J=18.0, 4.2, 1.4 Hz, 1H), 3.31 (dd, J=18.1, 11.0 Hz, 1H), 3.11 (ddd, J=18.0, 4.2, 1.4 Hz, 1H), 2.84 (dd, J=18.1, 11.0 Hz, 1H), 1.71-1.59 (m, 1H), 0.89-0.75 (m, 1H), 0.70-0.60 (m, 2H), 0.32-0.22 (m, 2H).

¹³C NMR (126 MHz, CDCl₃ and 3 drops of TFA-d) δ 203.57, 173.08, 163.09, 145.64, 140.08, 130.65, 126.55, 120.06, 117.64, 108.2, 43.3, 38.7, 34.3, 15.6, 3.9, 3.7.

### 5-chloro-3-cyclopropyl-3,4-dihydroacridine-1,9(2H,10H)-dione (117)

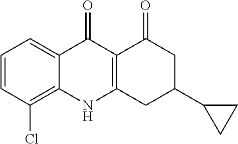 [SMILES: O=C1CC(C2CC2)Cc2[nH]c3c(Cl)cccc3c(=O)c21]

Following general procedure L, the title compound was prepared as a white powder.

LCMS (ESI-MS) m/z: [M+H]⁺ calcd for C₁₆H₁₅ClNO₂ 288.07; found 287.8. Retention time: 3.500 min.

TLC: (5% methanol in dichloromethane, R_{f}): 0.33 (UV, 254 nm, 280 nm).

¹H NMR (500 MHz, DMSO-d₆) b 8.08 (d, J=7.9 Hz, 1H), 7.61 (d, J=7.4 Hz, 1H), 7.10 (dd, J=7.9, 7.4 Hz, 1H), 3.01 (dd, J=15.7, 3.9 Hz, 1H), 2.78 (dd, J=16.3, 10.6 Hz, 1H), 2.50 (dd, J=15.7, 3.9 Hz, 1H), 2.36 (dd, J=16.3, 10.6 Hz, 1H), 1.37-1.26 (m, 1H), 0.78-0.67 (m, 1H), 0.47-0.35 (m, 2H), 0.24-0.11 (m, 2H).

### 3-cyclopropyl-8-methyl-3,4-dihydroacridine-1,9(2H,10H)-dione (118)

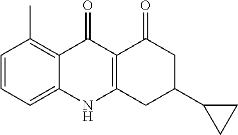 [SMILES: Cc1cccc2[nH]c3c(c(=O)c12)C(=O)CC(C1CC1)C3]

Following general procedure L, the title compound was prepared as a brown solid.

TLC: (5% methanol in dichloromethane, R_{f}): 0.32 (UV, 254 nm, 280 nm).

¹H NMR (500 MHz, DMSO-d₆) δ 11.81 (s, 1H), 7.46 (dd, J=8.1, 7.3 Hz, 1H), 7.33 (d, J=8.1 Hz, 1H), 7.04 (d, J=7.3 Hz, 1H), 2.98 (dd, J=16.5, 4.2 Hz, 1H), 2.85 (dd, J=16.5, 11.1 Hz, 1H), 2.74 (s, 3H), 2.45 (dd, J=16.5, 4.2 Hz, 1H), 2.33 (dd, J=16.5, 11.1 Hz, 1H), 1.44-1.34 (m, 1H), 0.79-0.69 (m, 1H), 0.48-0.38 (m, 2H), 0.26-0.10 (m, 2H).

### 3-cyclopropyl-7-methyl-3,4-dihydroacridine-1,9(2H,10H)-dione (119)

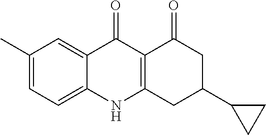 [SMILES: Cc1ccc2[nH]c3c(c(=O)c2c1)C(=O)CC(C1CC1)C3]

Following general procedure L, the title compound was prepared as a pale yellow solid.

TLC: (5% methanol in dichloromethane, R_{f}): 0.32 (UV, 254 nm, 280 nm).

¹H NMR (500 MHz, CDCl₃ and 3 drops of TFA-d) δ 8.28 (s, 1H), 7.99-7.89 (m, 2H), 3.58 (ddd, J=17.8, 4.2, 1.4 Hz, 1H), 3.28 (dd, J=17.9, 11.0 Hz, 1H), 3.11 (ddd, J=17.8, 4.2, 1.4 Hz, 1H), 2.84 (dd, J=17.9, 11.0 Hz, 1H), 2.64 (s, 3H), 1.71-1.59 (m, 1H), 0.85-0.76 (m, 1H), 0.71-0.59 (m, 2H), 0.34-0.22 (m, 2H).

¹³C NMR (126 MHz, CDCl₃ and 3 drops of TFA-d) δ 203.7, 172.9, 160.7, 140.8, 140.3, 137.7, 124.1, 120.1, 119.3, 108.0, 43.5, 38.8, 34.2, 21.6, 15.6, 3.8, 3.7.

### 3-cyclopropyl-6-methyl-3,4-dihydroacridine-1,9(2H,10H)-dione (120)

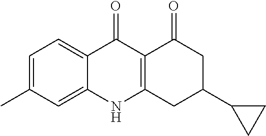 [SMILES: Cc1ccc2c(=O)c3c([nH]c2c1)CC(C1CC1)CC3=O]

Following general procedure L, the title compound was prepared as a white powder.

TLC: (5% methanol in dichloromethane, R_{f}): 0.27 (UV, 254 nm, 280 nm).

¹H NMR (500 MHz, CDCl₃ and 1 drop of TFA-d) δ 8.39 (d, J=8.5 Hz, 1H), 7.79 (s, 1H), 7.67 (dd, J=8.5, 1.2 Hz, 1H), 3.58 (ddd, J=17.9, 4.1, 1.3 Hz, 1H), 3.27 (dd, J=17.9, 11.1 Hz, 1H), 3.10 (ddd, J=17.9, 4.1, 1.3 Hz, 1H), 2.82 (dd, J=17.9, 11.1 Hz, 1H), 2.66 (s, 3H), 1.71-1.56 (m, 1H), 0.90-0.74 (m, 1H), 0.70-0.60 (m, 2H), 0.34-0.18 (m, 2H).

¹³C NMR (126 MHz, CDCl₃ and 1 drop of TFA-d) δ 203.5, 173.0, 161.5, 151.4, 139.8, 131.5, 124.9, 119.6, 117.1, 107.7, 43.4, 38.8, 34.2, 22.5, 15.6, 3.8, 3.7.

### 3-cyclopropyl-5-methyl-3,4-dihydroacridine-1,9(2H,10H)-dione (121)

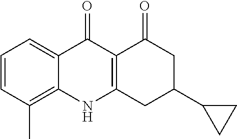 [SMILES: Cc1cccc2c(=O)c3c([nH]c12)CC(C1CC1)CC3=O]

Following general procedure L, the title compound was prepared as a pale yellow solid.

TLC: (5% methanol in dichloromethane, R_{f}): 0.40 (UV, 254 nm, 280 nm).

¹H NMR (500 MHz, CDCl₃ and 3 drops of TFA-d) δ 8.40 (d, J=8.2 Hz, 1H), 7.95 (d, J=7.3 Hz, 1H), 7.75 (dd, J=8.2, 7.3 Hz, 1H), 3.75 (ddd, J=17.8, 4.2, 1.4 Hz, 1H), 3.35 (dd, J=18.1, 10.2 Hz, 1H), 3.12 (ddd, J=17.8, 4.2, 1.4 Hz, 1H), 2.91-2.72 (m, 4H), 1.70-1.56 (m, 1H), 0.87-0.77 (m, 1H), 0.71-0.55 (m, 2H), 0.34-0.19 (m, 2H).

¹³C NMR (126 MHz, CDCl₃ and 3 drops of TFA-d) δ 203.7, 173.6, 162.5, 139.3, 138.4, 129.7, 129.1, 123.2, 119.7, 107.9, 43.3, 38.7, 34.1, 17.1, 15.6, 3.9, 3.6.

### 3-cyclopropyl-8-methoxy-3,4-dihydroacridine-1,9(2H,10H)-dione (122)

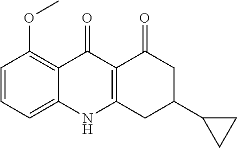 [SMILES: COc1cccc2[nH]c3c(c(=O)c12)C(=O)CC(C1CC1)C3]

Following general procedure L, the title compound was prepared as an off-white powder.

LCMS (ESI-MS) m/z: [M+H]⁺ calcd for C₁₇H₁₈NO₃ 284.12; found 283.9. Retention time: 2.839 min.

TLC: (5% methanol in dichloromethane, R_{f}): 0.19 (UV, 254 nm, 280 nm).

¹H NMR (500 MHz, CDCl₃ and 3 drops of TFA-d) δ 8.00 (dd, J=8.6, 7.9 Hz, 1H), 7.51 (d, J=7.9 Hz, 1H), 7.16 (d, J=8.6 Hz, 1H), 4.11 (s, 3H), 3.52 (ddd, J=17.9, 4.3, 1.4 Hz, 1H), 3.23 (dd, J=17.9, 11.1 Hz, 1H), 3.08 (ddd, J=17.9, 4.3, 1.4 Hz, 1H), 2.82 (dd, J=17.9, 11.1 Hz, 1H), 1.67-1.57 (m, 1H), 0.85-0.75 (m, 1H), 0.69-0.60 (m, 2H), 0.31-0.22 (m, 2H).

¹³C NMR (126 MHz, CDCl₃ and 3 drops of TFA-d) δ 203.8, 175.7, 161.7, 160.7, 141.1, 139.4, 111.7, 110.5, 109.4, 107.6, 57.0, 43.4, 38.6, 34.1, 15.6, 3.8, 3.6.

### 3-cyclopropyl-7-methoxy-3,4-dihydroacridine-1,9(2H,10H)-dione (123)

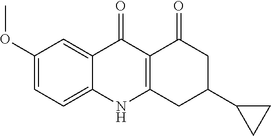 [SMILES: COc1ccc2[nH]c3c(c(=O)c2c1)C(=O)CC(C1CC1)C3]

Following general procedure L, the title compound was prepared as a white powder.

TLC: (5% methanol in dichloromethane, R_{f}): 0.19 (UV, 254 nm, 280 nm).

¹H NMR (500 MHz, DMSO-d₆) δ 12.04 (s, 1H), 7.52-7.46 (m, 2H), 7.30 (dd, J=8.9, 2.9 Hz, 1H), 3.83 (s, 3H), 3.01 (dd, J=16.1, 4.3 Hz, 1H), 2.91 (dd, J=16.1, 11.1 Hz, 1H), 2.47 (dd, J=16.1, 4.3 Hz, 1H), 2.36 (dd, J=16.1, 11.1 Hz, 1H), 1.49-1.37 (m, 1H), 0.79-0.70 (m, 1H), 0.49-0.35 (m, 2H), 0.26-0.14 (m, 2H).

### 3-cyclopropyl-6-methoxy-3,4-dihydroacridine-1,9(2H,10H)-dione (124)

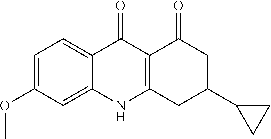 [SMILES: COc1ccc2c(=O)c3c([nH]c2c1)CC(C1CC1)CC3=O]

Following general procedure L, the title compound was prepared as a white powder.

LCMS (ESI-MS) m/z: [M+H]⁺ calcd for C₁₇H₁₈NO₃ 284.12; found 283.9. Retention time: 3.225 min.

TLC: (5% methanol in dichloromethane, R_{f}): 0.34 (UV, 254 nm, 280 nm).

¹H NMR (500 MHz, CDCl₃ and 3 drops of TFA-d) δ 8.35 (d, J=9.2 Hz, 1H), 7.37 (dd, J=9.2, 2.3 Hz, 1H), 7.33 (d, J=2.3 Hz, 1H), 4.03 (s, 3H), 3.55 (ddd, J=17.9, 4.3, 1.4 Hz, 1H), 3.26 (dd, J=17.8, 10.3 Hz, 1H), 3.07 (ddd, J=17.9, 4.3, 1.4 Hz, 1H), 2.80 (dd, J=17.8, 10.3 Hz, 1H), 1.69-1.58 (m, 1H), 0.90-0.76 (m, 1H), 0.70-0.59 (m, 2H), 0.33-0.21 (m, 2H).

¹³C NMR (126 MHz, CDCl₃ and 3 drops of TFA-d) δ 203.4, 171.9, 167.9, 161.6, 142.5, 126.8, 121.7, 113.1, 107.3, 99.9, 56.7, 43.4, 38.9, 34.2, 15.6, 3.8, 3.7.

### 3-cyclopropyl-5-methoxy-3,4-dihydroacridine-1,9(2H,10H)-dione (125)

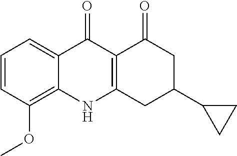 [SMILES: COc1cccc2c(=O)c3c([nH]c12)CC(C1CC1)CC3=O]

Following general procedure L, the title compound was prepared as a white powder.

LCMS (ESI-MS) m/z: [M+H]⁺ calcd for C₁₇H₁₈NO₃ 284.12; found 283.9. Retention time: 3.109 min.

TLC: (5% methanol in dichloromethane, R_{f}): 0.29 (UV, 254 nm, 280 nm).

¹H NMR (500 MHz, CDCl₃ and 3 drops of TFA-d) δ 8.04 (dd, J=8.4, 1.0 Hz, 1H), 7.76 (dd, J=8.4, 8.0 Hz, 1H), 7.49 (dd, J=8.0, 1.0 Hz, 1H), 4.08 (s, 3H), 3.75 (ddd, J=17.8, 4.2, 1.4 Hz, 1H), 3.35 (dd, J=18.1, 11.0 Hz, 1H), 3.12 (ddd, J=17.8, 4.2, 1.4 Hz, 1H), 2.84 (dd, J=18.1, 11.0 Hz, 1H), 1.70-1.58 (m, 1H), 0.81 (s, 1H), 0.71-0.58 (m, 2H), 0.34-0.21 (m, 2H).

¹³C NMR (126 MHz, CDCl₃ and 3 drops of TFA-d) δ 203.8, 173.4, 161.3, 149.3, 130.4, 129.8, 120.2, 116.2, 116.0, 108.4, 56.8, 43.4, 38.7, 34.1, 15.6, 3.8, 3.7.

### 3-cyclobutyl-3,4-dihydroacridine-1,9(2H,10H)-dione (126)

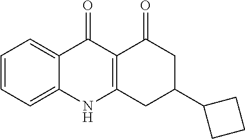 [SMILES: O=C1CC(C2CCC2)Cc2[nH]c3ccccc3c(=O)c21]

Following general procedure L, the title compound was prepared as a white powder.

LCMS (ESI-MS) m/z: [M+H]⁺ calcd for C₁₇H₁₈NO₂ 268.13; found 267.9. Retention time: 3.265 min.

TLC: (5% methanol in dichloromethane, R_{f}): 0.44 (UV, 254 nm, 280 nm).

¹H NMR (500 MHz, CDCl₃ and 3 drops of TFA-d) δ 8.51 (d, J=8.3 Hz, 1H), 8.18-8.03 (m, 2H), 7.85 (dd, J=8.3, 7.6 Hz, 1H), 3.50 (dd, J=18.1, 3.8 Hz, 1H), 3.09-2.92 (m, 2H), 2.55 (dd, J=17.7, 10.8 Hz, 1H), 2.46-2.27 (m, 2H), 2.21-2.08 (m, 2H), 2.03-1.70 (m, 4H).

¹³C NMR (126 MHz, CDCl₃ and 3 drops of TFA-d) δ 203.6, 173.3, 161.9, 139.5, 138.1, 129.4, 125.2, 120.5, 119.3, 108.0, 40.7, 39.6, 39.2, 31.5, 25.9, 25.8, 17.6.

### 3-cyclobutyl-8-methyl-3,4-dihydroacridine-1,9(2H,10H)-dione (127)

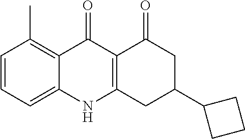 [SMILES: Cc1cccc2[nH]c3c(c(=O)c12)C(=O)CC(C1CCC1)C3]

Following general procedure L, the title compound was prepared as an off-white powder.

TLC: (5% methanol in dichloromethane, R_{f}): 0.25 (UV, 254 nm, 280 nm).

¹H NMR (500 MHz, DMSO-d₆) δ 11.65 (s, 1H), 7.42 (dd, J=8.2, 7.3 Hz, 1H), 7.27 (d, J=8.2 Hz, 1H), 7.00 (d, J=7.3 Hz, 1H), 2.82 (d, J=12.9 Hz, 1H), 2.69 (s, 3H), 2.55 (dd, J=16.8, 8.9 Hz, 1H), 2.30 (d, J=12.9 Hz, 1H), 2.16 (dd, J=16.8, 8.9 Hz, 1H), 2.07-1.88 (m, 4H), 1.82-1.59 (m, 4H).

### 3-cyclobutyl-7-methyl-3,4-dihydroacridine-1,9(2H,10H)-dione (128)

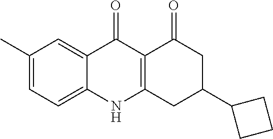 [SMILES: Cc1ccc2[nH]c3c(c(=O)c2c1)C(=O)CC(C1CCC1)C3]

Following general procedure L, the title compound was prepared as a white powder.

TLC: (5% methanol in dichloromethane, R_{f}): 0.19 (UV, 254 nm, 280 nm).

¹H NMR (500 MHz, CDCl₃ and 3 drops of TFA-d) δ 8.27 (s, 1H), 7.99-7.91 (m, 2H), 3.45 (ddd, J=17.9, 3.8, 1.6 Hz, 1H), 3.05-2.91 (m, 2H), 2.64 (s, 3H), 2.54 (dd, J=17.8, 10.9 Hz, 1H), 2.43-2.29 (m, 2H), 2.18-2.08 (m, 2H), 2.01-1.72 (m, 4H).

¹³C NMR (126 MHz, CDCl₃ and 3 drops of TFA-d) δ 203.7, 172.8, 160.7, 140.7, 140.1, 137.8, 124.0, 120.2, 119.3, 108.0, 40.7, 39.6, 39.2, 31.4, 26.0, 25.8, 21.6, 17.6.

### 3-cyclobutyl-6-methyl-3,4-dihydroacridine-1,9(2H,10H)-dione (129)

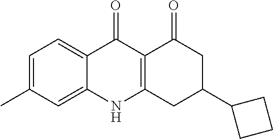 [SMILES: Cc1ccc2c(=O)c3c([nH]c2c1)CC(C1CCC1)CC3=O]

Following general procedure L, the title compound was prepared as a white powder.

LCMS (ESI-MS) m/z: [M+H]⁺ calcd for C₁₈H₂₀NO₂ 282.14; found 281.9. Retention time: 3.630 min.

TLC: (5% methanol in dichloromethane, R_{f}): 0.19 (UV, 254 nm, 280 nm).

¹H NMR (500 MHz, CDCl₃ and 3 drops of TFA-d) δ 8.39 (d, J=8.5 Hz, 1H), 7.79 (s, 1H), 7.67 (d, J=8.5 Hz, 1H), 3.43 (dd, J=17.9, 4.0 Hz, 1H), 3.04-2.91 (m, 2H), 2.66 (s, 3H), 2.54 (dd, J=17.7, 10.8 Hz, 1H), 2.44-2.28 (m, 2H), 2.18-2.09 (m, 2H), 2.00-1.71 (m, 4H).

¹³C NMR (126 MHz, CDCl₃ and 3 drops of TFA-d) δ 203.7, 173.0, 161.6, 151.6, 139.7, 131.6, 125.0, 119.5, 117.2, 107.8, 40.7, 39.6, 39.3, 31.5, 26.0, 25.8, 22.5, 17.7.

### 3-cyclobutyl-5-methyl-3,4-dihydroacridine-1,9(2H,10H)-dione (130)

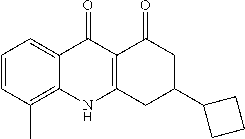 [SMILES: Cc1cccc2c(=O)c3c([nH]c12)CC(C1CCC1)CC3=O]

Following general procedure L, the title compound was prepared as a white powder.

LCMS (ESI-MS) m/z: [M+H]⁺ calcd for C₁₈H₂₀NO₂ 282.14; found 281.9. Retention time: 3.741 min.

TLC: (5% methanol in dichloromethane, R_{f}): 0.42 (UV, 254 nm, 280 nm).

¹H NMR (500 MHz, CDCl₃ and 3 drops of TFA-d) δ 8.40 (d, J=8.6 Hz, 1H), 7.93 (d, J=7.8 Hz, 1H), 7.75 (dd, J=8.6, 7.8 Hz, 1H), 3.63 (ddd, J=18.1, 3.7, 1.6 Hz, 1H), 3.08-2.92 (m, 2H), 2.77 (s, 3H), 2.54 (dd, J=17.7, 10.8 Hz, 1H), 2.41-2.28 (m, 2H), 2.17-2.07 (m, 2H), 2.02-1.69 (m, 4H).

¹³C NMR (126 MHz, CDCl₃ and 3 drops of TFA-d) δ 203.7, 173.6, 162.5, 139.3, 138.4, 129.7, 129.1, 123.2, 119.7, 108.0, 40.6, 39.6, 39.2, 31.4, 26.0, 25.8, 17.6, 17.1.

### 7-chloro-3-cyclobutyl-3,4-dihydroacridine-1,9(2H,10H)-dione (131)

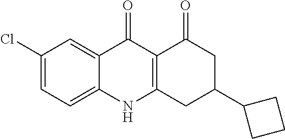 [SMILES: O=C1CC(C2CCC2)Cc2[nH]c3ccc(Cl)cc3c(=O)c21]

Following general procedure L, the title compound was prepared as a white powder.

LCMS (ESI-MS) m/z: [M+H]⁺ calcd for C₁₇H₁₇ClNO₂ 302.09; found 301.9. Retention time: 3.705 min.

TLC: (5% methanol in dichloromethane, R_{f}): 0.45 (UV, 254 nm, 280 nm).

¹H NMR (500 MHz, DMSO-d₆) δ 7.98 (s, 1H), 7.53-7.40 (m, 2H), 2.81 (d, J=16.4 Hz, 1H), 2.48 (d, J=10.9 Hz, 1H), 2.32 (d, J=16.4 Hz, 1H), 2.20-2.08 (m, 1H), 2.03 (dd, J=16.1, 10.9 Hz, 1H), 1.99-1.86 (m, 3H), 1.80-1.55 (m, 4H).

### 3-cyclobutyl-6-methoxy-3,4-dihydroacridine-1,9(2H,10H)-dione (132)

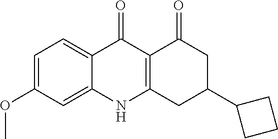 [SMILES: COc1ccc2c(=O)c3c([nH]c2c1)CC(C1CCC1)CC3=O]

Following general procedure L, the title compound was prepared as a white powder.

LCMS (ESI-MS) m/z: [M+H]⁺ calcd for C₁₈H₂₀NO₃ 298.14; found 297.9. Retention time: 3.481 min.

TLC: (5% methanol in dichloromethane, R_{f}): 0.39 (UV, 254 nm, 280 nm).

¹H NMR (500 MHz, CDCl₃ and 3 drops of TFA-d) δ 8.35 (d, J=9.2 Hz, 1H), 7.37 (dd, J=9.2, 2.3 Hz, 1H), 7.32 (d, J=2.3 Hz, 1H), 4.02 (s, 3H), 3.46-3.32 (m, 1H), 3.02-2.90 (m, 2H), 2.51 (dd, J=17.6, 10.7 Hz, 1H), 2.43-2.28 (m, 2H), 2.19-2.05 (m, 2H), 2.03-1.69 (m, 4H).

¹³C NMR (126 MHz, CDCl₃ and 3 drops of TFA-d) δ 203.5, 171.9, 167.9, 161.7, 142.5, 126.8, 121.7, 113.1, 107.3, 99.9, 56.7, 40.6, 39.6, 39.3, 31.5, 26.0, 25.8, 17.6.

### 3-cyclopentyl-3,4-dihydroacridine-1,9(2H,10H)-dione (133)

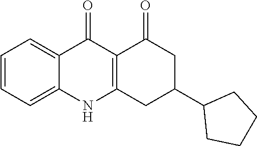 [SMILES: O=C1CC(C2CCCC2)Cc2[nH]c3ccccc3c(=O)c21]

Following general procedure L, the title compound was prepared as a white powder.

LCMS (ESI-MS) m/z: [M+H]⁺ calcd for C₁₈H₂₀NO₂ 282.14; found 281.9. Retention time: 3.521 min.

TLC: (5% methanol in dichloromethane, R_{f}): 0.39 (UV, 254 nm, 280 nm).

¹H NMR (500 MHz, CDCl₃ and 3 drops of TFA-d) δ 8.52 (d, J=8.3 Hz, 1H), 8.14 (dd, J=8.3, 7.7 Hz, 1H), 8.07 (d, J=8.4 Hz, 1H), 7.87 (dd, J=8.4, 7.7 Hz, 1H), 3.59 (ddd, J=17.8, 4.0, 1.4 Hz, 1H), 3.17 (dd, J=17.9, 11.7 Hz, 1H), 3.08 (ddd, J=17.8, 4.0, 1.4 Hz, 1H), 2.71 (dd, J=17.9, 11.7 Hz, 1H), 2.32-2.20 (m, 1H), 1.96-1.80 (m, 3H), 1.66 (dqd, J=48.0, 9.6, 8.1, 4.9 Hz, 4H), 1.30-1.17 (m, 2H).

¹³C NMR (126 MHz, CDCl₃ and 3 drops of TFA-d) δ 203.8, 173.3, 162.1, 139.5, 138.3, 129.5, 125.2, 120.5, 119.3, 108.0, 44.8, 42.8, 38.9, 33.6, 30.3, 30.2, 25.05, 25.02.

### 3-cyclopentyl-8-methyl-3,4-dihydroacridine-1,9(2H,10H)-dione (134)

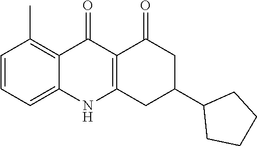 [SMILES: Cc1cccc2[nH]c3c(c(=O)c12)C(=O)CC(C1CCCC1)C3]

Following general procedure L, the title compound was prepared as a white powder.

LCMS (ESI-MS) m/z: [M+H]⁺ calcd for C₁₉H₂₂NO₂ 296.16; found 295.9. Retention time: 3.886 min.

TLC: (5% methanol in dichloromethane, R_{f}): 0.39 (UV, 254 nm, 280 nm).

¹H NMR (500 MHz, CDCl₃ and 3 drops of TFA-d) δ 8.35 (d, J=8.4 Hz, 1H), 7.90 (dd, J=8.4, 7.3 Hz, 1H), 7.53 (d, J=7.3 Hz, 1H), 3.90 (d, J=17.7 Hz, 1H), 3.21 (dd, J=17.7, 10.7 Hz, 1H), 2.97 (s, 4H), 2.69 (dd, J=17.7, 10.7 Hz, 1H), 2.29-2.16 (m, 1H), 2.02-1.92 (m, 1H), 1.92-1.79 (m, 2H), 1.76-1.53 (m, 4H), 1.34-1.15 (m, 2H).

### 3-cyclopentyl-7-methyl-3,4-dihydroacridine-1,9(2H,10H)-dione (135)

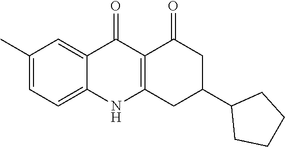 [SMILES: Cc1ccc2[nH]c3c(c(=O)c2c1)C(=O)CC(C1CCCC1)C3]

Following general procedure L, the title compound was prepared as a white powder.

TLC: (5% methanol in dichloromethane, R_{f}): 0.42 (UV, 254 nm, 280 nm).

¹H NMR (500 MHz, CDCl₃ and 3 drops of TFA-d) δ 8.26 (s, 1H), 8.01-7.90 (m, 2H), 3.56 (ddd, J=17.8, 4.0, 1.7 Hz, 1H), 3.14 (dd, J=17.9, 11.0 Hz, 1H), 3.06 (ddd, J=17.8, 4.0, 1.7 Hz, 1H), 2.74-2.60 (m, 4H), 2.30-2.18 (m, 1H), 1.95-1.78 (m, 3H), 1.78-1.54 (m, 4H), 1.27-1.16 (m, 2H).

¹³C NMR (126 MHz, CDCl₃ and 3 drops of TFA-d) δ 203.8, 172.7, 160.9, 140.6, 140.1, 137.8, 124.0, 120.2, 119.3, 108.0, 44.8, 42.8, 38.9, 33.5, 30.3, 30.2, 25.04, 25.02, 21.6.

### 3-cyclopentyl-6-methyl-3,4-dihydroacridine-1,9(2H,10H)-dione (136)

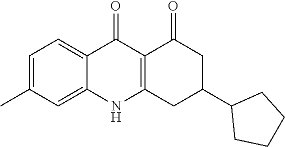 [SMILES: Cc1ccc2c(=O)c3c([nH]c2c1)CC(C1CCCC1)CC3=O]

Following general procedure L, the title compound was prepared as a white powder.

LCMS (ESI-MS) m/z: [M+H]⁺ calcd for C₁₉H₂₂NO₂ 296.16; found 295.9. Retention time: 3.676 min.

TLC: (5% methanol in dichloromethane, R_{f}): 0.42 (UV, 254 nm, 280 nm).

¹H NMR (500 MHz, CDCl₃ and 3 drops of TFA-d) δ 8.38 (d, J=8.5 Hz, 1H), 7.81 (s, 1H), 7.66 (d, J=8.5 Hz, 1H), 3.55 (ddd, J=17.8, 4.0, 1.5 Hz, 1H), 3.13 (dd, J=17.9, 11.0 Hz, 1H), 3.05 (ddd, J=17.8, 4.0, 1.5 Hz, 1H), 2.74-2.62 (m, 4H), 2.30-2.18 (m, 1H), 1.96-1.79 (m, 3H), 1.76-1.54 (m, 4H), 1.31-1.14 (m, 2H).

¹³C NMR (126 MHz, CDCl₃ and 3 drops of TFA-d) δ 203.8, 172.9, 161.8, 151.4, 139.8, 131.5, 124.9, 119.6, 117.1, 107.7, 44.8, 42.8, 38.9, 33.5, 30.3, 30.2, 25.04, 25.02, 22.5.

### 3-cyclopentyl-5-methyl-3,4-dihydroacridine-1,9(2H,10H)-dione (137)

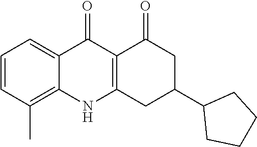 [SMILES: Cc1cccc2c(=O)c3c([nH]c12)CC(C1CCCC1)CC3=O]

Following general procedure L, the title compound was prepared as a white powder.

TLC: (5% methanol in dichloromethane, R_{f}): 0.42 (UV, 254 nm, 280 nm).

¹H NMR (500 MHz, CDCl₃ and 3 drops of TFA-d) δ 8.40 (d, J=8.2 Hz, 1H), 7.95 (d, J=7.2 Hz, 1H), 7.75 (dd, J=8.2, 7.2 Hz, 1H), 3.74 (ddd, J=17.9, 3.9, 1.7 Hz, 1H), 3.17 (dd, J=17.7, 11.1 Hz, 1H), 3.07 (dd, J=17.9, 3.9 Hz, 1H), 2.77 (s, 3H), 2.70 (dd, J=17.7, 11.1 Hz, 1H), 2.27-2.17 (m, 1H), 1.94-1.79 (m, 3H), 1.75-1.52 (m, 4H), 1.26-1.14 (m, 2H).

¹³C NMR (126 MHz, CDCl₃ and 3 drops of TFA-d) δ 203.9, 173.6, 162.7, 139.4, 138.3, 129.6, 129.1, 123.3, 119.7, 108.0, 44.8, 42.8, 38.9, 33.4, 30.3, 30.1, 25.05, 25.02, 17.1.

### 7-chloro-3-cyclopentyl-3,4-dihydroacridine-1,9(2H,10H)-dione (138)

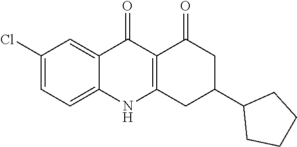 [SMILES: O=C1CC(C2CCCC2)Cc2[nH]c3ccc(Cl)cc3c(=O)c21]

Following general procedure L, the title compound was prepared as a white powder.

LCMS (ESI-MS) m/z: [M+H]⁺ calcd for C₁₈H₁₉ClNO₂ 316.10; found 315.8. Retention time: 3.934 min.

TLC: (5% methanol in dichloromethane, R_{f}): 0.39 (UV, 254 nm, 280 nm).

¹H NMR (500 MHz, CDCl₃ and 3 drops of TFA-d) δ 8.46 (s, 1H), 8.08-8.01 (m, 2H), 3.64-3.53 (m, 1H), 3.23-3.04 (m, 2H), 2.71 (dd, J=17.9, 11.8 Hz, 1H), 2.31-2.20 (m, 1H), 1.95-1.76 (m, 3H), 1.77-1.54 (m, 4H), 1.28-1.14 (m, 2H).

¹³C NMR (126 MHz, CDCl₃ and 3 drops of TFA-d) δ 203.7, 172.2, 162.2, 138.7, 137.9, 136.2, 124.1, 122.2, 120.2, 108.4, 44.8, 42.8, 38.8, 33.5, 30.3, 30.2, 25.01, 24.99.

### 3-cyclopentyl-6-methoxy-3,4-dihydroacridine-1,9(2H,10H)-dione (139)

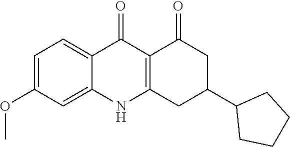 [SMILES: COc1ccc2c(=O)c3c([nH]c2c1)CC(C1CCCC1)CC3=O]

Following general procedure L, the title compound was prepared as a white powder.

TLC: (5% methanol in dichloromethane, R_{f}): 0.17 (UV, 254 nm, 280 nm).

¹H NMR (500 MHz, CDCl₃ and 3 drops of TFA-d) δ 8.35 (d, J=9.2 Hz, 1H), 7.37 (dd, J=9.2, 2.3 Hz, 1H), 7.31 (d, J=2.3 Hz, 1H), 4.02 (s, 3H), 3.49 (ddd, J=17.8, 3.9, 1.6 Hz, 1H), 3.11 (dd, J=17.8, 11.8 Hz, 1H), 3.04 (ddd, J=17.8, 3.9, 1.6 Hz, 1H), 2.66 (dd, J=17.8, 11.8 Hz, 1H), 2.29-2.16 (m, 1H), 1.99-1.77 (m, 3H), 1.77-1.53 (m, 4H), 1.29-1.13 (m, 2H).

¹³C NMR (126 MHz, CDCl₃ and 3 drops of TFA-d) δ 203.6, 171.8, 167.9, 161.9, 142.5, 126.8, 121.6, 113.1, 107.3, 99.9, 56.7, 44.8, 42.7, 39.0, 33.5, 30.3, 30.2, 25.04, 25.00.

### 3-cyclohexyl-7-methyl-3,4-dihydroacridine-1,9(2H,10H)-dione (140)

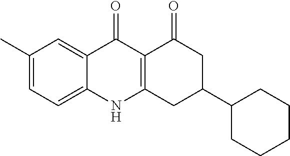 [SMILES: Cc1ccc2[nH]c3c(c(=O)c2c1)C(=O)CC(C1CCCCC1)C3]

Following general procedure L, the title compound was prepared as a white powder.

TLC: (5% methanol in dichloromethane, R_{f}): 0.22 (UV, 254 nm, 280 nm).

### 3-cyclohexyl-6-methyl-3,4-dihydroacridine-1,9(2H,10H)-dione (141)

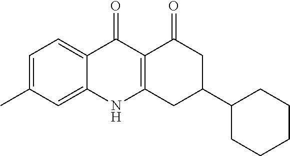 [SMILES: Cc1ccc2c(=O)c3c([nH]c2c1)CC(C1CCCCC1)CC3=O]

Following general procedure L, the title compound was prepared as an off-white powder.

LCMS (ESI-MS) m/z: [M+H]⁺ calcd for C₂₀H₂₄NO₂ 310.17; found 309.9. Retention time: 4.029 min.

TLC: (5% methanol in dichloromethane, R_{f}): 0.17 (UV, 254 nm, 280 nm).

¹H NMR (500 MHz, CDCl₃ and 3 drops of TFA-d) δ 8.37 (d, J=8.5 Hz, 1H), 7.85 (s, 1H), 7.65 (d, J=8.5 Hz, 1H), 3.52 (ddd, J=17.6, 4.0, 1.6 Hz, 1H), 3.16 (dd, J=17.8, 11.6 Hz, 1H), 2.99 (ddd, J=17.6, 4.0, 1.6 Hz, 1H), 2.73-2.62 (m, 4H), 2.33-2.23 (m, 1H), 1.86-1.69 (m, 5H), 1.50-1.41 (m, 1H), 1.33-1.01 (m, 5H).

¹³C NMR (126 MHz, CDCl₃ and 3 drops of TFA-d) δ 204.1, 172.8, 162.2, 151.2, 139.8, 131.4, 124.8, 119.7, 117.1, 107.7, 41.2, 41.0, 38.7, 31.8, 29.6, 29.5, 26.15, 26.09, 26.05, 22.5.

### 3-cyclohexyl-5-methyl-3,4-dihydroacridine-1,9(2H,10H)-dione (142)

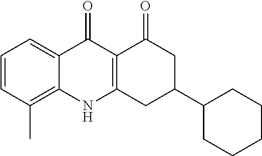 [SMILES: Cc1cccc2c(=O)c3c([nH]c12)CC(C1CCCCC1)CC3=O]

Following general procedure L, the title compound was prepared as a white powder.

TLC: (5% methanol in dichloromethane, R_{f}): 0.32 (UV, 254 nm, 280 nm).

¹H NMR (500 MHz, CDCl₃ and 3 drops of TFA-d) δ 8.40 (d, J=9.0 Hz, 1H), 7.95 (d, J=8.1 Hz, 1H), 7.75 (dd, J=9.0, 8.1 Hz, 1H), 3.67 (ddd, J=17.7, 4.0, 1.7 Hz, 1H), 3.17 (dd, J=18.0, 11.6 Hz, 1H), 3.01 (ddd, J=17.7, 4.0, 1.7 Hz, 1H), 2.79-2.66 (m, 4H), 2.31-2.21 (m, 1H), 1.86-1.67 (m, 5H), 1.49-1.39 (m, 1H), 1.34-1.11 (m, 3H), 1.10-0.99 (m, 2H).

¹³C NMR (126 MHz, CDCl₃ and 3 drops of TFA-d) δ 204.3, 173.6, 163.0, 139.4, 138.3, 129.5, 129.1, 123.3, 119.7, 108.0, 41.3, 41.1, 38.6, 31.7, 29.7, 29.5, 26.1, 26.0, 25.9, 16.9.

### 7-chloro-3-cyclohexyl-3,4-dihydroacridine-1,9(2H,10H)-dione (143)

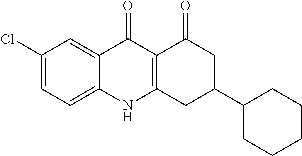 [SMILES: O=C1CC(C2CCCCC2)Cc2[nH]c3ccc(Cl)cc3c(=O)c21]

Following general procedure L, the title compound was prepared as a white powder.

TLC: (5% methanol in dichloromethane, R_{f}): 0.21 (UV, 254 nm, 280 nm).

¹H NMR (500 MHz, CDCl₃ and 3 drops of TFA-d) δ 8.46 (d, J=1.7 Hz, 1H), 8.12-7.99 (m, 2H), 3.52 (dd, J=18.0, 3.1 Hz, 1H), 3.18 (dd, J=17.9, 12.4 Hz, 1H), 3.03 (dd, J=18.0, 3.1 Hz, 1H), 2.72 (dd, J=17.9, 12.4 Hz, 1H), 2.36-2.24 (m, 1H), 1.89-1.67 (m, 5H), 1.54-1.42 (m, 1H), 1.36-0.99 (m, 5H).

¹³C NMR (126 MHz, CDCl₃ and 3 drops of TFA-d) δ 204.14, 172.2, 162.6, 138.7, 138.0, 136.1, 124.1, 122.2, 120.1, 108.4, 41.2, 41.0, 38.6, 31.8, 29.6, 29.4, 26.0.

### 3-cyclohexyl-6-methoxy-3,4-dihydroacridine-1,9(2H,10H)-dione (144)

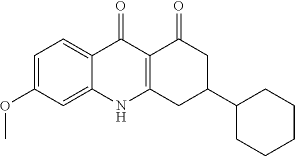 [SMILES: COc1ccc2c(=O)c3c([nH]c2c1)CC(C1CCCCC1)CC3=O]

Following general procedure L, the title compound was prepared as a white powder.

TLC: (5% methanol in dichloromethane, R_{f}): 0.36 (UV, 254 nm, 280 nm).

¹H NMR (500 MHz, CDCl₃ and 3 drops of TFA-d) δ 8.35 (d, J=9.2 Hz, 1H), 7.37 (dd, J=9.2, 2.3 Hz, 1H), 7.29 (d, J=2.3 Hz, 1H), 4.02 (s, 3H), 3.42 (ddd, J=17.8, 4.1, 1.7 Hz, 1H), 3.13 (dd, J=17.8, 12.4 Hz, 1H), 2.98 (ddd, J=17.8, 4.1, 1.7 Hz, 1H), 2.67 (dd, J=17.8, 12.4 Hz, 1H), 2.33-2.22 (m, 1H), 1.89-1.65 (m, 5H), 1.50-1.41 (m, 1H), 1.35-0.99 (m, 5H).

¹³C NMR (126 MHz, CDCl₃ and 3 drops of TFA-d) δ 204.0, 171.8, 167.8, 162.3, 142.5, 126.8, 121.6, 113.1, 107.3, 99.9, 56.6, 41.2, 40.9, 38.8, 31.8, 29.6, 29.5, 26.14, 26.09, 26.04.

### 7-chloro-3-(tetrahydrofuran-3-yl)-3,4-dihydroacridine-1,9(2H,10H)-dione (145)

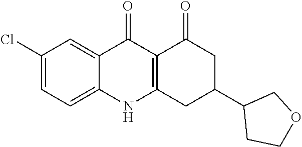 [SMILES: O=C1CC(C2CCOC2)Cc2[nH]c3ccc(Cl)cc3c(=O)c21]

Following general procedure L, the title compound was prepared as a white powder.

LCMS (ESI-MS) m/z: [M+H]⁺ calcd for C₁₇H₁₇ClNO₃ 318.08; found 317.8. Retention time: 2.777 min.

TLC: (5% methanol in dichloromethane, R_{f}): 0.21 (UV, 254 nm, 280 nm).

¹H NMR (500 MHz, CDCl₃ and 3 drops of TFA-d) δ 8.51-8.44 (m, 1H), 8.19-8.01 (m, 2H), 4.24-4.07 (m, 2H), 3.99-3.88 (m, 1H), 3.75-3.51 (m, 2H), 3.32-3.18 (m, 1H), 3.14-2.92 (m, 1H), 2.83-2.69 (m, 1H), 2.54-2.38 (m, 2H), 2.36-2.20 (m, 1H), 1.84-1.72 (m, 1H).

### 7-chloro-3-(tetrahydro-2H-pyran-4-yl)-3,4-dihydroacridine-1,9(2H,10H)-dione (146)

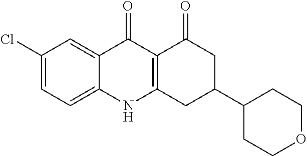 [SMILES: O=C1CC(C2CCOCC2)Cc2[nH]c3ccc(Cl)cc3c(=O)c21]

Following general procedure L, the title compound was prepared as a white powder.

LCMS (ESI-MS) m/z: [M+H]⁺ calcd for C₁₈H₁₉ClNO₃ 332.10; found 331.8. Retention time: 3.051 min.

TLC: (5% methanol in dichloromethane, R_{f}): 0.21 (UV, 254 nm, 280 nm).

¹H NMR (500 MHz, CDCl₃ and 3 drops of TFA-d) δ 8.48 (d, J=1.9 Hz, 1H), 8.12-8.01 (m, 2H), 4.33-4.14 (m, 2H), 3.67-3.49 (m, 3H), 3.21 (dd, J=17.9, 12.5 Hz, 1H), 3.08 (ddd, J=17.7, 3.8, 1.8 Hz, 1H), 2.70 (dd, J=17.9, 12.5 Hz, 1H), 2.42-2.29 (m, 1H), 1.86-1.72 (m, 3H), 1.64-1.47 (m, 2H).

¹³C NMR (126 MHz, CDCl₃ and 3 drops of TFA-d) δ 203.0, 172.3, 161.8, 138.9, 138.0, 136.4, 124.2, 122.2, 120.2, 108.3, 67.6, 67.5, 40.4, 38.2, 37.9, 31.6, 29.2, 28.7.

### 6-methoxy-3-(tetrahydro-2H-pyran-4-yl)-3,4-dihydroacridine-1,9(2H,10H)-dione (147)

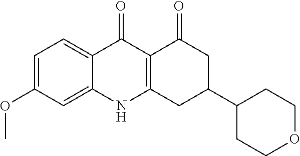 [SMILES: COc1ccc2c(=O)c3c([nH]c2c1)CC(C1CCOCC1)CC3=O]

Following general procedure L, the title compound was prepared as a white powder.

TLC: (5% methanol in dichloromethane, R_{f}): 0.27 (UV, 254 nm, 280 nm).

¹H NMR (500 MHz, CDCl₃ and 3 drops of TFA-d) δ 8.35 (d, J=9.3 Hz, 1H), 7.51 (d, J=2.0 Hz, 1H), 7.37 (dd, J=9.3, 2.0 Hz, 1H), 4.31-4.15 (m, 2H), 4.04 (s, 3H), 3.67 (d, J=17.2 Hz, 1H), 3.64-3.52 (m, 2H), 3.18 (dd, J=17.7, 12.4 Hz, 1H), 3.01 (dd, J=17.2, 3.6 Hz, 1H), 2.64 (dd, J=17.7, 12.4 Hz, 1H), 2.36-2.26 (m, 1H), 1.84-1.72 (m, 3H), 1.63-1.46 (m, 2H).

¹³C NMR (126 MHz, CDCl₃ and 3 drops of TFA-d) δ 202.8, 171.6, 167.9, 161.3, 142.7, 126.7, 121.8, 113.1, 107.1, 100.1, 67.7, 67.5, 56.8, 40.3, 38.2, 38.1, 31.6, 29.4, 28.7.

### 7-chloro-3-(thiophen-2-yl)-3,4-dihydroacridine-1,9(2H,10H)-dione (148)

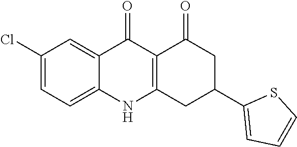 [SMILES: O=C1CC(c2cccs2)Cc2[nH]c3ccc(Cl)cc3c(=O)c21]

Following general procedure L, the title compound was prepared as a yellow powder.

LCMS (ESI-MS) m/z: [M+H]⁺ calcd for C₁₇H₁₃ClNO₂S 330.03; found 329.8. Retention time: 3.540 min.

TLC: (5% methanol in dichloromethane, R_{f}): 0.37 (UV, 254 nm, 280 nm).

¹H NMR (500 MHz, CDCl₃ and 3 drops of TFA-d) δ 8.47 (s, 1H), 8.09-8.05 (m, 2H), 7.28-7.24 (m, 1H), 6.98 (dd, J=5.1, 3.5 Hz, 1H), 6.94 (d, J=3.5 Hz, 1H), 4.14-4.05 (m, 1H), 3.89 (dd, J=17.9, 4.3 Hz, 1H), 3.71 (dd, J=17.9, 8.9 Hz, 1H), 3.38 (dd, J=17.9, 4.3 Hz, 1H), 3.27 (dd, J=17.9, 8.9 Hz, 1H).

¹³C NMR (126 MHz, CDCl₃ and 3 drops of TFA-d) δ 202.0, 172.3, 160.6, 142.5, 139.0, 138.0, 136.5, 127.5, 125.2, 125.0, 124.2, 122.2, 120.3, 108.3, 44.5, 35.9, 33.6.

### 7-fluoro-3-(thiophen-2-yl)-3,4-dihydroacridine-1,9(2H,10H)-dione (149)

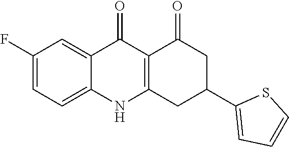 [SMILES: O=C1CC(c2cccs2)Cc2[nH]c3ccc(F)cc3c(=O)c21]

Following general procedure L, the title compound was prepared as a white powder.

TLC: (5% methanol in dichloromethane, R_{f}): 0.40 (UV, 254 nm, 280 nm).

¹H NMR (500 MHz, DMSO-d₆) δ 7.75 (d, J=8.9 Hz, 1H), 7.63-7.50 (m, 3H), 7.31 (s, 1H), 7.18 (d, J=4.9 Hz, 1H), 3.62-3.52 (m, 1H), 3.26-3.18 (m, 2H), 2.70 (d, J=7.8 Hz, 2H).

¹H NMR (500 MHz, CDCl₃ and 3 drops of TFA-d) δ 8.20-8.09 (m, 2H), 7.89 (ddd, J=9.3, 7.6, 2.8 Hz, 1H), 7.40 (dd, J=5.1, 2.9 Hz, 1H), 7.12 (dd, J=2.9, 1.4 Hz, 1H), 7.04 (dd, J=5.1, 1.4 Hz, 1H), 3.93-3.79 (m, 2H), 3.66 (dd, J=17.9, 10.0 Hz, 1H), 3.32 (dd, J=17.9, 4.0 Hz, 1H), 3.21 (dd, J=17.9, 10.0 Hz, 1H).

¹³C NMR (126 MHz, CDCl₃ and 3 drops of TFA-d) δ 202.82, 172.63 (d, J_{C—F}=4.3 Hz), 161.84 (d, J_{C—F}=257.1 Hz), 160.57 (d, J_{C—F}=2.3 Hz), 140.10, 136.42, 128.08 (d, J_{C—F}=26.0 Hz), 127.88, 125.68, 123.52 (d, J_{C—F}=8.8 Hz), 121.49, 120.90 (d, J_{C—F}=9.4 Hz), 109.88 (d, J_{C—F}=24.4 Hz), 108.07, 43.75, 34.91, 33.62.

¹⁹F NMR (471 MHz, CDCl₃ and 3 drops of TFA-d) δ −105.48-−105.55 (m).

### 6-methoxy-3-(thiophen-2-yl)-3,4-dihydroacridine-1,9(2H,10H)-dione (150)

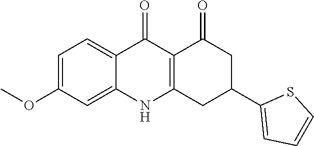 [SMILES: COc1ccc2c(=O)c3c([nH]c2c1)CC(c1cccs1)CC3=O]

Following general procedure L, the title compound was prepared as a white powder.

TLC: (5% methanol in dichloromethane, R_{f}): 0.43 (UV, 254 nm, 280 nm).

¹H NMR (500 MHz, DMSO-d₆) δ 11.92 (s, 1H), 8.01 (d, J=8.7 Hz, 1H), 7.56-7.50 (m, 1H), 7.31 (s, 1H), 7.18 (d, J=4.9 Hz, 1H), 6.99-6.90 (m, 2H), 3.86 (s, 3H), 3.60-3.52 (m, 1H), 3.26-3.14 (m, 2H), 2.75-2.64 (m, 2H).

¹H NMR (500 MHz, CDCl₃ and 3 drops of TFA-d) δ 8.37 (d, J=9.2 Hz, 1H), 7.46-7.34 (m, 2H), 7.31 (d, J=2.4 Hz, 1H), 7.12 (s, 1H), 7.04 (d, J=5.0 Hz, 1H), 4.02 (s, 3H), 3.90-3.80 (m, 1H), 3.75 (dd, J=17.9, 4.3 Hz, 1H), 3.59 (dd, J=17.9, 10.3 Hz, 1H), 3.26 (dd, J=17.9, 4.3 Hz, 1H), 3.15 (dd, J=17.9, 10.3 Hz, 1H).

¹³C NMR (126 MHz, CDCl₃ and 3 drops of TFA-d) δ 202.5, 171.8, 167.9, 160.8, 142.5, 140.3, 127.6, 126.8, 125.6, 121.7, 121.2, 113.1, 107.2, 99.9, 56.6, 43.6, 34.8, 33.7.

### 7-chloro-3-(thiophen-3-yl)-3,4-dihydroacridine-1,9(2H,10H)-dione (151)

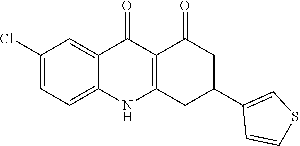 [SMILES: O=C1CC(c2ccsc2)Cc2[nH]c3ccc(Cl)cc3c(=O)c21]

Following general procedure L, the title compound was prepared as a yellow powder.

LCMS (ESI-MS) m/z: [M+H]⁺ calcd for C₁₇H₁₃ClNO₂S 330.03; found 329.8. Retention time: 3.534 min.

TLC: (5% methanol in dichloromethane, R_{f}): 0.37 (UV, 254 nm, 280 nm).

¹H NMR (500 MHz, CDCl₃ and 3 drops of TFA-d) δ 8.47 (d, J=1.9 Hz, 1H), 8.13-8.03 (m, 2H), 7.40 (dd, J=5.0, 2.6 Hz, 1H), 7.12 (dd, J=2.6, 1.5 Hz, 1H), 7.04 (dd, J=5.0, 1.5 Hz, 1H), 3.93-3.79 (m, 2H), 3.67 (dd, J=18.0, 9.5 Hz, 1H), 3.31 (ddd, J=18.0, 4.1, 1.4 Hz, 1H), 3.20 (dd, J=18.0, 9.5 Hz, 1H).

¹³C NMR (126 MHz, CDCl₃ and 3 drops of TFA-d) δ 202.7, 172.3, 161.1, 140.1, 138.9, 138.0, 136.4, 127.8, 125.7, 124.2, 122.2, 121.4, 120.2, 108.3, 43.7, 34.9, 33.5.

### 6-methoxy-3-(thiophen-3-yl)-3,4-dihydroacridine-1,9(2H,10H)-dione (152)

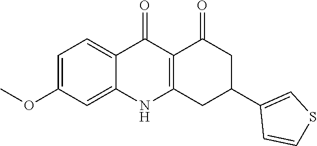 [SMILES: COc1ccc2c(=O)c3c([nH]c2c1)CC(c1ccsc1)CC3=O]

Following general procedure L, the title compound was prepared as a white powder.

TLC: (5% methanol in dichloromethane, R_{f}): 0.43 (UV, 254 nm, 280 nm).

¹H NMR (500 MHz, DMSO-d₆) δ 11.97 (s, 1H), 8.00 (d, J=8.9 Hz, 1H), 7.40 (dd, J=4.1, 2.1 Hz, 1H), 7.02-6.94 (m, 3H), 6.91 (d, J=2.1 Hz, 1H), 3.86 (s, 3H), 3.85-3.76 (m, 1H), 3.29-3.19 (m, 2H), 2.81-2.66 (m, 2H).

¹H NMR (500 MHz, CDCl₃ and 3 drops of TFA-d) δ 8.37 (d, J=9.2 Hz, 1H), 7.39 (dd, J=9.2, 2.3 Hz, 1H), 7.32 (d, J=2.3 Hz, 1H), 7.25 (dd, J=5.0, 1.5 Hz, 1H), 6.98 (dd, J=5.0, 3.4 Hz, 1H), 6.95 (d, J=3.4 Hz, 1H), 4.10-4.01 (m, 4H), 3.82 (ddd, J=17.8, 4.4, 1.1 Hz, 1H), 3.64 (dd, J=17.8, 9.1 Hz, 1H), 3.32 (ddd, J=17.8, 4.4, 1.1 Hz, 1H), 3.20 (dd, J=17.8, 9.1 Hz, 1H).

¹³C NMR (126 MHz, CDCl₃ and 3 drops of TFA-d) δ 201.9, 171.9, 168.0, 160.3, 142.9, 142.7, 127.5, 126.9, 125.1, 124.8, 121.9, 113.2, 107.3, 100.0, 56.7, 44.5, 35.8, 33.8.

### 7-chloro-3-(thiazol-2-yl)-3,4-dihydroacridine-1,9(2H,10H)-dione (153)

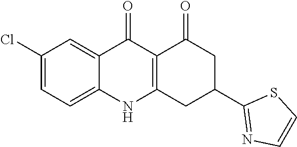 [SMILES: O=C1CC(c2nccs2)Cc2[nH]c3ccc(Cl)cc3c(=O)c21]

Following general procedure L, the title compound was prepared as a yellow powder.

TLC: (5% methanol in dichloromethane, R_{f}): 0.29 (UV, 254 nm, 280 nm).

¹H NMR (500 MHz, CDCl₃ and 3 drops of TFA-d) δ 8.53 (d, J=2.2 Hz, 1H), 8.24 (d, J=3.8 Hz, 1H), 8.12 (dd, J=9.0, 2.2 Hz, 1H), 8.01 (d, J=9.0 Hz, 1H), 7.94 (d, J=3.8 Hz, 1H), 4.91-4.80 (m, 1H), 4.12 (ddd, J=17.4, 4.2, 1.5 Hz, 1H), 3.99 (dd, J=17.3, 12.0 Hz, 1H), 3.55-3.39 (m, 2H).

¹³C NMR (126 MHz, CDCl₃ and 3 drops of TFA-d) δ 198.4, 173.9, 172.8, 158.3, 139.5, 137.9, 137.1, 135.6, 124.5, 123.3, 122.0, 120.4, 108.2, 34.2, 34.1, 33.1.

### 6-methoxy-3-(thiazol-2-yl)-3,4-dihydroacridine-1,9(2H,10H)-dione (154)

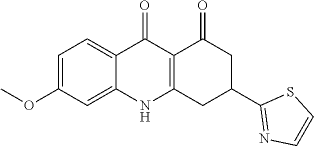 [SMILES: COc1ccc2c(=O)c3c([nH]c2c1)CC(c1nccs1)CC3=O]

Following general procedure L, the title compound was prepared as a white powder.

LCMS (ESI-MS) m/z: [M+H]⁺ calcd for C₁₇H₁₅N₂O₃S 327.07; found 326.8. Retention time: 2.959 min.

TLC: (5% methanol in dichloromethane, R_{f}): 0.29 (UV, 254 nm, 280 nm).

¹H NMR (500 MHz, CDCl₃ and 3 drops of TFA-d) δ 8.40 (d, J=9.3 Hz, 1H), 8.19 (d, J=3.8 Hz, 1H), 7.83 (d, J=3.8 Hz, 1H), 7.42 (dd, J=9.2, 2.3 Hz, 1H), 7.32 (d, J=2.3 Hz, 1H), 4.80-4.69 (m, 1H), 4.04 (s, 4H), 3.93 (dd, J=17.3, 11.6 Hz, 1H), 3.47-3.33 (m, 2H).

¹³C NMR (126 MHz, CDCl₃ and 3 drops of TFA-d) δ 199.2, 172.3, 171.8, 168.1, 158.6, 142.8, 138.5, 126.8, 122.0, 121.7, 113.3, 107.0, 100.1, 56.7, 35.0, 34.9, 33.3.

### 7-chloro-3-(thiazol-4-yl)-3,4-dihydroacridine-1,9(2H,10H)-dione (155)

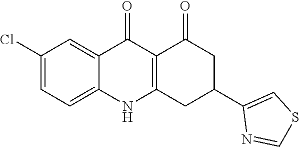 [SMILES: O=C1CC(c2cscn2)Cc2[nH]c3ccc(Cl)cc3c(=O)c21]

Following general procedure L, the title compound was prepared as a yellow powder.

LCMS (ESI-MS) m/z: [M+H]⁺ calcd for C₁₆H₁₂ClN₂O₂S 331.02; found 330.7. Retention time: 2.877 min.

TLC: (5% methanol in dichloromethane, R_{f}): 0.19 (UV, 254 nm, 280 nm).

¹H NMR (500 MHz, CDCl₃ and 3 drops of TFA-d) δ 9.81 (d, J=2.2 Hz, 1H), 8.50 (d, J=2.2 Hz, 1H), 8.15-8.01 (m, 2H), 7.91 (d, J=2.2 Hz, 1H), 4.46-4.35 (m, 1H), 3.97 (dd, J=17.6, 4.3 Hz, 1H), 3.88 (dd, J=17.6, 12.2 Hz, 1H), 3.39 (ddd, J=17.6, 4.3, 1.6 Hz, 1H), 3.31 (dd, J=17.6, 12.2 Hz, 1H).

¹³C NMR (126 MHz, CDCl₃ and 3 drops of TFA-d) δ 200.3, 172.6, 159.6, 158.2, 150.0, 139.1, 138.0, 136.6, 124.3, 122.1, 120.4, 119.5, 108.2, 32.7, 32.4, 32.3.

### 6-methoxy-3-(thiazol-4-yl)-3,4-dihydroacridine-1,9(2H,10H)-dione (156)

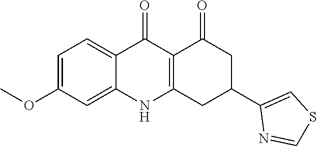 [SMILES: COc1ccc2c(=O)c3c([nH]c2c1)CC(c1cscn1)CC3=O]

Following general procedure L, the title compound was prepared as a white powder.

LCMS (ESI-MS) m/z: [M+H]⁺ calcd for C₁₇H₁₅N₂O₃S 327.07; found 326.8. Retention time: 1.838 min.

TLC: (5% methanol in dichloromethane, R_{f}): 0.16 (UV, 254 nm, 280 nm).

¹H NMR (500 MHz, CDCl₃ and 3 drops of TFA-d) δ 9.88 (d, J=2.1 Hz, 1H), 8.40 (d, J=9.2 Hz, 1H), 7.95 (d, J=2.1 Hz, 1H), 7.42 (dd, J=9.2, 2.4 Hz, 1H), 7.30 (d, J=2.4 Hz, 1H), 4.42-4.31 (m, 1H), 4.04 (s, 3H), 3.93 (dd, J=17.5, 4.0 Hz, 1H), 3.83 (dd, J=17.5, 11.6 Hz, 1H), 3.40-3.20 (m, 2H).

¹³C NMR (126 MHz, CDCl₃ and 3 drops of TFA-d) δ 199.8, 172.1, 168.2, 159.0, 158.4, 149.6, 142.6, 127.0, 122.0, 119.9, 113.3, 107.1, 99.9, 56.7, 41.4, 35.6, 32.4.

### 7-chloro-3-(thiazol-5-yl)-3,4-dihydroacridine-1,9(2H,10H)-dione (157)

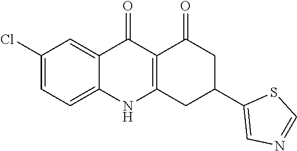 [SMILES: O=C1CC(c2cncs2)Cc2[nH]c3ccc(Cl)cc3c(=O)c21]

Following general procedure L, the title compound was prepared as a yellow powder.

TLC: (5% methanol in dichloromethane, R_{f}): 0.14 (UV, 254 nm, 280 nm).

¹H NMR (500 MHz, CDCl₃ and 3 drops of TFA-d) δ 9.90 (d, J=0.9 Hz, 1H), 8.56-8.47 (m, 2H), 8.23 (d, J=9.0 Hz, 1H), 8.11 (dd, J=9.0, 2.3 Hz, 1H), 4.39-4.17 (m, 2H), 3.85 (dd, J=17.6, 11.0 Hz, 1H), 3.51-3.29 (m, 2H).

¹³C NMR (126 MHz, CDCl₃ and 3 drops of TFA-d) δ 199.5, 172.6, 158.8, 156.9, 142.9, 139.4, 138.0, 137.0, 132.7, 124.4, 122.2, 120.4, 108.0, 42.9, 34.7, 31.5.

### 6-methoxy-3-(thiazol-5-yl)-3,4-dihydroacridine-1,9(2H,10H)-dione (158)

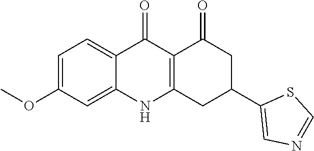 [SMILES: COc1ccc2c(=O)c3c([nH]c2c1)CC(c1cncs1)CC3=O]

Following general procedure L, the title compound was prepared as a off-white powder.

TLC: (5% methanol in dichloromethane, R_{f}): 0.19 (UV, 254 nm, 280 nm).

¹H NMR (500 MHz, DMSO-d₆) δ 9.00 (d, J=0.8 Hz, 1H), 8.01 (d, J=9.5 Hz, 1H), 7.80 (dd, J=1.3, 0.8 Hz, 1H), 7.01-6.96 (m, 2H), 3.97-3.89 (m, 1H), 3.87 (s, 3H), 3.38-3.24 (m, 2H), 2.84-2.67 (m, 2H).

### (E)-4-cyclopropylbut-3-en-2-one (172)

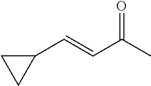

Following general procedure F, the title compound was prepared as a light-yellow oil.

LCMS (ESI-MS) m/z: [M+H]⁺ calcd for C₇H₁₁O 111.07; found 111.0. Retention time: 2.084 min.

TLC: (10% EtOAc in hexanes, R_{f}): 0.25 (UV, 254 nm, 280 nm).

¹H NMR (500 MHz, DMSO-d₆) δ 6.37 (dd, J=16.1, 9.9 Hz, 1H), 6.12 (d, J=16.1 Hz, 1H), 2.13 (s, 3H), 1.67-1.58 (m, 1H), 0.99-0.92 (m, 2H), 0.71-0.65 (m, 2H).

¹³C NMR (126 MHz, CDCl₃) δ 197.7, 153.5, 128.2, 26.9, 14.6, 9.0.

### (E)-4-cyclobutylbut-3-en-2-one (173)

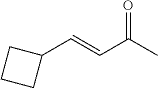

Following general procedure B and F, the title compound was prepared as a light-yellow oil.

LCMS (ESI-MS) m/z: [M+H]⁺ calcd for C₈H₁₃O 125.09; found 125.0. Retention time: 3.521 min.

TLC: (10% EtOAc in hexanes, R_{f}): 0.36 (UV, 254 nm, 280 nm).

¹H NMR (500 MHz, CDCl₃) δ 6.86 (dd, J=16.0, 6.7 Hz, 1H), 5.97 (dd, J=16.0, 1.4 Hz, 1H), 3.15-3.04 (m, 1H), 2.23 (s, 3H), 2.21-2.13 (m, 2H), 2.00-1.75 (m, 4H).

¹³C NMR (126 MHz, CDCl₃) δ 198.9, 151.7, 128.8, 37.7, 27.8, 26.8, 18.5.

### (E)-4-cyclopentylbut-3-en-2-one (174)

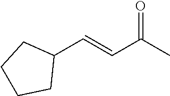

Following general procedure F, the title compound was prepared as a clear oil.

LCMS (ESI-MS) m/z: [M+H]⁺ calcd for C₉H₁₅O 139.10; found 139.0. Retention time: 3.961 min.

TLC: (10% EtOAc in hexanes, R_{f}): 0.36 (UV, 254 nm, 280 nm).

¹H NMR (500 MHz, DMSO-d₆) δ 6.83 (dd, J=16.3, 8.1 Hz, 1H), 6.00 (d, J=16.3 Hz, 1H), 2.66-2.56 (m, 1H), 2.19 (s, 3H), 1.85-1.76 (m, 2H), 1.71-1.52 (m, 4H), 1.45-1.33 (m, 2H).

¹³C NMR (126 MHz, DMSO-d₆) δ 198.5, 152.9, 129.6, 42.9, 32.4, 27.1, 25.3.

### (E)-4-cyclohexylbut-3-en-2-one (175)

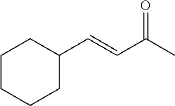

Following general procedure F, the title compound was prepared as a clear oil.

LCMS (ESI-MS) m/z: [M+H]⁺ calcd for C₁₀H₁₇O 153.12; found 153.2. Retention time: 3.452 min.

TLC: (10% EtOAc in hexanes, R_{f}): 0.36 (UV, 254 nm, 280 nm).

¹H NMR (500 MHz, CDCl₃) δ 6.73 (dd, J=16.1, 6.8 Hz, 1H), 6.02 (dd, J=16.1, 1.4 Hz, 1H), 2.24 (s, 3H), 2.19-2.11 (m, 1H), 1.80-1.65 (m, 5H), 1.35-1.10 (m, 5H).

¹³C NMR (126 MHz, CDCl₃) δ 199.2, 153.4, 128.8, 40.6, 31.7, 26.8, 25.9, 25.7.

### (E)-4-(tetrahydrofuran-3-yl)but-3-en-2-one (176)

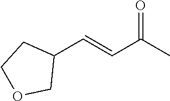

Following general procedure G, the title compound was prepared as a yellowish-brown oil.

LCMS (ESI-MS) m/z: [M+H]⁺ calcd for C₈H₁₃O₂ 141.08; found 141.1. Retention time: 1.216 min.

TLC: (33% EtOAc in hexanes, R_{f}): 0.27 (UV, 254 nm, 280 nm).

¹H NMR (500 MHz, CDCl₃) δ 6.72 (dd, J=15.9, 8.5 Hz, 1H), 6.13 (dd, J=15.9, 1.0 Hz, 1H), 4.00-3.91 (m, 2H), 3.87-3.80 (m, 1H), 3.57 (dd, J=8.7, 6.8 Hz, 1H), 3.02 (m, J=7.7 Hz, 1H), 2.26 (s, 3H), 2.24-2.13 (m, 1H), 1.87-1.76 (m, 1H).

¹³C NMR (126 MHz, CDCl₃) δ 198.27, 147.5, 131.1, 72.2, 68.1, 42.3, 32.5, 27.1.

### (E)-4-(tetrahydro-2H-pyran-4-yl)but-3-en-2-one (177)

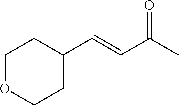

Following general procedure C and H, the title compound was prepared as a light brown oil.

LCMS (ESI-MS) m/z: [M+H]⁺ calcd for C₉H₁₅O₂ 155.10; found 155.0. Retention time: 1.163 min.

TLC: (20% EtOAc in hexanes, R_{f}): 0.32 (UV, 254 nm, 280 nm).

¹H NMR (500 MHz, CDCl₃) δ 6.72 (dd, J=15.8, 6.5 Hz, 1H), 6.06 (d, J=15.8 Hz, 1H), 4.00 (dd, J=11.5, 3.7 Hz, 2H), 3.45 (td, J=11.8, 3.7 Hz, 2H), 2.47-2.37 (m, 1H), 2.26 (s, 3H), 1.69 (d, J=11.5 Hz, 2H), 1.59-1.48 (m, 2H).

¹³C NMR (126 MHz, CDCl₃) δ 198.7, 150.8, 129.4, 67.3, 37.7, 31.3, 27.0.

### (E)-4-(thiophen-2-yl)but-3-en-2-one (178)

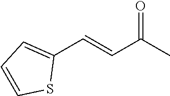

Following general procedure 1, the title compound was prepared as a dark brown oil.

LCMS (ESI-MS) m/z: [M+H]⁺ calcd for C₈H₉OS 153.03; found 152.9. Retention time: 3.147 min.

TLC: (20% EtOAc in hexanes, R_{f}): 0.34 (UV, 254 nm, 280 nm).

¹H NMR (500 MHz, CDCl₃) δ 7.63 (d, J=15.9 Hz, 1H), 7.40 (d, J=5.2 Hz, 1H), 7.29 (d, J=3.5 Hz, 1H), 7.07 (dd, J=5.2, 3.5 Hz, 1H), 6.53 (d, J=15.9 Hz, 1H), 2.34 (s, 3H).

¹³C NMR (126 MHz, CDCl₃) δ 197.7, 139.7, 135.7, 131.5, 128.9, 128.2, 125.8, 27.6.

### (E)-4-(thiophen-3-yl)but-3-en-2-one (179)

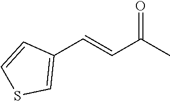

Following general procedure I, the title compound was prepared as an off-white and slightly brown powder.

LCMS (ESI-MS) m/z: [M+H]⁺ calcd for C₈H₉OS 153.03; found 152.9. Retention time: 3.289 min.

TLC: (20% EtOAc in hexanes, R_{f}): 0.34 (UV, 254 nm, 280 nm).

¹H NMR (500 MHz, DMSO-d₆) δ 7.98 (d, J=2.5 Hz, 1H), 7.67-7.59 (m, 2H), 7.53 (dd, J=5.1, 2.5 Hz, 1H), 6.65 (d, J=16.1 Hz, 1H), 2.31 (s, 3H).

¹³C NMR (126 MHz, DMSO-d₆) δ 198.7, 138.2, 137.5, 130.2, 128.2, 127.3, 126.1, 27.6.

### (E)-4-(6-(4-(trifluoromethoxy)phenyl)pyridin-3-yl)but-3-en-2-one (180)

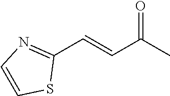

Following general procedure I, the title compound was prepared as a brown powder.

LCMS (ESI-MS) m/z: [M+H]⁺ calcd for C₇H₈NOS 154.02; found 153.9. Retention time: 1.074 min.

TLC: (20% EtOAc in hexanes, R_{f}): 0.17 (UV, 254 nm, 280 nm).

¹H NMR (500 MHz, CDCl₃) δ 7.94 (d, J=3.2 Hz, 1H), 7.64 (d, J=16.3 Hz, 1H), 7.46 (d, J=3.2 Hz, 1H), 6.94 (d, J=16.3 Hz, 1H), 2.40 (s, 3H).

¹³C NMR (126 MHz, CDCl₃) δ 197.6, 163.9, 144.8, 134.3, 130.7, 121.6, 27.8.

### (E)-4-(thiazol-4-yl)but-3-en-2-one (181)

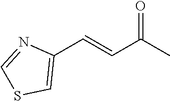

Following general procedure I, the title compound was prepared as a brown powder.

LCMS (ESI-MS) m/z: [M+H]⁺ calcd for C₇H₈NOS 154.02; found 153.9. Retention time: 1.784 min.

TLC: (33% EtOAc in hexanes, R_{f}): 0.23 (UV, 254 nm, 280 nm).

¹H NMR (500 MHz, DMSO-d₆) b 9.20 (s, 1H), 8.14 (s, 1H), 7.66 (d, J=16.2 Hz, 1H), 6.88 (d, J=16.2 Hz, 1H), 2.34 (s, 3H).

¹³C NMR (126 MHz, DMSO-d₆) δ 198.7, 156.16, 152.4, 135.8, 128.9, 124.2, 27.8.

### (E)-4-(thiazol-5-yl)but-3-en-2-one (182)

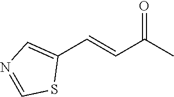

Following general procedure I, the title compound was prepared as a yellowish-brown powder.

LCMS (ESI-MS) m/z: [M+H]⁺ calcd C₇H₈NOS 154.02; found 153.9. Retention time: 0.828 min.

TLC: (33% EtOAc in hexanes, R_{f}): 0.17 (UV, 254 nm, 280 nm).

¹H NMR (500 MHz, DMSO-d₆) b 9.21 (s, 1H), 8.29 (s, 1H), 7.89 (d, J=15.7 Hz, 1H), 6.57 (d, J=15.7 Hz, 1H), 2.32 (s, 3H).

¹³C NMR (126 MHz, DMSO-d₆) δ 197.8, 157.3, 147.6, 135.4, 133.1, 129.5, 27.9.

### 5-cyclopropyl-3-hydroxycyclohex-2-en-1-one (194)

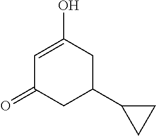 [SMILES: O=C1C=C(O)CC(C2CC2)C1]

Following general procedures J and K, the title compound was prepared as a yellow powder.

LCMS (ESI-MS) m/z: [M+H]⁺ calcd for C₉H₁₃O₂ 153.08; found 153.0. Retention time: 1.991 min.

TLC: (5% methanol in dichloromethane, R_{f}): 0.25 (UV, 254 nm, 280 nm).

¹H NMR (500 MHz, DMSO-d₆) δ 11.02 (s, 1H), 5.18 (s, 1H), 2.41-2.06 (m, 4H), 1.32-1.19 (m, 1H), 0.78-0.66 (m, 1H), 0.39 (dd, J=14.7, 5.3 Hz, 2H), 0.13 (dd, J=12.0, 5.2 Hz, 2H).

¹³C NMR (126 MHz, DMSO-d₆) δ 104.0, 38.9, 16.2, 3.5.

### 5-cyclobutyl-3-hydroxycyclohex-2-en-1-one (195)

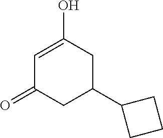 [SMILES: O=C1C=C(O)CC(C2CCC2)C1]

Following general procedures J and K, the title compound was prepared as a white powder.

LCMS (ESI-MS) m/z: [M+H]⁺ calcd for C₁₀H₁₅O₂ 167.10; found 167.0. Retention time: 3.183 min.

TLC: (5% methanol in dichloromethane, R_{f}): 0.25 (UV, 254 nm, 280 nm).

¹H NMR (500 MHz, DMSO-d₆) δ 10.97 (s, 1H), 5.18 (s, 1H), 2.28-2.11 (m, 3H), 2.01-1.86 (m, 5H), 1.86-1.76 (m, 1H), 1.75-1.57 (m, 3H).

¹³C NMR (126 MHz, DMSO-d₆) δ 103.9, 40.0, 39.8, 26.1, 17.9.

### 5-cyclopentyl-3-hydroxycyclohex-2-en-1-one (196)

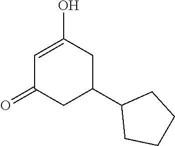 [SMILES: O=C1C=C(O)CC(C2CCCC2)C1]

Following general procedures J and K, the title compound was prepared as a white powder.

LCMS (ESI-MS) m/z: [M+H]⁺ calcd for C₁₁H₁₇O₂ 181.12; found 181.0. Retention time: 3.501 min.

TLC: (5% methanol in dichloromethane, R_{f}): 0.25 (UV, 254 nm, 280 nm).

¹H NMR (500 MHz, DMSO-d₆) δ 10.98 (s, 1H), 5.18 (s, 1H), 2.44-1.96 (m, 4H), 1.83-1.39 (m, 8H), 1.21-1.00 (m, 2H).

¹³C NMR (126 MHz, DMSO-d₆) δ 103.9, 45.2, 39.4, 30.3, 25.1.

### 5-hydroxy-[1,1′-bi(cyclohexan)]-4-en-3-one (197)

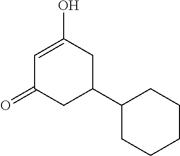 [SMILES: O=C1C=C(O)CC(C2CCCCC2)C1]

Following general procedures J and K, the title compound was prepared as a white powder.

LCMS (ESI-MS) m/z: [M+H]⁺ calcd for C₁₂H₁₉O₂ 195.13; found 195.0. Retention time: 3.765 min.

TLC: (5% methanol in dichloromethane, R_{f}): 0.25 (UV, 254 nm, 280 nm).

Keto Form: ¹H NMR (500 MHz, CD₃OD) δ 2.28 (dd, J=16.9, 4.4 Hz, 2H), 2.08 (dd, J=16.9, 11.9 Hz, 2H), 1.84-1.73 (m, 1H), 1.73-1.63 (m, 4H), 1.58 (d, J=12.5 Hz, 1H), 1.24-1.04 (m, 4H), 1.00-0.84 (m, 2H).

¹³C NMR (126 MHz, CD₃OD) δ 43.0, 40.7, 31.1, 27.6, 27.5.

Enol Form: ¹H NMR (500 MHz, DMSO-d₆) δ 10.98 (s, 1H), 5.18 (s, 1H), 2.22 (dd, J=16.6, 4.4 Hz, 2H), 2.15-1.97 (m, 2H), 1.84-1.75 (m, 1H), 1.75-1.65 (m, 4H), 1.61 (d, J=11.5 Hz, 1H), 1.27-1.05 (m, 4H), 1.01-0.88 (m, 2H).

¹³C NMR (126 MHz, DMSO-d₆) δ 103.88, 41.52, 39.13, 29.9, 26.5, 26.4.

IR (thin film, cm⁻¹): v=2921, 2850, 2534, 1613, 1518, 1475, 1317, 1221, 1144, 817, 591, 441.

### 3-hydroxy-5-(tetrahydrofuran-3-yl)cyclohex-2-en-1-one (198)

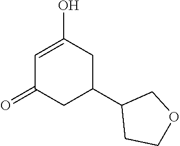 [SMILES: O=C1C=C(O)CC(C2CCOC2)C1]

Following general procedures J and K, the title compound was prepared as a yellow powder.

LCMS (ESI-MS) m/z: [M+H]⁺ calcd for C₁₀H₁₅O₃ 183.09; found 182.9. Retention time: 0.450 min.

TLC: (5% methanol in dichloromethane, R_{f}): 0.16 (UV, 254 nm, 280 nm).

### 3-hydroxy-5-(tetrahydro-2H-pyran-4-yl)cyclohex-2-en-1-one (199)

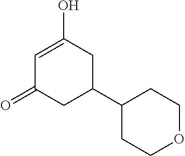 [SMILES: O=C1C=C(O)CC(C2CCOCC2)C1]

Following general procedures J and K, the title compound was prepared as an off-white and slightly yellow powder.

LCMS (ESI-MS) m/z: [M+H]⁺ calcd for C₁₁H₁₇O₃ 197.11; found 196.9. Retention time: 0.563 min.

TLC: (5% methanol in dichloromethane, R_{f}): 0.16 (UV, 254 nm, 280 nm).

¹H NMR (500 MHz, DMSO-d₆) δ 11.14 (s, 1H), 5.20 (s, 1H), 3.98-3.76 (m, 2H), 3.36-3.15 (m, 2H), 2.26 (dd, J=16.7, 4.4 Hz, 2H), 2.09 (dd, J=16.7, 11.8 Hz, 2H), 1.87-1.71 (m, 1H), 1.67-1.50 (m, 2H), 1.49-1.37 (m, 1H), 1.30-1.12 (m, 2H).

¹³C NMR (126 MHz, DMSO-d₆) δ 103.9, 67.5, 39.0, 38.7, 30.1.

### 5-(thiophen-2-yl)cyclohexane-1,3-dione (200)

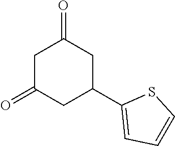 [SMILES: O=C1CC(=O)CC(c2cccs2)C1]

Following general procedures J and K, the title compound was prepared as a white solid.

LCMS (ESI-MS) m/z: [M+H]⁺ calcd for C₁₀H₁₁O₂S 195.04; found 194.8. Retention time: 2.921 min.

TLC: (5% methanol in dichloromethane, R_{f}): 0.19 (UV, 254 nm, 280 nm).

¹H NMR (500 MHz, CD₃OD) δ 7.27 (dd, J=3.7, 3.3 Hz, 1H), 7.02-6.89 (m, 2H), 4.91 (s, 2H), 3.78-3.65 (m, 1H), 2.79-2.71 (m, 2H), 2.70-2.62 (m, 2H).

¹³C NMR (126 MHz, CD₃OD) δ 148.0, 127.8, 124.6, 124.5, 36.0.

IR (thin film, cm⁻¹): v=2526, 1560, 1352, 1305, 1210, 1141, 1100, 865, 832, 777, 648, 626, 557, 486, 440.

### 5-(thiophen-3-yl)cyclohexane-1,3-dione (201)

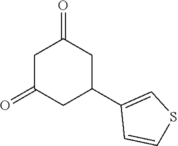 [SMILES: O=C1CC(=O)CC(c2ccsc2)C1]

Following general procedures J and K, the title compound was prepared as a white solid.

LCMS (ESI-MS) m/z: [M+H]⁺ calcd for C₁₀H₁₁O₂S 195.04; found 194.9. Retention time: 2.908 min.

TLC: (5% methanol in dichloromethane, R_{f}): 0.19 (UV, 254 nm, 280 nm).

¹H NMR (500 MHz, CD₃OD) δ 7.41-7.37 (m, 1H), 7.19 (d, J=2.9 Hz, 1H), 7.11 (d, J=4.9 Hz, 1H), 4.91 (s, 2H), 3.54-3.43 (m, 1H), 2.77-2.56 (m, 4H).

¹³C NMR (126 MHz, CD₃OD) δ 145.4, 127.5, 127.0, 121.0, 36.1.

IR (thin film, cm⁻¹): v=2890, 1566, 1401, 1352, 1302, 1221, 1140, 1103, 832, 778, 651, 559, 443.

### 5-(thiazol-2-yl)cyclohexane-1,3-dione (202)

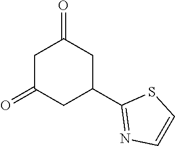 [SMILES: O=C1CC(=O)CC(c2nccs2)C1]

Following general procedures J and K, the title compound was prepared as a tan powder.

LCMS (ESI-MS) m/z: [M+H]⁺ calcd for C₉H₁₀NO₂S 196.04; found 195.8. Retention time: 0.506 min.

TLC: (10% methanol in dichloromethane, R_{f}): 0.29 (UV, 254 nm, 280 nm).

¹H NMR (500 MHz, CD₃OD) δ 7.75 (d, J=3.3 Hz, 1H), 7.52 (d, J=3.3 Hz, 1H), 4.93 (s, 2H), 3.94-3.83 (m, 1H), 2.89-2.77 (m, 4H).

¹³C NMR (126 MHz, CD₃OD) δ 173.9, 143.1, 120.2, 38.5.

IR (thin film, cm⁻¹): v=2942, 1724, 1647, 1602, 1499, 1382, 1220, 1140, 998, 872, 731.

### 3-hydroxy-5-(thiazol-4-yl)cyclohex-2-en-1-one (203)

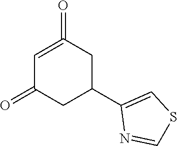

Following general procedures J and K, the title compound was prepared as a light brown powder.

LCMS (ESI-MS) m/z: [M+H]⁺ calcd for C₉H₁₀NO₂S 196.04; found 195.9. Retention time: 0.504 min.

TLC: (10% methanol in dichloromethane, R_{f}): 0.20 (UV, 254 nm, 280 nm).

¹H NMR (500 MHz, DMSO-d₆) δ 11.17 (s, 1H), 9.06 (d, J=1.9 Hz, 1H), 7.43 (d, J=1.9 Hz, 1H), 5.26 (s, 1H), 3.54 (p, J=7.6 Hz, 1H), 2.61 (s, 4H).

¹³C NMR (126 MHz, DMSO-d₆) δ 159.2, 154.4, 114.2, 104.1, 35.4.

### 5-(thiazol-5-yl)cyclohexane-1,3-dione (204)

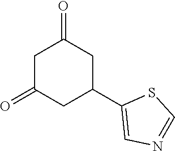 [SMILES: O=C1CC(=O)CC(c2cncs2)C1]

Following general procedures J and K, the title compound was prepared as a dark brown oil.

LCMS (ESI-MS) m/z: [M+H]⁺ calcd for C₉H₁₀NO₂S 196.04; found 195.9. Retention time: 0.438 min. [M+NH₄]⁺ calcd for C₉H₁₃N₂O₂S 213.07; found 213.8. Retention time: 0.438.

TLC: (10% methanol in dichloromethane, R_{f}): 0.23 (UV, 254 nm, 280 nm).

¹H NMR (500 MHz, CD₃OD) b 8.80 (s, 1H), 7.66 (s, 1H), 4.79 (s, 2H), 3.77-3.68 (m, 1H), 2.68 (dd, J=16.9, 4.8 Hz, 2H), 2.56 (dd, J=16.9, 10.1 Hz, 2H).

¹³C NMR (126 MHz, CD₃OD) δ 154.3, 143.4, 140.4, 33.5.

IR (thin film, cm⁻¹): v=2899, 1717, 1600, 1405, 1220, 1106, 871, 732, 605.

### 7-chloro-3-(2,4-dichlorophenyl)-3,4-dihydroacridine-1,9(2H,10H)-dione (1)

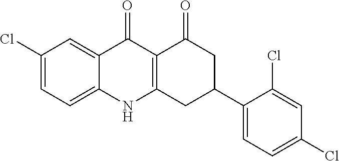 [SMILES: O=C1CC(c2ccc(Cl)cc2Cl)Cc2[nH]c3ccc(Cl)cc3c(=O)c21]

Following general procedure L, the title compound was prepared as a white solid.

TLC: (5% methanol in dichloromethane, R_{f}): 0.38 (UV, 254 nm, 280 nm).

¹H NMR (500 MHz, CDCl₃ and 3 drops of TFA-d) δ 8.50 (d, J=2.0 Hz, 1H), 8.12-8.01 (m, 2H), 7.50 (d, J=2.1 Hz, 1H), 7.34 (dd, J=8.4, 2.1 Hz, 1H), 7.25 (d, J=8.4 Hz, 1H), 4.21-4.08 (m, 1H), 3.76-3.60 (m, 2H), 3.23 (ddd, J=17.8, 4.0, 1.5 Hz, 1H), 3.14 (dd, J=17.8, 12.6 Hz, 1H).

¹³C NMR (126 MHz, CDCl₃ and 3 drops of TFA-d) δ 202.2, 172.4, 161.0, 139.0, 137.9, 136.5, 134.9, 134.8, 134.2, 130.4, 128.2, 127.6, 124.2, 122.1, 120.2, 108.1, 42.5, 34.6, 33.4.

### 7-chloro-3-(4-(4-(trifluoromethoxy)phenoxy)phenyl)-3,4-dihydroacridine-1,9(2H,10H)-dione (2)

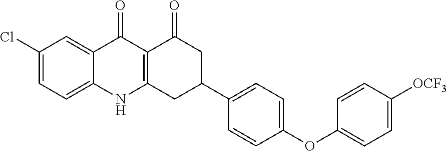 [SMILES: O=C1CC(c2ccc(Oc3ccc(OC(F)(F)F)cc3)cc2)Cc2[nH]c3ccc(Cl)cc3c(=O)c21]

Following general procedure L, the title compound was prepared as a white powder.

TLC: (5% methanol in dichloromethane, R_{f}): 0.42 (UV, 254 nm, 280 nm).

¹H NMR (500 MHz, CDCl₃ and 3 drops of TFA-d) δ 8.49 (s, 1H), 8.08 (s, 2H), 7.26-7.17 (m, 4H), 7.14-6.93 (m, 4H), 3.83-3.55 (m, 3H), 3.29-3.11 (m, 2H).

¹³C NMR (126 MHz, CDCl₃ and 3 drops of TFA-d) δ 202.8, 172.4, 161.4, 157.0, 155.1, 144.9 (q, J_{C—F}=2.0 Hz), 139.0, 138.0, 136.5, 134.2, 128.0, 124.2, 122.7, 122.3, 120.8 (q, J_{C—F}=256.8 Hz), 120.3, 120.1, 119.5, 108.3, 44.2, 37.7, 35.5.

¹⁹F NMR (471 MHz, CDCl₃ and 3 drops of TFA-d) δ −59.09.

### 7-fluoro-3-(4-(4-(trifluoromethoxy)phenoxy)phenyl)-3,4-dihydroacridine-1,9(2H,10H)-dione (3)

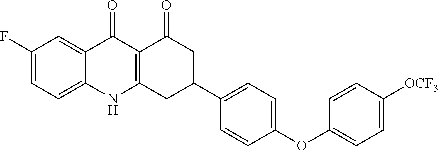 [SMILES: O=C1CC(c2ccc(Oc3ccc(OC(F)(F)F)cc3)cc2)Cc2[nH]c3ccc(F)cc3c(=O)c21]

Following general procedure L, the title compound was prepared as a white powder.

TLC: (5% methanol in dichloromethane, R_{f}): 0.42 (UV, 254 nm, 280 nm).

¹H NMR (500 MHz, CDCl₃ and 3 drops of TFA-d) δ 8.18-8.10 (m, 2H), 7.94-7.87 (m, 1H), 7.26-7.18 (m, 4H), 7.07-7.00 (m, 4H), 3.78-3.67 (m, 2H), 3.62 (dd, J=18.3, 12.1 Hz, 1H), 3.30-3.12 (m, 2H).

¹³C NMR (126 MHz, CDCl₃ and 3 drops of TFA-d) δ 202.9, 172.8 (d, J_{C—F}=4.2 Hz), 162.9, 160.9, 160.8 (d, J_{C—F}=1.9 Hz), 156.1 (d, J_{C—F}=251.4 Hz), 145.0 (q, J_{C—F}=2.4 Hz), 136.3, 134.2, 128.2 (d, J_{C—F}=25.9 Hz), 128.0, 123.4 (d, J_{C—F}=9.0 Hz), 122.8, 120.9 (d, J_{C—F}=9.6 Hz), 120.2, 119.9 (q, J=264.5 Hz), 119.57, 119.53, 109.9 (d, J_{C—F}=24.4 Hz), 108.0, 44.2, 37.7, 35.4.

¹⁹F NMR (471 MHz, CDCl₃ and 3 drops of TFA-d) δ −59.08, −105.31.

### 6-methoxy-3-(4-(4-(trifluoromethoxy)phenoxy)phenyl)-3,4-dihydroacridine-1,9(2H,10H)-dione (4)

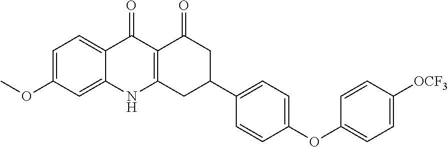 [SMILES: COc1ccc2c(=O)c3c([nH]c2c1)CC(c1ccc(Oc2ccc(OC(F)(F)F)cc2)cc1)CC3=O]

Following general procedure L, the title compound was prepared as a white powder.

TLC: (5% methanol in dichloromethane, R_{f}): 0.42 (UV, 254 nm, 280 nm).

¹H NMR (500 MHz, DMSO-d₆) δ 12.68 (s, 1H), 8.03 (d, J=8.9 Hz, 1H), 7.44 (d, J=8.4 Hz, 2H), 7.39 (d, J=8.4 Hz, 2H), 7.15-7.04 (m, 5H), 6.99 (dd, J=8.9, 2.4 Hz, 1H), 3.86 (s, 3H), 3.57-3.46 (m, 1H), 3.33-3.28 (m, 1H), 3.19 (d, J=16.2 Hz, 1H), 2.87-2.74 (m, 1H), 2.57 (d, J=16.2 Hz, 1H).

### 4-(4-(trifluoromethoxy)phenoxy)benzaldehyde (6)

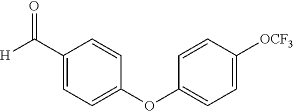 [SMILES: O=Cc1ccc(Oc2ccc(OC(F)(F)F)cc2)cc1]

Following general procedure D, the title compound was prepared as a faint yellow viscous oil.

LCMS (ESI-MS) m/z [M+H]⁺ calcd for C₁₄H₁₀F₃O₃ 283.05; found 282.8. Retention time: 4.718 min.

TLC: (10% EtOAc in hexanes, R_{f}): 0.30 (UV, 254 nm, 280 nm).

¹H NMR (500 MHz, CDCl₃) δ 9.94 (s, 1H), 7.87 (d, J=8.7 Hz, 2H), 7.26 (d, J=8.2 Hz, 2H), 7.13-7.06 (m, 4H).

¹³C NMR (126 MHz, CDCl₃) δ 190.6, 162.6, 153.6, 145.7 (q, J_{C—F}=1.9 Hz), 132.0, 131.7, 122.9, 121.3, 120.4 (q, J_{C—F}=257.2 Hz), 117.8.

¹⁹F NMR (471 MHz, CDCl₃) δ −58.21.

### (E)-4-(4-(4-(trifluoromethoxy)phenoxy)phenyl)but-3-en-2-one (7)

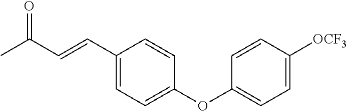 [SMILES: CC(=O)/C=C/c1ccc(Oc2ccc(OC(F)(F)F)cc2)cc1]

Following general procedure I, the title compound was prepared as a white powder.

LCMS (ESI-MS) m/z: [M+H]⁺ calcd for C₁₇H₁₄F₃O₃ 323.08; found 323.1. Retention time: 4.083 min.

TLC: (20% EtOAc in hexanes, R_{f}): 0.40 (UV, 254 nm, 280 nm).

¹H NMR (500 MHz, DMSO-d₆) δ 7.76 (d, J=8.4 Hz, 2H), 7.62 (d, J=16.5 Hz, 1H), 7.42 (d, J=8.9 Hz, 2H), 7.19 (d, J=8.9 Hz, 2H), 7.07 (d, J=8.4 Hz, 2H), 6.74 (d, J=16.5 Hz, 1H), 2.32 (s, 3H).

¹³C NMR (126 MHz, CDCl₃) δ 198.2, 159.1, 154.6, 145.1 (d, J_{C—F}=2.0 Hz), 142.4, 130.0, 129.7, 126.3, 122.7, 120.5, 120.4 (q, J_{C—F}=256.9 Hz), 118.7, 27.5.

¹⁹F NMR (471 MHz, CDCl₃) δ −58.23.

### 5-hydroxy-4′-(4-(trifluoromethoxy)phenoxy)-1,6-dihydro-[1,1′-biphenyl]-3(2H)-one (9)

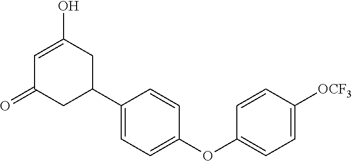 [SMILES: O=C1C=C(O)CC(c2ccc(Oc3ccc(OC(F)(F)F)cc3)cc2)C1]

Following general procedures J and K, the title compound was prepared as an off-white powder.

LCMS (ESI-MS) m/z: [M+H]⁺ calcd for C₁₉H₁₆F₃O₄ 365.09; found 364.8. Retention time: 4.418 min.

TLC: (2% methanol in dichloromethane, R_{f}): 0.23 (UV, 254 nm, 280 nm).

¹H NMR (500 MHz, DMSO-d₆) δ 11.19 (s, 1H), 7.39 (dd, J=9.2, 7.5 Hz, 4H), 7.13-7.06 (m, 2H), 7.05-6.97 (m, 2H), 5.30 (s, 1H), 3.44-3.25 (m, 1H), 2.81-2.52 (m, 2H), 2.49-2.11 (m, 2H).

¹³C NMR (126 MHz, DMSO-d₆) δ 156.3, 155.1, 144.0 (q, J_{C—F}=2.0 Hz), 139.8, 129.1, 123.4, 120.6 (q, J_{C—F}=257.5 Hz), 120.0, 119.5, 104.0, 38.5.

¹⁹F NMR (471 MHz, DMSO-d₆) δ −57.21.

IR (thin film, cm⁻¹): v=2896, 1596, 1498, 1240, 1189, 1013, 835, 515.

### 6-chloro-2H-benzo[d][1,3]oxazine-2,4(1H)-dione (13)

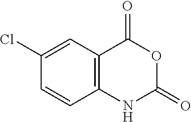 [SMILES: O=c1[nH]c2ccc(Cl)cc2c(=O)o1]

Following general procedure A, the title compound was prepared as a white powder.

LCMS (ESI-MS) m/z: [M+H]⁺ calcd for C₈H₅ClNO₃ 197.99; found 197.8. Retention time: 2.921 min.

TLC: (50% EtOAc in hexanes, R_{f}): 0.34 (UV, 254 nm, 280 nm).

¹H NMR (500 MHz, DMSO-d₆) δ 11.86 (s, 1H), 7.87 (d, J=2.4 Hz, 1H), 7.78 (dd, J=8.8, 2.4 Hz, 1H), 7.16 (d, J=8.8 Hz, 1H).

¹³C NMR (126 MHz, DMSO-d₆) δ 158.9, 146.7, 140.3, 136.6, 127.6, 127.1, 117.4, 112.0

### 6-fluoro-2H-benzo[d][1,3]oxazine-2,4(1H)-dione (14)

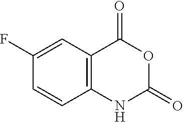 [SMILES: O=c1[nH]c2ccc(F)cc2c(=O)o1]

Following general procedure A, the title compound was prepared as a white powder.

LCMS (ESI-MS) m/z: [M+H]⁺ calcd for C₈H₅FNO₃ 182.02; found 181.8. Retention time: 1.194 min.

TLC: (50% EtOAc in hexanes, R_{f}): 0.35 (UV, 254 nm, 280 nm).

¹H NMR (500 MHz, DMSO-d₆) δ 11.80 (s, 1H), 7.74-7.60 (m, 2H), 7.19 (dd, J=9.2, 4.6 Hz, 1H).

¹³C NMR (126 MHz, DMSO-d₆) δ 159.6 (d, J_{C—F}=3.3 Hz), 157.9 (d, J_{C—F}=241.3 Hz), 147.2, 138.6 (d, J_{C—F}=1.5 Hz), 125.2 (d, J_{C—F}=24.5 Hz), 118.0 (d, J_{C—F}=8.4 Hz), 114.4 (d, J_{C—F}=24.3 Hz), 111.9 (d, J_{C—F}=24.4 Hz).

¹⁹F NMR (471 MHz, DMSO-d₆) δ −118.76-−118.85 (m).

### 7-methoxy-2H-benzo[d][1,3]oxazine-2,4(1H)-dione (15)

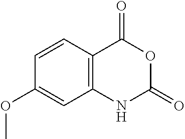

Following general procedure A, the title compound was prepared as an off-white, slightly tan powder.

LCMS (ESI-MS) m/z: [M+H]⁺ calcd for C₉H₈NO₄ 194.04; found 193.9. Retention time: 1.581 min.

TLC: (50% EtOAc in hexanes, R_{f}): 0.24 (UV, 254 nm, 280 nm).

¹H NMR (500 MHz, DMSO-d₆) δ 11.64 (s, 1H), 7.82 (d, J=8.9 Hz, 1H), 6.82 (dd, J=8.9, 2.4 Hz, 1H), 6.58 (d, J=2.4 Hz, 1H), 3.85 (s, 3H).

¹³C NMR (126 MHz, DMSO-d₆) δ 165.8, 159.2, 147.4, 143.6, 131.0, 111.6, 102.8, 98.5, 55.9.

### 7-chloro-3-(5-(4-(trifluoromethoxy)phenoxy)thiophen-2-yl)-3,4-dihydroacridine-1,9(2H,10H)-dione (16)

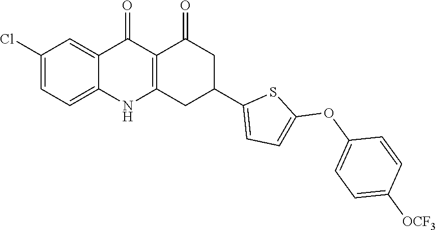 [SMILES: O=C1CC(c2ccc(Oc3ccc(OC(F)(F)F)cc3)s2)Cc2[nH]c3ccc(Cl)cc3c(=O)c21]

Following general procedure L, the title compound was prepared as a yellow powder.

TLC: (5% methanol in dichloromethane, R_{f}): 0.32 (UV, 254 nm, 280 nm).

¹H NMR (500 MHz, DMSO-d₆) δ 12.28 (s, 1H), 8.03 (d, J=2.5 Hz, 1H), 7.73 (dd, J=8.7, 2.5 Hz, 1H), 7.57 (d, J=8.7 Hz, 1H), 7.41 (d, J=9.1 Hz, 2H), 7.21 (d, J=9.1 Hz, 2H), 6.78 (d, J=3.8 Hz, 1H), 6.62 (d, J=3.8 Hz, 1H), 3.78-3.68 (m, 1H), 3.30-3.20 (m, 2H), 2.81-2.67 (m, 2H).

¹H NMR (500 MHz, CDCl₃ and 3 drops of TFA-d) δ 8.46 (d, J=2.3 Hz, 1H), 8.16 (d, J=8.9 Hz, 1H), 8.06 (dd, J=9.1, 2.3 Hz, 1H), 7.19 (d, J=8.8 Hz, 2H), 7.12-7.03 (m, 2H), 6.68 (d, J=3.8 Hz, 1H), 6.43-6.38 (m, 1H), 3.98-3.87 (m, 2H), 3.68 (dd, J=18.0, 9.5 Hz, 1H), 3.33 (dd, J=18.0, 3.8 Hz, 1H), 3.16 (dd, J=18.0, 9.5 Hz, 1H).

¹³C NMR (126 MHz, CDCl₃ and 3 drops of TFA-d) δ 202.0, 172.0, 160.5, 159.0, 156.8, 145.0 (q, J_{C—F}=2.3 Hz), 138.68, 138.62, 136.1, 133.9, 124.0, 122.9, 122.6, 122.5, 120.4 (q, J_{C—F}=256.9 Hz), 120.3, 118.1, 113.3, 108.2, 44.4, 35.3, 34.1.

¹⁹F NMR (471 MHz, CDCl₃ and 3 drops of TFA-d) δ −58.98.

### 6-methoxy-3-(5-(4-(trifluoromethoxy)phenoxy)thiophen-2-yl)-3,4-dihydroacridine-1,9(2H,10H)-dione (17)

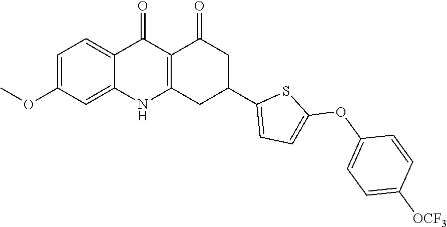 [SMILES: COc1ccc2c(=O)c3c([nH]c2c1)CC(c1ccc(Oc2ccc(OC(F)(F)F)cc2)s1)CC3=O]

Following general procedure L, the title compound was prepared as a light brown powder.

TLC: (5% methanol in dichloromethane, R_{f}): 0.27 (UV, 254 nm, 280 nm).

¹H NMR (500 MHz, DMSO-d₆) δ 11.92 (s, 1H), 8.01 (d, J=8.9 Hz, 1H), 7.44-7.37 (m, 2H), 7.24-7.17 (m, 2H), 6.97 (dd, J=8.9, 2.3 Hz, 1H), 6.90 (s, 1H), 6.77 (dd, J=3.8, 1.1 Hz, 1H), 6.62 (d, J=3.8 Hz, 1H), 3.86 (s, 3H), 3.77-3.67 (m, 1H), 3.30-3.17 (m, 2H), 2.79-2.64 (m, 2H).

### 7-chloro-3-(5-(3-(trifluoromethoxy)phenoxy)thiophen-2-yl)-3,4-dihydroacridine-1,9(2H,10H)-dione (18)

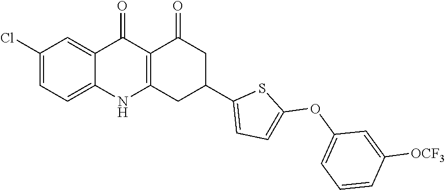 [SMILES: O=C1CC(c2ccc(Oc3cccc(OC(F)(F)F)c3)s2)Cc2[nH]c3ccc(Cl)cc3c(=O)c21]

Following general procedure L, the title compound was prepared as a white powder.

TLC: (5% methanol in dichloromethane, R_{f}): 0.32 (UV, 254 nm, 280 nm).

¹H NMR (500 MHz, DMSO-d₆) δ 12.25 (s, 1H), 8.02 (d, J=2.5 Hz, 1H), 7.73 (dd, J=9.0, 2.5 Hz, 1H), 7.59-7.49 (m, 2H), 7.19-7.11 (m, 2H), 7.09 (s, 1H), 6.79 (d, J=3.8 Hz, 1H), 6.66 (d, J=3.8 Hz, 1H), 3.80-3.70 (m, 1H), 3.31-3.21 (m, 2H), 2.82-2.67 (m, 2H).

### 6-methoxy-3-(5-(3-(trifluoromethoxy)phenoxy)thiophen-2-yl)-3,4-dihydroacridine-1,9(2H,10H)-dione (19)

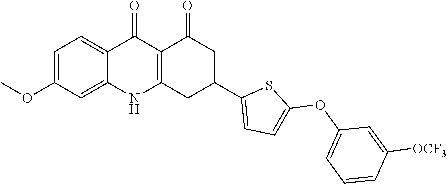 [SMILES: COc1ccc2c(=O)c3c([nH]c2c1)CC(c1ccc(Oc2cccc(OC(F)(F)F)c2)s1)CC3=O]

Following general procedure L, the title compound was prepared as a yellow powder.

TLC: (5% methanol in dichloromethane, R_{f}): 0.27 (UV, 254 nm, 280 nm).

¹H NMR (500 MHz, DMSO-d₆) δ 11.94 (s, 1H), 8.01 (d, J=9.1 Hz, 1H), 7.53 (dd, J=8.8, 8.2 Hz, 1H), 7.19-7.11 (m, 2H), 7.09 (s, 1H), 6.97 (d, J=9.1 Hz, 1H), 6.90 (s, 1H), 6.79 (d, J=3.8 Hz, 1H), 6.65 (d, J=3.8 Hz, 1H), 3.86 (s, 3H), 3.78-3.69 (m, 1H), 3.31-3.17 (m, 2H), 2.79-2.64 (m, 2H).

### 7-chloro-3-(6-(4-(trifluoromethoxy)phenoxy)pyridin-3-yl)-3,4-dihydroacridine-1,9(2H,10H)-dione (20)

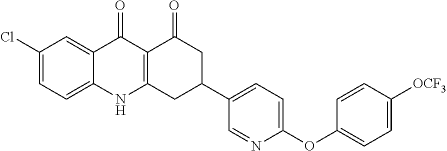 [SMILES: O=C1CC(c2ccc(Oc3ccc(OC(F)(F)F)cc3)nc2)Cc2[nH]c3ccc(Cl)cc3c(=O)c21]

Following general procedure L, the title compound was prepared as a yellow powder.

TLC: (5% methanol in dichloromethane, R_{f}): 0.33 (UV, 254 nm, 280 nm).

¹H NMR (500 MHz, DMSO-d₆) δ 12.27 (s, 1H), 8.16 (s, 1H), 8.03 (s, 1H), 7.94 (d, J=8.6 Hz, 1H), 7.73 (d, J=8.8 Hz, 1H), 7.59 (d, J=8.8 Hz, 1H), 7.42 (d, J=7.9 Hz, 2H), 7.27 (d, J=7.9 Hz, 2H), 7.12 (d, J=8.6 Hz, 1H), 3.62-3.50 (m, 1H), 3.42-3.37 (m, 1H), 3.10 (d, J=16.3 Hz, 1H), 2.86-2.74 (m, 1H), 2.59 (d, J=16.3 Hz, 1H).

### 6-methoxy-3-(6-(4-(trifluoromethoxy)phenoxy)pyridin-3-yl)-3,4-dihydroacridine-1,9(2H,10H)-dione (21)

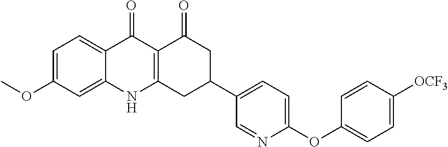 [SMILES: COc1ccc2c(=O)c3c([nH]c2c1)CC(c1ccc(Oc2ccc(OC(F)(F)F)cc2)nc1)CC3=O]

Following general procedure L, the title compound was prepared as a yellow powder.

TLC: (5% methanol in dichloromethane, R_{f}): 0.32 (UV, 254 nm, 280 nm).

¹H NMR (500 MHz, DMSO-d₆) δ 11.93 (s, 1H), 8.16 (s, 1H), 8.02 (d, J=9.0 Hz, 1H), 7.94 (d, J=8.2 Hz, 1H), 7.42 (d, J=8.5 Hz, 2H), 7.27 (d, J=8.4 Hz, 2H), 7.11 (d, J=8.2 Hz, 1H), 6.97 (d, J=9.0 Hz, 1H), 6.92 (s, 1H), 3.86 (s, 3H), 3.58-3.49 (m, 1H), 3.06 (d, J=16.4 Hz, 1H), 2.82-2.72 (m, 1H), 2.56 (d, J=16.4 Hz, 1H).

### 7-chloro-3-(6-(3-(trifluoromethoxy)phenoxy)pyridin-3-yl)-3,4-dihydroacridine-1,9(2H,10H)-dione (22)

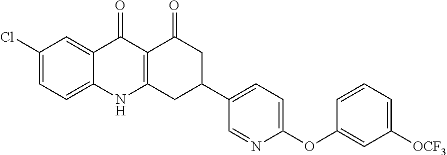 [SMILES: O=C1CC(c2ccc(Oc3cccc(OC(F)(F)F)c3)nc2)Cc2[nH]c3ccc(Cl)cc3c(=O)c21]

Following general procedure L, the title compound was prepared as a white powder.

TLC: (5% methanol in dichloromethane, R_{f}): 0.33 (UV, 254 nm, 280 nm).

¹H NMR (500 MHz, CDCl₃ and 3 drops of TFA-d) δ 8.73 (s, 1H), 8.52 (d, J=2.2 Hz, 1H), 8.42 (d, J=8.8 Hz, 1H), 8.09 (dd, J=9.1, 2.2 Hz, 1H), 8.02 (d, J=9.1 Hz, 1H), 7.61 (dd, J=9.1, 8.3 Hz, 1H), 7.34 (d, J=8.8 Hz, 1H), 7.23-7.10 (m, 3H), 4.07-3.90 (m, 1H), 3.90-3.65 (m, 2H), 3.40-3.18 (m, 2H).

¹³C NMR (126 MHz, CDCl₃ and 3 drops of TFA-d) δ 200.7, 172.6, 160.0, 159.3, 151.4, 150.8 (q, J_{C—F}=2.2 Hz), 148.3, 139.2, 138.9, 137.9, 136.8, 133.2, 132.3, 124.4, 122.0, 120.7, 120.3, 120.2 (q, J_{C-F}=259.2 Hz), 118.8, 113.8, 112.7, 108.1, 34.9, 34.8, 34.0.

¹⁹F NMR (471 MHz, CDCl₃ and 3 drops of TFA-d) δ −58.98.

### 6-methoxy-3-(6-(3-(trifluoromethoxy)phenoxy)pyridin-3-yl)-3,4-dihydroacridine-1,9(2H,10H)-dione (23)

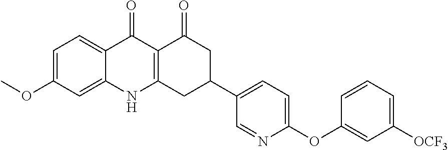 [SMILES: COc1ccc2c(=O)c3c([nH]c2c1)CC(c1ccc(Oc2cccc(OC(F)(F)F)c2)nc1)CC3=O]

Following general procedure L, the title compound was prepared as a white powder.

TLC: (5% methanol in dichloromethane, R_{f}): 0.33 (UV, 254 nm, 280 nm).

¹H NMR (500 MHz, CDCl₃ and 3 drops of TFA-d) δ 8.74 (d, J=2.4 Hz, 1H), 8.43-8.34 (m, 2H), 7.59 (dd, J=9.1, 8.3 Hz, 1H), 7.41 (dd, J=9.3, 2.3 Hz, 1H), 7.36-7.29 (m, 2H), 7.18 (dd, J=8.2, 2.1 Hz, 1H), 7.15-7.08 (m, 2H), 4.03 (s, 3H), 3.98-3.88 (m, 1H), 3.81 (dd, J=17.4, 3.1 Hz, 1H), 3.67 (dd, J=17.4, 12.1 Hz, 1H), 3.30-3.13 (m, 2H).

¹⁹F NMR (471 MHz, CDCl₃ and 3 drops of TFA-d) δ −58.83.

### 5-(4-(trifluoromethoxy)phenoxy)thiophene-2-carbaldehyde (26)

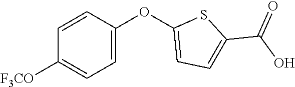 [SMILES: O=C(O)c1ccc(Oc2ccc(OC(F)(F)F)cc2)s1]

Following the general procedure D, the title compound was prepared as a brown solid.

LCMS (ESI-MS) m/z [M+H]⁺ calcd for C₁₂H₈F₃O₃S 289.01; found 288.8. Retention time: 3.774 min.

TLC: (7% EtOAc in hexanes, R_{f}): 0.18 (UV, 254 nm, 280 nm).

¹H NMR (500 MHz, CDCl₃) δ 9.73 (s, 1H), 7.55 (d, J=4.3 Hz, 1H), 7.29-7.24 (m, 2H), 7.24-7.18 (m, 2H), 6.54 (d, J=4.3 Hz, 1H).

¹³C NMR (126 MHz, CDCl₃) δ 182.7, 171.2, 155.1, 146.3 (q, J_{C—F}=1.9 Hz), 136.5, 133.4, 122.9, 120.4 (q, J_{C—F}=257.5 Hz), 120.3, 112.1.

¹⁹F NMR (471 MHz, CDCl₃) δ −58.21.

### 5-(3-(trifluoromethoxy)phenoxy)thiophene-2-carbaldehyde (27)

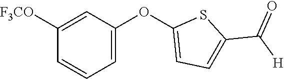 [SMILES: O=Cc1ccc(Oc2cccc(OC(F)(F)F)c2)s1]

Following the general procedure D, the title compound was prepared as a brown oil.

LCMS (ESI-MS) m/z [M+H]⁺ calcd for C₁₂H₈F₃O₃S 289.01; found 288.8. Retention time: 3.771 min.

TLC: (7% EtOAc in hexanes, R_{f}): 0.21 (UV, 254 nm, 280 nm).

¹H NMR (500 MHz, CDCl₃) δ 9.76 (s, 1H), 7.58 (d, J=4.2 Hz, 1H), 7.45 (dd, J=9.0, 8.3 Hz, 1H), 7.17-7.09 (m, 2H), 7.07 (s, 1H), 6.60 (d, J=4.2 Hz, 1H).

¹³C NMR (126 MHz, CDCl₃) δ 182.7, 170.2, 157.6, 150.1 (q, J=2.0 Hz), 136.4, 133.8, 131.0, 120.3 (q, J=258.3 Hz), 117.6, 116.9, 112.8, 111.9.

¹⁹F NMR (471 MHz, CDCl₃) δ −57.95.

### 6-(4-(trifluoromethoxy)phenoxy)nicotinaldehyde (28)

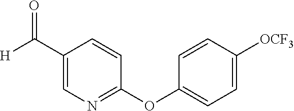 [SMILES: O=Cc1ccc(Oc2ccc(OC(F)(F)F)cc2)nc1]

Following general procedure D, the title compound was prepared as a faint yellow powder.

LCMS (ESI-MS) m/z [M+H]⁺ calcd for C₁₃H₉F₃NO₃ 284.05; found 283.8. Retention time: 4.384 min.

TLC: (10% EtOAc in hexanes, R_{f}): 0.13 (UV, 254 nm, 280 nm).

¹H NMR (500 MHz, CDCl₃) δ 10.0 (s, 1H), 8.6 (d, J=2.4 Hz, 1H), 8.2 (dd, J=8.8, 2.4 Hz, 1H), 7.3 (d, J=9.1 Hz, 2H), 7.2 (d, J=9.1 Hz, 2H), 7.1 (d, J=8.8 Hz, 1H).

¹⁹F NMR (471 MHz, CDCl₃) δ −58.06.

### 6-(3-(trifluoromethoxy)phenoxy)nicotinaldehyde (29)

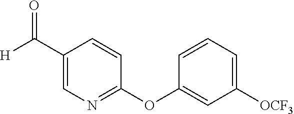 [SMILES: O=Cc1ccc(Oc2cccc(OC(F)(F)F)c2)nc1]

Following general procedure D, the title compound was prepared as a faint yellow powder.

LCMS (ESI-MS) m/z [M+H]⁺ calcd for C₁₃HsF₃NO₃ 284.05; found 283.8. Retention time: 4.391 min.

TLC: (10% EtOAc in hexanes, R_{f}): 0.13 (UV, 254 nm, 280 nm).

¹H NMR (500 MHz, CDCl₃) δ 10.02 (s, 1H), 8.65 (d, J=2.3 Hz, 1H), 8.24 (dd, J=8.9, 2.3 Hz, 1H), 7.48 (dd, J=9.3, 8.2 Hz, 1H), 7.19-7.08 (m, 4H).

¹³C NMR (126 MHz, CDCl₃) δ 189.1, 166.4, 153.8, 152.4, 149.9 (d, J_{C-F}=1.8 Hz), 139.0, 130.5, 128.2, 120.4 (q, J_{C—F}=257.9 Hz), 120.0, 117.8, 114.8, 112.4.

¹⁹F NMR (471 MHz, CDCl₃) δ −57.89.

### (E)-4-(5-(4-(trifluoromethoxy)phenoxy)thiophen-2-yl)but-3-en-2-one (30)

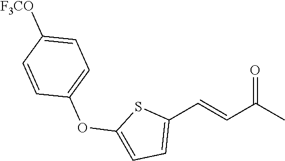 [SMILES: CC(=O)/C=C/c1ccc(Oc2ccc(OC(F)(F)F)cc2)s1]

Following general procedure H, the title compound was prepared as a dark brown oil.

LCMS (ESI-MS) m/z: [M+H]⁺ calcd for C₁₅H₁₂F₃O₃S 329.04; found 328.9. Retention time: 4.018 min.

TLC: (25% EtOAc in hexanes, R_{f}): 0.26 (UV, 254 nm, 280 nm).

¹H NMR (500 MHz, CDCl₃) δ 7.51 (d, J=15.7 Hz, 1H), 7.25-7.19 (m, 2H), 7.19-7.12 (m, 2H), 7.04 (d, J=4.0 Hz, 1H), 6.47 (d, J=4.0 Hz, 1H), 6.32 (d, J=15.7 Hz, 1H), 2.31 (s, 3H).

¹³C NMR (126 MHz, CDCl₃) δ 197.3, 163.3, 156.1, 145.5 (q, J_{C—F}=2.2 Hz), 136.3, 130.8, 130.6, 124.0, 122.7, 120.4 (q, J_{C—F}=257.3 Hz), 119.0, 113.5, 27.8.

¹⁹F NMR (471 MHz, CDCl₃) δ −58.24.

### (E)-4-(5-(3-(trifluoromethoxy)phenoxy)thiophen-2-yl)but-3-en-2-one (31)

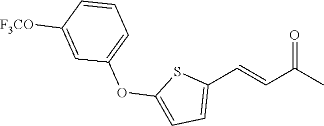 [SMILES: CC(=O)/C=C/c1ccc(Oc2cccc(OC(F)(F)F)c2)s1]

Following general procedure H, the title compound was prepared as a dark brown oil.

LCMS (ESI-MS) m/z: [M+H]⁺ calcd for C₁₅H₁₂F₃O₃S 329.08; found 328.9. Retention time: 3.968 min.

TLC: (25% EtOAc in hexanes, R_{f}): 0.24 (UV, 254 nm, 280 nm).

¹H NMR (399 MHz, CDCl₃) δ 7.52 (d, J=15.8 Hz, 1H), 7.38 (t, J=8.3 Hz, 1H), 7.10-6.98 (m, 4H), 6.51 (d, J=4.0 Hz, 1H), 6.34 (d, J=15.8 Hz, 1H), 2.32 (s, 3H).

¹³C NMR (126 MHz, CDCl₃) δ 197.3, 162.3, 158.6, 150.0 (q, J_{C—F}=2.1 Hz), 136.2, 131.1, 130.8-130.6 (m), 124.2, 120.3 (q, J_{C—F}=258.1 Hz), 116.6, 115.8, 114.2, 110.8, 27.7, 27.6.

¹⁹F NMR (471 MHz, CDCl₃) δ −57.92.

### (E)-4-(6-(4-(trifluoromethoxy)phenoxy)pyridin-3-yl)but-3-en-2-one (32)

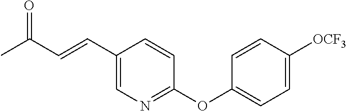 [SMILES: CC(=O)/C=C/c1ccc(Oc2ccc(OC(F)(F)F)cc2)nc1]

Following general procedure 1, the title compound was prepared as a white powder.

LCMS (ESI-MS) m/z: [M+H]⁺ calcd for C₁₆H₁₃F₃NO₃ 324.08; found 323.8. Retention time: 4.520 min.

TLC: (20% EtOAc in hexanes, R_{f}): 0.22 (UV, 254 nm, 280 nm).

¹H NMR (500 MHz, CDCl₃) δ 8.30 (d, J=2.5 Hz, 1H), 7.92 (dd, J=8.6, 2.5 Hz, 1H), 7.46 (d, J=16.3 Hz, 1H), 7.27 (d, J=8.8 Hz, 2H), 7.21-7.15 (m, 2H), 7.00 (d, J=8.6 Hz, 1H), 6.67 (d, J=16.3 Hz, 1H), 2.39 (s, 3H).

¹³C NMR (126 MHz, CDCl₃) δ 197.7, 164.4, 151.7, 148.6, 145.9 (q, J_{C-F}=1.7 Hz), 138.8, 137.6, 127.1, 125.8, 122.6, 122.4, 120.4 (q, J_{C—F}=257.2 Hz), 112.1, 27.6. [1233]¹⁹F NMR (471 MHz, CDCl₃) δ −58.07.

### (E)-4-(6-(3-(trifluoromethoxy)phenoxy)pyridin-3-yl)but-3-en-2-one (33)

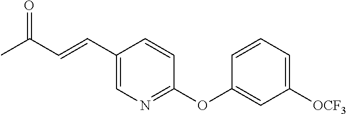 [SMILES: CC(=O)/C=C/c1ccc(Oc2cccc(OC(F)(F)F)c2)nc1]

Following general procedure 1, the title compound was prepared as an off-white powder.

LCMS (ESI-MS) m/z: [M+H]⁺ calcd for C₁₆H₁₃F₃NO₃ 324.08; found 323.8. Retention time: 3.685 min.

TLC: (20% EtOAc in hexanes, R_{f}): 0.22 (UV, 254 nm, 280 nm).

¹H NMR (500 MHz, CDCl₃) δ 8.31 (d, J=2.4 Hz, 1H), 7.93 (dd, J=8.6, 2.4 Hz, 1H), 7.50-7.40 (m, 2H), 7.15-7.08 (m, 2H), 7.06 (s, 1H), 7.00 (d, J=8.6 Hz, 1H), 6.68 (d, J=16.2 Hz, 1H), 2.39 (s, 3H).

¹³C NMR (126 MHz, CDCl₃) δ 197.7, 164.1, 154.3, 149.9 (q, J_{C—F}=1.9 Hz), 148.6, 138.8, 137.7, 130.4, 127.3, 126.0, 121.4 (q, J_{C—F}=257.3 Hz), 119.6, 117.3, 114.5, 112.2, 27.7. [1240]¹⁹F NMR (471 MHz, CDCl₃) δ −57.83.

### 3-hydroxy-5-(5-(4-(trifluoromethoxy)phenoxy)thiophen-2-yl)cyclohex-2-en-1-one (38)

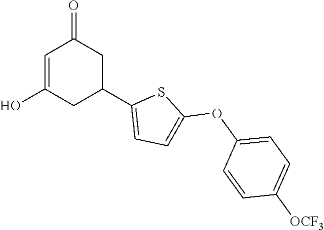 [SMILES: O=C1C=C(O)CC(c2ccc(Oc3ccc(OC(F)(F)F)cc3)s2)C1]

Following general procedures J and K, the title compound was prepared as a white powder.

LCMS (ESI-MS) m/z: [M+H]⁺ calcd for C₁₇H₁₄F₃O₄S 371.05; found 370.7. Retention time: 3.556 min.

TLC: (5% methanol in dichloromethane, R_{f}): 0.20 (UV, 254 nm, 280 nm).

¹H NMR (500 MHz, DMSO-d₆) δ 11.25 (s, 1H), 7.44-7.37 (m, 2H), 7.24-7.17 (m, 2H), 6.74 (dd, J=3.8, 1.0 Hz, 1H), 6.59 (d, J=3.8 Hz, 1H), 5.28 (s, 1H), 3.59-3.50 (m, 1H), 2.63-2.47 (m, 4H).

¹³C NMR (126 MHz, DMSO-d₆) δ 157.6, 156.6, 144.3 (q, J_{C—F}=2.0 Hz), 139.4, 123.4, 121.6, 121.5, 120.2 (q, J_{C—F}=255 Hz), 118.5, 114.4, 104.2, 34.8. [1247]¹⁹F NMR (471 MHz, DMSO-d₆) δ −57.24.

### 3-hydroxy-5-(5-(3-(trifluoromethoxy)phenoxy)thiophen-2-yl)cyclohex-2-en-1-one (39)

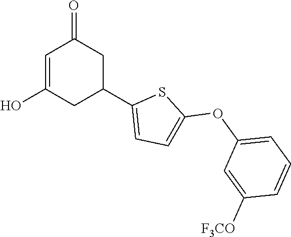 [SMILES: O=C1C=C(O)CC(c2ccc(Oc3cccc(OC(F)(F)F)c3)s2)C1]

Following general procedures J and K, the title compound was prepared as a white powder.

LCMS (ESI-MS) m/z: [M+H]⁺ calcd for C₁₇H₁₄F₃O₄S 371.05; found 370.9. Retention time: 3.527 min.

TLC: (5% methanol in dichloromethane, R_{f}): 0.20 (UV, 254 nm, 280 nm).

¹H NMR (500 MHz, DMSO-d₆) δ 11.26 (s, 1H), 7.52 (dd, J=8.6, 7.9 Hz, 1H), 7.16 (d, J=7.9 Hz, 1H), 7.12 (dd, J=8.6, 2.4 Hz, 1H), 7.09 (s, 1H), 6.76 (d, J=3.8 Hz, 1H), 6.63 (d, J=3.8 Hz, 1H), 5.27 (s, 1H), 3.60-3.50 (m, 1H), 2.77-2.51 (m, 4H).

¹³C NMR (126 MHz, DMSO-d₆) δ 159.8, 155.9, 149.6 (q, J_{C—F}=2.0 Hz), 139.8, 132.0, 121.7, 120.4 (q, J_{C—F}=256.9 Hz), 116.3, 115.8, 114.8, 110.1, 104.2, 34.8.

¹⁹F NMR (471 MHz, DMSO-d₆) δ −56.90.

### 3-hydroxy-5-(6-(4-(trifluoromethoxy)phenoxy)pyridin-3-yl)cyclohex-2-en-1-one (40)

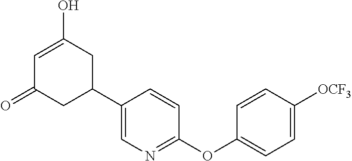 [SMILES: O=C1C=C(O)CC(c2ccc(Oc3ccc(OC(F)(F)F)cc3)nc2)C1]

Following general procedures J and K, the title compound was prepared as a yellow oil.

LCMS (ESI-MS) m/z: [M+H]⁺ calcd for C₁₈H₁₅F₃NO₄ 366.09; found 365.7. Retention time: 4.076 min. [M−H]⁻ calcd for C₁₈H₁₃F₃N O₄ 364.09; found 364.0. Retention time: 3.233.

TLC: (5% methanol in dichloromethane, R_{f}): 0.22 (UV, 254 nm, 280 nm).

¹H NMR (500 MHz, DMSO-d₆) δ 11.24 (s, 1H), 8.12 (d, J=2.5 Hz, 1H), 7.91 (dd, J=8.5, 2.5 Hz, 1H), 7.41 (d, J=8.5 Hz, 2H), 7.29-7.22 (m, 2H), 7.07 (d, J=8.5 Hz, 1H), 5.30 (s, 1H), 3.40-3.30 (m, 1H), 2.66-2.57 (m, 2H), 2.44-2.36 (m, 2H).

¹³C NMR (126 MHz, DMSO-d₆) b 162.0, 153.2, 146.3, 145.0 (q, J_{C—F}=1.8 Hz), 139.5, 134.9, 123.3, 122.9, 120.5 (q, J_{C-F}=255.8 Hz), 111.8, 104.0, 36.0.

¹⁹F NMR (471 MHz, DMSO-d₆) δ −57.08.

### 3-hydroxy-5-(6-(3-(trifluoromethoxy)phenoxy)pyridin-3-yl)cyclohex-2-en-1-one (41)

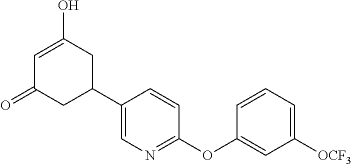 [SMILES: O=C1C=C(O)CC(c2ccc(Oc3cccc(OC(F)(F)F)c3)nc2)C1]

Following general procedures J and K, the title compound was prepared as a yellow oil.

LCMS (ESI-MS) m/z: [M+H]⁺ calcd for C₁₈H₁₅F₃NO₄ 366.09; found 365.7. Retention time: 4.080 min.

TLC: (5% methanol in dichloromethane, R_{f}): 0.16 (UV, 254 nm, 280 nm).

Keto Form: ¹H NMR (500 MHz, CD₃OD) b 8.15 (d, J=2.5 Hz, 1H), 7.91 (dd, J=8.5, 2.5 Hz, 1H), 7.50 (dd, J=9.1, 8.2 Hz, 1H), 7.18-7.08 (m, 2H), 7.07 (s, 1H), 7.04 (d, J=8.5 Hz, 1H), 4.89 (s, 2H), 3.52-3.42 (m, 1H), 2.72 (dd, J=16.9, 11.7 Hz, 2H), 2.60 (dd, J=17.0, 4.7 Hz, 2H).

Enol Form: ¹³C NMR (126 MHz, DMSO-d₆) δ 161.7, 155.4, 149.3 (q, J_{C—F}=1.9 Hz), 146.4, 139.5, 135.1, 131.4, 120.7, 120.4 (q, J_{C-F}=256.7 Hz), 117.2, 114.7, 112.0, 104.0, 36.0.

¹⁹F NMR (471 MHz, DMSO-d₆) δ −56.83.

### 2-bromo-5-(1,3-dioxolan-2-yl)thiazole (44)

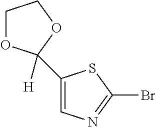 [SMILES: Brc1ncc(C2OCCO2)s1]

Following the general procedure B afforded 90% pure compound as a clear oil and was directly used without further purification.

LCMS (ESI-MS) m/z [M+H]⁺ calcd for C₆H₇BrNO₂S 235.93; found 236.0. Retention time: 2.440 min.

TLC: (20% EtOAc in hexanes, R_{f}): 0.27 (UV, 254 nm, 280 nm).

### 2-bromo-4-(1,3-dioxolan-2-yl)thiazole (45)

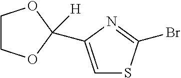 [SMILES: Brc1nc(C2OCCO2)cs1]

Following the general procedure B afforded 90% pure compound as a clear oil and was directly used without further purification.

LCMS (ESI-MS) m/z [M+H]⁺ calcd for C₆H₇BrNO₂S 235.93; found 235.9. Retention time: 2.024 min.

TLC: (20% EtOAc in hexanes, R_{f}): 0.27 (UV, 254 nm, 280 nm).

### 5-(1,3-dioxolan-2-yl)-2-(4-(trifluoromethoxy)phenoxy)thiazole (46)

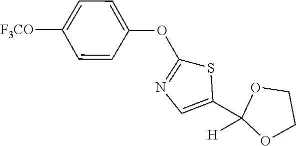 [SMILES: FC(F)(F)Oc1ccc(Oc2ncc(C3OCCO3)s2)cc1]

Following the general procedure D, the title compound was prepared as a white powder.

LCMS (ESI-MS) m/z [M+H]⁺ calcd for C₁₃H₁₁F₃NO₄S 334.03; found 333.7. Retention time: 4.504 min.

TLC: (20% EtOAc in hexanes, R_{f}): 0.31 (UV, 254 nm, 280 nm).

¹H NMR (500 MHz, CDCl₃) δ 7.34-7.29 (m, 2H), 7.29-7.24 (m, 3H), 5.98 (s, 1H), 4.14-4.05 (m, 2H), 4.05-3.97 (m, 2H).

¹³C NMR (126 MHz, CDCl₃) δ 173.7, 153.1, 146.5 (q, J_{C—F}=2.2 Hz), 136.4, 130.5, 122.6, 121.6, 120.4 (q, J_{C—F}=257.5 Hz), 98.9, 65.3.

¹⁹F NMR (471 MHz, CDCl₃) δ −58.13.

### 5-(1,3-dioxolan-2-yl)-2-(3-(trifluoromethoxy)phenoxy)thiazole (47)

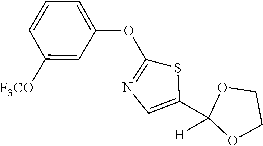 [SMILES: FC(F)(F)Oc1cccc(Oc2ncc(C3OCCO3)s2)c1]

Following the general procedure D, the title compound was prepared as a yellow oil.

LCMS (ESI-MS) m/z [M+H]⁺ calcd for C₁₃H₁₁F₃NO₄S 334.03; found 333.7. Retention time: 4.527 min.

TLC: (20% EtOAc in hexanes, R_{f}): 0.31 (UV, 254 nm, 280 nm).

¹H NMR (500 MHz, CDCl₃) δ 7.43 (dd, J=8.3, 7.8 Hz, 1H), 7.29 (s, 1H), 7.24 (ddd, J=8.3, 2.3, 0.9 Hz, 1H), 7.21-7.18 (m, 1H), 7.14-7.10 (m, 1H), 5.99 (s, 1H), 4.14-4.06 (m, 2H), 4.06-3.97 (m, 2H).

¹³C NMR (126 MHz, CDCl₃) δ 173.1, 155.5, 149.8 (q, J_{C—F}=2.2 Hz), 136.4, 130.8, 130.8, 120.3 (q, J_{C—F}=258.1 Hz), 118.4, 118.0, 113.3, 98.9, 65.3.

¹⁹F NMR (471 MHz, CDCl₃) δ −57.90.

### 4-(1,3-dioxolan-2-yl)-2-(4-(trifluoromethoxy)phenoxy)thiazole (48)

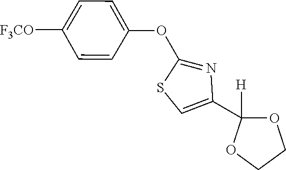 [SMILES: FC(F)(F)Oc1ccc(Oc2nc(C3OCCO3)cs2)cc1]

Following the general procedure D, the title compound was prepared as a white powder.

LCMS (ESI-MS) m/z [M+H]⁺ calcd for C₁₃H₁₁F₃NO₄S 334.03; found 334.1. Retention time: 3.78 min.

TLC: (20% EtOAc in hexanes, R_{f}): 0.31 (UV, 254 nm, 280 nm).

¹H NMR (500 MHz, CDCl₃) δ 7.37-7.32 (m, 2H), 7.25 (d, J=8.8 Hz, 2H), 6.96 (s, 1H), 5.84 (s, 1H), 4.17-4.09 (m, 2H), 4.05-3.97 (m, 2H).

¹³C NMR (126 MHz, CDCl₃) δ 172.8, 153.3, 148.5, 146.3 (q, J_{C—F}=1.8 Hz), 122.5, 121.2, 120.4 (q, J_{C—F}=257.4 Hz), 111.0, 99.8, 65.3.

### 4-(1,3-dioxolan-2-yl)-2-(3-(trifluoromethoxy)phenoxy)thiazole (49)

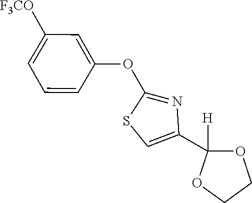 [SMILES: FC(F)(F)Oc1cccc(Oc2nc(C3OCCO3)cs2)c1]

Following the general procedure D afforded 90% pure compound as a white powder and was directly used without further purification.

LCMS (ESI-MS) m/z [M+H]⁺ calcd for C₁₃H₁₁F₃NO₄S 334.03; found 333.9. Retention time: 3.661 min.

TLC: (20% EtOAc in hexanes, R_{f}): 0.31 (UV, 254 nm, 280 nm).

### 2-(4-(trifluoromethoxy)phenoxy)thiazole-5-carbaldehyde (50)

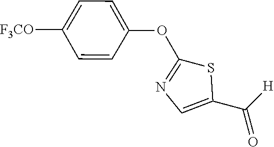 [SMILES: O=Cc1cnc(Oc2ccc(OC(F)(F)F)cc2)s1]

Following the general procedure C, the title compound was prepared as a faint yellow powder.

LCMS (ESI-MS) m/z [M+H]⁺ calcd for C₁₁H₇F₃NO₃S 290.00; found 289.8. Retention time: 3.526 min.

TLC: (20% EtOAc in hexanes, R_{f}): 0.38 (UV, 254 nm, 280 nm).

¹H NMR (500 MHz, CDCl₃) δ 9.87 (s, 1H), 7.91 (s, 1H), 7.41-7.28 (m, 4H).

¹³C NMR (126 MHz, CDCl₃) δ 181.9, 178.9, 152.2, 148.4, 147.2 (q, J_{C-F}=2.0 Hz), 134.3, 122.7, 121.9, 120.3 (q, J_{C—F}=258.0 Hz).

¹⁹F NMR (471 MHz, CDCl₃) δ −58.10.

### 2-(3-(trifluoromethoxy)phenoxy)thiazole-5-carbaldehyde (51)

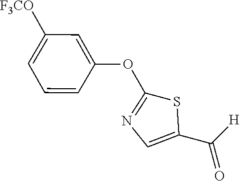 [SMILES: O=Cc1cnc(Oc2cccc(OC(F)(F)F)c2)s1]

Following the general procedure C, the title compound was prepared as a faint yellow oil.

LCMS (ESI-MS) m/z [M+H]⁺ calcd for C₁₁H₇F₃NO₃S 290.00; found 289.8. Retention time: 3.536 min.

TLC: (20% EtOAc in hexanes, R_{f}): 0.41 (UV, 254 nm, 280 nm).

¹H NMR (500 MHz, CDCl₃) δ 9.87 (s, 1H), 7.92 (s, 1H), 7.50 (dd, J=8.8, 8.3 Hz, 1H), 7.29 (ddd, J=8.3, 2.4, 1.0 Hz, 1H), 7.25-7.23 (m, 1H), 7.22-7.18 (m, 1H).

¹³C NMR (126 MHz, CDCl₃) δ 181.9, 178.4, 154.6, 149.9 (q, J_{C—F}=1.9 Hz), 148.3, 134.4, 130.9, 120.3 (q, J_{C—F}=258.7 Hz), 119.0, 118.8, 113.8.

¹⁹F NMR (471 MHz, CDCl₃) δ −57.95, −58.46.

### 2-(4-(trifluoromethoxy)phenoxy)thiazole-4-carbaldehyde (52)

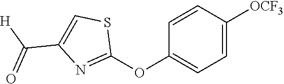 [SMILES: O=Cc1csc(Oc2ccc(OC(F)(F)F)cc2)n1]

Following general procedure C, the title compound was prepared as a white powder.

LCMS (ESI-MS) m/z [M+H]⁺ calcd for C₁₁H₇F₃NO₃S 290.00; found 289.7. Retention time: 4.254 min.

TLC: (20% EtOAc in hexanes, R_{f}): 0.35 (UV, 254 nm, 280 nm).

¹H NMR (500 MHz, CDCl₃) δ 9.81 (s, 1H), 7.78 (s, 1H), 7.45-7.38 (m, 2H), 7.31 (d, J=8.7 Hz, 2H).

¹³C NMR (126 MHz, CDCl₃) δ 183.8, 172.6, 152.7, 149.3, 146.6 (q, J_{C—F}=2.2 Hz), 122.7, 122.6, 121.4, 120.4 (q, J_{C—F}=257.8 Hz).

¹⁹F NMR (471 MHz, CDCl₃) δ −58.13.

### 2-(3-(trifluoromethoxy)phenoxy)thiazole-4-carbaldehyde (53)

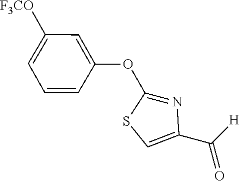 [SMILES: O=Cc1csc(Oc2cccc(OC(F)(F)F)c2)n1]

Following general procedure C, the title compound was prepared as a white powder.

LCMS (ESI-MS) m/z [M+H]⁺ calcd for C₁₁H₇F₃NO₃S 290.00; found 289.4. Retention time: 4.268 min.

TLC: (20% EtOAc in hexanes, R_{f}): 0.35 (UV, 254 nm, 280 nm).

¹H NMR (500 MHz, CDCl₃) δ 9.80 (s, 1H), 7.78 (s, 1H), 7.46 (dd, J=8.8, 8.3 Hz, 1H), 7.33 (dd, J=8.3, 2.3 Hz, 1H), 7.26 (d, J=2.3 Hz, 1H), 7.16 (d, J=8.8 Hz, 1H).

¹³C NMR (126 MHz, CDCl3) δ 183.8, 172.1, 155.1, 149.9 (q, J_{C—F}=2.1 Hz), 149.3, 130.8, 122.7, 120.3 (q, J_{C—F}=258.3 Hz), 118.3, 118.2, 113.2.

¹⁹F NMR (471 MHz, CDCl₃) δ −57.92.

### (E)-4-(2-(4-(trifluoromethoxy)phenoxy)thiazol-5-yl)but-3-en-2-one (54)

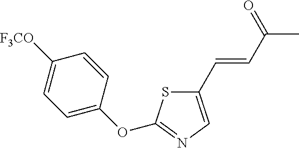 [SMILES: CC(=O)/C=C/c1cnc(Oc2ccc(OC(F)(F)F)cc2)s1]

Following general procedure 1, the title compound was prepared as a brown solid.

LCMS (ESI-MS) m/z: [M+H]⁺ calcd for C₁₄H₁₁F₃NO₃S 330.03; found 329.8. Retention time: 3.729 min.

TLC: (20% EtOAc in hexanes, R_{f}): 0.31 (UV, 254 nm, 280 nm).

¹H NMR (500 MHz, CDCl₃) δ 7.51 (d, J=15.8 Hz, 1H), 7.41 (s, 1H), 7.38-7.32 (m, 2H), 7.29 (d, J=8.8 Hz, 2H), 6.30 (d, J=15.8 Hz, 1H), 2.32 (s, 3H).

¹³C NMR (126 MHz, CDCl₃) δ 197.0, 173.9, 152.5, 146.8 (q, J_{C—F}=1.8 Hz), 142.2, 132.8, 129.9, 126.9, 122.6, 121.7, 120.4 (q, J_{C—F}=257.6 Hz), 27.8.

¹⁹F NMR (471 MHz, CDCl₃) δ −58.11.

### (E)-4-(2-(3-(trifluoromethoxy)phenoxy)thiazol-5-yl)but-3-en-2-one (55)

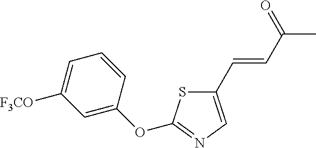 [SMILES: CC(=O)/C=C/c1cnc(Oc2cccc(OC(F)(F)F)c2)s1]

Following general procedure 1, the title compound was prepared as a brown solid.

LCMS (ESI-MS) m/z: [M+H]⁺ calcd for C₁₄H₁₁F₃NO₃S 330.03; found 329.8. Retention time: 3.702 min.

TLC: (20% EtOAc in hexanes, R_{f}): 0.28 (UV, 254 nm, 280 nm).

¹H NMR (500 MHz, CDCl₃) δ 7.52 (d, J=15.8 Hz, 1H), 7.47 (dd, J=8.6, 8.2 Hz, 1H), 7.42 (s, 1H), 7.28 (ddd, J=8.6, 2.3, 0.9 Hz, 1H), 7.25-7.21 (m, 1H), 7.19-7.14 (m, 1H), 6.30 (d, J=15.8 Hz, 1H), 2.33 (s, 3H).

¹³C NMR (126 MHz, CDCl₃) δ 197.0, 173.3, 154.9, 149.9 (q, J_{C—F}=2.0 Hz), 142.1, 132.8, 130.8, 130.0, 127.0, 120.3 (q, J_{C-F}=258.2 Hz), 118.6, 118.5, 113.6, 27.8.

¹⁹F NMR (471 MHz, CDCl₃) δ −57.92.

### (E)-4-(2-(4-(trifluoromethoxy)phenoxy)thiazol-4-yl)but-3-en-2-one (56)

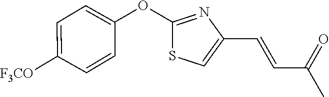 [SMILES: CC(=O)/C=C/c1csc(Oc2ccc(OC(F)(F)F)cc2)n1]

Following general procedure 1, the title compound was prepared as a brown powder.

LCMS (ESI-MS) m/z: [M+H]⁺ calcd for C₁₄H₁₁F₃NO₃S 330.03; found 330.1. Retention time: 3.711 min.

TLC: (20% EtOAc in hexanes, R_{f}): 0.23 (UV, 254 nm, 280 nm).

¹H NMR (500 MHz, CDCl₃) δ 7.44-7.39 (m, 2H), 7.33-7.29 (m, 2H), 7.26 (d, J=15.6 Hz, 1H), 7.06 (s, 1H), 6.85 (d, J=15.6 Hz, 1H), 2.35 (s, 3H).

¹³C NMR (126 MHz, CDCl₃) δ 198.4, 172.3, 152.9, 146.8, 146.5 (q, J_{C—F}=2.2 Hz), 134.3, 128.8, 122.5, 121.4, 120.4 (q, J_{C—F}=257.6 Hz), 116.9, 28.3.

¹⁹F NMR (471 MHz, CDCl₃) δ −58.11.

### (E)-4-(2-(3-(trifluoromethoxy)phenoxy)thiazol-4-yl)but-3-en-2-one (57)

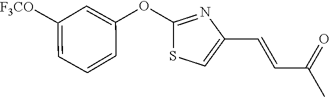 [SMILES: CC(=O)/C=C/c1csc(Oc2cccc(OC(F)(F)F)c2)n1]

Following general procedure 1, the title compound was prepared as a brown powder.

LCMS (ESI-MS) m/z: [M+H]⁺ calcd for C₁₄H₁₁F₃NO₃S 330.03; found 330.0. Retention time: 3.737 min.

TLC: (20% EtOAc in hexanes, R_{f}): 0.32 (UV, 254 nm, 280 nm).

¹H NMR (500 MHz, CDCl₃) δ 7.48 (dd, J=8.5, 8.1 Hz, 1H), 7.36-7.30 (m, 2H), 7.27 (d, J=15.5 Hz, 1H), 7.19-7.15 (m, 1H), 7.07 (s, 1H), 6.86 (d, J=15.6 Hz, 1H), 2.35 (s, 3H).

¹³C NMR (126 MHz, CDCl₃) δ 198.3, 171.8, 155.2, 149.7 (q, J_{C—F}=2.0 Hz), 146.8, 134.1, 130.6, 128.8, 120.3 (q, J_{C—F}=258.2 Hz), 118.2, 118.1, 117.0, 113.3, 28.4.

¹⁹F NMR (471 MHz, CDCl₃) δ −57.92.

### 3-hydroxy-5-(2-(4-(trifluoromethoxy)phenoxy)thiazol-5-yl)cyclohex-2-en-1-one (58)

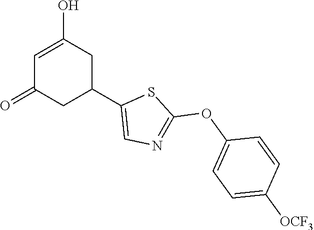 [SMILES: O=C1C=C(O)CC(c2cnc(Oc3ccc(OC(F)(F)F)cc3)s2)C1]

Following general procedures J and K, the title compound was prepared as a white powder.

LCMS (ESI-MS) m/z: [M+H]⁺ calcd for C₁₆H₁₃F₃NO₄S 372.04; found 371.7. Retention time: 4.047 min.

TLC: (5% methanol in dichloromethane, R_{f}): 0.17 (UV, 254 nm, 280 nm).

Keto Form: ¹H NMR (500 MHz, CD₃OD) δ 7.45-7.35 (m, 4H), 7.10 (d, J=0.9 Hz, 1H), 4.90 (s, 2H), 3.76-3.62 (m, 1H), 2.75 (dd, J=17.0, 4.7 Hz, 2H), 2.62 (dd, J=16.9, 9.9 Hz, 2H).

Enol Form: ¹H NMR (500 MHz, DMSO-d₆) δ 11.32 (s, 1H), 7.48 (s, 4H), 7.13 (d, J=1.0 Hz, 1H), 5.28 (s, 1H), 3.65-3.55 (m, 1H), 2.64-2.45 (m, 4H).

¹³C NMR (126 MHz, DMSO-d₆) δ 170.7, 153.8, 145.9 (q, J_{C—F}=2.1 Hz), 136.7, 133.6, 123.4, 122.5, 120.5 (q, J_{C—F}=256.4 Hz), 104.2, 32.5.

¹⁹F NMR (471 MHz, CD₃OD) 5-59.80.

IR (thin film, cm⁻¹): v=2885, 1600, 1545, 1499, 1367, 1284, 1221, 1174, 843.

### 3-hydroxy-5-(2-(3-(trifluoromethoxy)phenoxy)thiazol-5-yl)cyclohex-2-en-1-one (59)

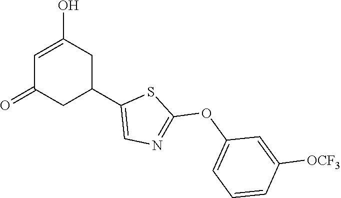 [SMILES: O=C1C=C(O)CC(c2cnc(Oc3cccc(OC(F)(F)F)c3)s2)C1]

Following general procedures J and K, the title compound was prepared as a white powder.

LCMS (ESI-MS) m/z: [M+H]⁺ calcd for C₁₆H₁₃F₃NO₄S 372.04; found 371.7. Retention time: 4.053 min.

TLC: (5% methanol in dichloromethane, R_{f}): 0.17 (UV, 254 nm, 280 nm).

¹H NMR (500 MHz, CD₃OD) δ 7.44 (dd, J=9.9, 8.2 Hz, 1H), 7.23-7.15 (m, 2H), 7.11 (d, J=8.2 Hz, 1H), 7.00 (s, 1H), 4.77 (s, 2H), 3.63-3.47 (m, 1H), 2.64 (dd, J=17.0, 4.7 Hz, 2H), 2.51 (dd, J=17.0, 10.0 Hz, 2H).

¹⁹F NMR (471 MHz, CD₃OD) 5-59.58.

### 5-(2-(4-(trifluoromethoxy)phenoxy)thiazol-4-yl)cyclohexane-1,3-dione (60)

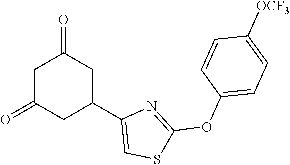 [SMILES: O=C1CC(=O)CC(c2csc(Oc3ccc(OC(F)(F)F)cc3)n2)C1]

Following general procedures J and K, the title compound was prepared as a white powder.

LCMS (ESI-MS) m/z: [M+H]⁺ calcd for C₁₆H₁₃F₃NO₄S 372.04; found 371.7. Retention time: 4.159 min.

TLC: (5% methanol in dichloromethane, R_{f}): 0.25 (UV, 254 nm, 280 nm).

¹H NMR (500 MHz, CD₃OD) δ 7.48-7.41 (m, 2H), 7.41-7.36 (m, 2H), 6.82 (d, J=1.0 Hz, 1H), 4.89 (s, 2H), 3.47-3.37 (m, 1H), 2.67 (d, J=7.6 Hz, 4H).

¹³C NMR (126 MHz, CD₃OD) δ 174.2, 155.2, 153.7, 147.7 (q, J_{C—F}=2.1 Hz), 123.8, 122.6, 121.8 (q, J_{C—F}=256.9 Hz), 109.1, 37.4.

¹⁹F NMR (471 MHz, CD₃OD) 5-59.79.

IR (thin film, cm⁻¹): v=2890, 1571, 1534, 1493, 1207, 1178, 844.

### 5-(2-(3-(trifluoromethoxy)phenoxy)thiazol-4-yl)cyclohexane-1,3-dione (61)

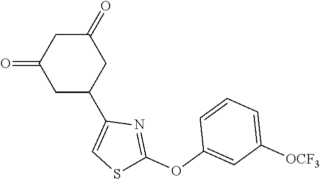 [SMILES: O=C1CC(=O)CC(c2csc(Oc3cccc(OC(F)(F)F)c3)n2)C1]

Following general procedures J and K, the title compound was prepared as a white powder.

LCMS (ESI-MS) m/z: [M+H]⁺ calcd for C₁₆H₁₃F₃NO₄S 372.04; found 371.7. Retention time: 4.159 min.

TLC: (5% methanol in dichloromethane, R_{f}): 0.22 (UV, 254 nm, 280 nm).

Keto Form: ¹H NMR (500 MHz, CD₃OD) δ 7.44 (dd, J=9.1, 8.3 Hz, 1H), 7.26-7.18 (m, 2H), 7.14-7.08 (m, 1H), 6.73 (s, 1H), 4.77 (s, 2H), 3.36-3.26 (m, 1H), 2.55 (d, J=7.6 Hz, 4H).

Enol Form: ¹H NMR (500 MHz, DMSO-d₆) δ 11.18 (s, 1H), 7.62 (dd, J=8.7, 8.1 Hz, 1H), 7.50 (s, 1H), 7.44 (dd, J=8.3, 2.3 Hz, 1H), 7.38-7.20 (m, 1H), 6.95 (d, J=1.0 Hz, 1H), 5.25 (s, 1H), 3.33-3.26 (m, 1H), 2.73-2.54 (m, 2H), 2.46-2.28 (m, 2H).

¹³C NMR (126 MHz, DMSO-d₆) δ 171.5, 155.9, 152.9, 149.3 (q, J_{C—F}=1.8 Hz), 132.1, 120.4 (q, J_{C—F}=257.0 Hz), 119.5, 118.8, 113.8, 109.0, 104.0, 35.8.

¹⁹F NMR (471 MHz, CD₃OD) 5-59.57.

IR (thin film, cm⁻¹): v=2895, 1598, 1483, 1250, 1208, 1163, 869.

### 6-methoxy-3-(2-(4-(trifluoromethoxy)phenoxy)thiazol-5-yl)-3,4-dihydroacridine-1,9(2H,10H)-dione (62)

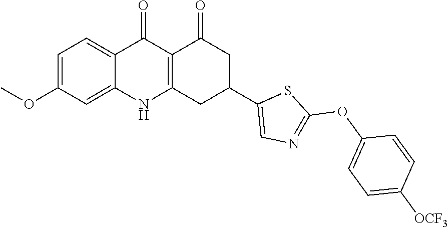 [SMILES: COc1ccc2c(=O)c3c([nH]c2c1)CC(c1cnc(Oc2ccc(OC(F)(F)F)cc2)s1)CC3=O]

Following general procedure L, the title compound was prepared as a brown powder.

TLC: (5% methanol in dichloromethane, R_{f}): 0.33 (UV, 254 nm, 280 nm).

¹H NMR (500 MHz, CDCl₃ and 3 drops of TFA-d) δ 8.38 (d, J=9.3 Hz, 1H), 7.44-7.30 (m, 7H), 4.03 (s, 3H), 3.99-3.89 (m, 1H), 3.86 (dd, J=17.7, 4.2 Hz, 1H), 3.57 (dd, J=17.6, 10.2 Hz, 1H), 3.28 (dd, J=17.7, 4.2 Hz, 1H), 3.12 (dd, J=17.6, 10.2 Hz, 1H).

¹³C NMR (126 MHz, CDCl₃ and 3 drops of TFA-d) δ 200.4, 175.4, 171.8, 168.0, 159.2, 152.5, 147.9, 142.8, 131.7, 131.6, 126.8, 123.1, 122.08, 122.00, 121.8, 120.3 (q, J_{C—F}=258.6 Hz), 113.2, 106.9, 100.1, 56.7, 43.1, 34.9, 32.0.

¹⁹F NMR (471 MHz, CDCl₃ and 3 drops of TFA-d) δ −59.00.

### 6-methoxy-3-(2-(3-(trifluoromethoxy)phenoxy)thiazol-5-yl)-3,4-dihydroacridine-1,9(2H,10H)-dione (63)

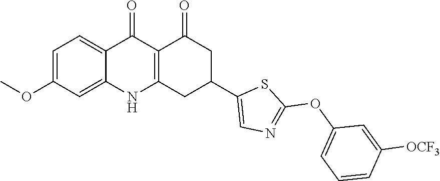 [SMILES: COc1ccc2c(=O)c3c([nH]c2c1)CC(c1cnc(Oc2cccc(OC(F)(F)F)c2)s1)CC3=O]

Following general procedure L, the title compound was prepared as an off-white, tan-colored powder.

TLC: (5% methanol in dichloromethane, R_{f}): 0.33 (UV, 254 nm, 280 nm).

¹H NMR (500 MHz, CDCl₃ and 3 drops of TFA-d) δ 8.37 (d, J=9.2 Hz, 1H), 7.51 (dd, J=9.2, 8.3 Hz, 1H), 7.42-7.33 (m, 2H), 7.28 (s, 1H), 7.27-7.19 (m, 3H), 7.16 (s, 1H), 4.04 (s, 3H), 3.99-3.90 (m, 1H), 3.87 (dd, J=17.8, 4.2 Hz, 1H), 3.58 (dd, J=17.6, 9.9 Hz, 1H), 3.29 (dd, J=17.8, 4.2 Hz, 1H), 3.11 (dd, J=17.6, 9.9 Hz, 1H).

¹³C NMR (126 MHz, CDCl₃ and 3 drops of TFA-d) δ 200.5, 174.3, 171.7, 168.0, 159.3, 154.9, 150.1 (q, J_{C—F}=2.1 Hz), 142.9, 132.5, 132.0, 131.4, 126.7, 121.9, 120.2 (q, J_{C—F}=258.7 Hz), 119.5, 118.5, 113.5, 113.2, 106.9, 100.2, 56.7, 43.3, 35.0, 32.0.

¹⁹F NMR (471 MHz, CDCl₃ and 3 drops of TFA-d) δ −58.82.

### 7-chloro-3-(2-(4-(trifluoromethoxy)phenoxy)thiazol-4-yl)-3,4-dihydroacridine-1,9(2H,10H)-dione (64)

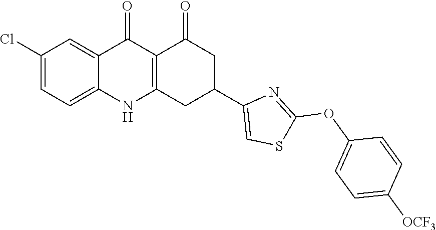 [SMILES: O=C1CC(c2csc(Oc3ccc(OC(F)(F)F)cc3)n2)Cc2[nH]c3ccc(Cl)cc3c(=O)c21]

Following general procedure L, the title compound was prepared as a white powder.

TLC: (5% methanol in dichloromethane, R_{f}): 0.34 (UV, 254 nm, 280 nm).

¹H NMR (500 MHz, CDCl₃ and 3 drops of TFA-d) δ 8.43 (s, 1H), 8.14-7.98 (m, 2H), 7.23-7.10 (m, 4H), 6.73 (s, 1H), 3.90-3.77 (m, 2H), 3.68 (dd, J=17.8, 7.8 Hz, 1H), 3.29-3.14 (m, 2H).

¹³C NMR (126 MHz, CDCl₃ and 3 drops of TFA-d) δ 202.2, 174.5, 171.4, 160.8, 152.8, 148.7, 146.7 (q, J=2.2 Hz), 138.3, 138.0, 136.1, 124.0, 122.3, 121.6, 120.3 (q, J=257.6 Hz), 120.1, 108.9, 108.8, 42.2, 34.5, 33.39, 33.37.

¹⁹F NMR (471 MHz, CDCl₃ and 3 drops of TFA-d) δ −58.84.

### 6-methoxy-3-(2-(4-(trifluoromethoxy)phenoxy)thiazol-4-yl)-3,4-dihydroacridine-1,9(2H,10H)-dione (65)

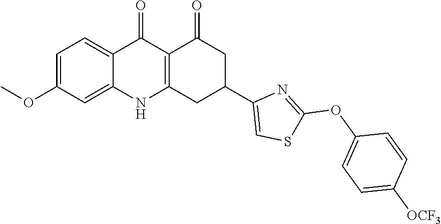 [SMILES: COc1ccc2c(=O)c3c([nH]c2c1)CC(c1csc(Oc2ccc(OC(F)(F)F)cc2)n1)CC3=O]

Following general procedure L, the title compound was prepared as an off-white, tan-colored powder.

TLC: (5% methanol in dichloromethane, R_{f}): 0.34 (UV, 254 nm, 280 nm).

¹H NMR (500 MHz, CDCl₃ and 3 drops of TFA-d) δ 8.36 (d, J=9.2 Hz, 1H), 7.39 (dd, J=9.2, 2.4 Hz, 1H), 7.32 (d, J=2.4 Hz, 1H), 7.28 (s, 4H), 6.82 (s, 1H), 4.03 (s, 3H), 3.95-3.86 (m, 1H), 3.78 (dd, J=17.6, 4.6 Hz, 1H), 3.67 (dd, J=17.6, 9.8 Hz, 1H), 3.24 (dd, J=17.6, 4.6 Hz, 1H), 3.16 (dd, J=17.6, 9.8 Hz, 1H).

¹³C NMR (126 MHz, CDCl₃ and 3 drops of TFA-d) δ 201.3, 176.7, 171.6, 167.9, 160.0, 152.7, 147.7 (q, J_{C—F}=1.8 Hz), 147.2, 142.5, 126.8, 122.9, 121.8, 121.6, 120.3 (q, J_{C—F}=258.1 Hz), 113.1, 108.9, 107.2, 99.8, 56.6, 41.8, 34.2, 32.8.

¹⁹F NMR (471 MHz, CDCl₃ and 3 drops of TFA-d) δ −58.90.

### 7-chloro-3-(2-(3-(trifluoromethoxy)phenoxy)thiazol-4-yl)-3,4-dihydroacridine-1,9(2H,10H)-dione (66)

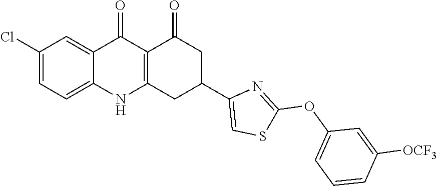 [SMILES: O=C1CC(c2csc(Oc3cccc(OC(F)(F)F)c3)n2)Cc2[nH]c3ccc(Cl)cc3c(=O)c21]

Following general procedure L, the title compound was prepared as a yellow solid.

TLC: (5% methanol in dichloromethane, R_{f}): 0.33 (UV, 254 nm, 280 nm).

¹H NMR (500 MHz, CDCl₃ and 3 drops of TFA-d) δ 8.44 (s, 1H), 8.07-8.03 (m, 2H), 7.38 (dd, J=9.4, 8.4 Hz, 1H), 7.16-7.10 (m, 2H), 7.07 (s, 1H), 6.77 (s, 1H), 3.93-3.85 (m, 1H), 3.82 (dd, J=17.8, 4.5 Hz, 1H), 3.69 (dd, J=17.8, 8.4 Hz, 1H), 3.26 (dd, J=17.8, 4.5 Hz, 1H), 3.19 (dd, J=17.8, 8.4 Hz, 1H).

¹³C NMR (126 MHz, CDCl₃ and 3 drops of TFA-d) δ 202.1, 174.4, 171.6, 160.8, 155.2, 149.8, 148.6, 138.5, 138.0, 136.1, 130.7, 124.1, 122.3, 120.28 (q, J_{C—F}=258.4 Hz), 120.23, 118.6, 118.5, 113.4, 109.2, 108.7 (q, J_{C—F}=1.9 Hz), 42.2, 34.5, 33.3.

¹⁹F NMR (471 MHz, CDCl₃ and 3 drops of TFA-d) δ −58.80.

### 7-chloro-3-(6-(2-fluoro-4-(trifluoromethyl)phenyl)pyridin-3-yl)-3,4-dihydroacridine-1,9(2H,10H)-dione (67)

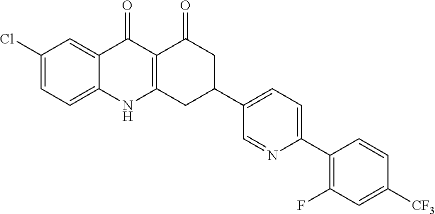 [SMILES: O=C1CC(c2ccc(-c3ccc(C(F)(F)F)cc3F)nc2)Cc2[nH]c3ccc(Cl)cc3c(=O)c21]

Following general procedure L, the title compound was prepared as a slightly yellow solid.

TLC: (5% methanol in dichloromethane, R_{f}): 0.21 (UV, 254 nm, 280 nm).

¹H NMR (500 MHz, CDCl₃ and 3 drops of TFA-d) δ 9.28 (s, 1H), 8.72 (dd, J=8.5, 2.0 Hz, 1H), 8.54 (d, J=2.0 Hz, 1H), 8.26 (d, J=8.3 Hz, 1H), 8.12 (dd, J=9.0, 2.2 Hz, 1H), 8.02 (d, J=8.5 Hz, 1H), 7.86 (dd, J=9.0, 7.6 Hz, 1H), 7.72 (d, J=8.3 Hz, 1H), 7.64 (d, J=10.3 Hz, 1H), 4.24-4.11 (m, 1H), 4.00-3.79 (m, 2H), 3.46-3.26 (m, 2H).

¹³C NMR (126 MHz, CDCl₃ and 3 drops of TFA-d) δ 200.3, 172.8, 159.9, 159.6 (d, J=256.8 Hz), 146.4, 146.2, 142.1, 140.2, 139.4, 137.8, 137.5 (qd, J=34.4, 8.3 Hz), 137.0, 131.2, 128.7 (d, J=4.5 Hz), 124.5, 123.0-122.8 (m), 122.3 (qd, J=276.2, 2.2 Hz), 121.9, 121.0 (d, J=11.7 Hz), 120.4, 115.0 (dq, J_{C—F}=24.8, 3.7 Hz), 108.2 (d, J_{C—F}=1.9 Hz), 41.9, 35.5, 33.7.

¹⁹F NMR (471 MHz, CDCl₃ and 3 drops of TFA-d) δ −64.47.

### 3-(6-(2-fluoro-4-(trifluoromethyl)phenyl)pyridin-3-yl)-6-methoxy-3,4-dihydroacridine-1,9(2H,10H)-dione (68)

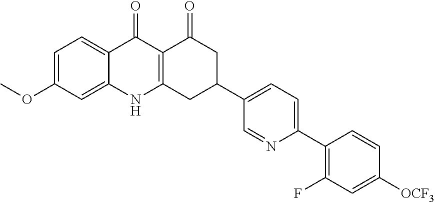 [SMILES: COc1ccc2c(=O)c3c([nH]c2c1)CC(c1ccc(-c2ccc(OC(F)(F)F)cc2F)nc1)CC3=O]

Following general procedure L, the title compound was prepared as a tan-colored solid.

TLC: (5% methanol in dichloromethane, R_{f}): 0.21 (UV, 254 nm, 280 nm).

¹H NMR (500 MHz, CDCl₃ and 3 drops of TFA-d) δ 9.37 (s, 1H), 8.71 (d, J=7.8 Hz, 1H), 8.42 (d, J=9.1 Hz, 1H), 8.25 (d, J=8.3 Hz, 1H), 7.91 (dd, J=10.2, 9.2 Hz, 1H), 7.73 (d, J=7.8 Hz, 1H), 7.64 (d, J=10.2 Hz, 1H), 7.43 (dd, J=9.3, 2.3 Hz, 1H), 7.40 (d, J=2.3 Hz, 1H), 4.21-4.09 (m, 1H), 4.09-3.95 (m, 4H), 3.83 (dd, J=17.4, 12.2 Hz, 1H), 3.38 (dd, J=17.4, 12.2 Hz, 1H), 3.29 (dd, J=17.4, 3.5 Hz, 1H).

¹⁹F NMR (471 MHz, CDCl₃ and 3 drops of TFA-d) δ −64.48.

### 7-chloro-3-(6-(4-(trifluoromethoxy)phenyl)pyridin-3-yl)-3,4-dihydroacridine-1,9(2H,10H)-dione (69)

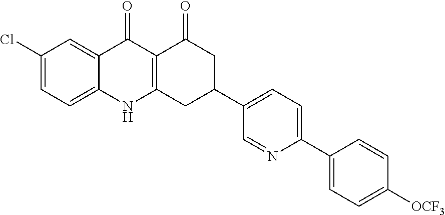 [SMILES: O=C1CC(c2ccc(-c3ccc(OC(F)(F)F)cc3)nc2)Cc2[nH]c3ccc(Cl)cc3c(=O)c21]

Following general procedure L, the title compound was prepared as an off-white, peach-colored solid.

TLC: (5% methanol in dichloromethane, R_{f}): 0.36 (UV, 254 nm, 280 nm).

¹H NMR (500 MHz, DMSO-d₆) δ 12.27 (s, 1H), 8.71 (s, 1H), 8.22 (d, J=8.5 Hz, 2H), 8.06-8.00 (m, 2H), 7.95 (dd, J=8.3, 2.2 Hz, 1H), 7.74 (dd, J=8.6, 2.5 Hz, 1H), 7.59 (d, J=8.6 Hz, 1H), 7.48 (d, J=8.5 Hz, 2H), 3.70-3.61 (m, 1H), 3.45 (dd, J=16.1, 11.6 Hz, 1H), 3.15 (dd, J=16.1, 4.1 Hz, 1H), 2.87 (dd, J=16.1, 11.6 Hz, 1H), 2.65 (dd, J=16.1, 4.1 Hz, 1H).

### 6-methoxy-3-(6-(4-(trifluoromethoxy)phenyl)pyridin-3-yl)-3,4-dihydroacridine-1,9(2H,10H)-dione (70)

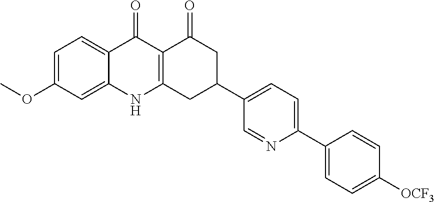 [SMILES: COc1ccc2c(=O)c3c([nH]c2c1)CC(c1ccc(-c2ccc(OC(F)(F)F)cc2)nc1)CC3=O]

Following general procedure L, the title compound was prepared as a tan-colored powder.

TLC: (5% methanol in dichloromethane, R_{f}): 0.36 (UV, 254 nm, 280 nm).

¹H NMR (500 MHz, CDCl₃ and 3 drops of TFA-d) δ 9.25 (s, 1H), 8.66 (d, J=8.4 Hz, 1H), 8.39 (d, J=9.3 Hz, 1H), 8.18 (d, J=8.3 Hz, 1H), 8.03-7.94 (m, 2H), 7.52-7.43 (m, 3H), 7.41 (dd, J=9.3, 2.2 Hz, 1H), 4.18-4.00 (m, 5H), 3.85 (dd, J=18.1, 12.9 Hz, 1H), 3.41 (dd, J=18.1, 12.9 Hz, 1H), 3.27 (d, J=16.5 Hz, 1H).

¹³C NMR (126 MHz, CDCl₃ and 3 drops of TFA-d) δ 200.4, 172.0, 168.0, 159.6, 153.0, 150.8, 146.3, 141.3, 139.0, 132.1, 129.9, 127.4, 126.9, 125.8, 121.9, 120.2 (q, J_{C—F}=259.8 Hz), 114.0, 113.3, 107.0, 100.1, 56.7, 42.0, 35.7, 33.7.

¹⁹F NMR (471 MHz, CDCl₃ and 3 drops of TFA-d) δ −58.51.

### 7-chloro-3-(6-(3-(trifluoromethoxy)phenyl)pyridin-3-yl)-3,4-dihydroacridine-1,9(2H,10H)-dione (71)

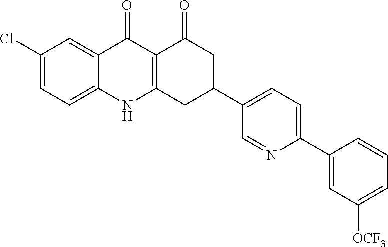 [SMILES: O=C1CC(c2ccc(-c3cccc(OC(F)(F)F)c3)nc2)Cc2[nH]c3ccc(Cl)cc3c(=O)c21]

Following general procedure L, the title compound was prepared as a white powder.

TLC: (5% methanol in dichloromethane, R_{f}): 0.24 (UV, 254 nm, 280 nm).

¹H NMR (500 MHz, CDCl₃ and 3 drops of TFA-d) δ 9.17 (d, J=2.1 Hz, 1H), 8.66 (dd, J=8.5, 2.1 Hz, 1H), 8.53 (d, J=2.2 Hz, 1H), 8.21 (d, J=8.5 Hz, 1H), 8.10 (dd, J=9.0, 2.2 Hz, 1H), 8.02 (d, J=9.0 Hz, 1H), 7.77 (d, J=8.7 Hz, 1H), 7.72 (dd, J=8.7, 8.3 Hz, 1H), 7.67 (s, 1H), 7.57 (d, J=8.3 Hz, 1H), 4.16-4.07 (m, 1H), 3.91 (ddd, J=17.6, 4.3, 1.7 Hz, 1H), 3.83 (dd, J=17.5, 13.1 Hz, 1H), 3.39 (dd, J=17.5, 13.1 Hz, 1H), 3.30 (ddd, J=17.6, 4.3, 1.7 Hz, 1H).

¹³C NMR (126 MHz, CDCl₃ and 3 drops of TFA-d) δ 200.5, 172.6, 159.9, 151.1, 150.4 (q, J_{C—F}=2.2 Hz), 146.2, 141.4, 139.3, 139.2, 137.9, 136.8, 132.1, 131.2, 126.4, 126.0, 125.5, 124.4, 122.0, 120.39, 120.33 (q, J_{C—F}=259.2 Hz), 120.32, 108.1, 35.4, 35.3, 33.7.

¹⁹F NMR (471 MHz, CDCl₃ and 3 drops of TFA-d) δ −58.86.

### 6-methoxy-3-(6-(3-(trifluoromethoxy)phenyl)pyridin-3-yl)-3,4-dihydroacridine-1,9(2H,10H)-dione (72)

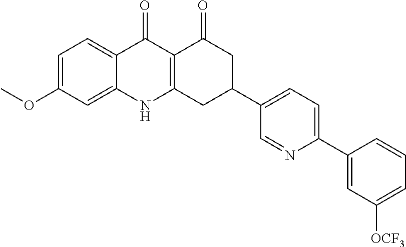 [SMILES: COc1ccc2c(=O)c3c([nH]c2c1)CC(c1ccc(-c2cccc(OC(F)(F)F)c2)nc1)CC3=O]

Following general procedure L, the title compound was prepared as a tan-colored powder.

TLC: (5% methanol in dichloromethane, R_{f}): 0.24 (UV, 254 nm, 280 nm).

¹H NMR (500 MHz, CDCl₃ and 3 drops of TFA-d) δ 9.17 (s, 1H), 8.67 (d, J=8.2 Hz, 1H), 8.43 (d, J=9.4 Hz, 1H), 8.22 (d, J=8.3 Hz, 1H), 7.82-7.65 (m, 3H), 7.58 (d, J=8.2 Hz, 1H), 7.44 (dd, J=9.4, 2.3 Hz, 1H), 7.30 (d, J=2.3 Hz, 1H), 4.21-4.02 (m, 4H), 3.93-3.72 (m, 2H), 3.41-3.22 (m, 2H).

¹⁹F NMR (471 MHz, CDCl₃ and 3 drops of TFA-d) δ −58.99.

### 7-chloro-3-(5-(4-(trifluoromethoxy)phenyl)pyridin-2-yl)-3,4-dihydroacridine-1,9(2H,10H)-dione (73)

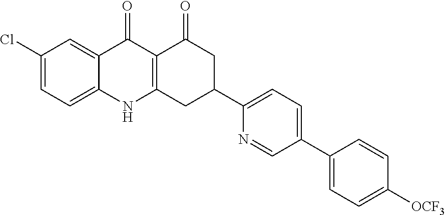 [SMILES: O=C1CC(c2ccc(-c3ccc(OC(F)(F)F)cc3)cn2)Cc2[nH]c3ccc(Cl)cc3c(=O)c21]

Following general procedure L, the title compound was prepared as a white powder.

TLC: (5% methanol in dichloromethane, R_{f}): 0.28 (UV, 254 nm, 280 nm).

¹H NMR (500 MHz, DMSO-d₆) δ 8.87 (d, J=2.4 Hz, 1H), 8.13-8.02 (m, 2H), 7.86 (d, J=8.7 Hz, 2H), 7.62-7.43 (m, 5H), 3.69-3.60 (m, 1H), 3.33 (dd, J=16.2, 11.2 Hz, 1H), 3.20 (d, J=15.7 Hz, 1H), 2.87 (dd, J=16.2, 11.2 Hz, 1H), 2.70 (d, J=15.7 Hz, 1H).

¹H NMR (500 MHz, CDCl₃ and 3 drops of TFA-d) δ 9.00 (d, J=2.3 Hz, 1H), 8.70 (dd, J=8.4, 2.3 Hz, 1H), 8.52 (d, J=2.3 Hz, 1H), 8.17 (d, J=8.4 Hz, 1H), 8.10 (dd, J=9.0, 2.3 Hz, 1H), 8.01 (d, J=9.0 Hz, 1H), 7.74-7.63 (m, 2H), 7.45 (d, J=8.3 Hz, 2H), 4.58-4.46 (m, 1H), 4.07-3.96 (m, 2H), 3.48-3.34 (m, 2H).

¹³C NMR (126 MHz, CDCl₃ and 3 drops of TFA-d) δ 199.3, 172.6, 159.1, 152.1, 151.4 (q, J_{C—F}=1.3 Hz), 145.3, 140.2, 139.7, 139.2, 137.9, 136.8, 130.6, 128.9, 126.0, 124.4, 122.2, 122.0, 120.43, 120.39 (q, J_{C—F}=258.9 Hz), 108.2, 41.1, 36.1, 32.4.

¹⁹F NMR (471 MHz, CDCl₃ and 3 drops of TFA-d) δ −58.45.

### 6-methoxy-3-(5-(4-(trifluoromethoxy)phenyl)pyridin-2-yl)-3,4-dihydroacridine-1,9(2H,10H)-dione (74)

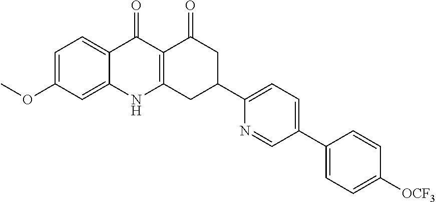 [SMILES: COc1ccc2c(=O)c3c([nH]c2c1)CC(c1ccc(-c2ccc(OC(F)(F)F)cc2)cn1)CC3=O]

Following general procedure L, the title compound was prepared as a white powder.

TLC: (5% methanol in dichloromethane, R_{f}): 0.28 (UV, 254 nm, 280 nm).

¹H NMR (500 MHz, DMSO-d₆) δ 8.87 (s, 1H), 8.10 (d, J=8.8 Hz, 1H), 8.01 (d, J=8.8 Hz, 1H), 7.87 (d, J=8.2 Hz, 2H), 7.59-7.42 (m, 3H), 6.95 (d, J=10.2 Hz, 2H), 3.86 (s, 3H), 3.78-3.64 (m, 1H), 3.45 (t, J=16.3, 10.2 Hz, 2H), 3.24 (dd, J=16.1, 4.5 Hz, 1H), 2.84 (t, J=16.3, 10.2 Hz, 1H), 2.73 (dd, J=16.1, 4.5 Hz, 1H).

### 7-chloro-3-(5-(3-(trifluoromethoxy)phenyl)pyridin-2-yl)-3,4-dihydroacridine-1,9(2H,10H)-dione (75)

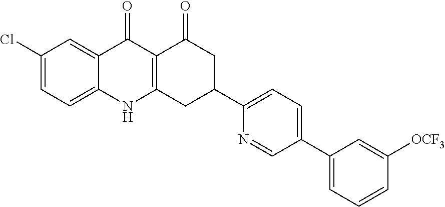 [SMILES: O=C1CC(c2ccc(-c3cccc(OC(F)(F)F)c3)cn2)Cc2[nH]c3ccc(Cl)cc3c(=O)c21]

Following general procedure L, the title compound was prepared as an off-white powder.

TLC: (5% methanol in dichloromethane, R_{f}): 0.25 (UV, 254 nm, 280 nm).

¹H NMR (500 MHz, DMSO-d₆) δ 8.91 (d, J=1.7 Hz, 1H), 8.15 (dd, J=8.3, 1.7 Hz, 1H), 8.04 (d, J=2.5 Hz, 1H), 7.80 (d, J=7.8 Hz, 1H), 7.77-7.69 (m, 2H), 7.67-7.51 (m, 3H), 7.42 (d, J=8.3 Hz, 1H), 3.81-3.72 (m, 1H), 3.48 (dd, J=16.7, 10.8 Hz, 2H), 3.26 (dd, J=16.3, 4.4 Hz, 1H), 2.86 (dd, J=16.7, 10.8 Hz, 1H), 2.76 (dd, J=16.3, 4.4 Hz, 1H).

### 6-methoxy-3-(5-(3-(trifluoromethoxy)phenyl)pyridin-2-yl)-3,4-dihydroacridine-1,9(2H,10H)-dione (76)

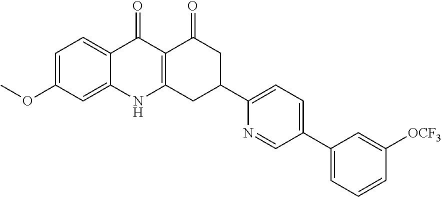 [SMILES: COc1ccc2c(=O)c3c([nH]c2c1)CC(c1ccc(-c2cccc(OC(F)(F)F)c2)cn1)CC3=O]

Following general procedure L, the title compound was prepared as a tan-colored solid.

TLC: (5% methanol in dichloromethane, R_{f}): 0.25 (UV, 254 nm, 280 nm).

¹H NMR (500 MHz, DMSO-d₆) δ 8.91 (d, J=2.4 Hz, 1H), 8.14 (dd, J=8.2, 2.4 Hz, 1H), 8.01 (d, J=8.8 Hz, 1H), 7.80 (d, J=7.8 Hz, 1H), 7.75 (s, 1H), 7.64 (dd, J=8.8, 8.0 Hz, 1H), 7.54 (d, J=8.2 Hz, 1H), 7.42 (d, J=7.8 Hz, 1H), 7.01-6.89 (m, 2H), 3.87 (s, 3H), 3.79-3.69 (m, 1H), 3.46 (dd, J=16.5, 10.8 Hz, 1H), 3.25 (dd, J=16.3, 4.4 Hz, 1H), 2.84 (dd, J=16.5, 10.8 Hz, 1H), 2.73 (dd, J=16.2, 4.4 Hz, 1H).

### 7-chloro-3-(5-(2-fluoro-4-(trifluoromethyl)phenyl)thiophen-2-yl)-3,4-dihydroacridine-1,9(2H,10H)-dione (77)

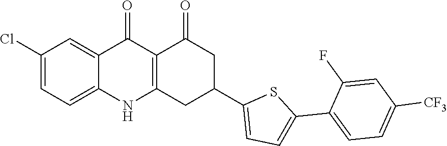 [SMILES: O=C1CC(c2ccc(-c3ccc(C(F)(F)F)cc3F)s2)Cc2[nH]c3ccc(Cl)cc3c(=O)c21]

Following general procedure L, the title compound was prepared as a yellow powder.

TLC: (5% methanol in dichloromethane, R_{f}): 0.37 (UV, 254 nm, 280 nm).

¹H NMR (500 MHz, CDCl₃ and 3 drops of TFA-d) δ 8.48 (d, J=1.9 Hz, 1H), 8.12-8.04 (m, 2H), 7.67 (dd, J=8.8, 7.8 Hz, 1H), 7.48-7.38 (m, 3H), 6.99 (d, J=3.8 Hz, 1H), 4.16-4.07 (m, 1H), 3.96 (dd, J=17.9, 3.9 Hz, 1H), 3.77 (dd, J=17.9, 9.1 Hz, 1H), 3.41 (dd, J=17.9, 3.9 Hz, 1H), 3.30 (dd, J=17.9, 9.1 Hz, 1H).

¹³C NMR (126 MHz, CDCl₃ and 3 drops of TFA-d) δ 201.9, 172.0, 160.4, 158.3 (d, J_{C—F}=252.6 Hz), 144.4 (d, J_{C—F}=4.3 Hz), 138.7, 138.5, 136.1, 135.3 (d, J_{C—F}=2.9 Hz), 130.9 (qd, J_{C—F}=33.5, 8.2 Hz), 128.9 (d, J_{C—F}=3.6 Hz), 127.9 (d, J_{C—F}=6.9 Hz), 125.9, 125.0 (d, J_{C—F}=12.5 Hz), 124.0, 123.1 (qd, J_{C—F}=272.2, 2.5 Hz), 122.9, 121.6-121.3 (m), 120.3, 113.9 (dq, J_{C—F}=25.8, 3.5 Hz), 108.3, 44.3, 35.4, 33.7.

¹⁹F NMR (471 MHz, CDCl₃ and 3 drops of TFA-d) δ −63.42, −112.24-−112.47 (m).

### 3-(5-(2-fluoro-4-(trifluoromethyl)phenyl)thiophen-2-yl)-6-methoxy-3,4-dihydroacridine-1,9(2H,10H)-dione (78)

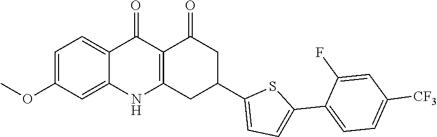 [SMILES: COc1ccc2c(=O)c3c([nH]c2c1)CC(c1ccc(-c2ccc(C(F)(F)F)cc2F)s1)CC3=O]

Following general procedure L, the title compound was prepared as a tan-colored powder.

TLC: (5% methanol in dichloromethane, R_{f}): 0.37 (UV, 254 nm, 280 nm).

¹H NMR (500 MHz, CDCl₃ and 3 drops of TFA-d) δ 8.36 (d, J=9.8 Hz, 1H), 7.68 (dd, J=8.0, 7.5 Hz, 1H), 7.46-7.35 (m, 5H), 7.00 (d, J=3.8 Hz, 1H), 4.13-4.03 (m, 1H), 4.03 (s, 3H), 3.90 (dd, J=17.6, 4.3 Hz, 1H), 3.70 (dd, J=17.8, 9.8 Hz, 1H), 3.35 (dd, J=17.6, 4.3 Hz, 1H), 3.23 (dd, J=17.8, 9.8 Hz, 1H).

¹³C NMR (126 MHz, CDCl₃ and 3 drops of TFA-d) δ 201.5, 171.6, 167.7, 160.0, 158.4 (d, J_{C—F}=252.5 Hz), 144.7 (d, J_{C—F}=4.4 Hz), 143.2, 135.1 (d, J_{C—F}=3.7 Hz), 130.8 (qd, J_{C—F}=33.6, 7.7 Hz), 128.9 (d, J_{C—F}=3.7 Hz), 127.9 (d, J_{C—F}=7.0 Hz), 126.5, 125.8, 125.1 (d, J_{C—F}=12.5 Hz), 123.1 (qd, J_{C—F}=272.1, 2.3 Hz), 121.8, 121.6-121.3 (m), 113.9 (dq, J_{C—F}=25.7, 3.6 Hz), 113.2, 107.1, 100.5, 56.7, 44.3, 35.2, 33.9.

¹⁹F NMR (471 MHz, CDCl₃ and 3 drops of TFA-d) δ −63.39, −112.03-−112.65 (m).

### 7-chloro-3-(5-(4-(trifluoromethoxy)phenyl)thiophen-2-yl)-3,4-dihydroacridine-1,9(2H,10H)-dione (79)

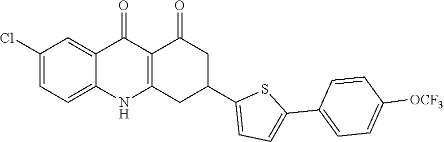 [SMILES: O=C1CC(c2ccc(-c3ccc(OC(F)(F)F)cc3)s2)Cc2[nH]c3ccc(Cl)cc3c(=O)c21]

Following general procedure L, the title compound was prepared as a yellow powder.

TLC: (5% methanol in dichloromethane, R_{f}): 0.47 (UV, 254 nm, 280 nm).

¹H NMR (500 MHz, CDCl₃ and 3 drops of TFA-d) δ 8.46 (d, J=2.2 Hz, 1H), 8.13 (d, J=9.0 Hz, 1H), 8.05 (dd, J=9.0, 2.2 Hz, 1H), 7.53 (d, J=8.3 Hz, 2H), 7.21 (d, J=8.3 Hz, 2H), 7.13 (d, J=3.7 Hz, 1H), 6.92 (d, J=3.7 Hz, 1H), 4.12-4.03 (m, 1H), 3.95 (dd, J=18.0, 4.3 Hz, 1H), 3.76 (dd, J=17.9, 8.8 Hz, 1H), 3.38 (dd, J=18.0, 4.3 Hz, 1H), 3.27 (dd, J=17.9, 8.8 Hz, 1H).

¹³C NMR (126 MHz, CDCl₃ and 3 drops of TFA-d) δ 202.0, 172.0, 160.5, 148.8 (q, J_{C-F}=1.9 Hz), 142.65, 142.61, 138.7, 138.5, 136.1, 132.3, 127.1, 126.1, 124.0, 123.8, 122.9, 121.4, 120.3, 120.1 (q, J_{C—F}=257.5 Hz), 108.3, 44.3, 35.4, 33.8.

¹⁹F NMR (471 MHz, CDCl₃ and 3 drops of TFA-d) δ −58.53.

### 6-methoxy-3-(5-(4-(trifluoromethoxy)phenyl)thiophen-2-yl)-3,4-dihydroacridine-1,9(2H,10H)-dione (80)

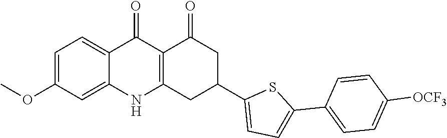 [SMILES: COc1ccc2c(=O)c3c([nH]c2c1)CC(c1ccc(-c2ccc(OC(F)(F)F)cc2)s1)CC3=O]

Following general procedure L, the title compound was prepared as a white powder.

TLC: (5% methanol in dichloromethane, R_{f}): 0.37 (UV, 254 nm, 280 nm).

¹H NMR (500 MHz, CDCl₃ and 3 drops of TFA-d) δ 8.36 (d, J=9.9 Hz, 1H), 7.58-7.51 (m, 2H), 7.41-7.35 (m, 2H), 7.22 (d, J=8.2 Hz, 2H), 7.14 (d, J=3.3 Hz, 1H), 6.92 (d, J=3.3 Hz, 1H), 4.10-4.01 (m, 4H), 3.88 (dd, J=17.8, 4.2 Hz, 1H), 3.69 (dd, J=17.8, 9.6 Hz, 1H), 3.34 (dd, J=17.8, 4.2 Hz, 1H), 3.21 (dd, J=17.8, 9.6 Hz, 1H).

¹³C NMR (126 MHz, CDCl₃ and 3 drops of TFA-d) δ 201.6, 171.6, 167.7, 160.1, 148.7 (q, J_{C—F}=2.8 Hz), 143.1, 142.9, 142.4, 132.4, 127.1, 126.5, 125.9, 123.7, 121.8, 121.4, 120.4 (q, J_{C—F}=257.5 Hz), 113.1, 107.1, 100.3, 56.7, 44.3, 35.3, 33.9.

¹⁹F NMR (471 MHz, CDCl₃ and 3 drops of TFA-d) δ −58.52.

### 7-chloro-3-(5-(3-(trifluoromethoxy)phenyl)thiophen-2-yl)-3,4-dihydroacridine-1,9(2H,10H)-dione (81)

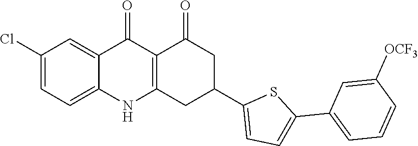 [SMILES: O=C1CC(c2ccc(-c3cccc(OC(F)(F)F)c3)s2)Cc2[nH]c3ccc(Cl)cc3c(=O)c21]

Following general procedure L, the title compound was prepared as a faint yellow powder.

TLC: (5% methanol in dichloromethane, R_{f}): 0.37 (UV, 254 nm, 280 nm).

¹H NMR (500 MHz, CDCl₃ and 3 drops of TFA-d) δ 8.47 (d, J=2.1 Hz, 1H), 8.13-8.04 (m, 2H), 7.48-7.34 (m, 3H), 7.20-7.12 (m, 2H), 6.93 (d, J=3.7 Hz, 1H), 4.14-4.05 (m, 1H), 3.95 (dd, J=17.9, 4.2 Hz, 1H), 3.76 (dd, J=17.9, 8.8 Hz, 1H), 3.40 (dd, J=17.9, 4.2 Hz, 1H), 3.29 (dd, J=17.9, 8.8 Hz, 1H).

¹³C NMR (126 MHz, CDCl₃ and 3 drops of TFA-d) δ 201.9, 172.1, 160.5, 149.7 (q, J_{C—F}=2.0 Hz), 142.9, 142.4, 138.7, 138.3, 136.3, 135.5, 130.4, 126.19, 126.17, 124.2, 124.1, 122.6, 120.4 (q, J_{C—F}=257.5 Hz), 120.3, 120.0, 118.2, 108.3, 44.3, 35.4, 33.8.

¹⁹F NMR (471 MHz, CDCl₃ and 3 drops of TFA-d) δ −58.48.

### 6-methoxy-3-(5-(3-(trifluoromethoxy)phenyl)thiophen-2-yl)-3,4-dihydroacridine-1,9(2H,10H)-dione (82)

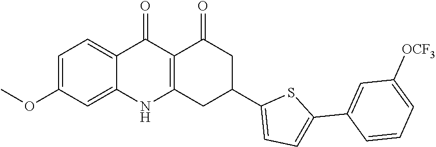 [SMILES: COc1ccc2c(=O)c3c([nH]c2c1)CC(c1ccc(-c2cccc(OC(F)(F)F)c2)s1)CC3=O]

Following general procedure L, the title compound was prepared as a tan-colored powder.

TLC: (5% methanol in dichloromethane, R_{f}): 0.37 (UV, 254 nm, 280 nm).

¹H NMR (500 MHz, DMSO-d₆) δ 11.96 (s, 1H), 8.01 (d, J=8.3 Hz, 1H), 7.62 (d, J=8.3 Hz, 1H), 7.59 (s, 1H), 7.56-7.50 (m, 2H), 7.29 (d, J=8.1 Hz, 1H), 7.04 (d, J=3.7 Hz, 1H), 6.97 (d, J=9.0 Hz, 1H), 6.91 (s, 1H), 3.91-3.79 (m, 4H), 3.32-3.27 (m, 2H), 2.83-2.69 (m, 2H).

### 7-chloro-3-(2-(4-(trifluoromethoxy)phenyl)thiazol-5-yl)-3,4-dihydroacridine-1,9(2H,10H)-dione (83)

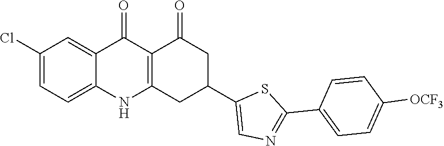 [SMILES: O=C1CC(c2cnc(-c3ccc(OC(F)(F)F)cc3)s2)Cc2[nH]c3ccc(Cl)cc3c(=O)c21]

Following general procedure L, the title compound was prepared as a tan-colored solid.

TLC: (5% methanol in dichloromethane, R_{f}): 0.31 (UV, 254 nm, 280 nm).

¹H NMR (500 MHz, DMSO-d₆) δ 12.31 (s, 1H), 8.07-7.97 (m, 3H), 7.82 (s, 1H), 7.74 (dd, J=8.8, 2.6 Hz, 1H), 7.59 (d, J=8.8 Hz, 1H), 7.49 (d, J=8.3 Hz, 2H), 4.04-3.90 (m, 1H), 3.41-3.34 (m, 2H), 2.90-2.73 (m, 2H).

### 6-methoxy-3-(2-(4-(trifluoromethoxy)phenyl)thiazol-5-yl)-3,4-dihydroacridine-1,9(2H,10H)-dione (84)

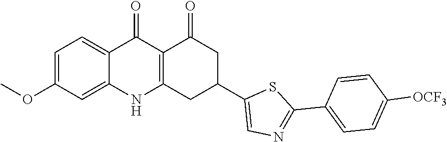 [SMILES: COc1ccc2c(=O)c3c([nH]c2c1)CC(c1cnc(-c2ccc(OC(F)(F)F)cc2)s1)CC3=O]

Following general procedure L, the title compound was prepared as a white powder.

TLC: (5% methanol in dichloromethane, R_{f}): 0.31 (UV, 254 nm, 280 nm).

¹H NMR (500 MHz, DMSO-d₆) δ 12.04 (s, 1H), 8.06-7.98 (m, 3H), 7.81 (s, 1H), 7.49 (d, J=8.3 Hz, 2H), 6.98 (dd, J=8.9, 2.4 Hz, 1H), 6.93 (d, J=2.4 Hz, 1H), 4.02-3.92 (m, 1H), 3.87 (s, 3H), 3.40-3.25 (m, 2H), 2.88-2.72 (m, 2H).

### 7-chloro-3-(2-(3-(trifluoromethoxy)phenyl)thiazol-5-yl)-3,4-dihydroacridine-1,9(2H,10H)-dione (85)

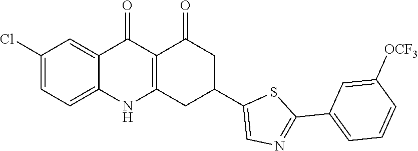 [SMILES: O=C1CC(c2cnc(-c3cccc(OC(F)(F)F)c3)s2)Cc2[nH]c3ccc(Cl)cc3c(=O)c21]

Following general procedure L, the title compound was prepared as a tan-colored solid.

TLC: (5% methanol in dichloromethane, R_{f}): 0.31 (UV, 254 nm, 280 nm).

¹H NMR (500 MHz, DMSO-d₆) δ 12.38 (s, 1H), 8.04 (d, J=2.5 Hz, 1H), 7.92 (d, J=7.8 Hz, 1H), 7.85 (s, 2H), 7.75 (dd, J=8.7, 2.5 Hz, 1H), 7.69-7.57 (m, 2H), 7.50 (d, J=8.7 Hz, 1H), 4.11-3.94 (m, 1H), 3.43-3.36 (m, 2H), 2.93-2.71 (m, 2H).

¹H NMR (500 MHz, CDCl₃ and 3 drops of TFA-d) δ 8.50 (d, J=2.1 Hz, 1H), 8.29 (s, 1H), 8.12-8.02 (m, 2H), 7.82 (d, J=8.0 Hz, 1H), 7.74 (s, 1H), 7.66 (dd, J=8.4, 8.0 Hz, 1H), 7.54 (d, J=8.4 Hz, 1H), 4.30-4.19 (m, 1H), 4.06 (dd, J=17.6, 3.5 Hz, 1H), 3.80 (dd, J=17.6, 10.8 Hz, 1H), 3.44 (dd, J=17.6, 3.5 Hz, 1H), 3.35 (dd, J=17.6, 10.8 Hz, 1H).

¹³C NMR (126 MHz, CDCl₃ and 3 drops of TFA-d) δ 200.0, 172.6, 170.0, 159.1, 150.2 (q, J_{C—F}=2.1 Hz), 140.8, 139.2, 138.0, 136.8, 134.6, 132.0, 128.1, 126.3, 125.9, 124.4, 122.2, 120.4, 120.3 (q, J_{C—F}=259.4 Hz), 119.8, 108.1, 42.8, 34.7, 31.5.

¹⁹F NMR (471 MHz, CDCl₃ and 3 drops of TFA-d) δ −58.82.

### 6-methoxy-3-(2-(3-(trifluoromethoxy)phenyl)thiazol-5-yl)-3,4-dihydroacridine-1,9(2H,10H)-dione (86)

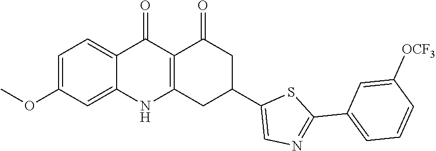 [SMILES: COc1ccc2c(=O)c3c([nH]c2c1)CC(c1cnc(-c2cccc(OC(F)(F)F)c2)s1)CC3=O]

Following general procedure L, the title compound was prepared as a tan-colored solid.

TLC: (5% methanol in dichloromethane, R_{f}): 0.36 (UV, 254 nm, 280 nm).

¹H NMR (500 MHz, DMSO-d₆) δ 8.05 (d, J=8.0 Hz, 1H), 7.91 (d, J=7.8 Hz, 1H), 7.88-7.82 (m, 2H), 7.65 (dd, J=9.0, 8.0 Hz, 1H), 7.48 (d, J=7.8 Hz, 1H), 7.13 (d, J=2.5 Hz, 1H), 7.04 (dd, J=9.0, 2.5 Hz, 1H), 4.04-3.97 (m, 1H), 3.88 (s, 3H), 3.51-3.34 (m, 2H), 2.94-2.79 (m, 2H).

¹H NMR (500 MHz, CDCl₃ and 3 drops of TFA-d) δ 8.43 (s, 1H), 8.39 (d, J=9.3 Hz, 1H), 7.92 (d, J=7.8 Hz, 1H), 7.80 (s, 1H), 7.70 (dd, J=8.5, 7.8 Hz, 1H), 7.58 (d, J=8.5 Hz, 1H), 7.50 (d, J=2.3 Hz, 1H), 7.41 (dd, J=9.3, 2.3 Hz, 1H), 4.31-4.23 (m, 1H), 4.20 (dd, J=17.9, 4.0 Hz, 1H), 4.05 (s, 3H), 3.83 (dd, J=17.9, 10.5 Hz, 1H), 3.41 (dd, J=17.9, 4.0 Hz, 1H), 3.33 (dd, J=17.9, 10.5 Hz, 1H).

¹³C NMR (126 MHz, CDCl₃ and 3 drops of TFA-d) δ 199.5, 171.9, 169.7, 168.2, 158.6, 150.2 (q, J_{C—F}=2.1 Hz), 142.7, 141.4, 133.4, 132.2, 127.1, 127.0, 126.8, 126.1, 122.1, 120.2 (q, J_{C—F}=259.6 Hz), 120.1, 113.3, 107.0, 100.1, 56.8, 42.6, 34.6, 31.9.

¹⁹F NMR (471 MHz, CDCl₃ and 3 drops of TFA-d) δ −58.96.

### 7-chloro-3-(2-(4-(trifluoromethoxy)phenyl)thiazol-4-yl)-3,4-dihydroacridine-1,9(2H,10H)-dione (87)

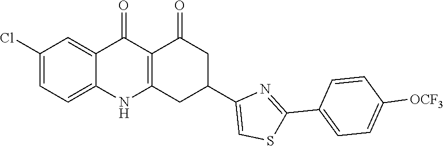 [SMILES: O=C1CC(c2csc(-c3ccc(OC(F)(F)F)cc3)n2)Cc2[nH]c3ccc(Cl)cc3c(=O)c21]

Following general procedure L, the title compound was prepared as a white powder.

TLC: (5% methanol in dichloromethane, R_{f}): 0.36 (UV, 254 nm, 280 nm).

¹H NMR (500 MHz, CDCl₃ and 3 drops of TFA-d) δ 8.52 (d, J=2.2 Hz, 1H), 8.11 (dd, J=9.0, 2.2 Hz, 1H), 8.02-7.96 (m, 3H), 7.87 (s, 1H), 7.47 (d, J=8.4 Hz, 2H), 4.42-4.33 (m, 1H), 4.01 (dd, 1H), 3.91 (dd, J=17.5, 11.7 Hz, 1H), 3.49-3.31 (m, 2H).

¹³C NMR (126 MHz, CDCl₃ and 3 drops of TFA-d) δ 200.0, 172.8, 172.4, 159.4, 154.1, 148.9, 139.3, 137.8, 136.9, 130.0, 124.5, 123.4, 121.9, 121.8, 120.5, 120.2 (q, J_{C—F}=260.4 Hz), 118.7, 108.3, 32.7, 32.28, 32.20.

¹⁹F NMR (471 MHz, CDCl₃ and 3 drops of TFA-d) δ −58.51.

### 6-methoxy-3-(2-(4-(trifluoromethoxy)phenyl)thiazol-4-yl)-3,4-dihydroacridine-1,9(2H,10H)-dione (88)

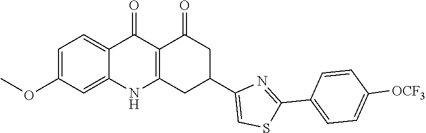 [SMILES: COc1ccc2c(=O)c3c([nH]c2c1)CC(c1csc(-c2ccc(OC(F)(F)F)cc2)n1)CC3=O]

Following general procedure L, the title compound was prepared as a pale yellow powder.

TLC: (5% methanol in dichloromethane, R_{f}): 0.34 (UV, 254 nm, 280 nm).

¹H NMR (500 MHz, DMSO-d₆) δ 8.11 (d, J=9.0 Hz, 1H), 8.08-8.02 (m, 2H), 7.60 (s, 1H), 7.50 (d, J=7.9 Hz, 2H), 7.25 (d, J=2.4 Hz, 1H), 7.11 (dd, J=9.0, 2.4 Hz, 1H), 3.90 (s, 3H), 3.88-3.79 (m, 1H), 3.58 (dd, J=16.8, 4.4 Hz, 1H), 3.48 (dd, J=16.8, 9.7 Hz, 1H), 3.03-2.90 (m, 2H).

¹H NMR (500 MHz, CDCl₃ and 3 drops of TFA-d) δ 8.40 (d, J=9.3 Hz, 1H), 8.10 (d, J=8.3 Hz, 2H), 7.88 (s, 1H), 7.46 (d, J=8.3 Hz, 2H), 7.41 (dd, J=9.3, 2.3 Hz, 1H), 7.33 (d, J=2.3 Hz, 1H), 4.52-4.33 (m, 1H), 4.12-4.00 (m, 4H), 4.00-3.87 (m, 1H), 3.47-3.35 (m, 2H).

¹³C NMR (126 MHz, CDCl₃ and 3 drops of TFA-d) δ 199.9, 172.07, 172.05, 168.1, 159.1, 153.9, 149.3, 142.5, 130.1, 127.0, 123.6, 122.0, 121.8, 120.2 (q, J_{C—F}=260.5 Hz), 118.5, 113.3, 107.2, 99.9, 56.6, 41.2, 32.7, 32.4.

¹⁹F NMR (471 MHz, CDCl₃ and 3 drops of TFA-d) δ −58.47.

### 7-chloro-3-(2-(3-(trifluoromethoxy)phenyl)thiazol-4-yl)-3,4-dihydroacridine-1,9(2H,10H)-dione (89)

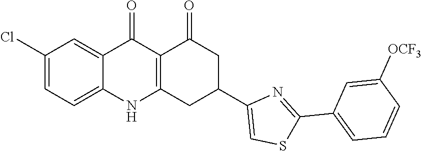 [SMILES: O=C1CC(c2csc(-c3cccc(OC(F)(F)F)c3)n2)Cc2[nH]c3ccc(Cl)cc3c(=O)c21]

Following general procedure L, the title compound was prepared as a white powder.

TLC: (5% methanol in dichloromethane, R_{f}): 0.34 (UV, 254 nm, 280 nm).

¹H NMR (500 MHz, DMSO-d₆) δ 12.33 (s, 1H), 8.02 (s, 1H), 7.94 (d, J=7.8 Hz, 1H), 7.86 (s, 1H), 7.73 (dd, J=8.9, 2.5 Hz, 1H), 7.68-7.56 (m, 3H), 7.50 (d, J=8.1 Hz, 1H), 3.82-3.72 (m, 1H), 3.50-3.36 (m, 2H), 2.92-2.74 (m, 2H).

¹H NMR (500 MHz, CDCl₃ and 3 drops of TFA-d) δ 8.50 (d, J=2.1 Hz, 1H), 8.11-8.01 (m, 2H), 7.84 (d, J=8.0 Hz, 1H), 7.77-7.73 (m, 2H), 7.64 (dd, J=9.2, 8.0 Hz, 1H), 7.51 (d, J=9.2 Hz, 1H), 4.39-4.30 (m, 1H), 4.02 (dd, J=17.6, 4.3 Hz, 1H), 3.93 (dd, J=17.6, 10.7 Hz, 1H), 3.46-3.34 (m, 2H).

¹³C NMR (126 MHz, CDCl₃ and 3 drops of TFA-d) δ 200.7, 172.5, 170.9, 159.9, 151.1, 150.1 (q, J_{C—F}=2.0 Hz), 139.0, 137.9, 136.6, 131.7, 129.2, 125.9, 125.8, 124.3, 122.0, 120.4, 120.3 (q, J_{C—F}=259.1 Hz), 119.8, 118.3, 108.5, 41.7, 35.6, 33.0.

¹⁹F NMR (471 MHz, CDCl₃ and 3 drops of TFA-d) δ −58.75.

### 6-methoxy-3-(2-(3-(trifluoromethoxy)phenyl)thiazol-4-yl)-3,4-dihydroacridine-1,9(2H,10H)-dione (90)

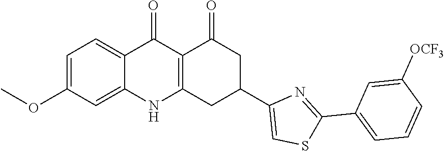 [SMILES: COc1ccc2c(=O)c3c([nH]c2c1)CC(c1csc(-c2cccc(OC(F)(F)F)c2)n1)CC3=O]

Following general procedure L, the title compound was prepared as a tan-colored solid.

TLC: (5% methanol in dichloromethane, R_{f}): 0.34 (UV, 254 nm, 280 nm).

¹H NMR (500 MHz, DMSO-d₆) δ 8.04 (d, J=9.0 Hz, 1H), 7.94 (d, J=8.0 Hz, 1H), 7.86 (s, 1H), 7.65 (dd, J=8.6, 8.0 Hz, 1H), 7.61 (s, 1H), 7.50 (d, J=8.6 Hz, 1H), 7.10 (d, J=2.4 Hz, 1H), 7.02 (dd, J=9.0, 2.4 Hz, 1H), 3.88 (s, 3H), 3.83-3.73 (m, 1H), 3.55-3.37 (m, 2H), 2.94-2.83 (m, 2H).

¹H NMR (500 MHz, CDCl₃ and 3 drops of TFA-d) δ 8.39 (d, J=9.2 Hz, 1H), 7.86 (d, J=7.8 Hz, 1H), 7.80-7.73 (m, 2H), 7.64 (dd, J=8.3, 7.8 Hz, 1H), 7.51 (d, J=8.3 Hz, 1H), 7.40 (dd, J=9.2, 2.3 Hz, 1H), 7.33 (d, J=2.3 Hz, 1H), 4.36-4.26 (m, 1H), 4.03 (s, 3H), 3.96 (dd, J=17.6, 4.2 Hz, 1H), 3.88 (dd, J=17.6, 10.7 Hz, 1H), 3.41-3.29 (m, 2H).

¹³C NMR (126 MHz, CDCl₃ and 3 drops of TFA-d) δ 200.5, 171.9, 170.7, 168.0, 159.5, 151.6, 150.1 (q, J_{C-F}=2.1 Hz), 142.6, 131.6, 129.5, 126.9, 125.9, 125.6, 121.9, 120.3 (q, J_{C—F}=259.2 Hz), 119.8, 118.1, 113.3, 107.3, 99.9, 56.6, 41.6, 32.9, 29.7.

¹⁹F NMR (471 MHz, CDCl₃ and 3 drops of TFA-d) δ −58.78.

### 6-(2-fluoro-4-(trifluoromethyl)phenyl)nicotinaldehyde (96)

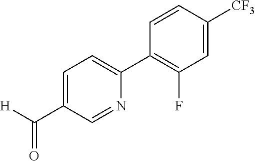 [SMILES: O=Cc1ccc(-c2ccc(C(F)(F)F)cc2F)nc1]

Following general procedure F, the title compound was prepared as a white powder.

LCMS (ESI-MS) m/z [M+H]⁺ calcd for C₁₃H₈F₄NO 270.05; found 269.8. Retention time: 4.342 min.

TLC: (10% EtOAc in hexanes, R_{f}): 0.18 (UV, 254 nm, 280 nm).

¹H NMR (500 MHz, DMSO-d₆) δ 10.19 (s, 1H), 9.25 (d, J=2.0 Hz, 1H), 8.41 (dd, J=8.1, 2.0 Hz, 1H), 8.23 (dd, J=8.7, 8.2 Hz, 1H), 8.10 (dd, J=8.2, 1.8 Hz, 1H), 7.90 (d, J=11.4 Hz, 1H), 7.78 (d, J=8.1 Hz, 1H).

¹³C NMR (126 MHz, DMSO-d₆) δ 192.5, 160.2 (d, J=252.3 Hz), 155.8 (d, J_{C—F}=2.6 Hz), 152.0, 137.6, 132.9 (d, J_{C—F}=2.8 Hz), 132.3 (d, J_{C—F}=8.6 Hz), 132.1 (d, J_{C—F}=8.6 Hz), 131.0, 130.3 (d, J_{C—F}=11.5 Hz), 125.3 (d, J_{C—F}=9.2 Hz), 122.4-122.0 (m), 114.6 (dd, J_{C—F}=26.6, 3.9 Hz).

¹⁹F NMR (471 MHz, CDCl₃) δ −62.97, −113.62-−113.72 (m).

### 6-(4-(trifluoromethoxy)phenyl)nicotinaldehyde (97)

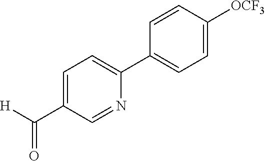 [SMILES: O=Cc1ccc(-c2ccc(OC(F)(F)F)cc2)nc1]

Following general procedure F, the title compound was prepared as a faint yellow powder.

LCMS (ESI-MS) m/z [M+H]⁺ calcd for C₁₃H₉F₃NO₂ 268.05; found 267.8. Retention time: 4.410 min.

TLC: (10% EtOAc in hexanes, R_{f}): 0.13 (UV, 254 nm, 280 nm).

¹H NMR (500 MHz, DMSO-d₆) δ 10.16 (s, 1H), 9.18 (d, J=2.1 Hz, 1H), 8.40-8.29 (m, 3H), 8.25 (d, J=8.1 Hz, 1H), 7.54 (d, J=8.8 Hz, 2H).

¹³C NMR (126 MHz, DMSO-d₆) b 192.4, 159.3, 152.1, 150.2 (q, J_{C—F}=2.1 Hz), 137.8, 137.0, 130.6, 129.8, 121.7, 121.2, 120.5 (q, J_{C—F}=256.8 Hz).

¹⁹F NMR (471 MHz, CDCl₃) δ −57.68.

### 6-(3-(trifluoromethoxy)phenyl)nicotinaldehyde (98)

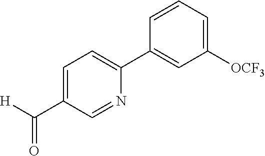 [SMILES: O=Cc1ccc(-c2cccc(OC(F)(F)F)c2)nc1]

Following general procedure F, the title compound was prepared as an off-white tan powder.

LCMS (ESI-MS) m/z [M+H]⁺ calcd for C₁₃H₉F₃NO₂ 268.05; found 267.8. Retention time: 4.4394 min.

TLC: (10% EtOAc in hexanes, R_{f}): 0.13 (UV, 254 nm, 280 nm).

¹H NMR (500 MHz, CDCl₃) δ 10.17 (s, 1H), 9.16 (d, J=1.8 Hz, 1H), 8.28 (dd, J=8.3, 1.8 Hz, 1H), 8.07-7.97 (m, 2H), 7.93 (d, J=8.3 Hz, 1H), 7.56 (dd, J=8.6, 8.0 Hz, 1H), 7.36 (d, J=8.0 Hz, 1H).

¹³C NMR (126 MHz, CDCl₃) δ 190.2, 160.3, 152.3, 149.9 (q, J_{C—F}=2.1 Hz), 140.0, 136.8, 130.3, 130.3, 125.7, 122.6, 120.7, 120.5 (q, J_{C—F}=257.5 Hz), 120.2.

¹⁹F NMR (471 MHz, CDCl₃) δ −57.70.

### 5-(4-(trifluoromethoxy)phenyl)picolinaldehyde (99)

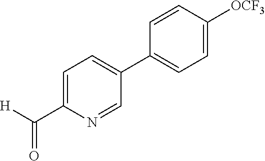 [SMILES: O=Cc1ccc(-c2ccc(OC(F)(F)F)cc2)cn1]

Following general procedure G, the title compound was prepared as a white powder.

LCMS (ESI-MS) m/z [M+H]⁺ calcd for C₁₃H₉F₃NO₂ 268.05; found 267.8. Retention time: 4.330 min.

TLC: (10% EtOAc in hexanes, R_{f}): 0.13 (UV, 254 nm, 280 nm).

¹H NMR (500 MHz, CDCl₃) δ 10.13 (s, 1H), 8.99 (s, 1H), 8.10-8.00 (m, 2H), 7.67 (d, J=9.3 Hz, 2H), 7.38 (d, J=9.3 Hz, 2H).

¹³C NMR (126 MHz, CDCl₃) δ 192.9, 151.8, 150.0 (q, J_{C—F}=1.9 Hz), 148.5, 139.3, 135.2, 135.2, 128.9, 121.8, 121.7, 120.4 (q, J_{C—F}=257.9 Hz).

¹⁹F NMR (471 MHz, CDCl₃) δ −57.78.

### 5-(3-(trifluoromethoxy)phenyl)picolinaldehyde (100)

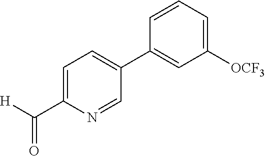 [SMILES: O=Cc1ccc(-c2cccc(OC(F)(F)F)c2)cn1]

Following general procedure F, the title compound was prepared as a yellow powder.

LCMS (ESI-MS) m/z [M+H]⁺ calcd for C₁₃H₉F₃NO₂ 268.05; found 267.8. Retention time: 4.329 min.

TLC: (10% EtOAc in hexanes, R_{f}): 0.13 (UV, 254 nm, 280 nm).

¹H NMR (500 MHz, CDCl₃) δ 10.12 (s, 1H), 8.99 (s, 1H), 8.10-8.01 (m, 2H), 7.62-7.52 (m, 2H), 7.48 (s, 1H), 7.33 (d, J=7.5 Hz, 1H).

¹³C NMR (126 MHz, CDCl₃) δ 192.9, 152.1, 150.0 (q, J_{C—F}=2.0 Hz), 148.6, 139.1, 138.7, 135.4, 130.8, 125.8, 121.8, 121.4, 120.4 (q, J_{C—F}=257.9 Hz), 120.0.

¹⁹F NMR (471 MHz, CDCl₃) δ −57.74.

### 5-(2-fluoro-4-(trifluoromethyl)phenyl)thiophene-2-carbaldehyde (101)

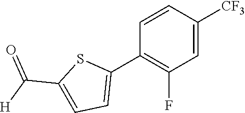 [SMILES: O=Cc1ccc(-c2ccc(C(F)(F)F)cc2F)s1]

Following the general procedure F, the title compound was prepared as a dark yellow solid.

LCMS (ESI-MS) m/z [M+H]⁺ calcd for C₁₂H₇F₄OS 275.01; found 274.8. Retention time: 3.791 min.

TLC: (7% EtOAc in hexanes, R_{f}): 0.21 (UV, 254 nm, 280 nm).

¹H NMR (500 MHz, CDCl₃) δ 9.96 (s, 1H), 7.84-7.78 (m, 2H), 7.64 (dd, J=4.0, 1.3 Hz, 1H), 7.53-7.44 (m, 2H).

¹³C NMR (126 MHz, CDCl₃) δ 182.8, 159.9, 157.9, 144.6 (d, J_{C—F}=4.0 Hz), 144.1 (d, J_{C—F}=3.7 Hz), 136.5, 132.4 (qd, J_{C—F}=33.9, 8.3 Hz), 129.6 (d, J_{C—F}=3.1 Hz), 128.4 (d, J_{C—F}=7.1 Hz), 122.9 (qd, J_{C—F}=272.5, 2.6 Hz), 121.7 (p, J_{C—F}=3.8 Hz), 114.2 (dq, J_{C—F}=25.8, 3.8 Hz).

¹⁹F NMR (471 MHz, CDCl₃) δ −62.97, −109.95-−110.16 (m).

### 5-(4-(trifluoromethoxy)phenyl)thiophene-2-carbaldehyde (102)

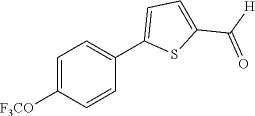 [SMILES: O=Cc1ccc(-c2ccc(OC(F)(F)F)cc2)s1]

Following the general procedure F, the title compound was prepared as a yellow solid.

LCMS (ESI-MS) m/z [M+H]⁺ calcd for C₁₂H₈F₃O₂S 273.01; found 272.7. Retention time: 3.813 min.

TLC: (7% EtOAc in hexanes, R_{f}): 0.18 (UV, 254 nm, 280 nm).

¹H NMR (500 MHz, CDCl₃) δ 9.90 (s, 1H), 7.75 (d, J=3.9 Hz, 1H), 7.72-7.67 (m, 2H), 7.39 (d, J=3.9 Hz, 1H), 7.31-7.27 (m, 2H).

¹³C NMR (126 MHz, CDCl₃) δ 182.7, 152.3, 149.8 (q, J_{C—F}=2.0 Hz), 143.0, 137.3, 131.8, 127.9, 124.6, 121.6, 120.4 (q, J_{C—F}=258.1 Hz).

¹⁹F NMR (471 MHz, CDCl₃) δ −57.80.

### 5-(3-(trifluoromethoxy)phenyl)thiophene-2-carbaldehyde (103)

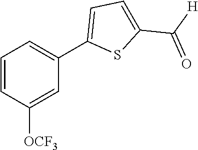 [SMILES: O=Cc1ccc(-c2cccc(OC(F)(F)F)c2)s1]

Following the general procedure F, the title compound was prepared as a beige powder.

LCMS (ESI-MS) m/z [M+H]⁺ calcd for C₁₂H₈F₃O₂S 273.01; found 272.9. Retention time: 3.761 min.

TLC: (7% EtOAc in hexanes, R_{f}): 0.21 (UV, 254 nm, 280 nm).

¹H NMR (500 MHz, DMSO-d₆) b 9.95 (s, 1H), 8.08 (d, J=4.0 Hz, 1H), 7.88 (d, J=4.0 Hz, 1H), 7.86-7.82 (m, 2H), 7.67-7.60 (m, 1H), 7.48-7.42 (m, 1H).

¹³C NMR (126 MHz, CDCl₃) δ 182.7, 151.9, 149.8 (q, J_{C—F}=2.3 Hz), 143.2, 137.1, 135.0, 130.7, 124.9, 124.8, 121.5, 120.4 (q, J_{C—F}=258.0 Hz), 118.8.

¹⁹F NMR (471 MHz, CDCl₃) δ −57.75.

### 2-(4-(trifluoromethoxy)phenyl)thiazole-5-carbaldehyde (104)

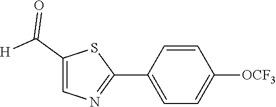 [SMILES: O=Cc1cnc(-c2ccc(OC(F)(F)F)cc2)s1]

Following general procedure E, the title compound was prepared as a white powder.

LCMS (ESI-MS) m/z [M+H]⁺ calcd for C₁₁H₇F₃NO₂S 274.01; found 273.8. Retention time: 4.449 min.

TLC: (10% EtOAc in hexanes, R_{f}): 0.13 (UV, 254 nm, 280 nm).

¹H NMR (500 MHz, CDCl₃) δ 10.08 (s, 1H), 8.46 (s, 1H), 8.10 (d, J=8.7 Hz, 2H), 7.36 (d, J=8.7 Hz, 2H).

¹³C NMR (126 MHz, CDCl₃) δ 182.0, 173.7, 152.2, 151.6 (q, J_{C—F}=1.9 Hz), 139.3, 131.0, 128.9, 122.6, 121.3, 120.2 (q, J_{C—F}=259.0 Hz), 116.1.

¹⁹F NMR (471 MHz, CDCl₃) δ −57.68.

### 2-(3-(trifluoromethoxy)phenyl)thiazole-5-carbaldehyde (105)

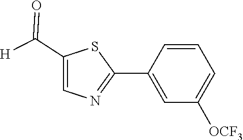 [SMILES: O=Cc1cnc(-c2cccc(OC(F)(F)F)c2)s1]

Following general procedure E, the title compound was prepared as a yellow powder.

LCMS (ESI-MS) m/z [M+H]⁺ calcd for C₁₁H₇F₃NO₂S 274.01; found 273.7. Retention time: 3.617 min.

TLC: (10% EtOAc in hexanes, R_{f}): 0.13 (UV, 254 nm, 280 nm).

¹H NMR (500 MHz, CDCl₃) δ 10.07 (s, 1H), 8.46 (s, 1H), 7.97-7.88 (m, 2H), 7.54 (dd, J=8.9, 8.1 Hz, 1H), 7.38 (d, J=8.1 Hz, 1H).

¹³C NMR (126 MHz, CDCl₃) δ 182.0, 173.3, 152.1, 149.8 (q, J_{C—F}=2.2 Hz), 139.5, 134.4, 130.7, 125.5, 123.9, 120.4 (q, J_{C—F}=258.3 Hz), 119.5.

¹⁹F NMR (471 MHz, CDCl₃) δ −57.80.

### 4-(4-(trifluoromethoxy)phenyl)thiazole-2-carbaldehyde (106)

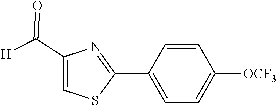 [SMILES: O=Cc1csc(-c2ccc(OC(F)(F)F)cc2)n1]

Following general procedure E afforded 94% pure compound as a brown powder and was directly used without further purification.

LCMS (ESI-MS) m/z [M+H]⁺ calcd for C₁₁H₇F₃NO₂S 274.01; found 273.8. Retention time: 4.317 min.

TLC: (10% EtOAc in hexanes, R_{f}): 0.18 (UV, 254 nm, 280 nm).

### 4-(3-(trifluoromethoxy)phenyl)thiazole-2-carbaldehyde (107)

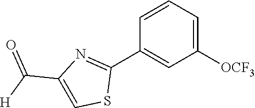 [SMILES: O=Cc1csc(-c2cccc(OC(F)(F)F)c2)n1]

Following general procedure E afforded 93% pure compound as a brown powder and was directly used without further purification.

LCMS (ESI-MS) m/z [M+H]⁺ calcd for C₁₁H₇F₃NO₂S 274.01; found 273.8. Retention time: 4.328 min.

TLC: (10% EtOAc in hexanes, R_{f}): 0.18 (UV, 254 nm, 280 nm).

### (E)-4-(6-(2-fluoro-4-(trifluoromethyl)phenyl)pyridin-3-yl)but-3-en-2-one (108)

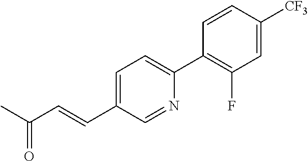 [SMILES: CC(=O)/C=C/c1ccc(-c2ccc(C(F)(F)F)cc2F)nc1]

Following general procedure I, the title compound was prepared as a white powder.

LCMS (ESI-MS) m/z: [M+H]⁺ calcd for C₁₆H₁₂F₄NO 310.08; found 309.8. Retention time: 4.527 min.

TLC: (20% EtOAc in hexanes, R_{f}): 0.22 (UV, 254 nm, 280 nm).

¹H NMR (500 MHz, CDCl₃) δ 8.88 (s, 1H), 8.23 (dd, J=9.2, 7.8 Hz, 1H), 7.97 (d, J=8.6 Hz, 1H), 7.91 (d, J=8.6 Hz, 1H), 7.61-7.52 (m, 2H), 7.46 (d, J=11.1 Hz, 1H), 6.85 (d, J=16.4 Hz, 1H), 2.44 (s, 3H).

¹³C NMR (126 MHz, CDCl₃) δ 197.6, 160.2 (d, J_{C—F}=252.9 Hz), 153.0 (d, J_{C—F}=2.8 Hz), 149.9, 138.7, 134.9, 132.9 (qd, J_{C—F}=33.4, 8.1 Hz), 131.8 (d, J_{C—F}=3.0 Hz), 129.8 (d, J_{C—F}=11.2 Hz), 129.7, 129.1, 124.7 (d, J_{C—F}=10.6 Hz), 123.1 (qd, J_{C—F}=272.5, 2.5 Hz), 121.5-121.3 (m), 113.9 (dq, J_{C—F}=26.6, 3.9 Hz), 27.8.

¹⁹F NMR (471 MHz, CDCl₃) δ −62.87, −114.06-−114.14 (m).

### (E)-4-(6-(4-(trifluoromethoxy)phenyl)pyridin-3-yl)but-3-en-2-one (109)

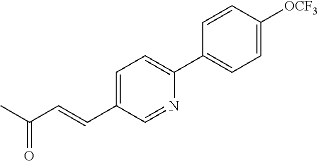 [SMILES: CC(=O)/C=C/c1ccc(-c2ccc(OC(F)(F)F)cc2)nc1]

Following general procedure I, the title compound was prepared as a white powder.

LCMS (ESI-MS) m/z: [M+H]⁺ calcd for C₁₆H₁₃F₃NO₂ 308.08; found 307.8. Retention time: 4.546 min.

TLC: (20% EtOAc in hexanes, R_{f}): 0.22 (UV, 254 nm, 280 nm).

¹H NMR (500 MHz, CDCl₃) δ 8.82 (d, J=2.0 Hz, 1H), 8.08 (d, J=8.5 Hz, 2H), 7.94 (dd, J=8.4, 2.0 Hz, 1H), 7.77 (d, J=8.4 Hz, 1H), 7.55 (d, J=16.1 Hz, 1H), 7.34 (d, J=8.5 Hz, 2H), 6.82 (d, J=16.1 Hz, 1H), 2.43 (s, 3H).

¹³C NMR (126 MHz, CDCl₃) δ 197.7, 157.2, 150.3 (q, J_{C—F}=1.8 Hz), 150.1, 139.1, 136.9, 135.1, 128.9, 128.5, 128.4, 121.1, 120.4 (q, J_{C—F}=257.7 Hz), 120.3, 27.7.

¹⁹F NMR (471 MHz, CDCl₃) δ −57.70.

### (E)-4-(6-(3-(trifluoromethoxy)phenyl)pyridin-3-yl)but-3-en-2-one (110)

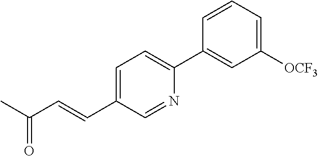 [SMILES: CC(=O)/C=C/c1ccc(-c2cccc(OC(F)(F)F)c2)nc1]

Following general procedure I, the title compound was prepared as a white powder.

LCMS (ESI-MS) m/z: [M+H]⁺ calcd for C₁₆H₁₃F₃NO₂ 308.08; found 307.8. Retention time: 3.705 min.

TLC: (20% EtOAc in hexanes, R_{f}): 0.22 (UV, 254 nm, 280 nm).

¹H NMR (500 MHz, CDCl₃) δ 8.85 (d, J=2.3 Hz, 1H), 8.04-7.89 (m, 3H), 7.80 (d, J=8.6 Hz, 1H), 7.60-7.49 (m, 2H), 7.32 (d, J=8.1 Hz, 1H), 6.83 (d, J=16.5 Hz, 1H), 2.44 (s, 3H).

¹³C NMR (126 MHz, CDCl₃) δ 197.7, 156.9, 150.1, 149.9 (q, J_{C—F}=2.3 Hz), 140.4, 139.0, 135.1, 130.2, 129.3, 128.6, 125.1, 121.9, 120.54 (q, J_{C-F}=257.4 Hz), 120.50, 119.6, 27.8.

¹⁹F NMR (471 MHz, CDCl₃) δ −57.67.

### (E)-4-(5-(4-(trifluoromethoxy)phenyl)pyridin-2-yl)but-3-en-2-one (111)

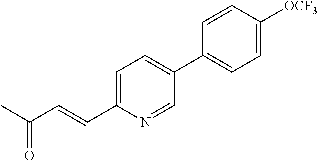 [SMILES: CC(=O)/C=C/c1ccc(-c2ccc(OC(F)(F)F)cc2)cn1]

Following general procedure I, the title compound was prepared as a white powder.

LCMS (ESI-MS) m/z: [M+H]⁺ calcd for C₁₆H₁₃F₃NO₂ 308.08; found 307.8. Retention time: 4.469 min.

TLC: (20% EtOAc in hexanes, R_{f}): 0.14 (UV, 254 nm, 280 nm).

¹H NMR (500 MHz, CDCl₃) δ 8.87 (d, J=2.4 Hz, 1H), 7.90 (dd, J=8.1, 2.4 Hz, 1H), 7.67-7.50 (m, 4H), 7.35 (d, J=8.2 Hz, 2H), 7.19 (d, J=15.8 Hz, 1H), 2.43 (s, 3H).

¹³C NMR (126 MHz, CDCl₃) δ 198.4, 152.3, 149.5 (q, J_{C—F}=1.9 Hz), 148.5, 141.2, 135.8, 134.9, 130.3, 128.5, 124.2 (2×C), 121.6, 120.4 (q, J_{C-F}=257.7 Hz), 28.1.

¹⁹F NMR (471 MHz, CDCl₃) δ −57.80.

### (E)-4-(5-(3-(trifluoromethoxy)phenyl)pyridin-2-yl)but-3-en-2-one (112)

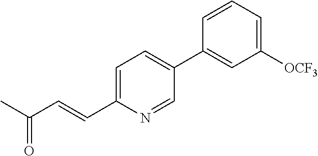 [SMILES: CC(=O)/C=C/c1ccc(-c2cccc(OC(F)(F)F)c2)cn1]

Following general procedure I, the title compound was prepared as an off-white and light brown powder.

LCMS (ESI-MS) m/z: [M+H]⁺ calcd for C₁₆H₁₃F₃NO₂ 308.08; found 307.8. Retention time: 4.468 min.

TLC: (33% EtOAc in hexanes, R_{f}): 0.38 (UV, 254 nm, 280 nm).

¹H NMR (500 MHz, CDCl₃) δ 8.88 (d, J=2.4 Hz, 1H), 7.91 (dd, J=8.4, 2.4 Hz, 1H), 7.62-7.50 (m, 4H), 7.45 (s, 1H), 7.29 (d, J=7.2 Hz, 1H), 7.20 (d, J=16.0 Hz, 1H), 2.44 (s, 3H).

¹³C NMR (126 MHz, CDCl₃) δ 198.4, 152.6, 149.9 (q, J_{C—F}=1.9 Hz), 148.6, 141.2, 139.2, 135.6, 135.0, 130.7, 130.4, 125.4, 124.2, 120.8, 120.4 (q, J_{C—F}=257.7 Hz), 119.6, 28.2.

¹⁹F NMR (471 MHz, CDCl₃) δ −57.72.

### (E)-4-(5-(3-(trifluoromethoxy)phenoxy)thiophen-2-yl)but-3-en-2-one (113)

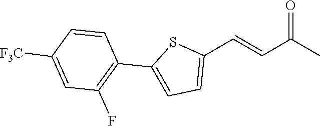 [SMILES: CC(=O)/C=C/c1ccc(-c2ccc(C(F)(F)F)cc2F)s1]

Following general procedure H, the title compound was prepared as a bright yellow powder.

LCMS (ESI-MS) m/z: [M+H]⁺ calcd for C₁₅H₁₁F₄OS 315.04; found 315.0. Retention time: 3.990 min.

TLC: (25% EtOAc in hexanes, R_{f}): 0.26 (UV, 254 nm, 280 nm).

¹H NMR (500 MHz, CDCl₃) δ 7.78-7.72 (m, 1H), 7.62 (d, J=15.9 Hz, 1H), 7.52 (dd, J=3.9, 1.2 Hz, 1H), 7.49-7.41 (m, 2H), 7.32 (d, J=3.9 Hz, 1H), 6.59 (d, J=15.9 Hz, 1H), 2.36 (s, 3H).

¹³C NMR (126 MHz, CDCl₃) δ 197.4, 159.6, 157.6, 140.9 (d, J_{C—F}=4.5 Hz), 138.4 (d, J_{C—F}=4.2 Hz), 135.0, 132.2, 131.3 (qd, J_{C—F}=33.8, 8.1 Hz), 129.0 (d, J_{C—F}=3.4 Hz), 128.6 (d, J_{C—F}=7.3 Hz), 126.4, 123.1 (qd, J_{C—F}=272.2, 2.6 Hz), 121.7-121.4 (m), 114.1 (dq, J_{C—F}=25.9, 3.9 Hz), 27.9.

¹⁹F NMR (471 MHz, CDCl₃) δ −62.82, −110.71-−110.79 (m).

### (E)-4-(5-(4-(trifluoromethoxy)phenyl)thiophen-2-yl)but-3-en-2-one (114)

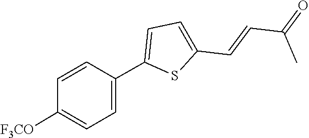 [SMILES: CC(=O)/C=C/c1ccc(-c2ccc(OC(F)(F)F)cc2)s1]

Following general procedure H, the title compound was prepared as a golden-yellow powder.

LCMS (ESI-MS) m/z: [M+H]⁺ calcd for C₁₅H₁₂F₃O₂S 313.04; found 312.7. Retention time: 4.032 min.

TLC: (25% EtOAc in hexanes, R_{f}): 0.24 (UV, 254 nm, 280 nm).

¹H NMR (500 MHz, DMSO-d₆) δ 7.87-7.82 (m, 2H), 7.79 (d, J=16.0 Hz, 1H), 7.63 (d, J=3.9 Hz, 1H), 7.58 (d, J=3.9 Hz, 1H), 7.48-7.42 (m, 2H), 6.51 (d, J=16.0 Hz, 1H), 2.32 (s, 3H).

¹³C NMR (126 MHz, CDCl₃) δ 197.5, 149.1 (q, J_{C—F}=1.9 Hz), 145.9, 139.5, 135.4, 132.9, 132.2, 127.3, 125.7, 124.7, 121.5, 120.4 (q, J_{C—F}=257.6 Hz), 27.8.

¹⁹F NMR (471 MHz, CDCl₃) δ −57.82.

### (E)-4-(5-(3-(trifluoromethoxy)phenyl)thiophen-2-yl)but-3-en-2-one (115)

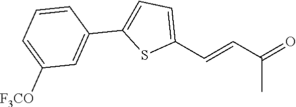 [SMILES: CC(=O)/C=C/c1ccc(-c2cccc(OC(F)(F)F)c2)s1]

Following general procedure H, the title compound was prepared as a dark brown oil.

LCMS (ESI-MS) m/z: [M+H]⁺ calcd for C₁₅H₁₂F₃O₂S 313.04; found 312.7. Retention time: 4.080 min.

TLC: (25% EtOAc in hexanes, R_{f}): 0.24 (UV, 254 nm, 280 nm).

¹H NMR (500 MHz, CDCl₃) δ 7.60 (d, J=15.9 Hz, 1H), 7.56-7.50 (m, 1H), 7.46-7.39 (m, 2H), 7.32-7.25 (m, 2H), 7.22-7.15 (m, 1H), 6.55 (d, J=15.8 Hz, 1H), 2.36 (s, 3H).

¹³C NMR (126 MHz, CDCl₃) δ 197.5, 149.7 (q, J_{C—F}=2.0 Hz), 145.6, 139.8, 135.5, 135.3, 132.7, 130.5, 125.9, 125.0, 124.3, 120.5, 120.4 (q, J_{C—F}=257.6 Hz), 118.4, 27.8.

¹⁹F NMR (471 MHz, CDCl₃) δ −57.72.

### (E)-4-(2-(4-(trifluoromethoxy)phenyl)thiazol-5-yl)but-3-en-2-one (116)

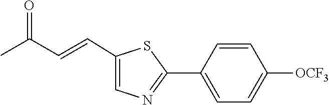 [SMILES: CC(=O)/C=C/c1cnc(-c2ccc(OC(F)(F)F)cc2)s1]

Following general procedure 1, the title compound was prepared as a white powder.

LCMS (ESI-MS) m/z: [M+H]⁺ calcd for C₁₄H₁₁F₃NO₂S 314.04; found 313.8. Retention time: 3.688 min.

TLC: (20% EtOAc in hexanes, R_{f}): 0.32 (UV, 254 nm, 280 nm).

¹H NMR (500 MHz, CDCl₃) δ 8.05-7.98 (m, 3H), 7.66 (d, J=16.0 Hz, 1H), 7.33 (d, J=8.4 Hz, 2H), 6.54 (d, J=16.0 Hz, 1H), 2.39 (s, 3H).

¹³C NMR (126 MHz, CDCl₃) δ 197.1, 168.3, 150.9 (q, J_{C—F}=1.8 Hz), 147.5, 135.5, 132.2, 131.5, 128.9, 128.3, 121.3, 120.3 (q, J_{C—F}=258.4 Hz), 27.8.

¹⁹F NMR (471 MHz, CDCl₃) δ −57.71.

### (E)-4-(2-(3-(trifluoromethoxy)phenyl)thiazol-5-yl)but-3-en-2-one (117)

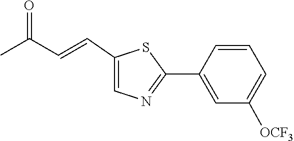 [SMILES: CC(=O)/C=C/c1cnc(-c2cccc(OC(F)(F)F)c2)s1]

Following general procedure 1, the title compound was prepared as a yellow powder.

LCMS (ESI-MS) m/z: [M+H]⁺ calcd for C₁₄H₁₁F₃NO₂S 314.04; found 313.8. Retention time: 3.807 min.

TLC: (20% EtOAc in hexanes, R_{f}): 0.32 (UV, 254 nm, 280 nm).

¹H NMR (500 MHz, CDCl₃) δ 7.99 (s, 1H), 7.89-7.80 (m, 2H), 7.65 (d, J=15.8 Hz, 1H), 7.49 (dd, J=8.9, 8.4 Hz, 1H), 7.32 (d, J=8.4 Hz, 1H), 6.54 (d, J=15.8 Hz, 1H), 2.37 (s, 3H).

¹³C NMR (126 MHz, CDCl₃) δ 197.0, 168.0, 149.8 (q, J_{C—F}=1.8, 1.4 Hz), 147.4, 135.8, 134.9, 132.0, 130.6, 129.0, 125.1, 123.0, 120.4 (q, J_{C-F}=256.0 Hz), 119.1, 27.8.

¹⁹F NMR (471 MHz, CDCl₃) δ −57.77.

### (E)-4-(4-(4-(trifluoromethoxy)phenyl)thiazol-2-yl)but-3-en-2-one (118)

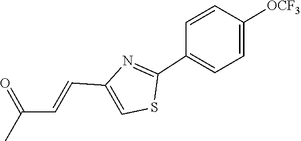 [SMILES: CC(=O)/C=C/c1csc(-c2ccc(OC(F)(F)F)cc2)n1]

Following general procedure 1, the title compound was prepared as an off-white and slightly yellow powder.

LCMS (ESI-MS) m/z: [M+H]⁺ calcd for C₁₄H₁₁F₃NO₂S 314.04; found 313.8. Retention time: 3.866 min.

TLC: (20% EtOAc in hexanes, R_{f}): 0.32 (UV, 254 nm, 280 nm).

¹H NMR (500 MHz, CDCl₃) δ 8.02 (d, J=7.9 Hz, 2H), 7.51-7.44 (m, 2H), 7.31 (d, J=7.9 Hz, 2H), 7.14 (d, J=15.7 Hz, 1H), 2.40 (s, 3H).

¹³C NMR (126 MHz, CDCl₃) δ 198.5, 167.1, 152.9, 150.7 (q, J_{C—F}=1.9 Hz), 134.3, 131.6, 129.2, 128.3, 122.1, 121.3, 120.3 (q, J_{C—F}=258.2 Hz), 28.2.

¹⁹F NMR (471 MHz, CDCl₃) δ −57.73.

### (E)-4-(4-(3-(trifluoromethoxy)phenyl)thiazol-2-yl)but-3-en-2-one (119)

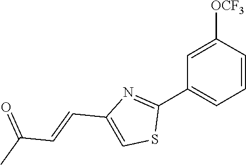 [SMILES: CC(=O)/C=C/c1csc(-c2cccc(OC(F)(F)F)c2)n1]

Following general procedure 1, the title compound was prepared as an off-white and slightly yellow powder.

LCMS (ESI-MS) m/z: [M+H]⁺ calcd for C₁₄H₁₁F₃NO₂S 314.04; found 313.8. Retention time: 3.860 min.

TLC: (20% EtOAc in hexanes, R_{f}): 0.32 (UV, 254 nm, 280 nm).

¹H NMR (500 MHz, CDCl₃) δ 7.90-7.86 (m, 2H), 7.53-7.45 (m, 3H), 7.34-7.29 (m, 1H), 7.15 (d, J=15.7 Hz, 1H), 2.40 (s, 3H).

¹³C NMR (126 MHz, CDCl₃) δ 198.4, 166.9, 152.9, 149.7 (q, J_{C—F}=1.9 Hz), 134.9, 134.2, 130.5, 129.3, 125.1, 122.7, 122.2, 120.4 (q, J_{C—F}=257.9 Hz), 119.2, 28.3.

¹⁹F NMR (471 MHz, CDCl₃) δ −57.74.

### 5-(6-(2-fluoro-4-(trifluoromethyl)phenyl)pyridin-3-yl)-3-hydroxycyclohex-2-en-1-one (120)

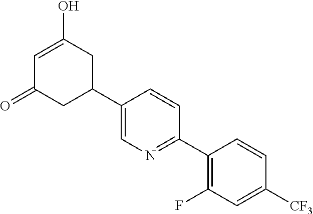 [SMILES: O=C1C=C(O)CC(c2ccc(-c3ccc(C(F)(F)F)cc3F)nc2)C1]

Following general procedures J and K, the title compound was prepared as a white powder.

LCMS (ESI-MS) m/z: [M+H]⁺ calcd for C₁₈H₁₄F₄NO₂ 352.09; found 351.8. Retention time: 3.970 min.

TLC: (5% methanol in dichloromethane, R_{f}): 0.14 (UV, 254 nm, 280 nm).

¹H NMR (500 MHz, DMSO-d₆) δ 11.34 (s, 1H), 8.77 (d, J=2.3 Hz, 1H), 8.16 (dd, J=8.6, 7.9 Hz, 1H), 7.99 (dd, J=8.3, 2.3 Hz, 1H), 7.88-7.77 (m, 2H), 7.72 (d, J=8.1 Hz, 1H), 5.35 (s, 1H), 3.54-3.42 (m, 1H), 2.71 (dd, J=16.5, 11.9 Hz, 2H), 2.50 (dd, J=16.0, 4.1 Hz, 2H).

¹³C NMR (126 MHz, DMSO-d₆) δ 159.9 (d, J_{C—F}=250.9 Hz), 149.8 (d, J_{C—F}=2.1 Hz), 149.6, 139.4, 135.9, 132.5 (d, J_{C—F}=3.3 Hz), 131.2 (d, J_{C—F}=11.9 Hz), 131.1 (qd, J_{C—F}=32.8, 8.4 Hz), 124.6 (d, J_{C-F}=8.5 Hz), 123.7 (qd, J_{C—F}=272.6, 2.5 Hz), 122.1-121.8 (m), 114.4 (dq, J_{C—F}=26.8, 3.9 Hz), 104.0, 36.5.

¹⁹F NMR (471 MHz, DMSO-d₆) δ −61.22, −114.65-−114.73 (m).

IR (thin film, cm⁻¹): v=2324, 1602, 1503, 1424, 1303, 1225, 1186, 1125, 1072, 907, 883, 831, 745, 725, 661, 608, 562, 443.

### 3-hydroxy-5-(6-(4-(trifluoromethoxy)phenyl)pyridin-3-yl)cyclohex-2-en-1-one (121) and 5-(6-(4-(trifluoromethoxy)phenyl)pyridin-3-yl)cyclohexane-1,3-dione (121′)

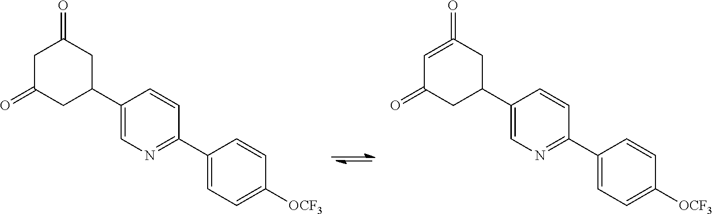

Following general procedures J and K, the title compound [exists in a tautomeric form in a 1.0:0.12 ratio (121:121′)] was prepared as an off-white and slightly yellow powder.

LCMS (ESI-MS) m/z: [M+H]⁺ calcd for C₁₈H₁₅F₃NO₃ 350.09; found 349.8. Retention time: 3.910 min.

TLC: (5% methanol in dichloromethane, R_{f}): 0.16 (UV, 254 nm, 280 nm).

Keto Form: ¹H NMR (500 MHz, DMSO-d₆) δ 8.82-8.76 (m, 1H), 8.37-8.17 (m, 4H), 7.61-7.52 (m, 2H), 3.75 (s, 2H), 3.64-3.53 (m, 1H), 2.96-2.86 (m, 2H), 2.67-2.60 (m, 2H).

Enol Form: ¹H NMR (500 MHz, DMSO-d₆) δ 8.82-8.76 (m, 1H), 8.37-8.17 (m, 4H), 7.61-7.52 (m, 2H), 5.42 (s, 1H), 3.64-3.53 (m, 1H), 2.80-2.70 (m, 2H), 2.55-2.46 (m, 2H).

Enol Form: ¹³C NMR (126 MHz, DMSO-d₆) δ 153.0, 148.8 (q, J_{C—F}=1.8 Hz), 148.7, 138.0, 137.7, 135.7, 128.3, 121.1, 120.1, 119.7 (q, J_{C—F}=256.6 Hz), 103.6, 36.0.

¹⁹F NMR (471 MHz, DMSO-d₆) δ −56.66.

### 5-(6-(3-(trifluoromethoxy)phenyl)pyridin-3-yl)cyclohexane-1,3-dione (122)

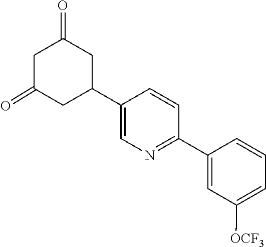 [SMILES: O=C1CC(=O)CC(c2ccc(-c3cccc(OC(F)(F)F)c3)nc2)C1]

Following general procedures J and K, the title compound was prepared as a white powder.

LCMS (ESI-MS) m/z: [M+H]⁺ calcd for C₁₈H₁₅F₃NO₃ 350.09; found 349.8. Retention time: 3.974 min.

TLC: (5% methanol in dichloromethane, R_{f}): 0.16 (UV, 254 nm, 280 nm).

¹H NMR (500 MHz, CD₃OD) δ 8.91 (d, J=2.1 Hz, 1H), 8.74 (dd, J=8.4, 2.1 Hz, 1H), 8.40 (d, J=8.4 Hz, 1H), 8.00 (d, J=7.8 Hz, 1H), 7.95 (s, 1H), 7.82 (dd, J=8.3, 7.8 Hz, 1H), 7.67 (d, J=8.3 Hz, 1H), 4.91 (s, 2H), 3.86-3.76 (m, 1H), 3.00 (dd, J=16.9, 11.3 Hz, 1H), 2.92-2.80 (m, 2H), 2.80-2.70 (m, 1H).

¹⁹F NMR (471 MHz, CD₃OD) 5-59.40.

### 3-hydroxy-5-(5-(4-(trifluoromethoxy)phenyl)pyridin-2-yl)cyclohex-2-en-1-one (123)

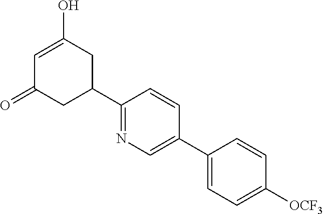 [SMILES: O=C1C=C(O)CC(c2ccc(-c3ccc(OC(F)(F)F)cc3)cn2)C1]

Following general procedures J and K, the title compound was prepared as a white powder.

LCMS (ESI-MS) m/z: [M+H]⁺ calcd for C₁₈H₁₅F₃NO₃ 350.09; found 349.8. Retention time: 3.955 min.

TLC: (5% methanol in dichloromethane, R_{f}): 0.14 (UV, 254 nm, 280 nm).

¹H NMR (500 MHz, DMSO-d₆) δ 8.89 (d, J=2.4 Hz, 1H), 8.13 (dd, J=8.2, 2.4 Hz, 1H), 7.90-7.84 (m, 2H), 7.54 (d, J=8.2 Hz, 1H), 7.50 (d, J=8.3 Hz, 2H), 5.31 (s, 1H), 3.64-3.54 (m, 1H), 2.74 (dd, J=16.7, 11.1 Hz, 2H), 2.55 (dd, J=16.8, 4.7 Hz, 2H).

¹³C NMR (126 MHz, DMSO-d₆) δ 161.2, 148.7 (q, J_{C—F}=1.6 Hz), 147.0, 136.6, 136.1, 133.1, 129.3, 122.6, 122.1, 120.5 (q, J_{C—F}=256.5 Hz), 104.0.

¹⁹F NMR (471 MHz, DMSO-d₆) δ −56.75.

IR (thin film, cm⁻¹): v=2737, 1597, 1557, 1482, 1221, 1163, 832.

### 3-hydroxy-5-(5-(3-(trifluoromethoxy)phenyl)pyridin-2-yl)cyclohex-2-en-1-one (124)

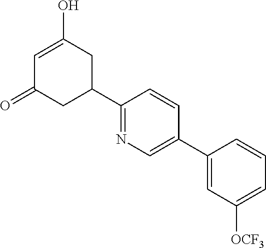 [SMILES: O=C1C=C(O)CC(c2ccc(-c3cccc(OC(F)(F)F)c3)cn2)C1]

Following general procedures J and K, the title compound was prepared as an off-white and slightly yellow powder.

LCMS (ESI-MS) m/z: [M+H]⁺ calcd for C₁₈H₁₅F₃NO₃ 350.09; found 349.8. Retention time: 3.968 min.

TLC: (5% methanol in dichloromethane, R_{f}): 0.14 (UV, 254 nm, 280 nm).

¹H NMR (500 MHz, DMSO-d₆) δ 11.35 (s, 1H), 8.90 (d, J=2.5 Hz, 1H), 8.11 (dd, J=8.1, 2.5 Hz, 1H), 7.79 (d, J=7.8 Hz, 1H), 7.74 (s, 1H), 7.65 (dd, J=8.2, 7.8 Hz, 1H), 7.50 (d, J=8.1 Hz, 1H), 7.42 (d, J=8.2 Hz, 1H), 5.33 (s, 1H), 3.64-3.50 (m, 1H), 2.73 (dd, J=16.7, 10.9 Hz, 2H), 2.55 (dd, J=16.7, 4.7 Hz, 2H).

¹³C NMR (126 MHz, DMSO-d₆) δ 162.0, 149.5 (q, J_{C—F}=1.6 Hz), 147.6, 139.8, 135.6, 132.6, 131.5, 126.4, 122.3, 120.7, 120.5 (q, J_{C—F}=256.4 Hz), 119.9, 104.0, 40.6. [1754]¹⁹F NMR (471 MHz, DMSO-d₆) δ −56.66.

IR (thin film, cm⁻¹): v=2896, 1584, 1474, 1213, 839, 791, 737, 698, 631, 450.

### 5-(5-(2-fluoro-4-(trifluoromethyl)phenyl)thiophen-2-yl)-3-hydroxycyclohex-2-en-1-one (125)

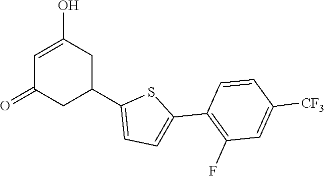 [SMILES: O=C1C=C(O)CC(c2ccc(-c3ccc(C(F)(F)F)cc3F)s2)C1]

Following general procedures J and K, the title compound was prepared as a white powder.

LCMS (ESI-MS) m/z: [M+H]⁺ calcd for C₁₇H₁₃F₄O₂S 357.05; found 357.1. Retention time: 3.527 min.

TLC: (5% methanol in dichloromethane, R_{f}): 0.20 (UV, 254 nm, 280 nm).

¹H NMR (500 MHz, DMSO-d₆) δ 11.30 (s, 1H), 7.98 (dd, J=9.1, 7.8 Hz, 1H), 7.79 (dd, J=11.5, 1.8 Hz, 1H), 7.62 (dd, J=7.8, 1.8 Hz, 1H), 7.60 (d, J=3.8 Hz, 1H), 7.10 (d, J=3.8 Hz, 1H), 5.30 (s, 1H), 3.76-3.66 (m, 1H), 2.71-2.56 (m, 4H).

¹³C NMR (126 MHz, DMSO-d₆) δ 157.9 (d, J_{C—F}=250.3 Hz) 150.5 (d, J_{C—F}=5.3 Hz), 132.3 (d, J_{C—F}=4.2 Hz), 129.6 (d, J_{C—F}=3.7 Hz), 129.1 (qd, J_{C—F}=33.1, 8.4 Hz), 128.5 (d, J_{C—F}=4.7 Hz), 125.9 (d, J_{C—F}=12.6 Hz), 125.4, 123.7 (qd, J_{C—F}=271.9, 2.5 Hz), 122.4-122.2 (m), 114.4 (dq, J_{C-F}=26.2, 3.8 Hz), 104.2, 34.5.

¹⁹F NMR (471 MHz, DMSO-d₆) δ −61.05, −112.16.

### 3-hydroxy-5-(5-(4-(trifluoromethoxy)phenyl)thiophen-2-yl)cyclohex-2-en-1-one (126)

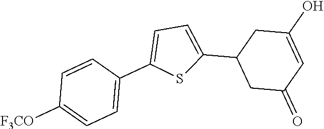 [SMILES: O=C1C=C(O)CC(c2ccc(-c3ccc(OC(F)(F)F)cc3)s2)C1]

Following general procedures J and K, the title compound was prepared as a white powder.

LCMS (ESI-MS) m/z: [M+H]⁺ calcd for C₁₇H₁₄F₃O₃S 355.05; found 354.7. Retention time: 3.554 min. [M−H]⁻ calcd for C₁₇H₁₂F₃O₃S 353.05; found 353.0. Retention time: 3.515.

TLC: (5% methanol in dichloromethane, R_{f}): 0.23 (UV, 254 nm, 280 nm).

¹H NMR (500 MHz, DMSO-d₆) δ 11.28 (s, 1H), 7.75-7.68 (m, 2H), 7.42-7.36 (m, 3H), 7.00 (d, J=3.6 Hz, 1H), 5.30 (s, 1H), 3.70-3.61 (m, 1H), 2.71-2.53 (m, 4H).

¹³C NMR (126 MHz, DMSO-d₆) δ 148.2, 147.8 (q, J_{C—F}=1.9 Hz), 139.7, 133.6, 127.3, 125.7, 124.8, 122.1, 120.5 (q, J_{C—F}=256.3 Hz), 104.2, 34.6. [1769]¹⁹F NMR (471 MHz, CD₃OD) 5-59.51.

### 3-hydroxy-5-(5-(3-(trifluoromethoxy)phenyl)thiophen-2-yl)cyclohex-2-en-1-one (127)

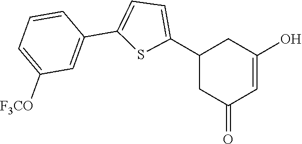 [SMILES: O=C1C=C(O)CC(c2ccc(-c3cccc(OC(F)(F)F)c3)s2)C1]

Following general procedures J and K, the title compound was prepared as a yellow solid.

LCMS (ESI-MS) m/z: [M+H]⁺ calcd for C₁₇H₁₄F₃O₃S 355.05; found 354.9. Retention time: 3.503 min. [M−H]⁻ calcd for C₁₇H₁₂F₃O₃S 353.05; found 353.0. Retention time: 3.503.

TLC: (5% methanol in dichloromethane, R_{f}): 0.23 (UV, 254 nm, 280 nm).

¹H NMR (500 MHz, DMSO-d₆) δ 11.28 (s, 1H), 7.65-7.60 (m, 1H), 7.58 (s, 1H), 7.53 (dd, J=8.6, 7.9 Hz, 1H), 7.50 (d, J=3.7 Hz, 1H), 7.31-7.25 (m, 1H), 7.01 (dd, J=3.7, 0.9 Hz, 1H), 5.30 (s, 1H), 3.71-3.61 (m, 1H), 2.74-2.53 (m, 4H).

¹³C NMR (126 MHz, DMSO-d₆) δ 149.4 (q, J_{C—F}=2.0 Hz), 148.6, 139.4, 136.5, 131.6, 125.8, 125.3, 124.6, 120.5 (q, J_{C-F}=256.5 Hz), 119.9, 117.8, 104.2, 34.6.

¹⁹F NMR (471 MHz, CD₃OD) 5-59.36.

### 3-hydroxy-5-(2-(4-(trifluoromethoxy)phenyl)thiazol-5-yl)cyclohex-2-en-1-one (128)

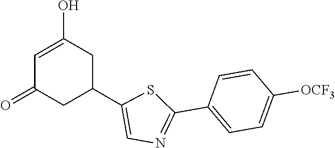 [SMILES: O=C1C=C(O)CC(c2cnc(-c3ccc(OC(F)(F)F)cc3)s2)C1]

Following general procedures J and K, the title compound was prepared as an off-white powder.

LCMS (ESI-MS) m/z: [M+H]⁺ calcd for C₁₆H₁₃F₃NO₃S 356.05; found 355.7. Retention time: 4.051 min.

TLC: (5% methanol in dichloromethane, R_{f}): 0.28 (UV, 254 nm, 280 nm).

Keto Form: ¹H NMR (500 MHz, CD₃OD) b 8.10-7.92 (m, 2H), 7.73 (d, J=0.9 Hz, 1H), 7.40 (dd, J=8.8, 1.2 Hz, 2H), 4.88 (s, 2H), 3.93-3.79 (m, 1H), 2.83 (dd, J=17.0, 4.8 Hz, 2H), 2.71 (dd, J=16.9, 10.0 Hz, 2H).

Enol Form: ¹H NMR (500 MHz, DMSO-d₆) b 8.05-7.98 (m, 2H), 7.78 (d, J=1.0 Hz, 1H), 7.48 (dd, J=9.0, 1.1 Hz, 2H), 5.31 (s, 1H), 3.83-3.73 (m, 1H), 2.68 (dd, J=16.6, 4.8 Hz, 2H), 2.59 (dd, J=16.6, 10.1 Hz, 2H).

¹³C NMR (126 MHz, DMSO-d₆) δ 164.1, 149.7 (q, J_{C—F}=2.0 Hz), 143.6, 141.0, 132.7, 128.3, 122.1, 120.4 (q, J_{C—F}=256.9 Hz), 104.1, 32.3.

¹⁹F NMR (471 MHz, CD₃OD) 5-59.42.

### 3-hydroxy-5-(2-(3-(trifluoromethoxy)phenyl)thiazol-5-yl)cyclohex-2-en-1-one (129)

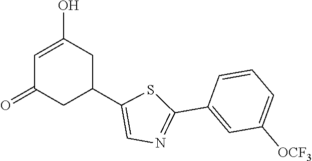 [SMILES: O=C1C=C(O)CC(c2cnc(-c3cccc(OC(F)(F)F)c3)s2)C1]

Following general procedures J and K, the title compound was prepared as a brownish-yellow powder

LCMS (ESI-MS) m/z: [M+H]⁺ calcd for C₁₆H₁₃F₃NO₃S 356.05; found 355.7. Retention time: 4.056 min.

TLC: (5% methanol in dichloromethane, R_{f}): 0.17 (UV, 254 nm, 280 nm).

Keto Form: ¹H NMR (500 MHz, CD₃OD) δ 7.89 (d, J=7.9 Hz, 1H), 7.85 (s, 1H), 7.75 (d, J=0.9 Hz, 1H), 7.59 (dd, J=8.9, 7.9 Hz, 1H), 7.42-7.35 (m, 1H), 4.88 (s, 2H), 3.92-3.81 (m, 1H), 2.84 (dd, J=17.0, 4.8 Hz, 2H), 2.72 (dd, J=16.9, 10.2 Hz, 2H).

Enol Form: ¹H NMR (500 MHz, DMSO-d₆) δ 11.35 (s, 1H), 7.92-7.87 (m, 1H), 7.83 (s, 1H), 7.81 (d, J=1.0 Hz, 1H), 7.65 (dd, J=8.8, 8.0 Hz, 1H), 7.50-7.45 (m, 1H), 5.31 (s, 1H), 3.95-3.70 (m, 1H), 2.81-2.53 (m, 4H).

¹³C NMR (126 MHz, DMSO-d₆) δ 163.8, 149.3 (q, J_{C—F}=1.7 Hz), 143.9, 141.1, 135.6, 132.0, 125.6, 122.8, 120.5 (q, J_{C—F}=256.7 Hz), 118.1, 104.2, 32.3.

¹⁹F NMR (471 MHz, CD₃OD) 5-59.46.

IR (thin film, cm⁻¹): v=2652, 1525, 1367, 1214, 1161, 832, 791, 683, 633, 448.

### 3-hydroxy-5-(2-(4-(trifluoromethoxy)phenyl)thiazol-4-yl)cyclohex-2-en-1-one (130)

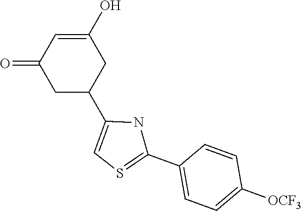

Following general procedures J and K, the title compound was prepared as a white powder.

LCMS (ESI-MS) m/z: [M+H]⁺ calcd for C₁₆H₁₃F₃NO₃S 356.05; found 355.7. Retention time: 4.210 min.

TLC: (5% methanol in dichloromethane, R_{f}): 0.17 (UV, 254 nm, 280 nm).

Keto Form: ¹H NMR (500 MHz, CD₃OD) δ 8.12-8.01 (m, 2H), 7.39 (d, J=8.2 Hz, 2H), 7.33 (d, J=0.8 Hz, 1H), 4.89 (s, 2H), 3.71-3.60 (m, 1H), 2.91-2.74 (m, 4H).

Enol Form: ¹H NMR (500 MHz, DMSO-d₆) δ 11.22 (s, 1H), 8.15-7.98 (m, 2H), 7.56-7.44 (m, 3H), 5.29 (s, 1H), 3.62-3.50 (m, 1H), 2.67 (d, J=5.1 Hz, 4H).

¹³C NMR (126 MHz, DMSO-d₆) δ 165.4, 159.8, 149.8 (q, J_{C—F}=1.9 Hz), 132.6, 128.5, 122.1, 120.4 (q, J_{C—F}=256.9 Hz), 115.6, 104.1, 35.6.

¹⁹F NMR (471 MHz, CD₃OD) 5-59.40.

IR (thin film, cm⁻¹): v=2889, 1558, 1518, 1299, 1258, 1221, 1162, 1006, 851.

### 3-hydroxy-5-(2-(3-(trifluoromethoxy)phenyl)thiazol-4-yl)cyclohex-2-en-1-one (131)

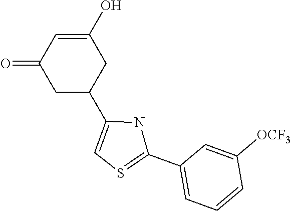

Following general procedures J and K, the title compound was prepared as a white powder.

LCMS (ESI-MS) m/z: [M+H]⁺ calcd for C₁₆H₁₃F₃NO₃S 356.05; found 355.7 Retention time: 3.856 min.

TLC: (5% methanol in dichloromethane, R_{f}): 0.31 (UV, 254 nm, 280 nm).

Keto Form: ¹H NMR (500 MHz, CD₃OD) δ 7.93 (d, J=8.0 Hz, 1H), 7.88 (s, 1H), 7.56 (dd, J=8.7, 8.0 Hz, 1H), 7.43-7.30 (m, 2H), 4.90 (s, 2H), 3.72-3.55 (m, 1H), 2.92-2.69 (m, 4H).

Enol Form: ¹H NMR (500 MHz, DMSO-d₆) δ 11.22 (s, 1H), 7.95 (d, J=7.8 Hz, 1H), 7.88 (s, 1H), 7.65 (dd, J=8.7, 7.8 Hz, 1H), 7.54 (s, 1H), 7.50 (d, J=8.7 Hz, 1H), 5.29 (s, 1H), 3.64-3.50 (m, 1H), 2.67 (d, J=7.4 Hz, 4H).

¹³C NMR (101 MHz, DMSO-d₆) δ 165.1, 159.9, 149.3 (q, J_{C—F}=2.0 Hz), 135.6, 131.9, 125.7, 122.8, 120.5 (q, J_{C—F}=256.8 Hz), 118.4, 115.9, 104.1, 35.6.

¹⁹F NMR (471 MHz, CD₃OD) 5-59.41.

### 6-chloro-7-methoxy-2H-benzo[d][1,3]oxazine-2,4(1H)-dione (133)

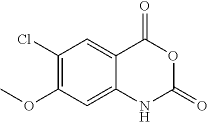

Following general procedure A, the title compound was prepared as a white powder.

LCMS (ESI) m/z [M+H]⁺ calcd for C₉H₇ClNO₄ 228.00; found 227.8. Retention time: 3.227 min.

TLC: (50% EtOAc in hexanes, R_{f}): 0.24 (UV, 254 nm, 280 nm).

¹H NMR (500 MHz, DMSO-d₆) δ 11.78 (s, 1H), 7.85 (s, 1H), 6.72 (s, 1H), 3.95 (s, 3H).

¹³C NMR (126 MHz, DMSO-d₆) δ 161.1, 158.9, 147.5, 143.0, 129.8, 117.4, 103.8, 98.7, 57.3.

### 7-chloro-3-(2,4-dichlorophenyl)-6-methoxy-3,4-dihydroacridine-1,9(2H,10H)-dione (134)

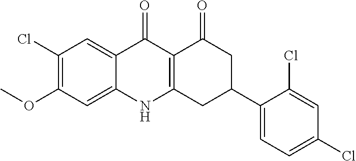 [SMILES: COc1cc2[nH]c3c(c(=O)c2cc1Cl)C(=O)CC(c1ccc(Cl)cc1Cl)C3]

Following general procedure L, the title compound was prepared as a white powder.

TLC: (5% methanol in dichloromethane, R_{f}): 0.38 (UV, 254 nm, 280 nm)

¹H NMR (500 MHz, CDCl₃ and 3 drops of TFA-d) δ 8.47 (s, 1H), 7.50 (d, J=2.1 Hz, 1H), 7.46 (s, 1H), 7.34 (dd, J=8.4, 2.1 Hz, 1H), 7.25 (d, J=8.4 Hz, 1H), 4.18-4.08 (m, 4H), 3.70-3.56 (m, 2H), 3.24-3.15 (m, 1H), 3.08 (dd, J=17.6, 12.7 Hz, 1H).

¹³C NMR (126 MHz, CDCl₃ and 3 drops of TFA-d) δ 202.0, 171.2, 163.2, 160.8, 141.0, 135.1, 134.8, 134.2, 130.4, 128.2 (2×C), 127.6, 125.7, 113.2, 107.3, 100.8, 57.7, 42.6, 34.7, 33.3.

### 7-chloro-6-methoxy-3-(thiophen-2-yl)-3,4-dihydroacridine-1,9(2H,10H)-dione (135)

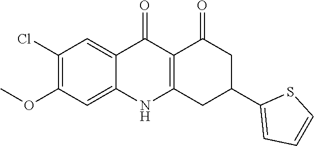 [SMILES: COc1cc2[nH]c3c(c(=O)c2cc1Cl)C(=O)CC(c1cccs1)C3]

Following general procedure L, the title compound was prepared as a yellow solid.

TLC: (5% methanol in dichloromethane, R_{f}): 0.38 (UV, 254 nm, 280 nm)

¹H NMR (500 MHz, CDCl₃ and 3 drops of TFA-d) δ 8.44 (s, 1H), 7.48 (s, 1H), 7.39 (dd, J=5.0, 2.9 Hz, 1H), 7.15-7.09 (m, 1H), 7.05 (dd, J=5.0, 1.5 Hz, 1H), 4.12 (s, 3H), 3.92-3.82 (m, 1H), 3.79 (dd, J=17.7, 3.7 Hz, 1H), 3.61 (dd, J=17.7, 9.7 Hz, 1H), 3.27 (dd, J=17.7, 3.7 Hz, 1H), 3.15 (dd, J=17.7, 9.7 Hz, 1H).

¹³C NMR (126 MHz, CDCl₃ and 3 drops of TFA-d) δ 202.5, 171.1, 163.2, 160.8, 140.9, 140.3, 128.1, 127.7, 125.8, 125.7, 121.3, 113.2, 107.5, 100.7, 57.7, 43.6, 34.9, 33.7.

### 7-chloro-6-methoxy-3-(6-(4-(trifluoromethoxy)phenyl)pyridin-3-yl)-3,4-dihydroacridine-1,9(2H,10H)-dione (136)

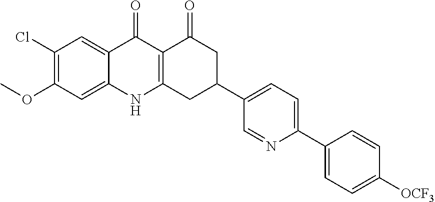 [SMILES: COc1cc2[nH]c3c(c(=O)c2cc1Cl)C(=O)CC(c1ccc(-c2ccc(OC(F)(F)F)cc2)nc1)C3]

Following general procedure L, the title compound was prepared as a brown powder.

TLC: (5% methanol in dichloromethane, R_{f}): 0.38 (UV, 254 nm, 280 nm)

¹H NMR (500 MHz, CDCl₃ and 3 drops of TFA-d) δ 9.25 (s, 1H), 8.61 (dd, J=8.3, 2.0 Hz, 1H), 8.48 (s, 1H), 8.17 (d, J=8.3 Hz, 1H), 7.93 (d, J=8.8 Hz, 2H), 7.51 (s, 1H), 7.48 (d, J=8.8 Hz, 2H), 4.16-4.03 (m, 4H), 3.96 (d, J=15.1 Hz, 1H), 3.81 (dd, J=17.5, 13.0 Hz, 1H), 3.37 (dd, J=17.5, 13.0 Hz, 1H), 3.25 (d, J=15.1 Hz, 1H).

¹³C NMR (126 MHz, CDCl₃ and 3 drops of TFA-d) δ 200.4, 171.3, 163.2, 159.7, 152.8 (q, J_{C—F}=2.1 Hz), 151.2, 145.7, 141.4, 141.0, 138.8, 129.8, 128.2, 127.6, 125.9, 125.8, 121.8, 120.8 (q, J_{C—F}=260.3 Hz), 113.3, 107.2, 100.6, 57.5, 41.9, 35.5, 33.6.

¹⁹F NMR (471 MHz, CDCl₃ and 3 drops of TFA-d) δ −58.34.

### 7-chloro-6-methoxy-3-(5-(4-(trifluoromethoxy)phenyl)pyridin-2-yl)-3,4-dihydroacridine-1,9(2H,10H)-dione (137)

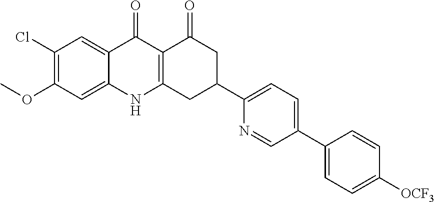 [SMILES: COc1cc2[nH]c3c(c(=O)c2cc1Cl)C(=O)CC(c1ccc(-c2ccc(OC(F)(F)F)cc2)cn1)C3]

Following general procedure L, the title compound was prepared as a brown powder.

TLC: (5% methanol in dichloromethane, R_{f}): 0.38 (UV, 254 nm, 280 nm)

¹H NMR (500 MHz, CDCl₃ and 3 drops of TFA-d) δ 9.04 (d, J=2.1 Hz, 1H), 8.66 (dd, J=8.4, 2.1 Hz, 1H), 8.47 (s, 1H), 8.15 (d, J=8.4 Hz, 1H), 7.71 (d, J=8.3 Hz, 2H), 7.54 (s, 1H), 7.44 (d, J=8.3 Hz, 2H), 4.57-4.47 (m, 1H), 4.18-4.03 (m, 5H), 3.51 (dd, J=17.3, 13.3 Hz, 1H), 3.35 (dd, J=17.3, 3.2 Hz, 1H).

¹³C NMR (126 MHz, CDCl₃ and 3 drops of TFA-d) δ 199.2, 171.2, 163.3, 158.8, 152.1, 151.3, 145.0, 140.9, 140.1, 139.5, 130.7, 128.9, 128.4, 126.3, 125.8, 122.2, 120.3 (q, J_{C-F}=259.0 Hz), 113.3, 107.3, 100.6, 57.7, 41.2, 36.4, 32.4.

¹⁹F NMR (471 MHz, CDCl₃ and 3 drops of TFA-d) δ −58.45.

### 7-chloro-6-methoxy-3-(5-(3-(trifluoromethoxy)phenyl)pyridin-2-yl)-3,4-dihydroacridine-1,9(2H,10H)-dione (138)

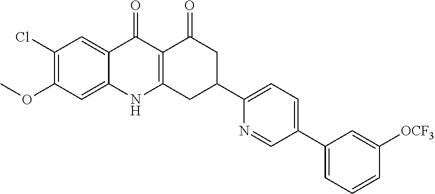 [SMILES: COc1cc2[nH]c3c(c(=O)c2cc1Cl)C(=O)CC(c1ccc(-c2cccc(OC(F)(F)F)c2)cn1)C3]

Following general procedure L, the title compound was prepared as an off-white powder.

TLC: (5% methanol in dichloromethane, R_{f}): 0.34 (UV, 254 nm, 280 nm)

¹H NMR (500 MHz, CDCl₃ and 3 drops of TFA-d) δ 9.07 (d, J=2.1 Hz, 1H), 8.67 (dd, J=8.3, 2.0 Hz, 1H), 8.47 (s, 1H), 8.17 (d, J=8.3 Hz, 1H), 7.65 (dd, J=9.0, 8.5 Hz, 1H), 7.60 (d, J=9.0 Hz, 1H), 7.55 (s, 1H), 7.51 (s, 1H), 7.45 (d, J=8.5 Hz, 1H), 4.59-4.48 (m, 1H), 4.18-4.02 (m, 5H), 3.59-3.46 (m, 1H), 3.40-3.29 (m, 1H).

¹³C NMR (126 MHz, CDCl₃ and 3 drops of TFA-d) δ 199.3, 171.2, 163.2, 158.9, 152.6, 150.3 (q, J_{C—F}=1.8 Hz), 145.1, 141.0, 140.4, 139.2, 134.3, 131.7, 128.3, 126.3, 125.8, 125.5, 123.2, 120.3 (q, J_{C—F}=258.6 Hz), 119.8, 113.4, 107.3 (q, J_{C—F}=2.0 Hz), 100.7, 57.6, 41.1, 36.4, 32.4.

¹⁹F NMR (471 MHz, CDCl₃ and 3 drops of TFA-d) δ −58.68.

### 7-chloro-6-methoxy-3-(6-(4-(trifluoromethoxy)phenoxy)pyridin-3-yl)-3,4-dihydroacridine-1,9(2H,10H)-dione (139)

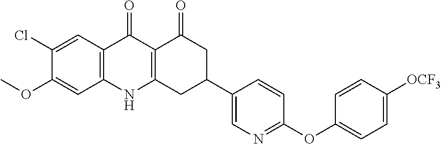 [SMILES: COc1cc2[nH]c3c(c(=O)c2cc1Cl)C(=O)CC(c1ccc(Oc2ccc(OC(F)(F)F)cc2)nc1)C3]

Following general procedure L, the title compound was prepared as an off-white peach-colored powder.

TLC: (5% methanol in dichloromethane, R_{f}): 0.40 (UV, 254 nm, 280 nm)

¹H NMR (500 MHz, CDCl₃ and 3 drops of TFA-d) δ 8.69 (s, 1H), 8.46 (s, 1H), 8.24 (dd, J=9.0, 2.4 Hz, 1H), 7.48 (s, 1H), 7.37 (d, J=8.6 Hz, 2H), 7.25 (d, J=8.6 Hz, 2H), 7.06 (d, J=9.0 Hz, 1H), 4.12 (s, 3H), 3.95-3.80 (m, 2H), 3.67 (dd, J=17.5, 12.0 Hz, 1H), 3.30-3.15 (m, 2H).

¹³C NMR (126 MHz, CDCl₃ and 3 drops of TFA-d) δ 171.2, 163.2, 160.3, 159.8, 149.6, 147.9, 146.0, 141.0, 140.3, 132.6, 128.3, 125.8, 123.5, 122.2, 120.3 (q, J_{C—F}=258.3 Hz), 113.2, 112.4, 107.2, 100.7, 57.6, 42.3, 35.1, 35.0, 34.0.

¹⁹F NMR (471 MHz, CDCl₃ and 3 drops of TFA-d) δ −58.79.

### 7-chloro-6-methoxy-3-(5-(4-(trifluoromethoxy)phenyl)thiophen-2-yl)-3,4-dihydroacridine-1,9(2H,10H)-dione (140)

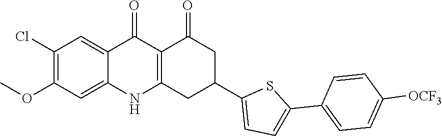 [SMILES: COc1cc2[nH]c3c(c(=O)c2cc1Cl)C(=O)CC(c1ccc(-c2ccc(OC(F)(F)F)cc2)s1)C3]

Following general procedure L, the title compound was prepared as a tan-colored solid.

TLC: (5% methanol in dichloromethane, R_{f}): 0.36 (UV, 254 nm, 280 nm)

¹H NMR (500 MHz, CDCl₃ and 3 drops of TFA-d) δ 8.45 (s, 1H), 7.54 (d, J=8.7 Hz, 2H), 7.46 (s, 1H), 7.22 (d, J=8.7 Hz, 2H), 7.13 (d, J=3.4 Hz, 1H), 6.92 (d, J=3.4 Hz, 1H), 4.13 (s, 3H), 4.10-4.02 (m, 1H), 3.87 (dd, J=17.8, 4.3 Hz, 1H), 3.69 (dd, =17.8, 8.9 Hz, 1H), 3.35 (dd, J=17.8, 4.3 Hz, 1H), 3.24 (dd, J=17.8, 8.9 Hz, 1H).

¹³C NMR (126 MHz, CDCl₃ and 3 drops of TFA-d) δ 201.6, 171.1, 163.3, 160.2, 148.8 (q, J_{C—F}=1.6 Hz), 142.7, 142.5, 140.9, 132.4, 128.4, 127.1, 126.1, 125.8, 123.8, 121.5, 119.8 (q, J_{C—F}=260.0 Hz), 113.2, 107.5, 100.6, 57.7, 44.2, 35.6, 33.9.

¹⁹F NMR (471 MHz, CDCl₃ and 3 drops of TFA-d) δ −58.64.

### 7-chloro-6-methoxy-3-(5-(3-(trifluoromethoxy)phenyl)thiophen-2-yl)-3,4-dihydroacridine-1,9(2H,10H)-dione (141)

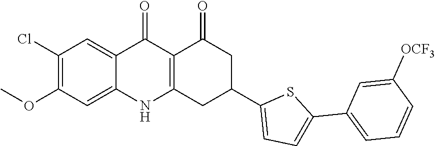 [SMILES: COc1cc2[nH]c3c(c(=O)c2cc1Cl)C(=O)CC(c1ccc(-c2cccc(OC(F)(F)F)c2)s1)C3]

Following general procedure L, the title compound was prepared as a brown solid.

TLC: (5% methanol in dichloromethane, R_{f}): 0.36 (UV, 254 nm, 280 nm)

¹H NMR (500 MHz, CDCl₃ and 3 drops of TFA-d) δ 8.45 (s, 1H), 7.50 (s, 1H), 7.45 (d, J=7.9 Hz, 1H), 7.42-7.33 (m, 2H), 7.18 (d, J=3.6 Hz, 1H), 7.14 (d, J=8.0 Hz, 1H), 6.93 (d, J=3.7 Hz, 1H), 4.13 (s, 3H), 4.10-4.02 (m, 1H), 3.89 (dd, J=17.8, 4.3 Hz, 1H), 3.70 (dd, J=17.8, 9.0 Hz, 1H), 3.35 (dd, J=17.8, 4.3 Hz, 1H), 3.23 (dd, J=17.8, 9.0 Hz, 1H).

¹³C NMR (126 MHz, CDCl₃ and 3 drops of TFA-d) δ 201.5, 171.0, 163.1, 160.1, 149.7 (q, J_{C—F}=1.4 Hz), 143.0, 142.3, 141.0, 135.5, 130.4, 128.1, 126.0, 125.7, 124.15, 124.12, 120.4 (q, J_{C—F}=257.5 Hz), 120.0, 118.2, 113.2, 107.4, 100.8, 57.7, 44.2, 35.4, 33.9.

¹⁹F NMR (471 MHz, CDCl₃ and 3 drops of TFA-d) δ −57.84.

### 7-chloro-3-(5-(2-fluoro-4-(trifluoromethyl)phenyl)thiophen-2-yl)-6-methoxy-3,4-dihydroacridine-1,9(2H,10H)-dione (142)

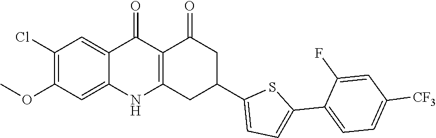 [SMILES: COc1cc2[nH]c3c(c(=O)c2cc1Cl)C(=O)CC(c1ccc(-c2ccc(C(F)(F)F)cc2F)s1)C3]

Following general procedure L, the title compound was prepared as a light brown solid.

TLC: (5% methanol in dichloromethane, R_{f}): 0.36 (UV, 254 nm, 280 nm)

¹H NMR (500 MHz, CDCl₃ and 3 drops of TFA-d) δ 8.45 (s, 1H), 7.68 (dd, J=8.6, 8.0 Hz, 1H), 7.49 (s, 1H), 7.45-7.36 (m, 3H), 6.99 (d, J=3.8 Hz, 1H), 4.16-4.02 (m, 4H), 3.90 (dd, J=17.8, 4.3 Hz, 1H), 3.71 (dd, J=17.8, 9.2 Hz, 1H), 3.37 (dd, J=17.8, 4.3 Hz, 1H), 3.25 (dd, J=17.8, 9.2 Hz, 1H).

¹³C NMR (126 MHz, CDCl₃ and 3 drops of TFA-d) δ 201.4, 171.0, 163.2, 160.2 (q, J_{C—F}=42.4 Hz), 160.0, 158.4 (d, J_{C—F}=252.5 Hz), 144.3 (d, J_{C—F}=4.3 Hz), 141.0, 135.3 (d, J_{C—F}=3.8 Hz), 128.9 (d, J_{C—F}=3.6 Hz), 128.3, 127.8 (d, J_{C—F}=6.7 Hz), 125.8 (d, J_{C—F}=5.8 Hz), 125.0 (d, J_{C—F}=12.8 Hz), 123.1 (qd, J_{C—F}=271.2, 4.6 Hz), 121.5-121.3 (m), 114.5 (q, J_{C—F}=285.7 Hz), 113.9 (qd, J_{C—F}=25.8, 4.1 Hz), 113.2, 107.4, 100.7, 57.6, 44.1, 35.4, 33.8.

¹⁹F NMR (471 MHz, CDCl₃ and 3 drops of TFA-d) δ −63.60.

### 7-chloro-6-methoxy-3-(2-(4-(trifluoromethoxy)phenoxy)thiazol-4-yl)-3,4-dihydroacridine-1,9(2H,10H)-dione (143)

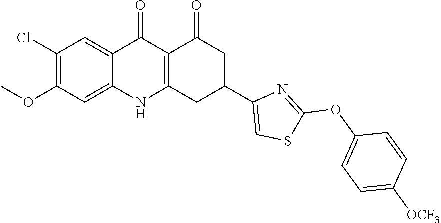 [SMILES: COc1cc2[nH]c3c(c(=O)c2cc1Cl)C(=O)CC(c1csc(Oc2ccc(OC(F)(F)F)cc2)n1)C3]

Following general procedure L, the title compound was prepared as a tan-colored powder.

TLC: (5% methanol in dichloromethane, R_{f}): 0.34 (UV, 254 nm, 280 nm)

¹H NMR (500 MHz, CDCl₃ and 3 drops of TFA-d) δ 8.45 (s, 1H), 7.42 (s, 1H), 7.30 (s, 4H), 6.85 (s, 1H), 4.13 (s, 3H), 3.98-3.89 (m, 1H), 3.80 (dd, J=17.8, 4.5 Hz, 1H), 3.69 (dd, J=17.7, 10.5 Hz, 1H), 3.26 (dd, J=17.8, 4.5 Hz, 1H), 3.18 (dd, J=17.7, 10.5 Hz, 1H).

¹³C NMR (126 MHz, CDCl₃ and 3 drops of TFA-d) δ 201.1, 177.4, 171.0, 163.3, 160.0, 152.7, 148.0, 146.6, 140.8, 128.3, 125.9, 123.1, 121.7, 120.3 (q, J_{C-F}=258.4 Hz), 113.3, 109.0, 107.6, 100.5, 57.6, 41.7, 34.1, 32.9.

¹⁹F NMR (471 MHz, CDCl₃ and 3 drops of TFA-d) δ −58.88.

### 7-chloro-6-methoxy-3-(2-(3-(trifluoromethoxy)phenoxy)thiazol-4-yl)-3,4-dihydroacridine-1,9(2H,10H)-dione (144)

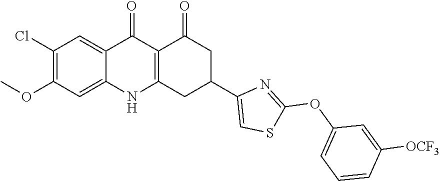 [SMILES: COc1cc2[nH]c3c(c(=O)c2cc1Cl)C(=O)CC(c1csc(Oc2cccc(OC(F)(F)F)c2)n1)C3]

Following general procedure L, the title compound was prepared as a tan-colored solid.

TLC: (5% methanol in dichloromethane, R_{f}): 0.34 (UV, 254 nm, 280 nm)

¹H NMR (500 MHz, CDCl₃ and 3 drops of TFA-d) δ 8.42 (s, 1H), 7.48-7.37 (m, 2H), 7.21-7.12 (m, 2H), 7.10 (s, 1H), 6.76 (s, 1H), 4.12 (s, 3H), 3.90-3.81 (m, 1H), 3.76 (dd, J=17.9, 4.7 Hz, 1H), 3.64 (dd, J=17.9, 9.3 Hz, 1H), 3.22 (dd, J=17.9, 4.7 Hz, 1H), 3.15 (dd, J=17.9, 9.3 Hz, 1H).

¹³C NMR (126 MHz, CDCl₃ and 3 drops of TFA-d) δ 201.8, 174.7, 170.8, 162.9, 160.4, 155.2, 149.8 (q, J_{C—F}=1.7 Hz), 148.5, 140.9, 130.9, 127.9, 125.7, 120.2 (q, J_{C—F}=258.4 Hz), 118.8, 118.5, 113.4, 113.2, 109.1, 107.7, 100.7, 57.6, 42.0, 34.6, 33.1. [1880]¹⁹F NMR (471 MHz, CDCl₃ and 3 drops of TFA-d) δ −58.81.

### 7-chloro-6-methoxy-3-(2-(4-(trifluoromethoxy)phenyl)thiazol-4-yl)-3,4-dihydroacridine-1,9(2H,10H)-dione (145)

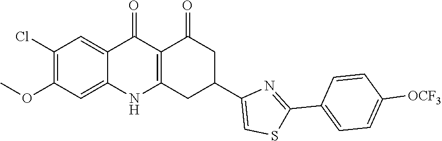 [SMILES: COc1cc2[nH]c3c(c(=O)c2cc1Cl)C(=O)CC(c1csc(-c2ccc(OC(F)(F)F)cc2)n1)C3]

Following general procedure L, the title compound was prepared as a faint yellow solid.

TLC: (5% methanol in dichloromethane, R_{f}): 0.40 (UV, 254 nm, 280 nm)

¹H NMR (500 MHz, CDCl₃ and 3 drops of TFA-d) δ 8.48 (s, 1H), 7.98 (d, J=8.9 Hz, 2H), 7.77 (s, 1H), 7.44 (d, J=8.9 Hz, 2H), 7.40 (s, 1H), 4.36-4.26 (m, 1H), 4.13 (s, 3H), 3.95 (dd, J=17.6, 4.4 Hz, 1H), 3.86 (dd, J=17.6, 11.2 Hz, 1H), 3.49-3.20 (m, 2H).

¹³C NMR (126 MHz, CDCl₃ and 3 drops of TFA-d) δ 201.1, 171.1, 170.0, 163.1, 159.9, 152.9, 152.1, 141.0, 129.0, 128.2, 125.8, 121.5, 120.3 (q, J_{C—F}=259.2 Hz), 116.8, 113.3, 107.6, 100.6, 57.6, 41.9, 33.6, 33.2, 20.6.

¹⁹F NMR (471 MHz, CDCl₃ and 3 drops of TFA-d) δ −58.45.

### 7-chloro-6-methoxy-3-(2-(3-(trifluoromethoxy)phenyl)thiazol-4-yl)-3,4-dihydroacridine-1,9(2H,10H)-dione (146)

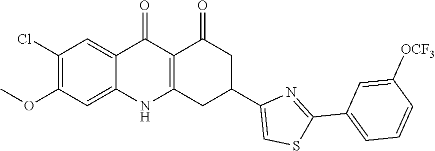 [SMILES: COc1cc2[nH]c3c(c(=O)c2cc1Cl)C(=O)CC(c1csc(-c2cccc(OC(F)(F)F)c2)n1)C3]

Following general procedure L, the title compound was prepared as a faint yellow solid.

TLC: (5% methanol in dichloromethane, R_{f}): 0.36 (UV, 254 nm, 280 nm)

¹H NMR (500 MHz, CDCl₃ and 3 drops of TFA-d) δ 8.47 (s, 1H), 7.98 (d, J=7.5 Hz, 1H), 7.85 (s, 1H), 7.82 (s, 1H), 7.66 (dd, J=8.0, 7.5 Hz, 1H), 7.58-7.50 (m, 2H), 4.45-4.36 (m, 1H), 4.17-4.06 (m, 4H), 4.01 (dd, J=17.2, 11.5 Hz, 1H), 3.50-3.36 (m, 2H).

¹³C NMR (126 MHz, CDCl₃ and 3 drops of TFA-d) δ 200.1, 171.2, 163.3, 159.3, 150.1, 140.8, 131.9, 128.4, 128.1, 128.0, 126.46, 126.43, 126.2, 125.9, 120.3 (q, J_{C—F}=259.6 Hz), 120.2, 118.6, 113.3, 107.5, 100.5, 57.6, 41.5, 32.8 (2×C).

¹⁹F NMR (471 MHz, CDCl₃ and 3 drops of TFA-d) δ −58.85.

### Single-Dose Murine PK Profile of all Tested Substances

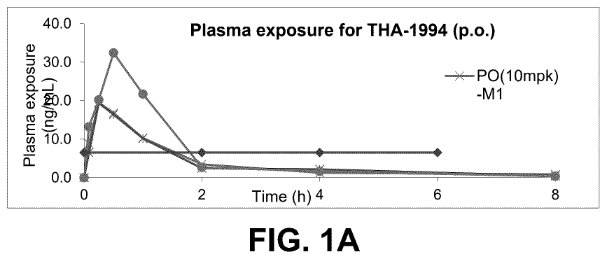

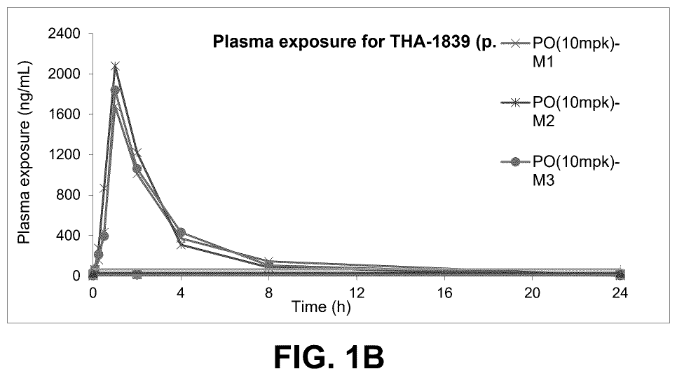

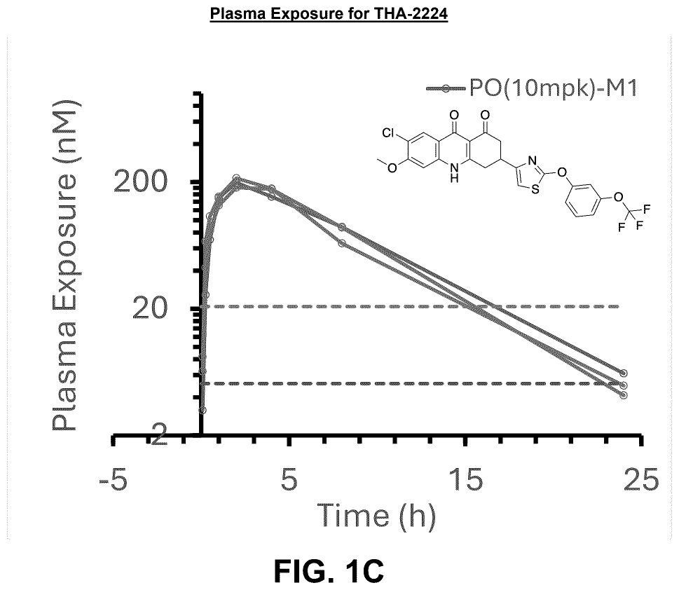

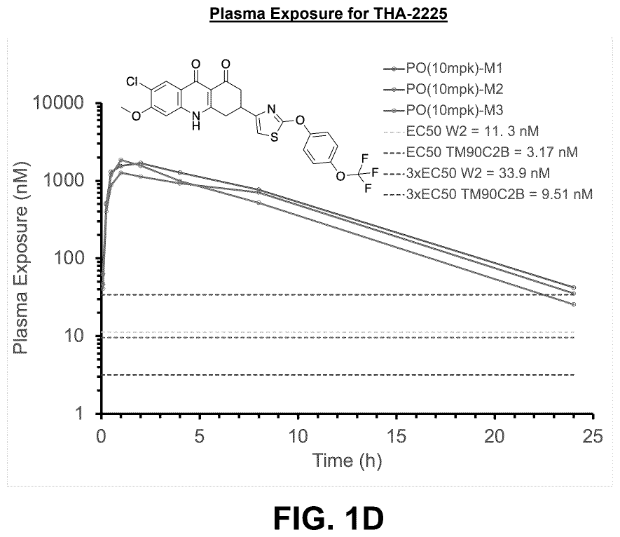

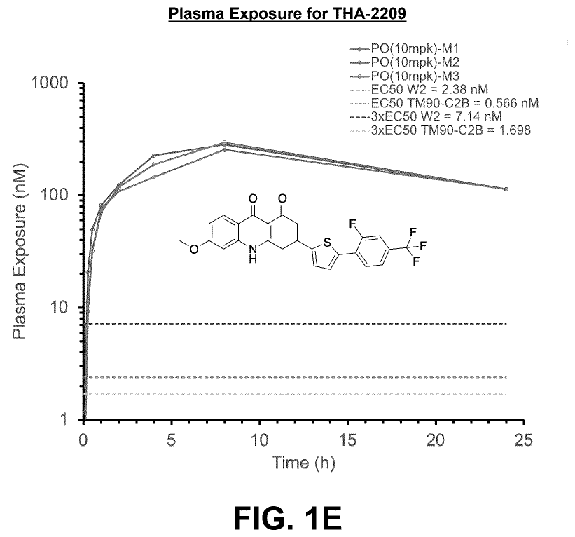

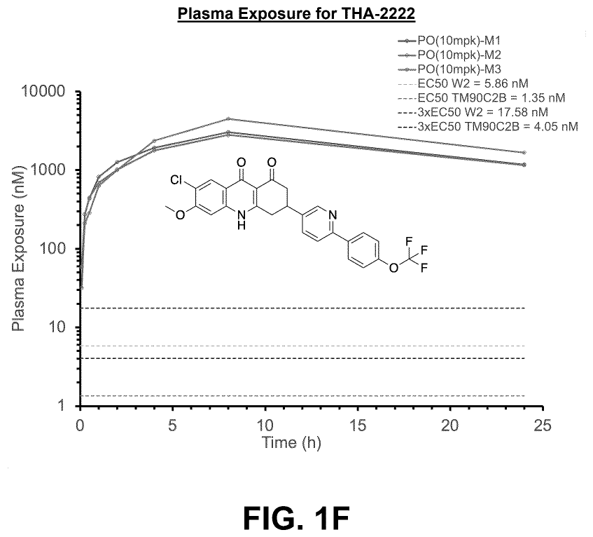

Pharmacokinetic studies were conducted at TCG LifeScience (TCGLS), Kolkata, India, in an AAALAC-accredited facility operating under CCSEA and NRC (2011) guidelines. Animals were housed in IVC racks under a 12 h light/dark cycle, 23±3° C., and 50±20% RH, with food and water provided ad libitum. All procedures were approved by the Institutional Animal Ethics Committee (IAEC). Male CD-1 mice (6-8 weeks) were acclimatized for 5-7 days and monitored throughout the studies to confirm good health. Plasma pharmacokinetics were evaluated using single-compound dosing 10 or 30 mg/kg oral gavage. Blood (−40 μL) was collected from the saphenous vein of conscious, restrained animals at 5, 15, and 30 min, and 1, 2, 4, 8, and 24 h (n=3 mice per study). Samples were drawn into K₂EDTA capillary tubes, transferred to microcentrifuge tubes, and centrifuged at 4000 rpm for 10 min at 4° C. within 30 min of collection. Plasma was stored at −20° C. until analysis. Compound concentrations were quantified by LC-MS/MS (AP6500 or API4000 with Shimadzu LC and CTC-PAL autosampler; YMC Triart C18, 2.0×30 mm). Samples were prepared by protein precipitation (70/30 acetonitrile/methanol, 1:6) followed by aqueous dilution (1:1). Results are provided in FIGS. 1A-1F.

### Biological Data of Example Substances

### Experimental

Biological assay data was gathered against the asexual blood stage of the W2 (PMID: 3284758) and TM90-C2B (PMID: 7729473) strains of Plasmodium falciparum as well as the liver stage of Plasmodium berghei (PMID: 22990796). All compounds were counter-screened for cytotoxicity using the human liver cell line HepG2 (RRID: CVCL_0027) cultured in galactose media to circumvent the Crabtree effect (PMID: 17361016). Following synthesis, powder of each test compound was diluted to 10 mM in DMSO and put into a 12-point, 1:3 serial dilution in a source 384-well microtiter plate, leading to a dose response ranging from 10 mM to 52 nM. Potency against blood stage parasites was determined by seeding 384-well microtiter plates with 40 μL per well of culture media and human blood infected with ring stage parasites at 2% parasitemia. Plates were then treated using by transferring 40 nL of compound from the source plate into the assay plate using a pin tool. After 72 hrs, plates were fixed, stained, and parasite growth quantified via high-content imaging using the methods previously described (PMID: 33792305). Potency against liver stage parasites was determined by seeding 17,500 HepG2 cells in 40 μL per well of collagen-coated 384-well microtiter plates and, after 24 hrs, infecting with at least 2,000 P. berghei sporozoites obtained using methods previously described (PMID: 36100902). After 3 hrs, plates were treated with a source plate using a pin tool as described above. After another 48 hrs, plates were fixed, immunofluorescence-stained with the parasite-specific GAPDH antibody (PMID: 6350871), and liver stage parasite growth quantified via high-content imaging using the methods previously described (PMID: 22096101). Cytotoxicity was determined by seeding 2,000 HepG2 cells in 40 μL per well of collagen-coated 384-well microtiter plates and, after 24 hrs, treating with a source plate using a pin tool as described above. After 72 hrs, plates were fixed, stained, and HepG2 growth quantified via high-content imaging using the methods previously described (PMID: 33792305). Raw data from all assays were normalized to the positive controls (dihydroartemisinin for P. falciparum blood stage assays, MMV390048 for P. berghei liver stage assays, and puromycin for HepG2 cytotoxicity assays) and the negative control (DMSO alone) and IC₅₀'s calculated using the curve fit software in CDD Vault.

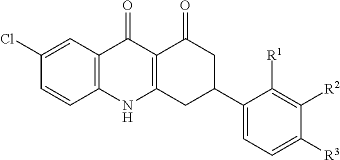

| | | | | W2 | TM90- | | RI | | | |
|---|---|---|---|---|---|---|---|---|---|---|
| | | | | EC₅₀ | C2B EC₅₀ | | (TM90/ | RL v9 | PbLS | HepG2DMEMGal |
| Cpd | R¹ | R² | R³ | (μM) | (μM) | LogP | W2) | (μM) | (μM) | (μM) |
| A-1829/ | —Cl | —H | —Cl | 0.0564 | 0.0909 | 4.89 | 1.61 | 0.656 | 0.0215 | >10 |
| R243246 | | | | | | | | | | |
| HA-1830 | —Cl | —H | —H | 0.1371 | 0.1182 | 4.33 | 0.862 | 0.592 | 0.0148 | >10 |
| THA-1831 | —H | —H | —Cl | 0.0261 | 0.0605 | 4.33 | 2.32 | N/A | N/A | >3.89 |
| THA-1839 | —OMe | —H | —OMe | 0.0145 | 0.0334 | 3.52 | 2.30 | 0.0305 | 0.000493 | >10 |
| THA-1838 | —OMe | —H | —H | 0.140 | 0.0850 | 3.72 | 0.607 | N/A | N/A | >10 |
| THA-1832 | —H | —H | —OMe | 0.0149 | 0.116 | 3.65 | 7.78 | N/A | N/A | >7.00 |
| THA-1840 | —Me | —H | —Me | 0.0107 | 0.0421 | 4.58 | 3.93 | N/A | N/A | >9.74 |
| THA-1837 | —Me | —H | —H | 0.132 | 0.210 | 4.15 | 1.59 | 0.363 | 0.0192 | >10 |
| THA-1833 | —H | —H | —Me | 0.00474 | 0.0659 | 4.15 | 13.9 | N/A | N/A | >10 |
| THA-1834 | —H | —Cl | —H | 0.0983 | 0.0493 | 4.33 | 0.502 | N/A | N/A | >3.36 |
| THA-1835 | —H | —OMe | —H | 0.0496 | 0.0785 | 3.65 | 1.58 | 0.263 | 0.0367 | >10 |
| THA-1836 | —H | —Me | —H | 0.0536 | 0.0378 | 4.15 | 0.705 | 0.270 | 0.0381 | >9.57 |
| THA-1948 | —H | —H | —H | 0.129 | 0.368 | 3.72 | 2.85 | N/A | N/A | >10 |

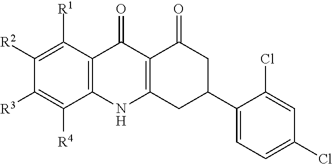

| | | | | | | TM90- | | | | | |
|---|---|---|---|---|---|---|---|---|---|---|---|
| | | | | | W2 | C2B | | RI | | | |
| | | | | | EC₅₀ | EC₅₀ | | (TM90/ | RL v9 | PbLS | HepG2DMEMGal |
| Cpd | R¹ | R² | R³ | R⁴ | (μM) | (μM) | cLogP | W2) | (μM) | (μM) | (μM) |
| THA-1842 | —H | —H | —H | —H | 0.0147 | 0.0178 | 4.33 | 1.21 | 0.0629 | 0.00391 | >3.33 |
| THA-1844 | —Cl | —H | —H | —H | 0.00843 | 0.162 | 4.89 | 19.2 | N/A | N/A | 3.34 |
| THA-1845 | —H | —H | —Cl | —H | 0.326 | 0.476 | 4.89 | 1.46 | >10 | 0.0758 | >10 |
| THA-1846 | —H | —H | —H | —Cl | 0.878 | 2.784 | 4.89 | 3.17 | N/A | N/A | >3.33 |
| THA-1852 | —OMe | —H | —H | —H | 1.67 | 3.81 | 4.25 | 2.28 | N/A | N/A | >10 |
| THA-1843 | —H | —OMe | —H | —H | 0.0354 | 0.803 | 4.25 | 22.7 | N/A | N/A | >10 |
| THA-1847 | —H | —H | —OMe | —H | 0.0267 | 0.0112 | 4.25 | 0.419 | N/A | 0.00217 | >1.11 |
| THA-1848 | —H | —H | —H | —OMe | 0.00420 | 0.0287 | 4.25 | 6.83 | N/A | N/A | 2.06 |
| THA-1851 | —Me | —H | —H | —H | 0.00391 | 0.0376 | 4.76 | 9.62 | N/A | N/A | >10 |
| THA-1841 | —H | —Me | —H | —H | 0.00962 | 0.0690 | 4.76 | 7.17 | N/A | N/A | 2.63 |
| THA-1849 | —H | —H | —Me | —H | 0.0287 | 0.115 | 4.76 | 4.01 | N/A | N/A | >10 |
| THA-1850 | —H | —H | —H | —Me | 0.0121 | 0.0433 | 4.76 | 3.58 | N/A | N/A | >3.33 |
| THA-2105 | —H | —I | —H | —H | 0.596 | 0.521 | 4.97 | 0.874 | N/A | N/A | >10 |
| THA-2104 | —H | —Br | —H | —H | 0.0587 | 0.0765 | 4.93 | 1.30 | N/A | N/A | >10 |
| THA-2102 | —H | —F | —H | —H | 0.00470 | 0.0176 | 4.44 | 3.74 | N/A | N/A | >10 |
| THA-2200 | —H | —Cl | —OMe | —H | 0.00350 | 0.00134 | 4.80 | 0.383 | N/A | N/A | >10 |

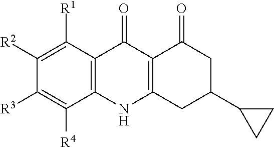

| | | | | | | TM90- | | | | | |
|---|---|---|---|---|---|---|---|---|---|---|---|
| | | | | | W2 | C2B | | RI | | | |
| | | | | | EC₅₀ | EC₅₀ | | (TM90/ | RL v9 | PbLS | HepG2DMEMGal |
| Cpd | R¹ | R² | R³ | R⁴ | (μM) | (μM) | cLogP | W2) | (μM) | (μM) | (μM) |
| THA-1950 | —H | —H | —H | —H | 1.288 | >1.01 | 2.42 | N/A | N/A | N/A | >10 |
| THA-1999 | —Cl | —H | —H | —H | 2.39 | 4.704 | 3.05 | 1.97 | N/A | N/A | >10 |
| THA-1985 | —H | —Cl | —H | —H | >3.88 | >5.72 | 3.05 | N/A | N/A | N/A | >10 |
| THA-1988 | —H | —H | —Cl | —H | >5.26 | >7.17 | 3.05 | N/A | N/A | N/A | >10 |
| THA-1989 | —H | —H | —H | —Cl | >3.35 | >10 | 3.05 | N/A | N/A | N/A | >10 |
| THA-1993 | —OMe | —H | —H | —H | >10 | >10 | 2.35 | N/A | N/A | N/A | >10 |
| THA-1949 | —H | —OMe | —H | —H | 6.806 | >10 | 2.35 | N/A | N/A | N/A | >10 |
| THA-1951 | —H | —H | —OMe | —H | 1.30 | 0.657 | 2.35 | 0.505 | N/A | N/A | >10 |
| THA-1992 | —H | —H | —H | —OMe | >1.65 | >8.46 | 2.35 | N/A | N/A | N/A | >10 |
| THA-1990 | —Me | —H | —H | —H | 0.678 | 2.20 | 2.87 | 3.24 | N/A | N/A | >10 |
| THA-1986 | —H | —Me | —H | —H | 0.908 | >2.03 | 2.87 | N/A | N/A | N/A | >10 |
| THA-1987 | —H | —H | —Me | —H | 1.49 | >6.62 | 2.87 | N/A | N/A | N/A | >10 |
| THA-1991 | —H | —H | —H | —Me | 0.272 | >3.35 | 2.87 | N/A | N/A | N/A | >10 |

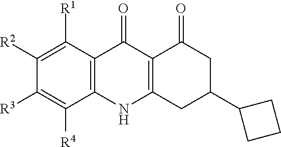

| | | | | | | TM90- | | | | | |
|---|---|---|---|---|---|---|---|---|---|---|---|
| | | | | | W2 | C2B | | RI | | | |
| | | | | | EC₅₀ | EC₅₀ | | (TM90/ | RL v9 | PbLS | HepG2DMEM |
| Cpd | R¹ | R² | R³ | R⁴ | (μM) | (μM) | cLogP | W2) | (μM) | (μM) | Gal (μM) |
| THA- | —H | —H | —H | —H | 0.113 | >3.10 | 2.77 | N/A | N/A | N/A | >10 |
| 2015 | | | | | | | | | | | |
| THA- | —Me | —H | —H | —H | 0.346 | >7.59 | 3.22 | N/A | N/A | N/A | >10 |
| 2004 | | | | | | | | | | | |
| THA- | —H | —Me | —H | —H | 0.134 | >10 | 3.22 | N/A | N/A | N/A | >10 |
| 2007 | | | | | | | | | | | |
| THA- | —H | —H | —Me | —H | 0.656 | >10 | 3.22 | N/A | N/A | N/A | >10 |
| 2006 | | | | | | | | | | | |
| THA- | —H | —H | —H | —Me | 0.129 | 2.96 | 3.22 | 22.9 | N/A | N/A | >10 |
| 2005 | | | | | | | | | | | |
| THA- | —H | —Cl | —H | —H | 0.544 | >9.80 | 3.40 | N/A | N/A | N/A | >10 |
| 2001 | | | | | | | | | | | |
| THA- | —H | —H | —OMe | —H | 0.997 | 2.07 | 2.70 | 2.08 | N/A | N/A | >10 |
| 2003 | | | | | | | | | | | |

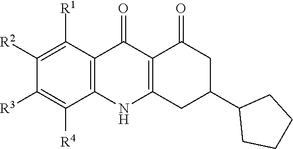

| | | | | | | TM90- | | | | | |
|---|---|---|---|---|---|---|---|---|---|---|---|
| | | | | | W2 | C2B | | RI | | | |
| | | | | | EC₅₀ | EC₅₀ | | (TM90/ | RL v9 | PbLS | HepG2DMEM |
| Cpd | R¹ | R² | R³ | R⁴ | (μM) | (μM) | cLogP | W2) | (μM) | (μM) | Gal (μM) |
| THA- | —H | —H | —H | —H | 0.0124 | >1.98 | 3.13 | N/A | N/A | N/A | >10 |
| 2020 | | | | | | | | | | | |
| THA- | —Me | —H | —H | —H | 0.0554 | 0.792 | 3.58 | 14.3 | N/A | N/A | >10 |
| 2034 | | | | | | | | | | | |
| THA- | —H | —Me | —H | —H | 0.0164 | >5.85 | 3.58 | N/A | N/A | N/A | >10 |
| 2013 | | | | | | | | | | | |
| THA- | —H | —H | —Me | —H | 0.0576 | >4.92 | 3.58 | N/A | N/A | N/A | >10 |
| 2012 | | | | | | | | | | | |
| THA- | —H | —H | —H | —Me | 0.0106 | 0.698 | 3.58 | 65.8 | N/A | N/A | >6.17 |
| 2016 | | | | | | | | | | | |
| THA- | —H | —Cl | —H | —H | 0.0254 | >10 | 3.75 | N/A | N/A | N/A | >10 |
| 2000 | | | | | | | | | | | |
| THA- | —H | —H | —OMe | —H | 0.506 | 0.939 | 1.86 | 1.86 | N/A | N/A | >10 |
| 2002 | | | | | | | | | | | |

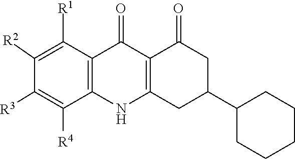

| | | | | | | TM90- | | | | | |
|---|---|---|---|---|---|---|---|---|---|---|---|
| | | | | | W2 | C2B | | RI | | | |
| | | | | | EC₅₀ | EC₅₀ | | (TM90/ | RL v9 | PbLS | HepG2DMEM |
| Cpd | R¹ | R² | R³ | R⁴ | (μM) | (μM) | cLogP | W2) | (μM) | (μM) | Gal (μM) |
| THA- | —H | —Me | —H | —H | 0.00581 | 0.412 | 4.13 | 70.9 | N/A | N/A | >10 |
| 2031 | | | | | | | | | | | |
| THA- | —H | —H | —Me | —H | 0.0187 | 1.44 | 4.13 | 77.1 | N/A | N/A | >5.77 |
| 2018 | | | | | | | | | | | |
| THA- | —H | —H | —H | —Me | 0.00248 | 0.178 | 4.13 | 71.8 | N/A | N/A | >5.77 |
| 2019 | | | | | | | | | | | |
| THA- | —H | —Cl | —H | —H | 0.000536 | >4.82 | 4.28 | N/A | N/A | 0.00172 | >10 |
| 2017 | | | | | | | | | | | |
| THA- | —H | —H | —OMe | —H | 0.0205 | 0.0201 | 3.61 | 0.979 | 0.201 | 0.170 | >6.94 |
| 1994 | | | | | | | | | | | |

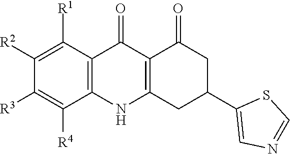

| | | | | | | TM90- | | | | | |
|---|---|---|---|---|---|---|---|---|---|---|---|
| | | | | | W2 | C2B | | RI | | | |
| | | | | | EC₅₀ | EC₅₀ | | (TM90/ | RL v9 | PbLS | HepG2DMEM |
| Cpd | R¹ | R² | R³ | R⁴ | (μM) | (μM) | cLogP | W2) | (μM) | (μM) | Gal (μM) |
| THA- | —H | —Cl | —H | —H | >10 | >10 | 3.00 | N/A | N/A | N/A | 3.04 |
| 2036 | | | | | | | | | | | |
| THA- | —H | —H | —OMe | —H | 0.583 | 1.61 | 2.41 | 2.76 | N/A | N/A | >10 |
| 2032 | | | | | | | | | | | |

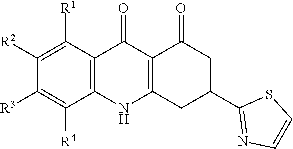

| | | | | | | TM90- | | | | | |
|---|---|---|---|---|---|---|---|---|---|---|---|
| | | | | | W2 | C2B | | RI | | | |
| | | | | | EC₅₀ | EC₅₀ | | (TM90/ | RL v9 | PbLS | HepG2DMEM |
| Cpd | R¹ | R² | R³ | R⁴ | (μM) | (μM) | cLogP | W2) | (μM) | (μM) | Gal (μM) |
| THA- | —H | —Cl | —H | —H | >10 | >10 | 3.00 | N/A | N/A | N/A | >10 |
| 2040 | | | | | | | | | | | |
| THA- | —H | —H | —OMe | —H | 0.486 | 0.374 | 2.41 | 0.769 | 1.608 | 1.506 | >10 |
| 2039 | | | | | | | | | | | |

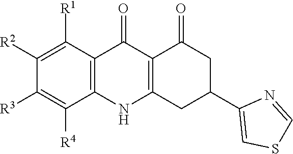

| | | | | | | TM90- | | | | | |
|---|---|---|---|---|---|---|---|---|---|---|---|
| | | | | | W2 | C2B | | RI | | | |
| | | | | | EC₅₀ | EC₅₀ | | (TM90/ | RL v9 | PbLS | HepG2DMEM |
| Cpd | R¹ | R² | R³ | R⁴ | (μM) | (μM) | cLogP | W2) | (μM) | (μM) | Gal (μM) |
| THA- | —H | —Cl | —H | —H | >0 | >10 | 3.00 | N/A | N/A | N/A | >3.33 |
| 2037 | | | | | | | | | | | |
| THA- | —H | —H | —OMe | —H | 1.14 | 0.797 | 2.41 | 0.699 | N/A | | >10 |
| 2038 | | | | | | | | | | | |

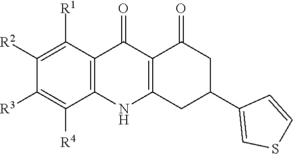

| | | | | | | TM90- | | | | | |
|---|---|---|---|---|---|---|---|---|---|---|---|
| | | | | | W2 | C2B | | RI | | | |
| | | | | | EC₅₀ | EC₅₀ | | (TM90/ | RL v9 | PbLS | HepG2DMEMGal |
| Cpd | R¹ | R² | R³ | R⁴ | (μM) | (μM) | cLogP | W2) | (μM) | (μM) | (μM) |
| THA- | —H | —Cl | —H | —H | 0.111 | >1.47 | 3.46 | N/A | N/A | N/A | >10 |
| 2107 | | | | | | | | | | | |
| THA- | —H | —H | —OMe | —H | 0.0308 | 0.0136 | 2.85 | 0.442 | 0.0455 | 0.0816 | >10 |
| 2110 | | | | | | | | | | | |

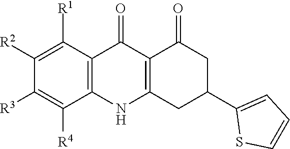

| | | | | | | TM90- | | | | | |
|---|---|---|---|---|---|---|---|---|---|---|---|
| | | | | | W2 | C2B | | RI | | | |
| | | | | | EC₅₀ | EC₅₀ | | (TM90/ | RL v9 | PbLS | HepG2DMEMGal |
| Cpd | R¹ | R² | R³ | R⁴ | (μM) | (μM) | cLogP | W2) | (μM) | (μM) | (μM) |
| THA- | —H | —Cl | —H | —H | 0.317 | 1.09 | 3.46 | 3.44 | N/A | N/A | >10 |
| 2106 | | | | | | | | | | | |
| THA- | —H | —F | —H | —H | 0.0198 | 0.0707 | 3.10 | 3.57 | 0.0375 | 0.0231 | >10 |
| 2111 | | | | | | | | | | | |
| THA- | —H | —H | —OMe | —H | 0.0384 | 0.0163 | 2.85 | 0.424 | N/A | 0.122 | >10 |
| 2109 | | | | | | | | | | | |
| THA- | —H | —Cl | —OMe | —H | 0.0192 | 0.00806 | 3.42 | 0.421 | 10.0330 | 0.00630 | >0.1 |
| 2220 | | | | | | | | | | | |

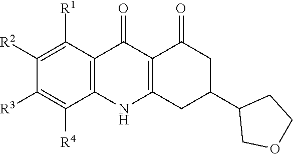

| | | | | | | TM90- | | | | | |
|---|---|---|---|---|---|---|---|---|---|---|---|
| | | | | | W2 | C2B | | RI | | | |
| | | | | | EC₅₀ | EC₅₀ | | (TM90/ | RL v9 | PbLS | HepG2DMEMGal |
| Cpd | R¹ | R² | R³ | R⁴ | (μM) | (μM) | cLogP | W2) | (μM) | (μM) | (μM) |
| THA- | —H | —Cl | —H | —H | >1.90 | >10 | 2.60 | N/A | N/A | N/A | >10 |
| 2099 | | | | | | | | | | | |

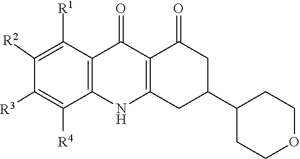

| | | | | | | TM90- | | | | | |
|---|---|---|---|---|---|---|---|---|---|---|---|
| | | | | | W2 | C2B | | RI | | | |
| | | | | | EC₅₀ | EC₅₀ | | (TM90/ | RL v9 | PbLS | HepG2DMEMGal |
| Cpd | R¹ | R² | R³ | R⁴ | (μM) | (μM) | cLogP | W2) | (μM) | (μM) | (μM) |
| THA- | —H | —H | —OMe | —H | >3.97 | 2.411 | 2.24 | N/A | N/A | N/A | >10 |
| 2098 | | | | | | | | | | | |
| THA- | —H | —Cl | —H | —H | 0.0988 | >6.93 | 2.95 | N/A | N/A | N/A | >10 |
| 2097 | | | | | | | | | | | |

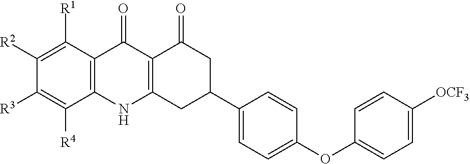

| | | | | | | TM90- | | | | | |
|---|---|---|---|---|---|---|---|---|---|---|---|
| | | | | | W2 | C2B | | RI | | | |
| | | | | | EC₅₀ | EC₅₀ | | (TM90/ | RL v9 | PbLS | HepG2DMEMGal |
| Cpd | R¹ | R² | R³ | R⁴ | (μM) | (μM) | cLogP | W2) | (μM) | (μM) | (μM) |
| THA- | —H | —Cl | —H | —H | 0.00640 | 0.0109 | 5.52 | 1.69 | 0.0783 | <0.00457 | >10 |
| 2035 | | | | | | | | | | | |
| THA- | —H | —H | —OMe | —H | 0.00743 | 0.00208 | 4.89 | 0.279 | 0.0404 | 0.0084 | 0.677 |
| 2033 | | | | | | | | | | | |
| THA- | —H | —F | —H | —H | 0.000330 | 0.00268 | 5.20 | 8.12 | N/A | N/A | >10 |
| 2103 | | | | | | | | | | | |

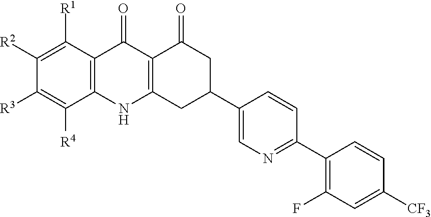

| | | | | | | TM90- | | | | | |
|---|---|---|---|---|---|---|---|---|---|---|---|
| | | | | | W2 | C2B | | RI | | | |
| | | | | | EC₅₀ | EC₅₀ | | (TM90/ | RL v9 | PbLS | HepG2DMEMGal |
| Cpd | R¹ | R² | R³ | R⁴ | (μM) | (μM) | cLogP | W2) | (μM) | (μM) | (μM) |
| THA- | —H | —Cl | —H | —H | 0.0116 | 0.0109 | 5.03 | 0.940 | >0.100 | 0.0112 | >10 |
| 2101 | | | | | | | | | | | |
| THA- | —H | —H | —OMe | —H | 0.00290 | 0.00140 | 4.50 | 0.483 | 0.0188 | 0.00805 | >10 |
| 2112 | | | | | | | | | | | |

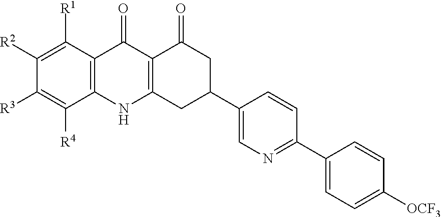

| | | | | | | TM90- | | | | | |
|---|---|---|---|---|---|---|---|---|---|---|---|
| | | | | | W2 | C2B | | RI | | | |
| | | | | | EC₅₀ | EC₅₀ | | (TM90/ | RL v9 | PbLS | HepG2DMEMGal |
| Cpd | R¹ | R² | R³ | R⁴ | (μM) | (μM) | cLogP | W2) | (μM) | (μM) | (μM) |
| THA- | —H | —H | —OMe | —H | 0.0161 | 0.00710 | 4.13 | 0.441 | 0.253 | 0.0129 | >10 |
| 2100 | | | | | | | | | | | |
| THA- | —H | —Cl | —H | —H | 0.0510 | 0.0251 | 4.70 | 0.492 | N/A | 0.00834 | >10 |
| 2108 | | | | | | | | | | | |
| THA- | —H | —Cl | —OMe | —H | 0.00586 | 0.00135 | 4.56 | 0.230 | 0.0261 | 0.000195 | >0.1 |
| 2222 | | | | | | | | | | | |

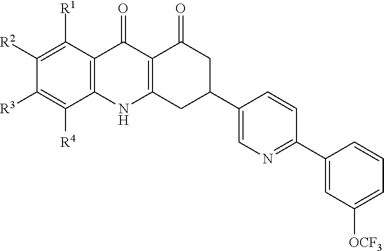

| | | | | | | TM90- | | | | | |
|---|---|---|---|---|---|---|---|---|---|---|---|
| | | | | | W2 | C2B | | RI | | | |
| | | | | | EC₅₀ | EC₅₀ | | (TM90/ | RL v9 | PbLS | HepG2DMEMGal |
| Cpd | R¹ | R² | R³ | R⁴ | (μM) | (μM) | cLogP | W2) | (μM) | (μM) | (μM) |
| THA- | —H | —H | —OMe | —H | 0.0101 | 0.00484 | 4.13 | 0.479 | N/A | 0.0124 | >10 |
| 2116 | | | | | | | | | | | |
| THA- | —H | —Cl | —H | —H | 0.0169 | 0.0248 | 4.70 | 1.47 | >0.100 | 0.00210 | >10 |
| 2115 | | | | | | | | | | | |

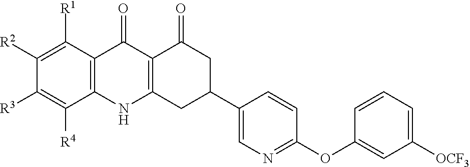

| | | | | | | TM90- | | | | | |
|---|---|---|---|---|---|---|---|---|---|---|---|
| | | | | | W2 | C2B | | RI | | | |
| | | | | | EC₅₀ | EC₅₀ | | (TM90/ | RL v9 | PbLS | HepG2DMEMGal |
| Cpd | R¹ | R² | R³ | R⁴ | (μM) | (μM) | cLogP | W2) | (μM) | (μM) | (μM) |
| THA- | —H | —Cl | —H | —H | 0.00310 | 0.00470 | 4.52 | 1.52 | >0.0330 | 0.00200 | >10 |
| 2114 | | | | | | | | | | | |
| THA- | —H | —H | —OMe | —H | 0.00350 | 0.00254 | 3.93 | 0.726 | N/A | 0.0216 | 0.839 |
| 2113 | | | | | | | | | | | |

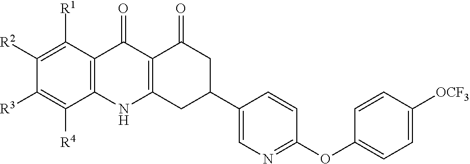

| | | | | | | TM90- | | | | | |
|---|---|---|---|---|---|---|---|---|---|---|---|
| | | | | | W2 | C2B | | RI | | | |
| | | | | | EC₅₀ | EC₅₀ | | (TM90/ | RL v9 | PbLS | HepG2DMEMGal |
| Cpd | R¹ | R² | R³ | R⁴ | (μM) | (μM) | cLogP | W2) | (μM) | (μM) | (μM) |
| THA- | —H | —Cl | —H | —H | 0.0294 | 0.0249 | 4.63 | 0.847 | | 0.00376 | >4.34 |
| 2149 | | | | | | | | | | | |
| THA- | —H | —H | —OMe | —H | 0.0130 | 0.00661 | 4.03 | 0.508 | | 0.0209 | 1.17 |
| 2148 | | | | | | | | | | | |
| THA- | —H | —Cl | —OMe | —H | 0.00571 | 0.00105 | 4.47 | 0.184 | | 0.000471 | >0.1 |
| 2227 | | | | | | | | | | | |

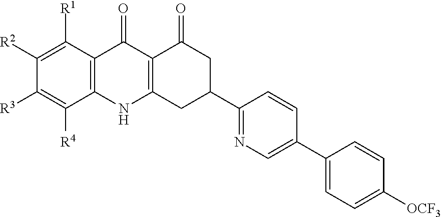

| | | | | | | TM90- | | | | | |
|---|---|---|---|---|---|---|---|---|---|---|---|
| | | | | | W2 | C2B | | RI | | | |
| | | | | | EC₅₀ | EC₅₀ | | (TM90/ | RL v9 | PbLS | HepG2DMEMGal |
| Cpd | R¹ | R² | R³ | R⁴ | (μM) | (μM) | cLogP | W2) | (μM) | (μM) | (μM) |
| THA- | —H | —H | —OMe | —H | 0.00570 | 0.00146 | 4.13 | 0.256 | 0.0396 | 0.00130 | >10.0 |
| 2150 | | | | | | | | | | | |
| THA- | —H | —Cl | —H | —H | 0.00608 | 0.00603 | 4.70 | 0.992 | N/A | 0.00107 | 5.10 |
| 2151 | | | | | | | | | | | |
| THA- | —H | —Cl | —OMe | —H | 0.00323 | 0.00102 | 4.56 | 0.316 | 0.0114 | 0.000147 | >0.1 |
| 2221 | | | | | | | | | | | |

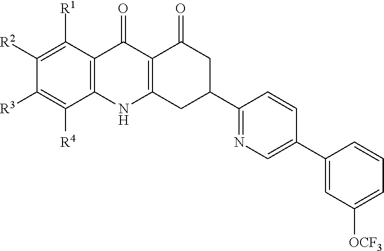

| | | | | | | TM90- | | | | | |
|---|---|---|---|---|---|---|---|---|---|---|---|
| | | | | | W2 | C2B | | RI | | | |
| | | | | | EC₅₀ | EC₅₀ | | (TM90/ | RL v9 | PbLS | HepG2DMEMGal |
| Cpd | R¹ | R² | R³ | R⁴ | (μM) | (μM) | cLogP | W2) | (μM) | (μM) | (μM) |
| THA- | —H | —H | —OMe | —H | 0.00510 | 0.00284 | 4.13 | 0.557 | N/A | 0.00100 | >2.45 |
| 2152 | | | | | | | | | | | |
| THA- | —H | —Cl | —H | —H | 0.0245 | 0.0465 | 4.70 | 1.90 | N/A | 0.00146 | >10 |
| 2153 | | | | | | | | | | | |
| THA- | —H | —Cl | —OMe | —H | 0.00219 | 0.000895 | 4.56 | 0.409 | 0.0105 | 0.000109 | >0.1 |
| 2223 | | | | | | | | | | | |

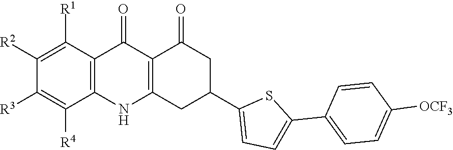

| | | | | | | TM90- | | | | | |
|---|---|---|---|---|---|---|---|---|---|---|---|
| | | | | | W2 | C2B | | RI | | | |
| | | | | | EC₅₀ | EC₅₀ | | (TM90/ | RL v9 | PbLS | HepG2DMEMGal |
| Cpd | R¹ | R² | R³ | R⁴ | (μM) | (μM) | cLogP | W2) | (μM) | (μM) | (μM) |
| THA- | —H | —H | —OMe | —H | 0.00104 | 0.00158 | 4.90 | 1.52 | N/A | N/A | 1.113 |
| 2147 | | | | | | | | | | | |
| THA- | —H | —Cl | —H | —H | 0.000984 | 0.00255 | 5.47 | 2.59 | N/A | 0.000373 | >10 |
| 2206 | | | | | | | | | | | |
| THA- | —H | —Cl | —OMe | —H | 0.00119 | 0.000190 | 5.33 | 0.160 | 0.00254 | 0.0000594 | >0.1 |
| 2229 | | | | | | | | | | | |

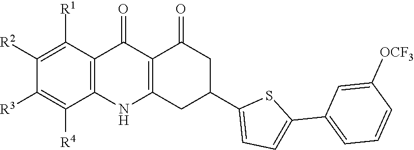

| | | | | | | TM90- | | | | | |
|---|---|---|---|---|---|---|---|---|---|---|---|
| | | | | | W2 | C2B | | RI | | | |
| | | | | | EC₅₀ | EC₅₀ | | (TM90/ | RL v9 | PbLS | HepG2DMEMGal |
| Cpd | R¹ | R² | R³ | R⁴ | (μM) | (μM) | cLogP | W2) | (μM) | (μM) | (μM) |
| THA- | —H | —H | —OMe | —H | 0.00237 | 0.00101 | 4.90 | 0.426 | N/A | N/A | 1.077 |
| 2146 | | | | | | | | | | | |
| THA- | —H | —Cl | —H | —H | 0.00241 | 0.00958 | 5.47 | 3.97 | N/A | 0.00121 | >10 |
| 2207 | | | | | | | | | | | |
| THA- | —H | —Cl | —OMe | —H | 0.00103 | 0.000232 | 5.33 | 0.225 | 0.00193 | 0.0000878 | >0.1 |
| 2228 | | | | | | | | | | | |

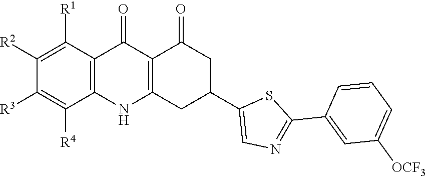

| | | | | | | TM90- | | | | | |
|---|---|---|---|---|---|---|---|---|---|---|---|
| | | | | | W2 | C2B | | RI | | | |
| | | | | | EC₅₀ | EC₅₀ | | (TM90/ | RL v9 | PbLS | HepG2DMEMGal |
| Cpd | R¹ | R² | R³ | R⁴ | (μM) | (μM) | cLogP | W2) | (μM) | (μM) | (μM) |
| THA- | —H | —H | —OMe | —H | 0.0110 | 0.00187 | 4.49 | 0.171 | N/A | 0.00487 | >2.70 |
| 2169 | | | | | | | | | | | |
| THA- | —H | —Cl | —H | —H | 0.0142 | 0.00387 | 5.04 | 0.273 | >0.100 | 0.00146 | >10 |
| 2168 | | | | | | | | | | | |

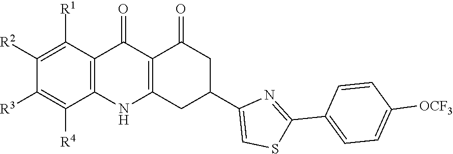

| | | | | | | TM90- | | | | | |
|---|---|---|---|---|---|---|---|---|---|---|---|
| | | | | | W2 | C2B | | RI | | | |
| | | | | | EC₅₀ | EC₅₀ | | (TM90/ | RL v9 | PbLS | HepG2DMEMGal |
| Cpd | R¹ \| | R² | R³ | R⁴ | (μM) | (μM) | cLogP | W2) | (μM) | (μM) | (μM) |
| THA- | —H | —H | —OMe | —H | 0.0278 | 0.00924 | 4.49 | 0.332 | N/A | 0.00870 | 1.52 |
| 2172 | | | | | | | | | | | |
| THA- | —H | —Cl | —H | —H | 0.0560 | 0.412 | 5.04 | 7.36 | N/A | 0.000718 | >10 |
| 2170 | | | | | | | | | | | |
| THA- | —H | —Cl | —OMe | —H | 0.0122 | 0.00277 | 4.92 | 0.227 | 0.0572 | 0.000253 | >0.1 |
| 2226 | | | | | | | | | | | |

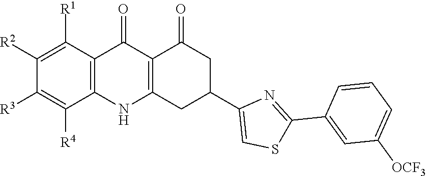

| | | | | | | TM90- | | | | | |
|---|---|---|---|---|---|---|---|---|---|---|---|
| | | | | | W2 | C2B | | RI | | | |
| | | | | | EC₅₀ | EC₅₀ | | (TM90/ | RL v9 | PbLS | HepG2DMEMGal |
| Cpd | R¹ | R² | R³ | R⁴ | (μM) | (μM) | cLogP | W2) | (μM) | (μM) | (μM) |
| THA- | —H | —H | —OMe | —H | 0.0229 | 0.0103 | 4.49 | 0.450 | N/A | 0.00839 | 1.32 |
| 2173 | | | | | | | | | | | |
| THA- | —H | —Cl | —H | —H | 0.0135 | 0.0556 | 5.04 | 4.12 | N/A | 0.000115 | >10 |
| 2171 | | | | | | | | | | | |
| THA- | —H | —Cl | —OMe | —H | 0.0161 | 0.00591 | 4.92 | 0.367 | 0.0564 | 0.000982 | >0.1 |
| 2231 | | | | | | | | | | | |

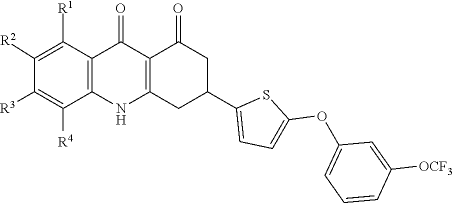

| | | | | | | TM90- | | | | | |
|---|---|---|---|---|---|---|---|---|---|---|---|
| | | | | | W2 | C2B | | RI | | | |
| | | | | | EC₅₀ | EC₅₀ | | (TM90/ | RL v9 | PbLS | HepG2DMEMGal |
| Cpd | R¹ | R² | R³ | R⁴ | (μM) | (μM) | cLogP | W2) | (μM) | (μM) | (μM) |
| THA- | —H | —H | —OMe | —H | 0.00140 | 0.000661 | 4.69 | 0.472 | N/A | N/A | 0.391 |
| 2174 | | | | | | | | | | | |
| THA- | —H | —Cl | —H | —H | 0.00339 | 0.00992 | 5.28 | 2.93 | N/A | N/A | >10 |
| 2208 | | | | | | | | | | | |

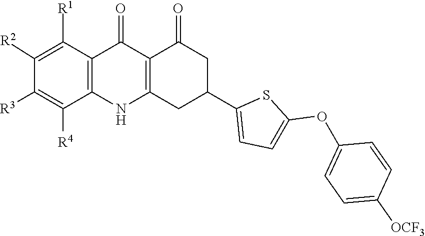

| | | | | | | TM90- | | | | | |
|---|---|---|---|---|---|---|---|---|---|---|---|
| | | | | | W2 | C2B | | RI | | | |
| | | | | | EC₅₀ | EC₅₀ | | (TM90/ | RL v9 | PbLS | HepG2DMEMGal |
| Cpd | R¹ | R² | R³ | R⁴ | (μM) | (μM) | cLogP | W2) | (μM) | (μM) | (μM) |
| THA- | —H | —H | —OMe | —H | 0.00291 | 0.00237 | 4.79 | 0.814 | N/A | N/A | 0.473 |
| 2175 | | | | | | | | | | | |
| THA- | —H | —Cl | —H | —H | 0.00353 | 0.00939 | 5.39 | 2.66 | N/A | N/A | >2.98 |
| 2176 | | | | | | | | | | | |

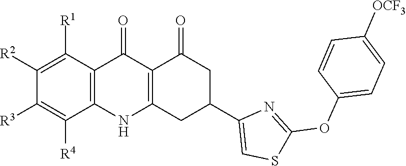

| | | | | | | TM90- | | | | | |
|---|---|---|---|---|---|---|---|---|---|---|---|
| | | | | | W2 | C2B | | RI | | | |
| | | | | | EC₅₀ | EC₅₀ | | (TM90/ | RL v9 | PbLS | HepG2DMEMGal |
| Cpd | R¹ | R² | R³ | R⁴ | (μM) | (μM) | cLogP | W2) | (μM) | (μM) | (μM) |
| THA- | —H | —Cl | —H | —H | 0.00229 | 0.00561 | 4.97 | 2.45 | N/A | 0.00128 | >10 |
| 2201 | | | | | | | | | | | |
| THA- | —H | —H | —OMe | —H | 0.0242 | 0.0129 | 4.39 | 0.533 | N/A | 0.0111 | 3.12 |
| 2202 | | | | | | | | | | | |
| THA- | —H | —Cl | —OMe | —H | 0.0113 | 0.00317 | 4.81 | 0.281 | >0.0330 | 0.00107 | >0.1 |
| 2225 | | | | | | | | | | | |

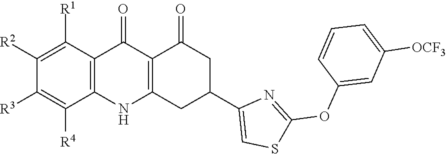

| | | | | | | TM90- | | | | | |
|---|---|---|---|---|---|---|---|---|---|---|---|
| | | | | | W2 | C2B | | RI | | | |
| | | | | | EC₅₀ | EC₅₀ | | (TM90/ | RL v9 | PbLS | HepG2DMEMGal |
| Cpd | R¹ | R² | R³ | R⁴ | (μM) | (μM) | cLogP | W2) | (μM) | (μM) | (μM) |
| THA- | —H | —Cl | —H | —H | 0.0399 | 0.524 | 4.86 | 13.1 | N/A | 0.00457 | >10 |
| 2203 | | | | | | | | | | | |
| THA- | —H | —Cl | —OMe | —H | 0.0208 | 0.00513 | 4.71 | 0.247 | >0.100 | 0.00111 | >0.1 |
| 2224 | | | | | | | | | | | |

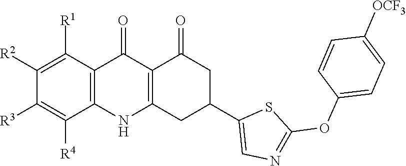

| | | | | | | TM90- | | | | | |
|---|---|---|---|---|---|---|---|---|---|---|---|
| | | | | | W2 | C2B | | RI | | | |
| | | | | | EC₅₀ | EC₅₀ | | (TM90/ | RL v9 | PbLS | HepG2DMEMGal |
| Cpd | R¹ | R² | R³ | R⁴ | (μM) | (μM) | cLogP | W2) | (μM) | (μM) | (μM) |
| THA- | | | —OMe | | 0.0179 | 0.00350 | 4.39 | 0.196 | 0.0658 | 0.00374 | >5.45 |
| 2204 | | | | | | | | | | | |

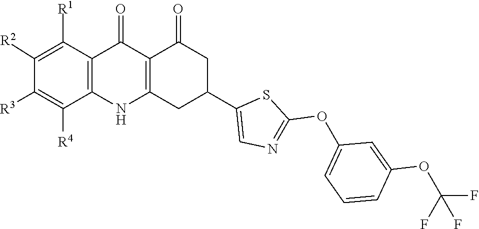

| | | | | | | TM90- | | | | | |
|---|---|---|---|---|---|---|---|---|---|---|---|
| | | | | | W2 | C2B | | RI | | | |
| | | | | | EC₅₀ | EC₅₀ | | (TM90/ | RL v9 | PbLS | HepG2DMEMGal |
| Cpd | R¹ | R² | R³ | R⁴ | (μM) | (μM) | cLogP | W2) | (μM) | (μM) | (μM) |
| THA- | | | —OMe | | 0.00739 | 0.00195 | 4.38 | 0.264 | N/A | 0.00559 | >4.97 |
| 2205 | | | | | | | | | | | |

| | | | | TM90- | |
|---|---|---|---|---|---|
| | | | W2 | C2B | HepG2 |
| | | Peaks | (EC₅₀ | (EC₅₀ | (EC₅₀ |
| Cpd | Structure | amount | nM) | nM) | nM) |
| THA-1829 | 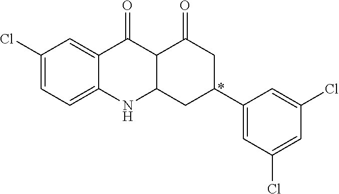 [SMILES: O=C1CC(c2cc(Cl)cc(Cl)c2)CC2Nc3ccc(Cl)cc3C(=O)C12] | R enantiomer S enantiomer Racemic | 77.3 12.5 8.95 | 49.5 64.2 41.4 | >10 >10 >10 |
| THA-1839 | 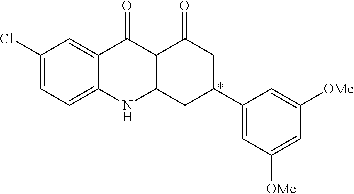 [SMILES: COc1cc(OC)cc(C2CC(=O)C3C(=O)c4cc(Cl)ccc4NC3C2)c1] | R enantiomer S enantiomer Racemic | 66.4 6.29 5.42 | 59.6 111 77.4 | >10 >10 >10 |
| THA-1994 | 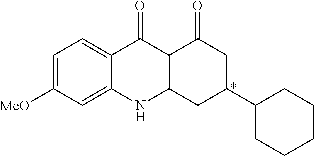 [SMILES: COc1ccc2c(c1)NC1CC(C3CCCCC3)CC(=O)C1C2=O] | R enantiomer S enantiomer Racemic | 60.1 59.6 52.9 | 40.7 112.6 59.9 | >10 >3.33 >10 |
| THA-2220 | 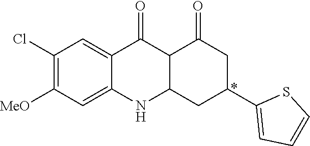 [SMILES: COc1cc2c(cc1Cl)C(=O)C1C(=O)CC(c3cccs3)CC1N2] | R enantiomer S enantiomer Racemic | 67.9 168 59.9 | 14.6 114.4 27.4 | >10 >3.33 >10 |
| THA-2222 | 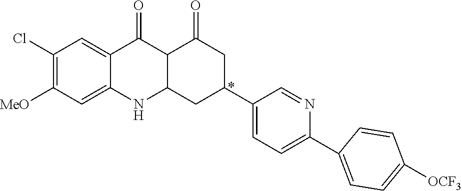 [SMILES: COc1cc2c(cc1Cl)C(=O)C1C(=O)CC(c3ccc(-c4ccc(OC(F)(F)F)cc4)nc3)CC1N2] | R enantiomer S enantiomer Racemic | 43.6 19.6 11.9 | 7.6 <60E-06 <60E-06 | >10 >10 >10 |
| THA-2225 | 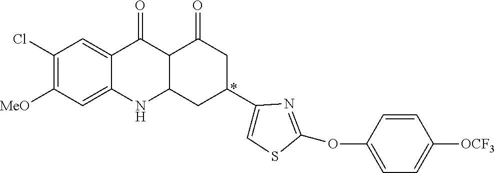 [SMILES: COc1cc2c(cc1Cl)C(=O)C1C(=O)CC(c3csc(Oc4ccc(OC(F)(F)F)cc4)n3)CC1N2] | R enantiomer S enantiomer Racemic | 47.1 1.93 1.57 | 19.3 3.91 2.05 | >10 >10 >10 |
| THA-2224 | 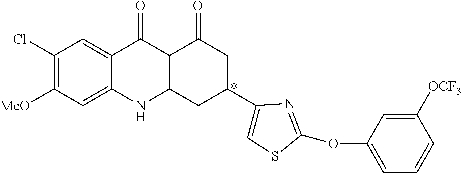 [SMILES: COc1cc2c(cc1Cl)C(=O)C1C(=O)CC(c3csc(Oc4cccc(OC(F)(F)F)c4)n3)CC1N2] | R enantiomer S enantiomer Racemic | 1.59 54.3 11.1 | 34.25 37.6 30.5 | >10 >10 >10 |
| THA-2209 | 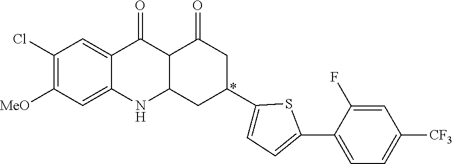 [SMILES: COc1cc2c(cc1Cl)C(=O)C1C(=O)CC(c3ccc(-c4ccc(C(F)(F)F)cc4F)s3)CC1N2] | R enantiomer S enantiomer Racemic | <60E-06 4.19 4.6 | <60E-06 17.9 <60E-06 | >10 >10 >10 |

| | | | Rat | | |
|---|---|---|---|---|---|
| | Human liver | | hepatocytes | | Solubility |
| | microsomes | | | CLint, app | in PBS |
| | T-half | CLint, app | T-half | (μL/min/10⁶ | pH 7.4 |
| Compound | (min) | (μL/min/mg) | (min) | cells) | μM |
| THA-1839 | 46.8 | 29.6 | 60.5 | 11.5 | 47.7 |
| THA-1840 | 57.6 | 24.1 | 48.5 | 14.3 | 13.9 |
| THA-1994 | 15.1 | 91.5 | 12.8 | 54.0 | 7.77 |
| THA-2111 | 6.6 | 212 | 24.8 | 27.9 | 5.05 |
| THA-2220 | 5.8 | 240 | 17.0 | 40.8 | 3.79 |
| THA-2222 | 626 | 2.2 | 936 | 0.70 | 13.4 |
| THA-2225 | 234 | 5.9 | 129 | 5.40 | 3.59 |
| THA-2224 | 146 | 9.5 | 69.6 | 10.0 | 18.0 |
| THA-2209 | 734 | 1.9 | 1250 | 0.60 | 6.11 |

It should be emphasized that the above-described embodiments of the present disclosure are merely possible examples of implementations set forth for a clear understanding of the principles of the disclosure. Many variations and modifications may be made to the above-described embodiment(s) without departing substantially from the spirit and principles of the disclosure. All such modifications and variations are intended to be included herein within the scope of this disclosure and protected by the following claims.

1. A compound having a formula represented by a structure I or the pharmaceutically acceptable salt thereof: 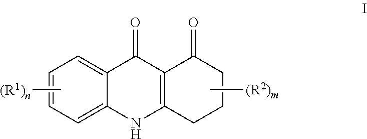
wherein
n is an integer from 1 to 4, where each R¹ is independently hydrogen, a halide, a substituted or unsubstituted linear or branched alkyl group, or an alkoxy group, and
m is an integer from 1 to 3, where each R² is hydrogen, a substituted or unsubstituted cycloalkyl group, a substituted or unsubstituted heterocycloalkyl group, a substituted or unsubstituted aryl group, or a substituted or unsubstituted heteroaryl group.

2. The compound of claim 1, wherein n is 1 or 2 and m is 1.

3. The compound of claim 1, wherein R¹ is a C1-C6 alkyl group, a halide, or a C1-C6 alkoxy group.

4. The compound of claim 1, wherein the compound has the structure II 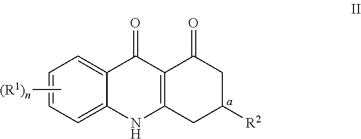
wherein the stereochemistry at carbon a is racemic or has an enantiomeric excess of at least 95% of the R or S enantiomer.

5. The compound of claim 4, wherein R² is an unsubstituted phenyl group or a phenyl group substituted with a halide, a substituted or unsubstituted alkyl group, an alkoxy group, or an aryloxy group.

6. The compound of claim 4, wherein R² is a 5- or 6-membered substituted or unsubstituted heterocycloalkyl group.

7. The compound of claim 4, wherein the unsubstituted heterocycloalkyl group is a tetrahydrofuran group or a tetrahydropyran group.

8. The compound of claim 4, wherein R² is a substituted or unsubstituted heteroaryl group, wherein the heteroaryl group is a pyridine group, a thiophene group, or a thiazole group.

9. The compound of claim 4, wherein R² is a heteroaryl group that is substituted with a substituted or unsubstituted phenyl group.

10. The compound of claim 9, where the phenyl group is substituted with a halide, a substituted or unsubstituted alkyl group, an alkoxy group, a fluoroalkoxy group, or a fluoroalkyl group.

11. The compound of claim 4, wherein R² is a C3-C6 cycloalkyl group.

12. The compound of claim 4, wherein R² is 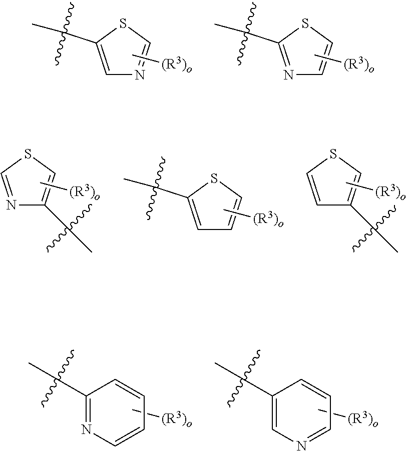
wherein o is 1 or 2 and R³ is hydrogen, a substituted or unsubstituted cycloalkyl group, a substituted or unsubstituted heterocycloalkyl group, a substituted or unsubstituted aryl group, a substituted or unsubstituted heteroaryl group, or a substituted or unsubstituted aryloxy group.

13. The compound of claim 12, wherein R³ is a phenyl group substituted with a fluoroalkyl group or fluoroalkoxy group.

14. The compound of claim 12, wherein R³ is a phenyl group substituted with —F, —CF₃, —OCF₃, or any combination thereof.

15. The compound of claim 1, wherein the compound has one of the following structures 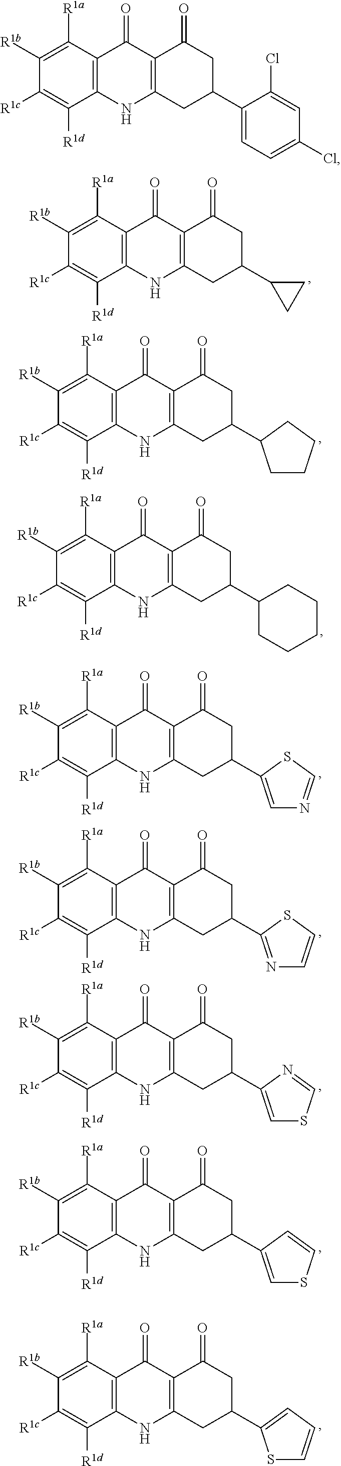 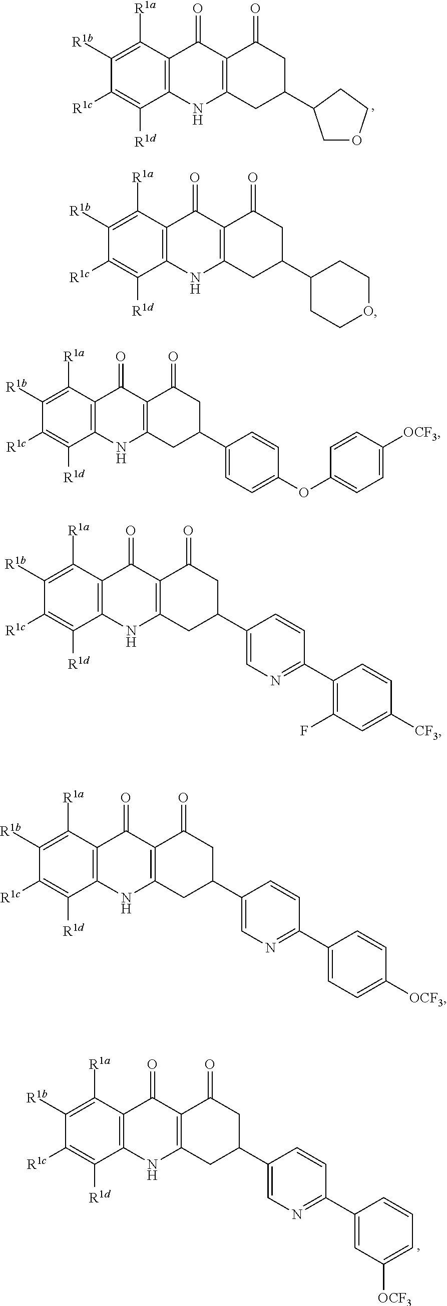 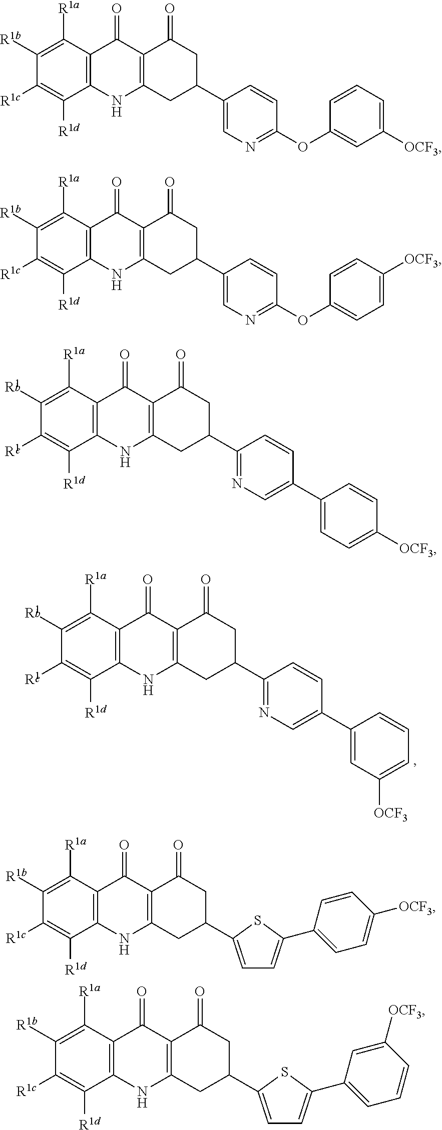 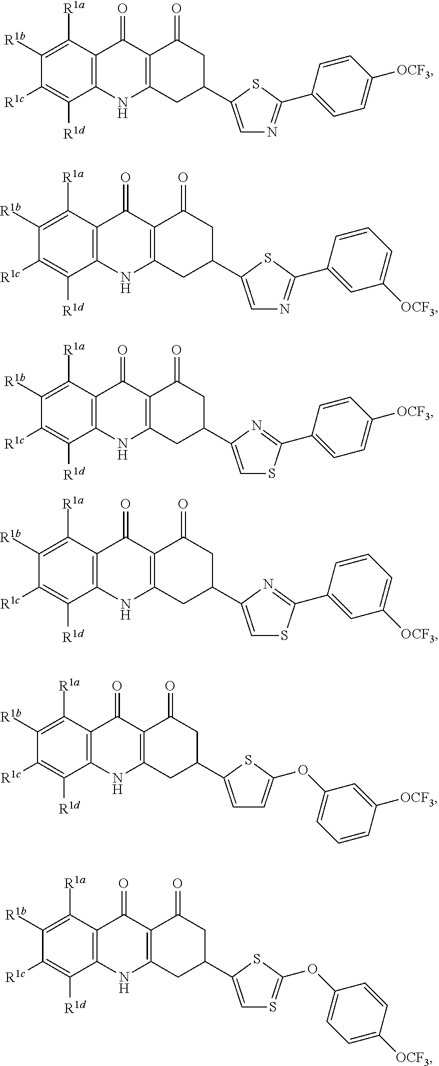 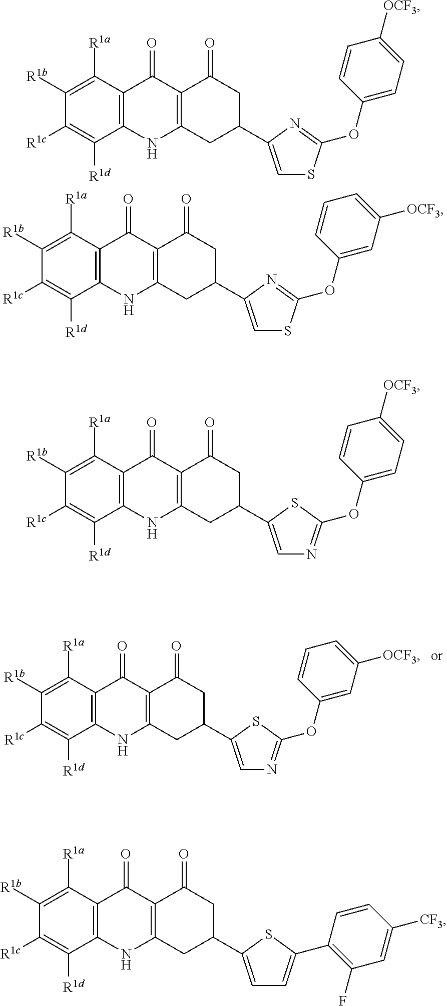
wherein R^{1a}, R^{1b}, R^{1c}, and R^{1d}, are each independently hydrogen, a halide, a substituted or unsubstituted linear or branched alkyl group, or an alkoxy group.

16. The compound of claim 1, wherein the compound has the structure 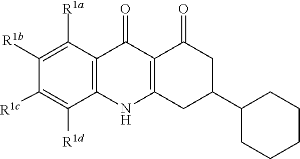
wherein R^{1c} is a methoxy group, or 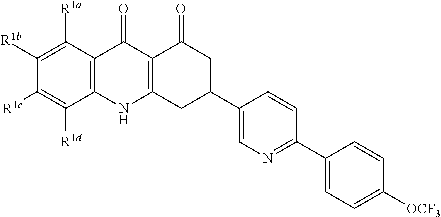
wherein R^{1b} is chloride and R^{1c} is a methoxy group, or 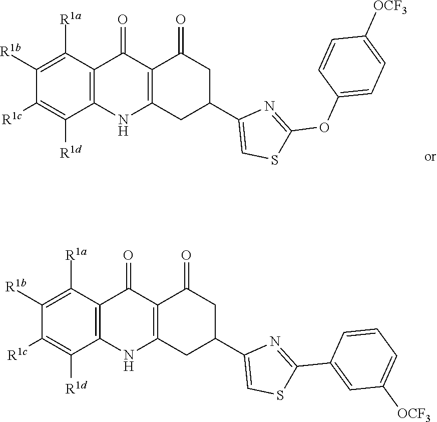
wherein R^{1b} is chloride and R^{1c} is a methoxy group, or 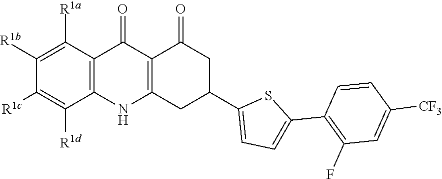
wherein R^{1c} is a methoxy group, or 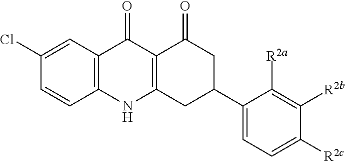
wherein R^{2a}, R^{2b}, and R^{2c}, are each independently hydrogen, a halide, a substituted or unsubstituted linear or branched alkyl group, or an alkoxy group.

17. The compound of claim 16, wherein the alkyl group is a methyl group, the alkoxy group is a methoxy group and the halide is chloride.

18. The compound of claim 1, wherein the compound is THA-1839, THA-1840, THA-1994, THA-2111, THA-2209, THA-2220, THA-2224, THA-2224, or THA-2225.

19. A pharmaceutical composition comprising the compound of claim 1 and a pharmaceutically-acceptable carrier.

20. A method for treating or preventing malaria in a subject, the method comprising administering to the subject an effective amount of the compound of claim 1.
